(19) United States
(12) Patent Application Publication
(10) Pub. No.: US 2025/0171802 A1
(43) Pub. Date: May 29, 2025

(54) NUCLEIC ACID COMPOSITIONS FOR DELIVERING EXOGENOUS POLYNUCLEOTIDES AND METHODS OF USE

(71) Applicant: Vyro Bio Inc., Newark, DE (US)

(72) Inventors: Carolini Kaid DAVILA, Osasco, São Paulo (BR); Mauro Ferreira DE AZEVEDO, São Paulo, São Paulo (BR)

(21) Appl. No.: 18/840,022

(22) PCT Filed: Feb. 21, 2023
(86) PCT No.: PCT/US2023/062966

§ 371 Date: Aug. 20, 2024

Related U.S. Application Data
(60) Provisional application No. 63/312,785, filed on Feb. 22, 2022.

(51) Int. Cl. C12N 15/86

(52) U.S. Cl. CPC C12N 15/86; C12N 2770/24071; C12N 2770/24123; C12N 2770/24143

(57) ABSTRACT

Disclosed herein are nucleic acid compositions for delivering exogenous polynucleotides, and methods of use.

### CROSS-REFERENCE TO RELATED APPLICATION(S)

This application claims the benefit of U.S. Application No. 63/312,785, filed Feb. 22, 2022, the entirety of which is incorporated by reference herein.

### SEQUENCE LISTING

This application contains a Sequence Listing which has been submitted in XML format and is hereby incorporated by reference in its entirety. The XML copy, created on Feb. 21, 2023, is named 61771-703_601_SL.xml, and is 2,964,424 bytes in size.

### BACKGROUND OF THE INVENTION

The cellular delivery of various biologically active compounds, such as an antigen and a protein of interest, is of high value both for basic research and industrial applications. However, it is usually compromised by two limitations, one being high toxicity to normal tissues, the other being low delivery efficiency, given that living cells are highly restricted by the complex membrane systems of the cell. Special difficulties have been seen in delivery efforts with the central nervous system (CNS) as a target.

### SUMMARY OF THE INVENTION

In one aspect, to address the need for a new delivery approach to a target cell, provided herein are recombinant zika viruses and zika-virus-like particles carrying polynucleotides of interest. In some embodiments, the viruses and virus-like particles comprise zika structural proteins. Methods of use involve applying such recombinant zika viruses or such zika-virus-like particles to the target cell. In some cases, such methods of use treat disorders of the central nervous system considering its favorable CNS tropism. In some cases, the viruses and virus-like particles do not comprise replicating zika virus.

In another aspect, to address the need for a new delivery approach to a target cell, provided herein are transcripts of nucleic acid compositions carrying polynucleotides of interest (e.g., an exogenous polynucleotide) and regulatory regions from a zika genome. Example methods of use involve encapsulating such transcripts with lipid-based reagents and applying such compound to the target cell. In some embodiments, the nucleic acid compositions comprise a zika 5′ UTR, polynucleotide of interest, and a zika 3′ UTR. In some embodiments, the nucleic acid compositions do not encode for a zika structural protein. In some embodiments, the nucleic acid compositions do not encode for a zika non-structural protein. In some embodiments, the polynucleotide of interest has a size of up to about 5700, 5600, 5500, 5400, 5300, 5200, 5100, 5000, 4900, 4800, 4700, 4600, or 4500 nucleotides. In some embodiments, the polynucleotide of interest has a size of about 10, 20, 30, 40, 50, 100, 150, 200, 250, 300, 350, 400, 450, or 500 nucleotides up to about 5700, 5600, 5500, 5400, 5300, 5200, 5100, 5000, 4900, 4800, 4700, 4600, or 4500 nucleotides.

In some embodiments, a nucleic acid composition comprises (i) a polynucleotide encoding a zika virus capsid protein (C) or the derivative of the zika virus C, a polynucleotide encoding a zika virus viral membrane protein (prM/M) or the derivative of the zika virus prM/M, a polynucleotide encoding a zika virus viral envelope protein (E) or the derivative of the zika virus E, or any combination thereof; and (ii) a polynucleotide exogenous to the zika virus. In some embodiments, the nucleic acid composition comprises the derivative of the zika virus C, where the derivative encodes at least or about 5, 10, 15, 20, 25, or 30 amino acids of C. In some embodiments, the nucleic acid composition comprises the derivative of the zika virus C, where the derivative encodes about 5, 10, 15, 20, or 25 to about 30 amino acids of C. In some embodiments, the nucleic acid composition comprises a polynucleotide encoding C. In some embodiments, the nucleic acid composition does not encode a polynucleotide encoding a zika virus viral membrane protein (prM/M) or the derivative of the zika virus prM/M, and a polynucleotide encoding a zika virus viral envelope protein (E) or the derivative of the zika virus E. In some embodiments, the exogenous polynucleotide has a size of up to about 5700, 5600, 5500, 5400, 5300, 5200, 5100, 5000, 4900, 4800, 4700, 4600, or 4500 nucleotides. In some embodiments, the exogenous polynucleotide has a size of about 10, 20, 30, 40, 50, 100, 150, 200, 250, 300, 350, 400, 450, or 500 nucleotides up to about 5700, 5600, 5500, 5400, 5300, 5200, 5100, 5000, 4900, 4800, 4700, 4600, or 4500 nucleotides. In some embodiments, the nucleic acid composition is a delivery vector for delivering the exogenous polynucleotide to a target cell in a subject. The target cell may be a cell in the central nervous system.

In some embodiments, the nucleic acid composition comprises the polynucleotide encoding the zika virus C or the derivative of the zika virus C, the polynucleotide encoding the zika virus prM/M or the derivative of the zika virus prM/M, and the polynucleotide encoding the zika virus E or the derivative of the zika virus E. In some embodiments, the polynucleotide encoding the zika virus C or the derivative of the zika virus C, the polynucleotide encoding the zika virus prM/M or the derivative of the zika virus prM/M, and the polynucleotide encoding the zika virus E or the derivative of the zika virus E are expressed on one or more separate nucleic acids.

In some embodiments, the polynucleotide encoding the derivative of the zika virus C comprises at least one substitution, at least one deletion, and/or at least one insertion as compared to a wild-type zika virus C. In some embodiments, the zika virus C or the derivative of the zika virus C comprises an amino acid sequence at least 90%, 91%, 92%, 93%, 94%, 95%, 96%, 97%, 98%, 99%, or 100% identical to a sequence of Table 1A, Table 1D, Table 1I, Table 2, or Table 3. In some embodiments, the derivative of the zika virus C comprises about 5, 10, 15, 20, or 25 to about 30 amino acids of a sequence of Table 1A, Table 1D, Table 1I, Table 2, or Table 3.

In some embodiments, the polynucleotide encoding the derivative of the zika virus prM/M comprises at least one substitution, at least one deletion, and/or at least one insertion as compared to a wild-type zika virus prM/M. In some embodiments, the zika virus prM/M or the derivative of the zika virus prM/M comprises a sequence at least 90%, 91%, 92%, 93%, 94%, 95%, 96%, 97%, 98%, 99%, or 100% identical to a sequence of Table 1A, Table 1F, or Table 1K.

In some embodiments, the polynucleotide encoding the derivative of the zika virus E comprises at least one substitution, at least one deletion, and/or at least one insertion as compared to a wild-type zika virus E. In some embodiments, the polynucleotide encoding E is translated into a wild zika virus type E. In some embodiments, the zika virus E or the derivative of the zika virus E comprises a sequence at least 90%, 91%, 92%, 93%, 94%, 95%, 96%, 97%, 98%, 99%, or 100% identical to sequence of Table 1A, Table IE, or Table 1J.

In some embodiments, the nucleic acid composition comprises a 5′ untranslated region (5′ UTR) of the zika virus. In some embodiments, the zika virus 5′UTR, or the derivative of the zika virus 5′UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to sequence of Table 1A, Table 1C, Table 1H, or Table 2.

In some embodiments, the nucleic acid composition comprises a 3′ untranslated region (3′ UTR) of the zika virus. In some embodiments, the zika virus 3′UTR, or the derivative of the zika virus 3′UTR comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to sequence of Table 1A, Table 1B, Table 1G, or Table 2.

In some embodiments, the nucleic acid composition does not comprise a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). In other embodiments, the nucleic acid composition comprises polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). In some embodiments, the NS1 is a zika virus NS1, the NS2A is a zika virus NS2A, the NS2B is a zika virus NS2B, the NS3 is a zika virus NS3, the NS4A is a zika virus NS4A, the NS4B is a zika virus NS4B, or the NS5 is a zika virus NS5, or any combination of two or more thereof.

In some embodiments, the components of the nucleic acid composition comprise African zika virus components, Asian zika virus components, or Brazilian zika virus components, or a combination thereof. In one specific embodiment, the zika virus is African MR766 strain.

In some embodiments, the exogenous polynucleotide is an antigen or an antigenic epitope thereof. In some embodiments, the antigen or an antigenic epitope thereof is disease-associated. In specific embodiments, the antigen or an antigenic epitope thereof is from a pathogen.

In some embodiments, the pathogen is a virus. In specific embodiments, the virus is a human SARS coronavirus, an influenza A virus, an influenza B virus, an influenza C virus, an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

In some embodiments, the pathogen is a bacterium. In specific embodiments, the bacterium is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

In some embodiments, the pathogen is a fungus, an amoeba, or a parasite. In specific embodiments, the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In some embodiments, the exogeneous polynucleotide encodes a gene editing tool. In specific embodiments, the gene editing tool is selected from a group consisting of meganuclease associated agents, CRISPR associated agents, TALEN associated agents, and zinc finger associated agents.

In some embodiments, the exogeneous polynucleotide is a small interfering RNA (siRNA), antisense RNA, micro RNA (miRNA), small or short hairpin RNA (shRNA), guide RNA (gRNA), clustered regularly interspaced short palindromic repeat RNA (crRNA), trans-activating clustered regularly interspaced short palindromic repeat RNA (tracrRNA), immune-stimulating oligonucleotide, antisense nucleic acid, or ribozyme. In specific embodiments, the exogeneous polynucleotide targets beta-secretase 1 (BACE1) and/or amyloid precursor protein (APP).

In some embodiments, the exogeneous polynucleotide encodes a polypeptide associated with a genetic disorder. In specific embodiments, the polypeptide is brain-derived neurotrophin factor (BDNF), nerve growth factor (NGF), Neprilysin inhibitors (NEP), endothelin converting enzyme (ECE), cathepsin B (CTSB), apolipoprotein E 2 (APOE2), SH3 and multiple ankyrin repeat domains protein (SHANK), neurturin (NRTN), glial cell-derived neurotrophic factor (GDNF), cerebral dopamine neurotrophic factor (CDNF), vascular endothelial growth factor A (VEGF-A), or aromatic L-amino acid decarboxylase (AADC).

In some embodiments, the exogeneous polynucleotide encodes a therapeutic agent or a diagnostic agent. In specific embodiments, the therapeutic agent is an antibody-based therapeutic agent, a hormone, a cytokine, an inhibitor or antagonist of an immune checkpoint regulator, an immune stimulatory molecule, or an agonist of an immune co-stimulatory molecule. In further embodiments, the inhibitor or antagonist of an immune checkpoint regulator is an anti-PD1 antibody. In another embodiments, the antibody-based therapeutic agent is an antibody, a functional fragment of an antibody, a chimeric antigen receptor (CAR), or a T cell receptor (TCR). In still other embodiments, the cytokine comprises lymphokine, monokine, polypeptide hormone, growth hormone, parathyroid hormone, thyroxine, insulin, proinsulin, relaxin, prorelaxin, glycoprotein hormone, follicle stimulating hormone (FSH), thyroid stimulating hormone (TSH), luteinizing hormone (LH), hepatic growth factor, fibroblast growth factor, prolactin, placental lactogen, tumor necrosis factor-a and -β, mullerian-inhibiting substance, mouse gonadotropin-associated peptide, inhibin, activin, vascular endothelial growth factor, integrin, thrombopoietin (TPO), nerve growth factor, NGF-β, platelet growth factor, transforming growth factor (TGF), TGF-a, TGF-β, insulin-like growth factor-I and -II, erythropoietin (EPO), osteoinductive factor, interferon, such as interferon-a, -β and -γ, colony stimulating factor (CSF), macrophage-CSF (M-CSF), granulocyte-macrophage-CSF (GM-CSF), granulocyte-CSF (GCSF), interleukin (IL), tumor necrosis factor, TNF-a, TNF-β, LIF, or kit ligand (KL), or a combination of two or more thereof.

In some embodiments, the exogeneous polynucleotide is about or up to about 10000, 9000, 8000, 7000, 6000, or 5000 nucleotides long. For example, the exogenous polynucleotide is about 50 to about 10000, 9000, 8000, 7000, 6000, or 5000 nucleotides long.

In some embodiments, components of the nucleic acid composition described herein are expressed on one nucleic acid or two or more separate nucleic acids. In some embodiments, the nucleic acid composition further comprises one or more expression control elements in operable linkage that confers expression of the nucleic acid composition in vitro or in vivo. In some embodiments, the expression control element is a promoter that drives expression of the nucleic acid complex in vitro. In specific embodiments, the promoter is T7, T3, SP6 or any phage promoter.

In some embodiments, the expression control element is a promoter that drives expression of the nucleic acid complex in a target cell. In some specific embodiments, the promoter is CMV, SV40 or any eukaryotic promoter. In some embodiments, the target cell is a neuron, or a non-neuron cell, VERO, COS, CHO, C6/36, HeLa, HEK, HepG2. In some embodiments, the target cell is an oligodendrocyte, microglia, or astrocyte.

In another aspect, provided herein is a pharmaceutical composition comprising the nucleic acid composition described herein with a pharmaceutically acceptable salt or derivative thereof.

In another aspect, provided herein is a recombinant zika virus or a zika-virus-like particle that is generated from expressing the nucleic acid composition described herein in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus.

In some embodiments, the second zika virus is a wild-type zika virus. In some embodiments, the wild-type zika virus is an African, an Asian and a Brazilian strain. In other embodiments, the second zika virus is a modified zika virus. In some specific embodiments, the modified zika virus comprises one or more microRNA-based gene-silencing machineries. In some specific embodiments, the one or more microRNA-based gene-silencing machineries control viral replication.

In another aspect, provided herein is a recombinant zika virus or a zika-virus-like particle that is generated from expressing the nucleic acid composition as described herein in a producer cell, without an infection of a second zika virus, wherein the zika virus C or the derivative of the zika virus C, the zika virus prM/M or the derivative of the zika virus prM/M, and the zika virus E or the derivative of the zika virus E are present in the recombinant zika virus or the zika-virus-like particle.

In some embodiment, the producer cell is a Vero E6, HEK, HEK 293T, HEK 293TT, FreeStyle™ 293-F Cell, HEK-293.2sus or C6/36 cell.

In some embodiments, the recombinant zika virus is replication competent. In other embodiments, the recombinant zika virus is replication incompetent without lowering the vector titer or impairing expression of the exogeneous polynucleotide. In some embodiments, the recombinant zika virus has decreased insertional mutagenesis. In other embodiments, the recombinant zika virus has decreased immune response.

In another aspect, provided herein is a pharmaceutical composition comprising the recombinant zika virus or the zika-virus-like particle described herein with a pharmaceutically acceptable salt or derivative thereof.

In another aspect, provided herein is a method of delivering an exogeneous polynucleotide to a target cell, the method comprising administering to the target cell the recombinant zika virus or the zika-virus-like particle described herein comprising the exogeneous polynucleotide.

In some embodiments, the target cell is a neuron or a non-neuron cell. In specific embodiments, the neuron is an oligodendrocyte, microglia, or astrocyte. In another specific embodiments, the non-neuron cell is a prostate epithelial cell, a urethra epithelial cell, a Sertoli cell, a Leydig cell, a spermatogonium cell or a retinal cell.

In some embodiments, the method is carried out in vitro, ex vivo, or in vivo.

In some embodiments, the target cell transiently expresses the exogeneous polynucleotide after delivery. In other embodiments, the target cell persistently expresses the exogeneous polynucleotide after delivery.

In another aspect, provided herein is a method of treating Alzheimer's disease in a subject in need thereof, and the method comprises administering to the subject an effective amount of the recombinant zika virus or the zika-virus-like particle as described herein comprising a polynucleotide encoding a brain-derived neurotrophin factor (BDNF).

In another aspect, provided herein is a method of treating autism in a subject in need thereof, and the method comprises administering to the subject an effective amount of the recombinant zika virus or the zika-virus-like particle as described herein comprising a polynucleotide encoding SH3 and multiple ankyrin repeat domains protein (SHANK).

In yet another aspect, provided herein is a method of treating Parkinson's disease in a subject in need thereof, the method comprising administering the subject an effective amount of the recombinant zika virus or the zika-virus-like particle as described herein comprising a polynucleotide encoding aromatic L-amino acid decarboxylase (AADC).

In some embodiments, the administrating is via systemic delivery.

In some embodiments, the administrating is performed intravenously and/or intratumorally.

In some embodiments, the administrating targets the cerebral spinal fluid in intracerebroventricular, cisterna magna, subpial, and/or intrathecal. In other embodiments, the administrating is not performed intraparenchymal.

In some embodiments, a nucleic acid composition comprises (i) a polynucleotide encoding a ribozyme (ii) 5′untranslated region (UTR) of a zika virus; (iii) 3′UTR of the zika virus; and (iv) a polynucleotide exogenous to the zika virus. In some embodiments, the ribozyme is a hammerhead ribozyme or a hepatitis delta virus (HDV) ribozyme. In some embodiments, the nucleic acid further comprises a polynucleotide encoding a second ribozyme. In some specific embodiments, the second ribozyme is a hammerhead ribozyme or a hepatitis delta virus (HDV) ribozyme. In some embodiments, the polynucleotide encoding the ribozyme encodes a hammerhead ribozyme and the polynucleotide encoding the second ribozyme encodes an HDV ribozyme. In some embodiments, the nucleic acid composition further comprises a C-partial. A C-partial may refer to a polynucleotide that encodes for a portion of C, e.g., about 5, 10, 15, 20, or 25 amino acids to about 30 amino acids of C. In some embodiments, the exogenous polynucleotide has a size of up to about 5700, 5600, 5500, 5400, 5300, 5200, 5100, 5000, 4900, 4800, 4700, 4600, or 4500 nucleotides. In some embodiments, the exogenous polynucleotide has a size of about 10, 20, 30, 40, 50, 100, 150, 200, 250, 300, 350, 400, 450, or 500 nucleotides up to about 5700, 5600, 5500, 5400, 5300, 5200, 5100, 5000, 4900, 4800, 4700, 4600, or 4500 nucleotides. In some embodiments, the nucleic acid composition is a delivery vector for delivering the exogenous polynucleotide to a target cell in a subject. The target cell may be a cell in the central nervous system.

In some embodiments, the zika virus is an African zika virus, an Asian zika virus, a Brazilian zika virus component, or a combination of one or two thereof. In specific embodiments, the zika virus is African MR766 strain.

In some embodiments, the exogeneous polynucleotide is an antigen or an antigenic epitope thereof. In some embodiments, the antigen or an antigenic epitope thereof is disease associated. In specific embodiments, the antigen or an antigenic epitope thereof is from a pathogen.

In some embodiments, the pathogen is a virus. In specific embodiments, the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

In some embodiments, the pathogen is a bacterium. In specific embodiments, the bacterium is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

In some embodiments, the pathogen is a fungus, an amoeba, or a parasite. In specific embodiments, the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In some embodiments, the exogeneous polynucleotide encodes a gene editing tool. In specific embodiments, the gene editing tool is selected from a group consisting of meganuclease associated agents, CRISPR associated agents, TALEN associated agents, and zinc finger associated agents.

In some embodiments, the exogeneous polynucleotide is a small interfering RNA (siRNA), antisense RNA, micro RNA (miRNA), small or short hairpin RNA (shRNA), guide RNA (gRNA), clustered regularly interspaced short palindromic repeat RNA (crRNA), trans-activating clustered regularly interspaced short palindromic repeat RNA (tracrRNA), immune-stimulating oligonucleotide, antisense nucleic acid, or ribozyme. In specific embodiments, the exogeneous polynucleotide targets beta-secretase 1 (BACE1) and/or amyloid precursor protein (APP).

In some embodiments, the exogeneous polynucleotide encodes a polypeptide associated with a genetic disorder. In specific embodiments, the polypeptide is brain-derived neurotrophin factor (BDNF), nerve growth factor (NGF), Neprilysin inhibitors (NEP), endothelin converting enzyme (ECE), cathepsin B (CTSB), apolipoprotein E 2 (APOE2), SH3 and multiple ankyrin repeat domains protein (SHANK), neurturin (NRTN), glial cell-derived neurotrophic factor (GDNF), cerebral dopamine neurotrophic factor (CDNF), vascular endothelial growth factor A (VEGF-A), or aromatic L-amino acid decarboxylase (AADC).

In some embodiments, the exogeneous polynucleotide encodes a therapeutic agent or a diagnostic agent. In specific embodiments, the therapeutic agent is an antibody-based therapeutic agent, a hormone, a cytokine, an inhibitor or antagonist of an immune checkpoint regulator, an immune stimulatory molecule, or an agonist of an immune co-stimulatory molecule. In further embodiments, the inhibitor or antagonist of an immune checkpoint regulator is an anti-PD1 antibody. In other embodiments, the antibody-based therapeutic agent is an antibody, a functional fragment of an antibody, a chimeric antigen receptor (CAR), or a T cell receptor (TCR). In still other embodiments, the cytokine comprises lymphokine, monokine, polypeptide hormone, growth hormone, parathyroid hormone, thyroxine, insulin, proinsulin, relaxin, prorelaxin, glycoprotein hormone, follicle stimulating hormone (FSH), thyroid stimulating hormone (TSH), luteinizing hormone (LH), hepatic growth factor, fibroblast growth factor, prolactin, placental lactogen, tumor necrosis factor-a and -β, mullerian-inhibiting substance, mouse gonadotropin-associated peptide, inhibin, activin, vascular endothelial growth factor, integrin, thrombopoietin (TPO), nerve growth factor, NGF-β, platelet growth factor, transforming growth factor (TGF), TGF-a, TGF-β, insulin-like growth factor-I and -II, erythropoietin (EPO), osteoinductive factor, interferon, such as interferon-a, -β and -γ, colony stimulating factor (CSF), macrophage-CSF (M-CSF), granulocyte-macrophage-CSF (GM-CSF), granulocyte-CSF (GCSF), interleukin (IL), tumor necrosis factor, TNF-a, TNF-β, LIF, or kit ligand (KL), or a combination of two or more thereof.

In some embodiments, the exogeneous polynucleotide is about or up to about 10000, 9000, 8000, 7000, 6000, or 5000 nucleotides long. For example, the exogenous polynucleotide is about 50 to about 10000, 9000, 8000, 7000, 6000, or 5000 nucleotides long.

In some embodiments, the nucleic acid composition further comprises one or more expression control elements in operable linkage that confers expression of the nucleic acid composition in vitro. In some embodiments, the expression control element is a promoter that drives expression of the nucleic acid complex in vitro. In specific embodiments, the promoter is T7, T3, SP6 or any phage promoter.

In another aspect, provided herein is a pharmaceutical composition comprising the nucleic acid composition described herein with a pharmaceutically acceptable salt or derivative thereof.

In another aspect, provided herein is method of delivering an exogeneous polynucleotide to a target cell, the method comprising applying to the target cell a compound produced from the nucleic acid composition as described herein comprising the exogeneous polynucleotide.

In some embodiments, the compound is produced by encapsulating a transcript produced from the nucleic acid composition described herein with a lipid-based agent. In specific embodiments, the transcript is capless. In another specific embodiments, the transcript is produced by in vitro transcribing the nucleic acid composition.

In some embodiments, the lipid-based agent is a lipofectamine-related reagent, a liposome, or a lipid nanoparticle.

In another aspect, provided herein is a method of triggering or boosting an immune response in a subject, the method comprising administering to the subject an effective amount of a compound produced from the nucleic acid composition as described herein comprising an antigen or an antigenic epitope thereof.

In some embodiments, the compound is produced by encapsulating a transcript produced from the nucleic acid composition described herein with a lipid-based agent. In specific embodiments, the transcript is capless. In another specific embodiments, the transcript is produced by in vitro transcribing the nucleic acid composition.

In some embodiments, the lipid-based agent is a lipofectamine-related reagent, a liposome, or a lipid nanoparticle.

In some embodiments, the administrating is performed intramuscularly.

In some embodiments, the antigen or an antigenic epitope thereof is from a pathogen.

In some embodiments, the pathogen is a virus. In specific embodiments, the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

In some embodiments, the pathogen is a bacterium. In specific embodiments, the bacterium is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

In some embodiments, the pathogen is a fungus, an amoeba, or a parasite. In specific embodiments, the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

Certain embodiments include a method of delivering a polynucleotide to a target cell in a subject, the method comprising administering to the subject a zika virus or a zika-virus-like particle comprising the polynucleotide, wherein the polynucleotide is not a zika virus polynucleotide, and the target cell is a cell of the central nervous system. Certain embodiments include a method of delivering a polynucleotide to a subject, the method comprising administering to the subject a zika virus or a zika-virus-like particle comprising the polynucleotide, wherein the polynucleotide is not a zika virus polynucleotide, and the zika virus or zika-virus-like particle does not comprise a replicating zika virus. Certain embodiments include a method of delivering a polynucleotide to a subject, the method comprising administering to the subject a zika virus or a zika-virus-like particle comprising the polynucleotide, wherein the polynucleotide is not a zika virus polynucleotide, and the polynucleotide has a length up to about 10000 nucleotides. In some embodiments, the subject has a disease or disorder of the central nervous system. In some embodiments, the polynucleotide has a length greater than about 6000 nucleotides. In some embodiments, the subject is a human or non-human animal.

Certain embodiments include a zika virus or zika-virus-like particle comprising a polynucleotide exogenous to zika virus having a length of about 6,000 nucleotides to about 10,000 nucleotides, optionally wherein the polynucleotide does not encode a full-length viral structural protein or a full-length viral nonstructural protein. In some embodiments, the zika virus or zika-virus-like particle of claim 7, wherein the zika virus or zika-virus-like particle is produced from a nucleic acid comprising the polynucleotide. In some embodiments, the nucleic acid further comprises a zika virus 5′ UTR and a zika virus 3′ UTR. In some embodiments, the zika virus 5′ UTR is at least 90% homologous or identical to a zika virus 5′ UTR of Table 2, Table 1A, Table 1C, or Table 1H. In some embodiments, the zika virus 3′ UTR is at least 90% homologous or identical to a zika virus 3′ UTR of Table 2, Table 1A, Table 1B, or Table 1G. In some embodiments, the nucleic acid further comprises a polynucleotide encoding a first ribozyme. In some embodiments, the nucleic acid further comprises a polynucleotide encoding a second ribozyme. In some embodiments, the nucleic acid comprises a polynucleotide encoding a derivative of the zika virus C, wherein the derivative of the zika virus C is a truncation of about or less than about 5, 10, 15, 20, 25, or 30 amino acids of zika virus C. In some embodiments, the nucleic acid does not comprise a polynucleotide encoding a zika virus capsid protein (C) or a derivative of the zika virus C. In some embodiments, the nucleic acid does not comprise a polynucleotide encoding a zika virus viral membrane protein (prM/M) or a derivative of the zika virus prM/M. In some embodiments, the nucleic acid does not comprise a polynucleotide encoding a zika virus viral envelope protein (E) or a derivative of the zika virus E. In some embodiments, the nucleic acid does not comprise a polynucleotide encoding one or more non-structural (NS) proteins: (i) a NS1, (ii) a NS2A, (iii) a NS2B, (iv) a NS3, (v) a NS4A, (vi) a NS4B, (vii) a NS5, or (viii) two or more of (i)-(vii). In some embodiments, the nucleic acid further comprises a promoter. In some embodiments, the nucleic acid comprises a sequence of Tables 1A-1K, Table 2, or Table 3.

Certain embodiments include a nucleic acid composition comprising (i) a 5′ untranslated region (5′ UTR) of a first zika virus, (ii) a 3′ UTR of a second zika virus, wherein the first zika virus is optionally the same as the second zika virus, and (iii) a polynucleotide exogenous to the first zika virus and the second zika virus. In some embodiments, the polynucleotide is about 5000 bases to about 10000 bases in length. In some embodiments, the nucleic acid does not comprise a polynucleotide encoding a zika virus viral envelope protein (E) or a derivative of the zika virus E; and the nucleic acid does not comprise a polynucleotide encoding one or more non-structural (NS) proteins: (i) a NS1, (ii) a NS2A, (iii) a NS2B, (iv) a NS3, (v) a NS4A, (vi) a NS4B, (vii) a NS5, or (viii) two or more of (i)-(vii). In some embodiments, the nucleic acid composition comprises a sequence of Tables 1A-1K, Table 2 or Table 3. In some embodiments, the polynucleotide exogenous to the first zika virus and the second zika virus is an exogenous polynucleotide described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-E illustrates a diagram of an exemplary replicon described in the present disclosure, with different example applications. FIG. 1A shows the replicon structure, which comprises a vector, or a construct, or an mRNA, as a gene expressing without caping. It can include a C-partial protein. The replicon contains 2 report genes (GFP and Nluc) for proof-of-concept assay. FIG. 1B shows four possibilities of encapsulating the replicon in the delivery. Options 1 is the encapsulation by liposome or nanoparticle for use as a vaccine platform. To keep the CNS natural tropism, the replicon is derived from a wildtype (option 2), or a modified zika virus (option 3), or a eukaryotic expression vector transfection (option 4) comprising a second construct that contains the other structural ZIKV proteins. FIG. 1C shows encapsulating options 2 and 3 that contain both replicon and the zika virus genome, comprising a recombinant zika virus that can be used as anti-cancer therapies. FIG. 1D shows encapsulating options 2 and 3 that contain only the replicon (without zika virus genome), comprising the virus-like particle that can be used as a viral vector for CNS gene therapy. FIG. 1D shows the virus-like particle, containing only the Replicon RNA, derived from a eukaryotic expression vector transfection (option 4). This encapsulating process is virus-independent.

FIG. 2 illustrates a diagram of another exemplary replicon described in the present disclosure, in comparison with the wild-type zika virus genome.

FIG. 3 shows an exemplary imaging result of a cell transfected with plasmids carrying a GFP construct.

FIGS. 4A-4F shows a work flowchart of the generation of a recombinant zika virus from infecting construct-expressing cells with a wild-type zika virus, and microscopy results in bright field.

FIG. 5 shows a plasmid construct comprising a hammerhead ribozyme of a zika virus; (ii) a 5′untranslated region (UTR) of the zika virus; (iii) a C-partial of the zika virus; (iv) a 3′UTR of the zika virus; (v) a hepatitis delta virus (HDV) ribozyme of the zika virus, and two reporter genes (eGFP) and Nluc.

FIG. 6 shows a work flowchart of RNA transfection of in-vitro transcription products, and quantification of the expression of the exogenous reporter gene Nluc in cell lysate or supernatant of the transfected cells.

FIG. 7 shows production (steps 1-3) and delivery and expression of an exogenous gene (step 4) in Daoy and Vero cells using a zika virus.

FIG. 8 shows RT PCR from samples after 5 days post-infection of VERO cells with the ZIKV viral vector. Quantification of the RNA copies numbers present in the viral vector (pREP+ZIKV), are compared to ZIKV (submitted only to ZIKV infection), with pREP-specific TaqMan primers (black bars) and ZIKV (gray bars).

FIG. 9 shows the tropism of ZIKVV and the expression of the pREP delivered NanoNuc reporter gene in different types of cells. The control group has no viral infection, the ZIKV wild type group was infected with wild type ZIKA virus, and ZIKVV group was infected with the novel ZIKA virus viral vector.

FIG. 10 is an example construct comprising polycistronic luciferase and beta-galactosidase genes within a zika virus 5′ UTR and a zika virus 3′ UTR.

FIG. 11 is an example construct comprising a gene encoding a SARS-CoV-2 protein within a zika virus 5′ UTR and a zika virus 3′ UTR.

FIG. 12 provides graphs showing Luciferase and Beta-galactosidase quantification in Vero cells after transfection with pREP-Luc/Gal vector, compared to control cells (transfected only with lipofectamine). The high level of NanoLuc and Beta-galactosidase expression confirms the capacity of a ZIKV construct to deliver multiples genes.

FIG. 13 shows the ELISA results for detection of the recombinant SARS-CoV-2 Spike protein produced in Vero cells after the transfection with pREP mRNA construction containing the gene of interest that codifies the Spike protein.

FIGS. 14A-14E are example constructs having different lengths of partial capsid gene: 15 bp (FIG. 14A), 30 bp (FIG. 14B), 45 bp (FIG. 14C), 60 bp (FIG. 14D), and 75 bp (FIG. 14E).

FIGS. 15A-15B show biodistribution and tropism of ZIKVV in mice 6 hours after ZIKVV IP injection (FIG. 15A), and 48 hours after ZIKVV IP injection (FIG. 15B).

FIG. 16A shows pREP RNA encapsulation in all plasmids tested (FIGS. 14A-14E, C-partial with 15, 30, 45, 60 and 75 bp, respectively). The pREP RNA encapsulation was higher in plasmids with C-partial with 15, 30, 45, and 60 bp, indicating that ZIKVV carrying pREP RNA with Capsid with 20 or fewer amino acids is more efficiently encapsulated into the viral vector.

FIG. 16B shows that the encapsulation derived from DNA transfection was efficient and the plasmid with C-partial 45 bp had a higher amount of pREP RNA.

FIGS. 17A-17B show Nanoluc quantification in Vero cells after contact with viral vector (pREP+ZIKV) carrying 5, 10, 15, 20 and 25 amino acids, derived from both RNA (FIG. 17A) and DNA (FIG. 17B) transfection, compared to ZIKV (only in ZIKV infection). A higher level of NanoLuc expression is demonstrated in viral vectors of all capsid sequence sizes compared to the control group. These results indicate that ZIKVV is able to encapsulate pREP RNA with C-partial (15, 30, 45, 60 and 75 bp) and delivery NanoLuc, confirmed by the expression of the reporter gene in the target cells.

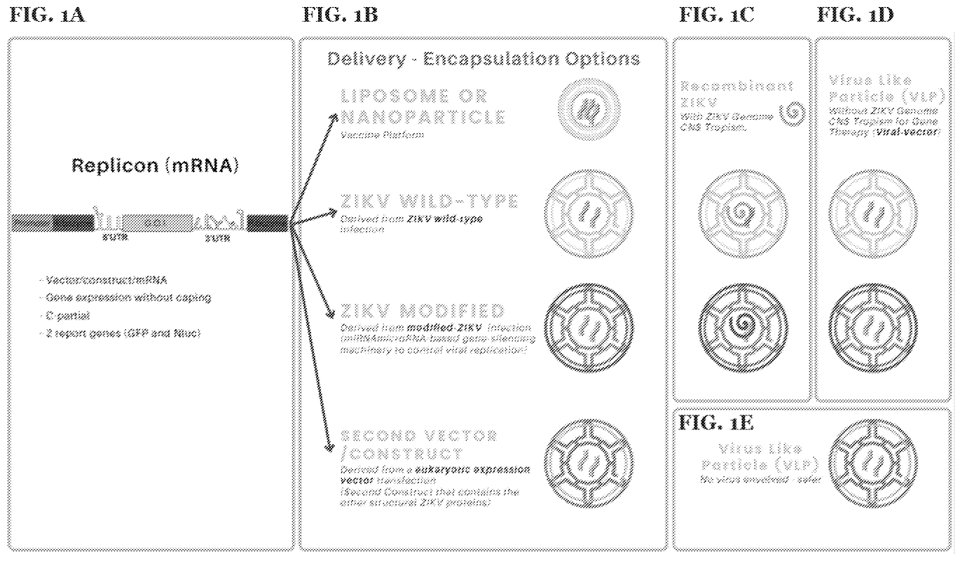

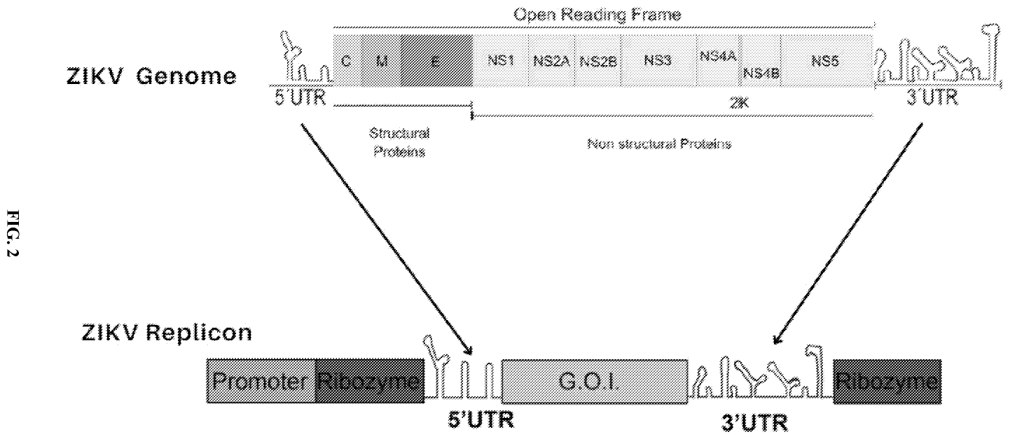

### DETAILED DESCRIPTION OF THE INVENTION

Various nucleic acid compositions are provided herein comprising an exogenous polynucleotide. The nucleic acid compositions may be useful for delivering the exogenous polynucleotide to a target cell. The target cell may be a cell of the central nervous system. The target cell may be present in a subject, where the nucleic acid compositions deliver the exogenous polynucleotide to the target cell in the subject. Certain nucleic acid compositions comprise an exogenous polynucleotide up to about 6,000 or 10,000 nucleotides in length, e.g., about 5 nucleotides to about 6,000 nucleotides. The nucleic acid compositions may comprise a 5′untranslated region (UTR) of a zika virus and a 3′UTR of the zika virus, and optionally do not comprise a polynucleotide encoding one or more nonstructural proteins of a virus, and further optionally do not comprise a polynucleotide encoding one or more structural proteins of a virus.

In one aspect, provided herein is a nucleic acid composition comprising (i) a polynucleotide encoding a ribozyme (ii) 5′untranslated region (UTR) of a zika virus; (iii) 3′UTR of the zika virus; and (iv) a polynucleotide exogenous to the zika virus. In some embodiments, the ribozyme is a hammerhead ribozyme or a hepatitis delta virus (HDV) ribozyme. In some embodiments, the nucleic acid further comprises a polynucleotide encoding a second ribozyme. In some specific embodiments, the second ribozyme is a hammerhead ribozyme or a hepatitis delta virus (HDV) ribozyme. In some embodiments, the polynucleotide encoding the ribozyme encodes a hammerhead ribozyme and the polynucleotide encoding the second ribozyme encodes an HDV ribozyme. In some embodiments, the nucleic acid composition further comprises a C-partial. For example, the C-partial is a derivative of C, where the derivative encodes a truncation (e.g., about or less than about 30, 25, 20, 15, 10, or 5 amino acids) of C.

In one aspect, provided herein is a nucleic acid composition comprising a sequence encoding one of the structural proteins from zika virus or its derivative with an exogenous polynucleotide. In a non-limiting example, the nucleic acid composition comprises a derivative of zika capsid protein (C), where the derivative encodes a truncation (e.g., about or less than about 30, 25, 20, 15, 10, or 5 amino acids) of C.

Non-limiting example nucleic acids herein may comprise one or more sequences from Tables 1A-1K, Table 2 and Table 3.

Further provided herein is a recombinant zika virus or a zika-virus-like particle that comprises all three structural proteins from zika virus or their derivative (a zika virus capsid protein (C) or the derivative of the zika virus C, a polynucleotide encoding a zika virus viral membrane protein (prM/M) or the derivative of the zika virus prM/M, a polynucleotide encoding a zika virus viral envelope protein (E) or the derivative of the zika virus E). Further provided herein is a method of using such recombinant zika virus or such zika-virus-like particle to deliver exogenous polynucleotides to a target cell. In preferred embodiments, such recombinant zika virus effectively crosses the blood brain barrier, resulting in a tropism targeting central nervous system (CNS). Accordingly, in preferred embodiments, such recombinant zika virus is used to deliver exogenous polynucleotides to CNS.

Before the present methods and compositions are described, it is to be understood that this disclosure is not limited to a particular method or composition described, and as such may vary. Unless defined otherwise, all technical and scientific terms used herein have the same meaning as commonly understood by one of ordinary skill in the art. All publications mentioned herein are incorporated by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Where a range of values is provided, unless otherwise indicated, each intervening value to the tenth of the unit of the lower limit between the upper and lower limits of that range is also specifically disclosed. Each smaller range between any stated value or intervening value in a stated range is encompassed herein. The upper and lower limits of these smaller ranges may independently be included or excluded in the range, and each range where either, neither or both limits are included in the smaller ranges is also encompassed herein, subject to any specifically excluded limit in the stated range. Where the stated range includes one or both of the limits, ranges excluding either or both of those included limits are also included.

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The terms “individual,” “patient,” or “subject” can be used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). In some embodiments, patients, subjects, or individuals can be under the supervision of a health care worker.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The terms “inhibiting,” “reducing” or “prevention,” or any variation of these terms, referred to herein, can include any measurable decrease or complete inhibition to achieve a desired result.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

The term “percentage identical to,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject, or to a non-human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” can refer to the amount of a compound that, when administered, can be sufficient to prevent development of, or alleviate to some extent, one or more of the symptoms of the disorder, disease, or condition being treated. The term “therapeutically effective amount” can also refer to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” can refer to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” can refer to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “zika-virus-like particle” can refer to a nanoscale multiprotein structure that is made up of assembled zika viral proteins and mimic the organization and conformation of the zika virus but without viral genetic material and are therefore non-infectious. The zika-virus-like particle could closely resemble zika virus.

In some embodiments, as used here a “derivative” of a polypeptide or polynucleotide refers to a sequence at least 80% identical to the polypeptide or polynucleotide, respectively. In some embodiments, as used here a “derivative” of a polypeptide or polynucleotide refers to a sequence at least 80% homologous to the polypeptide or polynucleotide, respectively. In some embodiments, as used herein, a “derivative” of a polypeptide or polynucleotide refers to a sequence with no or no more than 1, 2, 3, 4, 5, 6, 7, 8, 9, or 10 amino acid or nucleotide substitutions, insertions, or deletions as compared to the polypeptide or polynucleotide, respectively.

### Nucleic Acid Compositions

In one aspect, provided herein is a nucleic acid composition comprising (i) a polynucleotide encoding a zika virus capsid protein (C) or the derivative of the zika virus C, a polynucleotide encoding a zika virus viral membrane protein (prM/M) or the derivative of the zika virus prM/M, a polynucleotide encoding a zika virus viral envelope protein (E) or the derivative of the zika virus E, or any combination thereof; and (ii) a polynucleotide exogenous to the zika virus.

In another aspect, provided herein is a nucleic acid composition comprising a zika virus 5′ UTR, a zika virus 3′ UTR, and a polynucleotide exogenous to a zika virus. In some embodiments, the nucleic acid does not comprise: a polynucleotide encoding a zika virus capsid protein (C) or the derivative of the zika virus C, a polynucleotide encoding a zika virus viral membrane protein (prM/M) or the derivative of the zika virus prM/M, a polynucleotide encoding a zika virus viral envelope protein (E) or the derivative of the zika virus E, or any combination thereof.

Structural and Non-Structural Proteins from Zika Virus

A wild-type zika virus genome comprises a 10.8-kilobase single-stranded positive-sense RNA that codes for three structural proteins (C, prM/M, and envelope E) and seven non-structural proteins (NS1, NS2A, NS2B, NS3, NS4A, NS4B, and NS5). In addition, there are short UTRs on both the 5′ and 3′ ends of the genome. Provided herein are various embodiments to generate a recombinant zika virus that comprises at least one or all of the three structural proteins. In some embodiments, a nucleic acid is provided for generating the recombinant zika virus, wherein the nucleic acid is lacking a polynucleotide encoding for one or more, or all, of the zika virus structural proteins and zika virus nonstructural proteins. The nucleic acid may comprise a zika virus 5′ UTR and a zika virus 3′ UTR, and an exogenous nucleic acid.

In some embodiments, the nucleic acid composition comprises the polynucleotide encoding the zika virus C, the polynucleotide encoding the zika virus prM/M, and the polynucleotide encoding the zika virus E. When the three structural proteins are expressed as the nucleic acid composition described herein, in some embodiments, they could be expressed in one nucleic acid. When three structural proteins are expressed as the nucleic acid composition described herein, in other embodiments, they could be expressed in more than one nucleic acids.

In some embodiments, the polynucleotide encoding the derivative of the zika virus C comprises at least one substitution, at least one deletion, and/or at least one insertion as compared to a wild-type zika virus C. In specific embodiments, the zika virus C or the derivative of the zika virus C comprises an amino acid sequence at least 90%, 91%, 92%, 93%, 94%, 95%, 96%, 97%, 98%, 99%, or 100% identical to a sequence of Table 1A, Table 1D, Table 1I, Table 2, or Table 3. In specific embodiments, the nucleic acid composition comprises a derivative of the zika virus C encoding an amino acid sequence at least 90%, 91%, 92%, 93%, 94%, 95%, 96%, 97%, 98%, 99%, or 100% identical to a wild type C sequence. In specific embodiments, the nucleic acid composition comprises a derivative of the zika virus C encoding an amino acid sequence at least 95% identical to a wild type C sequence. In specific embodiments, the nucleic acid composition comprises a derivative of the zika virus C encoding an amino acid sequence at least 96% identical to a wild type C sequence. In specific embodiments, the nucleic acid composition comprises a derivative of the zika virus C encoding an amino acid sequence at least 97% identical to a wild type C sequence. In specific embodiments, the nucleic acid composition comprises a derivative of the zika virus C encoding an amino acid sequence at least 98% identical to a wild type C sequence. In specific embodiments, the nucleic acid composition comprises a derivative of the zika virus C encoding an amino acid sequence at least 99% identical to a wild type C sequence. In some embodiments, the nucleic acid composition comprises a derivative of zika virus C encoding about 5 to about 30 amino acids of a sequence at least 90%, 91%, 92%, 93%, 94%, 95%, 96%, 97%, 98%, 99%, or 100% identical to a sequence of Table 1A, Table 1D, Table 1I, Table 2, Table 3, or a wild-type zika virus.

In some embodiments, the polynucleotide encoding the derivative of the zika virus prM/M comprises at least one substitution, at least one deletion, and/or at least one insertion as compared to a wild-type zika virus prM/M. In some embodiments, the zika virus prM/M or the derivative of the zika virus prM/M comprises a sequence at least 90%, 91%, 92%, 93%, 94%, 95%, 96%, 97%, 98%, 99%, or 100% identical to a sequence of Table 1A, Table 1F, or Table 1K. In specific embodiments, the nucleic acid composition comprises a derivative of the zika virus prM/M encoding an amino acid sequence at least 95% identical to a wild type prM/M sequence. In specific embodiments, the nucleic acid composition comprises a derivative of the zika virus prM/M encoding an amino acid sequence at least 96% identical to a wild type prM/M sequence. In specific embodiments, the nucleic acid composition comprises a derivative of the zika virus prM/M encoding an amino acid sequence at least 97% identical to a wild type prM/M sequence. In specific embodiments, the nucleic acid composition comprises a derivative of the zika virus prM/M encoding an amino acid sequence at least 98% identical to a wild type prM/M sequence. In specific embodiments, the nucleic acid composition comprises a derivative of the zika virus prM/M encoding an amino acid sequence at least 99% identical to a wild type prM/M sequence.

In some embodiments, the polynucleotide encoding the derivative of the zika virus E comprises at least one substitution, at least one deletion, and/or at least one insertion as compared to a wild-type zika virus E. In some embodiments, the polynucleotide encoding E is translated into a wild zika virus type E. In some embodiments, the zika virus E or the derivative of the zika virus E comprises a sequence at least 90%, 91%, 92%, 93%, 94%, 95%, 96%, 97%, 98%, 99%, or 100% identical to sequence of Table 1A, Table IE, or Table 1J. In specific embodiments, the nucleic acid composition comprises a derivative of the zika E encoding an amino acid sequence at least 95% identical to a wild type E sequence. In specific embodiments, the nucleic acid composition comprises a derivative of the zika virus E encoding an amino acid sequence at least 96% identical to a wild type E sequence. In specific embodiments, the nucleic acid composition comprises a derivative of the zika virus E encoding an amino acid sequence at least 97% identical to a wild type E sequence. In specific embodiments, the nucleic acid composition comprises a derivative of the zika virus E encoding an amino acid sequence at least 98% identical to a wild type E sequence. In specific embodiments, the nucleic acid composition comprises a derivative of the zika virus E encoding an amino acid sequence at least 99% identical to a wild type E sequence.

In some embodiments, the nucleic acid composition comprises a 5′ untranslated region (5′ UTR) of the zika virus. In some embodiments, the nucleic acid composition comprises a 3′ untranslated region (3′ UTR) of the zika virus. Non-limiting example untranslated sequences are provided in Table 1A, Table 1B, Table 1C, Table 1G, Table 1H, or Table 2. In some embodiments, the zika virus 5′ UTR comprises a sequence at least 90%, 91%, 92%, 93%, 94%, 95%, 96%, 97%, 98%, 99%, or 100% identical to sequence of Table 1A, Table 1C, Table 1H, or Table 2. In some embodiments, the zika virus 3′ UTR comprises a sequence at least 90%, 91%, 92%, 93%, 94%, 95%, 96%, 97%, 98%, 99%, or 100% identical to sequence of Table 1A, Table 1B, Table 1G, or Table 2.

In some embodiments, the nucleic acid composition does not comprise a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii). For example, if the nucleic acid does not comprise such polynucleotide(s), there is additional space in the composition to accommodate a large exogenous polynucleotide (e.g., up to about 10000 or 6000 bases).

In other embodiments, the nucleic acid composition comprises polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii).

In certain embodiments, the nucleic acid composition comprises a polynucleotide encoding a NS1 protein. In certain embodiments, the nucleic acid composition comprises a polynucleotide encoding a NS2A protein. In certain embodiments, the nucleic acid composition comprises a polynucleotide encoding a NS2B protein. In certain embodiments, the nucleic acid composition comprises a polynucleotide encoding a NS3 protein. In certain embodiments, the nucleic acid composition comprises a polynucleotide encoding a NS4A protein. In certain embodiments, the nucleic acid composition comprises a polynucleotide encoding a NS4B protein. In certain embodiments, the nucleic acid composition comprises a polynucleotide encoding a NS5 protein.

In certain embodiments, the nucleic acid composition comprises a polynucleotide encoding a NS1 and a NS2A. In certain embodiments, the nucleic acid composition comprises a polynucleotide encoding a NS1 and a NS2B. In certain embodiments, the nucleic acid composition comprises a polynucleotide encoding a NS1 and a NS3. In certain embodiments, the nucleic acid composition comprises a polynucleotide encoding a NS1 and a NS4A. In certain embodiments, the nucleic acid composition comprises a polynucleotide encoding a NS1 and a NS4B. In certain embodiments, the nucleic acid composition comprises a polynucleotide encoding a NS1 and a NS5.

In certain embodiments, the nucleic acid composition comprises a polynucleotide encoding a NS2A and a NS2B. In certain embodiments, the nucleic acid composition comprises a polynucleotide encoding a NS2A and a NS3. In certain embodiments, the nucleic acid composition comprises a polynucleotide encoding a NS2A and a NS4A. In certain embodiments, the nucleic acid composition comprises a polynucleotide encoding a NS2A and a NS4B. In certain embodiments, the nucleic acid composition comprises a polynucleotide encoding a NS2A and a NS5.

In certain embodiments, the nucleic acid composition comprises a polynucleotide encoding a NS2B and a NS3. In certain embodiments, the nucleic acid composition comprises a polynucleotide encoding a NS2B and a NS4A. In certain embodiments, the nucleic acid composition comprises a polynucleotide encoding a NS2B and a NS4B. In certain embodiments, the nucleic acid composition comprises a polynucleotide encoding a NS2B and a NS5.

In certain embodiments, the nucleic acid composition comprises a polynucleotide encoding a NS3 and a NS4A. In certain embodiments, the nucleic acid composition comprises a polynucleotide encoding a NS3 and a NS4B. In certain embodiments, the nucleic acid composition comprises a polynucleotide encoding a NS3 and a NS5.

In certain embodiments, the nucleic acid composition comprises a polynucleotide encoding a NS4A and a NS4B. In certain embodiments, the nucleic acid composition comprises a polynucleotide encoding a NS4A and a NS5.

In certain embodiments, the nucleic acid composition comprises a polynucleotide encoding a NS4B and a NS5.

In certain embodiments, the nucleic acid composition comprises a polynucleotide encoding a NS1, a NS2A, and NS2B. In certain embodiments, the nucleic acid composition comprises a polynucleotide encoding a NS1, a NS2A, and NS3. In certain embodiments, the nucleic acid composition comprises a polynucleotide encoding a NS1, a NS2A, and NS4A. In certain embodiments, the nucleic acid composition comprises a polynucleotide encoding a NS1, a NS2A, and NS4B. In certain embodiments, the nucleic acid composition comprises a polynucleotide encoding a NS1, a NS2A, and NS5.

In certain embodiments, the nucleic acid composition comprises a polynucleotide encoding a NS1, a NS2B, and NS3. In certain embodiments, the nucleic acid composition comprises a polynucleotide encoding a NS1, a NS2B, and NS4A. In certain embodiments, the nucleic acid composition comprises a polynucleotide encoding a NS1, a NS2B, and NS4B. In certain embodiments, the nucleic acid composition comprises a polynucleotide encoding a NS1, a NS2B, and NS5.

In certain embodiments, the nucleic acid composition comprises a polynucleotide encoding a NS1, a NS3, and NS4A. In certain embodiments, the nucleic acid composition comprises a polynucleotide encoding a NS1, a NS3, and NS4B. In certain embodiments, the nucleic acid composition comprises a polynucleotide encoding a NS1, a NS3, and NS5.

In certain embodiments, the nucleic acid composition comprises a polynucleotide encoding a NS1, a NS4A, and a NS4B. In certain embodiments, the nucleic acid composition comprises a polynucleotide encoding a NS1, a NS4A, and a NS5.

In certain embodiments, the nucleic acid composition comprises a polynucleotide encoding a NS1, a NS4B, and a NS5.

In certain embodiments, the nucleic acid composition comprises a polynucleotide encoding a NS2A, a NS2B, and a NS3. In certain embodiments, the nucleic acid composition comprises a polynucleotide encoding a NS2A, a NS2B, and a NS4A. In certain embodiments, the nucleic acid composition comprises a polynucleotide encoding a NS2A, a NS2B, and a NS4B. In certain embodiments, the nucleic acid composition comprises a polynucleotide encoding a NS2A, a NS2B, and a NS5.

In certain embodiments, the nucleic acid composition comprises a polynucleotide encoding a NS2A, a NS3, and a NS4A. In certain embodiments, the nucleic acid composition comprises a polynucleotide encoding a NS2A, a NS3, and a NS4B. In certain embodiments, the nucleic acid composition comprises a polynucleotide encoding a NS2A, a NS3, and a NS5.

In certain embodiments, the nucleic acid composition comprises a polynucleotide encoding a NS2A, a NS4A, and a NS4B. In certain embodiments, the nucleic acid composition comprises a polynucleotide encoding a NS2A, a NS4A, and a NS5.

In certain embodiments, the nucleic acid composition comprises a polynucleotide encoding a NS2A, a NS4B, and a NS5.

In certain embodiments, the nucleic acid composition comprises a polynucleotide encoding a NS2B, a NS3, and a NS4A. In certain embodiments, the nucleic acid composition comprises a polynucleotide encoding a NS2B, a NS3, and a NS4B. In certain embodiments, the nucleic acid composition comprises a polynucleotide encoding a NS2B, a NS3, and a NS5.

In certain embodiments, the nucleic acid composition comprises a polynucleotide encoding a NS2B, a NS4A, and NS5.

In certain embodiments, the nucleic acid composition comprises a polynucleotide encoding a NS2B, a NS4B, and a NS5.

In certain embodiments, the nucleic acid composition comprises a polynucleotide encoding a NS3 and a NS4A, and a NS5.

In certain embodiments, the nucleic acid composition comprises a polynucleotide encoding a NS3, a NS4B, and a NS5.

In certain embodiments, the nucleic acid composition comprises a polynucleotide encoding a NS4A, a NS4B, and a NS5.

In certain embodiments, the nucleic acid composition comprises a polynucleotide encoding all NS proteins except a NS1, a NS2A, and NS2B. In certain embodiments, the nucleic acid composition comprises a polynucleotide encoding all NS proteins except a NS1, a NS2A, and NS3. In certain embodiments, the nucleic acid composition comprises a polynucleotide encoding all NS proteins except a NS1, a NS2A, and NS4A. In certain embodiments, the nucleic acid composition comprises a polynucleotide encoding all NS proteins except a NS1, a NS2A, and NS4B. In certain embodiments, the nucleic acid composition comprises a polynucleotide encoding all NS proteins except a NS1, a NS2A, and NS5.

In certain embodiments, the nucleic acid composition comprises a polynucleotide encoding all NS proteins except a NS1, a NS2B, and NS3. In certain embodiments, the nucleic acid composition comprises a polynucleotide encoding all NS proteins except a NS1, a NS2B, and NS4A. In certain embodiments, the nucleic acid composition comprises a polynucleotide encoding all NS proteins except a NS1, a NS2B, and NS4B. In certain embodiments, the nucleic acid composition comprises a polynucleotide encoding all NS proteins except a NS1, a NS2B, and NS5.

In certain embodiments, the nucleic acid composition comprises a polynucleotide encoding all NS proteins except a NS1, a NS3, and NS4A. In certain embodiments, the nucleic acid composition comprises a polynucleotide encoding all NS proteins except a NS1, a NS3, and NS4B. In certain embodiments, the nucleic acid composition comprises a polynucleotide encoding all NS proteins except a NS1, a NS3, and NS5.

In certain embodiments, the nucleic acid composition comprises a polynucleotide encoding all NS proteins except a NS1, a NS4A, and a NS4B. In certain embodiments, the nucleic acid composition comprises a polynucleotide encoding all NS proteins except a NS1, a NS4A, and a NS5.

In certain embodiments, the nucleic acid composition comprises a polynucleotide encoding all NS proteins except a NS1, a NS4B, and a NS5.

In certain embodiments, the nucleic acid composition comprises a polynucleotide encoding all NS proteins except a NS2A, a NS2B, and a NS3. In certain embodiments, the nucleic acid composition comprises a polynucleotide encoding all NS proteins except a NS2A, a NS2B, and a NS4A. In certain embodiments, the nucleic acid composition comprises a polynucleotide encoding all NS proteins except a NS2A, a NS2B, and a NS4B. In certain embodiments, the nucleic acid composition comprises a polynucleotide encoding all NS proteins except a NS2A, a NS2B, and a NS5.

In certain embodiments, the nucleic acid composition comprises a polynucleotide encoding all NS proteins except a NS2A, a NS3, and a NS4A. In certain embodiments, the nucleic acid composition comprises a polynucleotide encoding all NS proteins except a NS2A, a NS3, and a NS4B. In certain embodiments, the nucleic acid composition comprises a polynucleotide encoding all NS proteins except a NS2A, a NS3, and a NS5.

In certain embodiments, the nucleic acid composition comprises a polynucleotide encoding all NS proteins except a NS2A, a NS4A, and a NS4B. In certain embodiments, the nucleic acid composition comprises a polynucleotide encoding all NS proteins except a NS2A, a NS4A, and a NS5.

In certain embodiments, the nucleic acid composition comprises a polynucleotide encoding all NS proteins except a NS2A, a NS4B, and a NS5.

In certain embodiments, the nucleic acid composition comprises a polynucleotide encoding all NS proteins except a NS2B, a NS3, and a NS4A. In certain embodiments, the nucleic acid composition comprises a polynucleotide encoding all NS proteins except a NS2B, a NS3, and a NS4B. In certain embodiments, the nucleic acid composition comprises a polynucleotide encoding all NS proteins except a NS2B, a NS3, and a NS5.

In certain embodiments, the nucleic acid composition comprises a polynucleotide encoding all NS proteins except a NS2B, a NS4A, and NS5.

In certain embodiments, the nucleic acid composition comprises a polynucleotide encoding all NS proteins except all NS proteins except a NS2B, a NS4B, and a NS5.

In certain embodiments, the nucleic acid composition comprises a polynucleotide encoding all NS proteins except a NS3 and a NS4A, and a NS5.

In certain embodiments, the nucleic acid composition comprises a polynucleotide encoding all NS proteins except a NS3, a NS4B, and a NS5.

In certain embodiments, the nucleic acid composition comprises a polynucleotide encoding all NS proteins except a NS4A, a NS4B, and a NS5.

In certain embodiments, the nucleic acid composition comprises a polynucleotide encoding all NS proteins except a NS1 and a NS2A. In certain embodiments, the nucleic acid composition comprises a polynucleotide encoding all NS proteins except a NS1 and a NS2B. In certain embodiments, the nucleic acid composition comprises a polynucleotide encoding all NS proteins except a NS1 and a NS3. In certain embodiments, the nucleic acid composition comprises a polynucleotide encoding all NS proteins except all NS proteins except a NS1 and a NS4A. In certain embodiments, the nucleic acid composition comprises a polynucleotide encoding all NS proteins except a NS1 and a NS4B. In certain embodiments, the nucleic acid composition comprises a polynucleotide encoding all NS proteins except a NS1 and a NS5.

In certain embodiments, the nucleic acid composition comprises a polynucleotide encoding all NS proteins except a NS2A and a NS2B. In certain embodiments, the nucleic acid composition comprises a polynucleotide encoding all NS proteins except a NS2A and a NS3. In certain embodiments, the nucleic acid composition comprises a polynucleotide encoding all NS proteins except a NS2A and a NS4A. In certain embodiments, the nucleic acid composition comprises a polynucleotide encoding all NS proteins except a NS2A and a NS4B. In certain embodiments, the nucleic acid composition comprises a polynucleotide encoding all NS proteins except a NS2A and a NS5.

In certain embodiments, the nucleic acid composition comprises a polynucleotide encoding all NS proteins except a NS2B and a NS3. In certain embodiments, the nucleic acid composition comprises a polynucleotide encoding all NS proteins except a NS2B and a NS4A. In certain embodiments, the nucleic acid composition comprises a polynucleotide encoding all NS proteins except a NS2B and a NS4B. In certain embodiments, the nucleic acid composition comprises a polynucleotide encoding all NS proteins except a NS2B and a NS5.

In certain embodiments, the nucleic acid composition comprises a polynucleotide encoding all NS proteins except a NS3 and a NS4A. In certain embodiments, the nucleic acid composition comprises a polynucleotide encoding all NS proteins except a NS3 and a NS4B. In certain embodiments, the nucleic acid composition comprises a polynucleotide encoding all NS proteins except a NS3 and a NS5.

In certain embodiments, the nucleic acid composition comprises a polynucleotide encoding all NS proteins except a NS4A and a NS4B. In certain embodiments, the nucleic acid composition comprises a polynucleotide encoding all NS proteins except a NS4A and a NS5.

In certain embodiments, the nucleic acid composition comprises a polynucleotide encoding all NS proteins except a NS4B and a NS5.

In certain embodiments, the nucleic acid composition comprises a polynucleotide encoding all NS proteins except a NS1. In certain embodiments, the nucleic acid composition comprises a polynucleotide encoding all NS proteins except a NS2A. In certain embodiments, the nucleic acid composition comprises a polynucleotide encoding all NS proteins except a NS2B. In certain embodiments, the nucleic acid composition comprises a polynucleotide encoding all NS proteins except a NS3. In certain embodiments, the nucleic acid composition comprises a polynucleotide encoding all NS proteins except a NS4A. In certain embodiments, the nucleic acid composition comprises a polynucleotide encoding all NS proteins except a NS4B. In certain embodiments, the nucleic acid composition comprises a polynucleotide encoding all NS proteins except a NS5.

In some embodiments, the NS1 is a zika virus NS1. In some embodiments, the NS2A is a zika virus NS2A. In some embodiments, the NS2B is a zika virus NS2B. In some embodiments, the NS3 is a zika virus NS3. In some embodiments, the NS4A is a zika virus NS4A. In some embodiments, the NS4B is a zika virus NS4B. In some embodiments, the NS5 is a zika virus NS5.

In some embodiments, the nucleic acid composition comprises a derivative of NS1 that comprises an amino acid sequence at least 80%, at least 90%, at least 95%, at least 96%, at least 97%, at least 98%, at least 99% identical to a wild-type NS1 sequence of Table 1A. In some embodiments, the nucleic acid composition comprises a derivative of NS2A that comprises an amino acid sequence at least 80%, at least 90%, at least 95%, at least 96%, at least 97%, at least 98%, at least 99% identical to a wild-type NS2A sequence of Table 1A. In some embodiments, the nucleic acid composition comprises a derivative of NS2B that comprises an amino acid sequence at least 80%, at least 90%, at least 95%, at least 96%, at least 97%, at least 98%, at least 99% identical to a wild-type NS2B sequence of Table 1A. In some embodiments, the nucleic acid composition comprises a derivative of NS3 that comprises an amino acid sequence at least 80%, at least 90%, at least 95%, at least 96%, at least 97%, at least 98%, at least 99% identical to a wild-type NS3 sequence of Table 1A. In some embodiments, the nucleic acid composition comprises a derivative of NS4A that comprises an amino acid sequence at least 80%, at least 90%, at least 95%, at least 96%, at least 97%, at least 98%, at least 99% identical to a wild-type NS4A sequence of Table 1A. In some embodiments, the nucleic acid composition comprises a derivative of NS4B that comprises an amino acid sequence at least 80%, at least 90%, at least 95%, at least 96%, at least 97%, at least 98%, at least 99% identical to a wild-type NS4B sequence of Table 1A. In some embodiments, the nucleic acid composition comprises a derivative of NS5 that comprises an amino acid sequence at least 80%, at least 90%, at least 95%, at least 96%, at least 97%, at least 98%, at least 99% identical to a wild-type NS5 sequence.

In some embodiments, the components of the nucleic acid composition comprise African zika virus components, Asian zika virus components, or Brazilian zika virus components, or a combination thereof. In one specific embodiment, the zika virus is African MR766 strain.

### Exogenous Polynucleotides

In some embodiments, the exogeneous polynucleotide is about or up to about 10000, 9000, 8000, 7000, 6000, or 5000 nucleotides long. For example, the exogenous polynucleotide is about 50 to about 10000, 9000, 8000, 7000, 6000, or 5000 nucleotides long. The exogenous polynucleotide may be about 100 to about 10000, 9000, 8000, 7000, 6000, or 5000 nucleotides long. The exogenous polynucleotide may be about 50 to about 6000 nucleotides long. The exogenous polynucleotide may be about 100 to about 6000 nucleotides long. The exogenous polynucleotide may be about 50 to about 5000 nucleotides long. The exogenous polynucleotide may be about 100 to about 5000 nucleotides long.

In some embodiments, the exogeneous polynucleotide is about 0.1 kb nucleotides long. In other embodiments, the exogenous sequence is about 0.2 kb long. In other embodiments, the exogenous sequence is about 0.3 kb long. In other embodiments, the exogenous sequence is about 0.4 kb long. In other embodiments, the exogenous sequence is about 0.5 kb long. In other embodiments, the exogenous sequence is about 0.6 kb long. In other embodiments, the exogenous sequence is about 0.7 kb long. In other embodiments, the exogenous sequence is about 0.8 kb long. In other embodiments, the exogenous sequence is about 0.9 kb long. In other embodiments, the exogenous sequence is about 1 kb nucleotides long. In other embodiments, the exogeneous polynucleotide is about 2 kb nucleotides long. In some embodiments, the exogeneous polynucleotide is about 3 kb nucleotides long. In some embodiments, the exogeneous polynucleotide is about 4 kb nucleotides long. In some embodiments, the exogeneous polynucleotide is about 5 kb nucleotides long. In some embodiments, the exogeneous polynucleotide is about 6 kb nucleotides long. In some embodiments, the exogeneous polynucleotide is about 7 kb nucleotides long. In some embodiments, the exogeneous polynucleotide is about 8 kb nucleotides long. In some embodiments, the exogeneous polynucleotide is about 9 kb nucleotides long. In some embodiments, the exogeneous polynucleotide is about 10 kb nucleotides long.

In some embodiments, the exogeneous polynucleotide is an antigen or an antigenic epitope thereof. In some embodiments, the antigen or an antigenic epitope thereof is disease associated. In specific embodiments, the antigen or an antigenic epitope thereof is from a pathogen.

In some embodiments, the pathogen is a virus. In specific embodiments, the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

In some embodiments, the pathogen is a bacterium. In specific embodiments, the bacteria are Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

In some embodiments, the pathogen is a fungus, an amoeba, or a parasite. In specific embodiments, the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In some embodiments, the exogeneous polynucleotide encodes a gene editing tool. In specific embodiments, the gene editing tool is selected from a group consisting of meganuclease associated agents, CRISPR associated agents, TALEN associated agents, and zinc finger associated agents.

In some embodiments, the exogeneous polynucleotide is a small interfering RNA (siRNA), antisense RNA, micro RNA (miRNA), small or short hairpin RNA (shRNA), guide RNA (gRNA), clustered regularly interspaced short palindromic repeat RNA (crRNA), trans-activating clustered regularly interspaced short palindromic repeat RNA (tracrRNA), immune-stimulating oligonucleotide, antisense nucleic acid, or ribozyme. In specific embodiments, the exogeneous polynucleotide targets beta-secretase 1 (BACE1) and/or amyloid precursor protein (APP).

In some embodiments, the exogeneous polynucleotide encodes a polypeptide associated with a genetic disorder. In specific embodiments, the polypeptide is brain-derived neurotrophin factor (BDNF), nerve growth factor (NGF), Neprilysin inhibitors (NEP), endothelin converting enzyme (ECE), cathepsin B (CTSB), apolipoprotein E 2 (APOE2), SH3 and multiple ankyrin repeat domains protein (SHANK), neurturin (NRTN), glial cell-derived neurotrophic factor (GDNF), cerebral dopamine neurotrophic factor (CDNF), vascular endothelial growth factor A (VEGF-A), or aromatic L-amino acid decarboxylase (AADC).

In some embodiments, the exogeneous polynucleotide encodes a therapeutic agent or a diagnostic agent. In specific embodiments, the therapeutic agent is an antibody-based therapeutic agent, a hormone, a cytokine, an inhibitor or antagonist of an immune checkpoint regulator, an immune stimulatory molecule, or an agonist of an immune co-stimulatory molecule. In further embodiments, the inhibitor or antagonist of an immune checkpoint regulator is an anti-PD1 antibody. In another embodiments, the antibody-based therapeutic agent is an antibody, a functional fragment of an antibody, a chimeric antigen receptor (CAR), or a T cell receptor (TCR). In still other embodiments, the cytokine comprises lymphokine, monokine, polypeptide hormone, growth hormone, parathyroid hormone, thyroxine, insulin, proinsulin, relaxin, prorelaxin, glycoprotein hormone, follicle stimulating hormone (FSH), thyroid stimulating hormone (TSH), luteinizing hormone (LH), hepatic growth factor, fibroblast growth factor, prolactin, placental lactogen, tumor necrosis factor-a and -β, mullerian-inhibiting substance, mouse gonadotropin-associated peptide, inhibin, activin, vascular endothelial growth factor, integrin, thrombopoietin (TPO), nerve growth factor, NGF-β, platelet growth factor, transforming growth factor (TGF), TGF-a, TGF-β, insulin-like growth factor-I and -II, erythropoietin (EPO), osteoinductive factor, interferon, such as interferon-a, -β and -γ, colony stimulating factor (CSF), macrophage-CSF (M-CSF), granulocyte-macrophage-CSF (GM-CSF), granulocyte-CSF (GCSF), interleukin (IL), tumor necrosis factor, TNF-a, TNF-β, LIF, or kit ligand (KL), or a combination of two or more thereof.

### Additional Example Features

In some embodiments, components of the nucleic acid composition are expressed on one nucleic acid, or two or more separate nucleic acids. In some embodiments, the nucleic acid composition further comprises one or more expression control elements in operable linkage that confers expression of the nucleic acid composition in vitro or in vivo. In some embodiments, the expression control element is a promoter that drives expression of the nucleic acid complex in vitro. In specific embodiments, the promoter is T7, T3, SP6 or any phage promoter.

In some embodiments, the expression control element is a promoter that drives expression of the nucleic acid complex in a target cell. In some specific embodiments, the promoter is CMV, SV40 or any eukaryotic promoter.

In some embodiments, the target cell is a neuron, or a non-neuron cell, VERO, COS, CHO, C6/36, HeLa, HEK, or HepG2. In some embodiments, the target cell is an oligodendrocyte, microglia, or astrocyte.

In some embodiments, provided herein is a pharmaceutical composition comprising the nucleic acid composition described above with a pharmaceutically acceptable salt or derivative thereof.

### A Recombinant Zika Virus or a Zika-Virus-Like Particle

Viral vectors are widely used in fields such as gene therapy. Currently, the three key vectors are based on adenoviruses, adeno-associated viruses, and lentiviruses. However, limitations for each vectors exist. Provided herein is a recombinant zika virus or a zika-virus-like particle which in some embodiments provides more flexible packaging strategies, similar or better delivery efficiency, decreased insertional mutagenesis, decreased immune response, safety, or special tropism, or any combination thereof.

In some embodiments, the recombinant zika virus or the zika-virus-like particle is generated from expressing the nucleic acid composition described herein in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In specific embodiments, the recombinant zika virus or a zika-virus-like particle is produced from a nucleic acid composition comprising a polynucleotide encoding a zika virus C or the derivative of the zika virus C only. In further specific embodiments, the recombinant zika virus or a zika-virus-like particle is produced from a nucleic acid composition that does not encode a zika virus C or derivative of zika virus C, does not encode a zika virus prM/M or derivative of zika virus prM/M, or does not encode a zika virus E or derivative of zika virus E, or any combination of two or more thereof. Accordingly, in some embodiments the recombinant zika virus described herein is generated from expressing such nucleic acid composition in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In specific embodiments, the recombinant zika virus or a zika-virus-like particle is produced from a nucleic acid composition comprising a polynucleotide encoding a zika virus prM/M or the derivative of the zika virus prM/M only. Accordingly, in some embodiments the recombinant zika virus described herein is generated from expressing such nucleic acid composition in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In specific embodiments, the recombinant zika virus or a zika-virus-like particle is produced from a nucleic acid composition comprising a polynucleotide encoding a zika virus viral E or the derivative of the zika virus E only. Accordingly, in some embodiments the recombinant zika virus described herein is generated from expressing such nucleic acid composition in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In specific embodiments, the recombinant zika virus or a zika-virus-like particle is produced from a nucleic acid composition comprising a polynucleotide encoding a zika virus C or the derivative of the zika virus C and a polynucleotide encoding a zika virus prM/M or the derivative of the zika virus prM/M only. Accordingly, in some embodiments the recombinant zika virus described herein is generated from expressing such nucleic acid composition in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In specific embodiments, the recombinant zika virus or a zika-virus-like particle is produced from a nucleic acid composition comprising a polynucleotide encoding a zika virus C or the derivative of the zika virus C and a polynucleotide encoding a zika virus viral E or the derivative of the zika virus E only. Accordingly, in some embodiments the recombinant zika virus described herein is generated from expressing such nucleic acid composition in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In specific embodiments, the recombinant zika virus or a zika-virus-like particle is produced from a nucleic acid composition comprising a polynucleotide encoding a zika virus prM/M or the derivative of the zika virus prM/M and a polynucleotide encoding a zika virus viral E or the derivative of the zika virus E only. Accordingly, in some embodiments the recombinant zika virus described herein is generated from expressing such nucleic acid composition in a producer cell, wherein the producer cell is further infected by a second zika virus, so that the zika virus C or the derivative of the zika virus C, the zika virus prM/M or the derivative of the zika virus prM/M, and the zika virus E or the derivative of the zika virus E are present in the recombinant zika virus or the zika-virus-like particle.

In some embodiments, the second zika virus used in the further infection is a wild-type zika virus. In some embodiments, the wild-type zika virus is an African, an Asian and a Brazilian strain. In other embodiments, the second zika virus used in the further infection is a modified zika virus. In some specific embodiments, the modified zika virus comprises one or more microRNA-based gene-silencing machineries. In some specific embodiments, the one or more microRNA-based gene-silencing machineries control viral replication.

In some embodiments, the recombinant zika virus or the zika-virus-like particle is generated from expressing the nucleic acid described herein in a producer cell, without an infection of a second zika virus, wherein the zika virus C or the derivative of the zika virus C, the zika virus prM/M or the derivative of the zika virus prM/M, and the zika virus E or the derivative of the zika virus E are present in the recombinant zika virus or the zika-virus-like particle.

In some embodiment, the producer cell is a Vero E6, HEK, HEK 293T, HEK 293TT, FreeStyle™ 293-F Cell, HEK-293.2sus or C6/36 cell.

In some embodiments, the recombinant zika virus is replication competent. In other embodiments, the recombinant zika virus is replication incompetent without lowering the vector titer or impairing expression of the exogeneous polynucleotide. In some embodiments, the recombinant zika virus or the zika-virus-like particle has decreased insertional mutagenesis. In some embodiments, the recombinant zika virus or the zika-virus-like particle has decreased immune response. In some embodiments, the zika virus or the zika-virus-like particle has tropism for a cell of the central nervous system.

### Methods Related to the Recombinant Zika Virus or the Zika-Virus-Like Particle Comprising Three Structural Zika Proteins

In another aspect, provided herein is a method of delivering an exogeneous polynucleotide to a target cell, the method comprising applying to the target cell the recombinant zika virus or the zika-virus-like particle described herein comprising the exogeneous polynucleotide.

In some embodiments, the target cell is a neuron or a non-neuron cell. In specific embodiments, the neuron is an oligodendrocyte, microglia, or astrocyte. In another specific embodiments, the non-neuron cell is a prostate epithelial cell, a urethra epithelial cell, a Sertoli cell, a Leydig cell, a spermatogonium cell or a retinal cell.

In some embodiments, the method is carried out in vitro, ex vivo, or in vivo.

In some embodiments, the target cell transiently expresses the exogeneous polynucleotide after delivery. In other embodiments, the target cell persistently expresses the exogeneous polynucleotide after delivery.

In another aspect, provided herein is a method of treating Alzheimer's disease in a subject in need thereof, and the method comprises administering to the subject an effective amount of the recombinant zika virus or the zika-virus-like particle as described herein comprising a polynucleotide encoding a brain-derived neurotrophin factor (BDNF).

In another aspect, provided herein is a method of treating Alzheimer's disease in a subject in need thereof, and the method comprises administering to the subject an effective amount of the recombinant zika virus or the zika-virus-like particle as described herein comprising a polynucleotide targeting beta-site amyloid precursor protein cleaving enzyme 1 (BACE1).

In another aspect, provided herein is a method of treating Alzheimer's disease in a subject in need thereof, and the method comprises administering to the subject an effective amount of the recombinant zika virus or the zika-virus-like particle as described herein comprising a polynucleotide targeting amyloid precursor protein (APP).

In another aspect, provided herein is a method of treating Alzheimer's disease in a subject in need thereof, and the method comprises administering to the subject an effective amount of the recombinant zika virus or the zika-virus-like particle as described herein comprising a polynucleotide targeting Tau.

In another aspect, provided herein is a method of treating autism in a subject in need thereof, and the method comprises administering to the subject an effective amount of the recombinant zika virus or the zika-virus-like particle as described herein comprising a polynucleotide encoding SH3 and multiple ankyrin repeat domains protein (SHANK).

In yet another aspect, provided herein is a method of treating Parkinson's disease in a subject in need thereof, the method comprising administering the subject an effective amount of the recombinant zika virus or the zika-virus-like particle as described herein comprising a polynucleotide encoding aromatic L-amino acid decarboxylase (AADC).

In another aspect, provided herein is a method of treating Parkinson's disease in a subject in need thereof, and the method comprises administering to the subject an effective amount of the recombinant zika virus or the zika-virus-like particle as described herein comprising a polynucleotide encoding a human aromatic L-amino acid decarboxylase.

In another aspect, provided herein is a method of treating Parkinson's disease in a subject in need thereof, and the method comprises administering to the subject an effective amount of the recombinant zika virus or the zika-virus-like particle as described herein comprising a polynucleotide encoding glial cell line-derived neurotrophic factor.

In another aspect, provided herein is a method of treating/managing Down's Syndrome in a subject in need thereof, and the method comprises administering to the subject an effective amount of the recombinant zika virus or the zika-virus-like particle as described herein comprising a polynucleotide targets the additional copy of HSA21.

In another aspect, provided herein is a method of treating choroideremia-blindness in a subject in need thereof, and the method comprises administering to the subject an effective amount of the recombinant zika virus or the zika-virus-like particle as described herein comprising a polynucleotide encoding rab-escort protein 1 (REP1).

In another aspect, provided herein is a method of treating leber congenital amaurosis in a subject in need thereof, and the method comprises administering to the subject an effective amount of the recombinant zika virus or the zika-virus-like particle as described herein comprising a polynucleotide encoding an RPE65.

In another aspect, provided herein is a method of treating Parkinson's disease in a subject in need thereof, and the method comprises administering to the subject an effective amount of the recombinant zika virus or the zika-virus-like particle as described herein comprising a polynucleotide encoding a glutamic acid decarboxylase.

In another aspect, provided herein is a method of treating ornithine transcarbamylase (OTC) deficiency in a subject in need thereof, and the method comprises administering to the subject an effective amount of the recombinant zika virus or the zika-virus-like particle as described herein comprising a polynucleotide encoding ornithine transcarbamylase.

In another aspect, provided herein is a method of treating multiple sclerosis in a subject in need thereof, and the method comprises administering to the subject an effective amount of the recombinant zika virus or the zika-virus-like particle as described herein comprising a polynucleotide encoding an interferon-beta-1a and 1b.

In another aspect, provided herein is a method of treating Pompe Disease in a subject in need thereof, and the method comprises administering to the subject an effective amount of the recombinant zika virus or the zika-virus-like particle as described herein comprising a polynucleotide encoding an acid alpha-glucosidase.

In another aspect, provided herein is a method of treating depression in a subject in need thereof, and the method comprises administering to the subject an effective amount of the recombinant zika virus or the zika-virus-like particle as described herein comprising a polynucleotide encoding a selective serotonin reuptake inhibitor.

In some embodiments, the administrating is via systemic delivery.

In some embodiments, the administrating is performed intravenously and/or intratumorly.

In some embodiments, the administrating targets the cerebral spinal fluid in intracerebroventricular, cisterna magna, subpial, and/or intrathecal. In other embodiments, the administrating is not performed intraparenchymally.

### Dosing and Treatment Regimens

Administration frequencies for a pharmaceutical composition comprising the recombinant zika virus or the zika-virus-like particle provided herein may vary based on the method being practiced, the physical characteristics of the subject, the severity of the cancer, cancer type, and the formulation and the means used to administer the composition.

The duration of treatment will be based on the condition being treated and may be determined by the attending physician. The duration of administration, in many instances, varies depending on a number of factors. Exemplary factors include, without limitation, patient response, severity of symptoms, and cancer type. Under some conditions, treatment is continued for a number of days, weeks, or months. Under other conditions, complete treatment is achieve through administering one, two or three dose of the pharmaceutical composition over the entire course of treatment. In certain aspects, complete treatment can be achieved using a single dose of the pharmaceutical composition.

In certain embodiments wherein a patient's status does improve, the dose of the recombinant zika virus or the zika-virus-like particle or pharmaceutical composition thereof described herein being administered may be temporarily reduced or temporarily suspended for a certain length of time (i.e. a “drug holiday”).

In certain embodiments the dose of the composition being administered is temporarily reduced or temporarily suspended for a certain length of time (i.e. a “drug diversion”).

In some embodiments, once improvement of the patient's conditions has occurred, a maintenance dose is administered if necessary. Subsequently, in certain embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of the recombinant zika virus or the zika-virus-like particle provided herein varies depending upon factors such as the particular virus, disease condition and its severity, the identity (e.g., weight, sex) of the subject in need of treatment, but can nevertheless be determined according to the particular circumstances surrounding the case. In some embodiments, the desired dose is conveniently presented in a single dose or in divided doses administered simultaneously (or over a short period of time) or at appropriate intervals, for example as two, three, four or more sub-doses per day.

In some embodiments, administration of the recombinant zika virus or the zika-virus-like particle provided herein depending on the titer, and the titer of the recombinant zika virus or the zika-virus-like particle is about 10⁶ PFU/mL to about 10¹⁰ PFU/mL.

In some embodiments, amount of the recombinant zika virus or the zika-virus-like particle of this disclosure, administered to a subject can be between about 10³ and 10¹² infectious viral particles or plaque forming units (PFU).

In some embodiments, the recombinant zika virus or the zika-virus-like particle of this disclosure can be administered at a dose that can comprise about 10³ viral particles/dose to about 10¹⁴ viral particles/dose.

In some embodiments, the recombinant zika virus or the zika-virus-like particle of this disclosure can be administered at a dose that can comprise about 10³ PFU/kg to about 10¹⁴ PFU/kg.

In some embodiments, the recombinant zika virus or the zika-virus-like particle of this disclosure can be administered at a dose that can comprise about 10³ viral particles/kg to about 10¹⁴ viral particles/kg.

### Pharmaceutical Compositions and Formulations of the Recombinant Zika Virus or the Zika-Virus-Like Particle

Provided herein are recombinant zika viruses or zika-virus-like particles formulated into pharmaceutical compositions.

Pharmaceutical compositions are formulated in a conventional manner using one or more pharmaceutically acceptable inactive ingredients that facilitate processing of the active agent into preparations that can be used pharmaceutically. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure. For some virus delivery methods, a pharmaceutically acceptable vehicle is selected from known pharmaceutically acceptable vehicles for delivery, and should be one in which the virus is stable.

Further provided herein are pharmaceutical compositions that include a virus; a pharmaceutically acceptable inactive ingredient; other medicinal or pharmaceutical agent; carrier; adjuvant; preserving, stabilizing, wetting or emulsifying agent; solution promoter; salt; buffer; excipients; binder; filling agent; suspending agent; flavoring agent; sweetening agents; disintegrating agent; dispersing agent; surfactants; lubricant; colorant; diluent; solubilizer; moistening agent; plasticizers; penetration enhancer; anti-foam agent; antioxidant; preservative; or a combination thereof.

Another aspect of the present disclosure provides a pharmaceutical composition comprising a recombinant zika virus as described herein. In some embodiments, the pharmaceutical composition can comprise a solubilizing agent and an excipient. In some embodiments, the excipient can comprise one or more of a buffering agent, a stabilizer, an antioxidant, a binder, a diluent, a dispersing agent, a rate controlling agent, a lubricant, a glidant, a disintegrant, a plasticizer, a preservative, or any combinations thereof. In some embodiments, the excipient can comprise di-sodium hydrogen phosphate dihydrate, sodium dihydrogen phosphate dihydrate, sodium chloride, myo-inositol, sorbitol, or any combinations thereof. In some embodiments, the pharmaceutical composition does not comprise a preservative. In some embodiments, the pharmaceutical composition can comprise one or more of a preservative, a diluent, and a carrier. In some embodiments, the pharmaceutical composition can comprise an additional active ingredient or a salt thereof. In some embodiments, the solubilizing agent can be sterile water. In some embodiments, the pharmaceutical composition can comprise an additional active ingredient, wherein the additional active ingredient can be a further oncolytic virus.

Another aspect of the present disclosure provides a method of enhancing therapeutic effect of an oncolytic virus upon systemic delivery of the virus to a subject, comprising a systemic administration of the recombinant zika virus or the zika-virus-like particle as disclosed herein, the recombinant zika virus or the zika-virus-like particle as described herein, or a pharmaceutical composition as disclosed herein.

Pharmaceutical compositions containing a modified virus, the recombinant zika virus or the zika-virus-like particle as described herein, can be prepared as solutions, dispersions in glycerol, liquid polyethylene glycols, in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein can comprise a stabilizer and a buffer. In some embodiments, a pharmaceutical composition as described herein can comprise a solubilizer, such as sterile water, Tris-buffer. In some embodiments, a pharmaceutical composition as described herein can comprise an excipient. An excipient can be an excipient described in the Handbook of Pharmaceutical Excipients, American Pharmaceutical Association (1986). Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. In some embodiments an excipient can comprise a preservative. Non-limiting examples of suitable preservatives can include antioxidants and antimicrobials. In some embodiments a pharmaceutical composition as described herein can comprise a binder as an excipient. In some embodiments a pharmaceutical composition as described herein can comprise a lubricant as an excipient. In some embodiments a pharmaceutical formulation can comprise a dispersion enhancer as an excipient. In some embodiments a pharmaceutical composition as described herein can comprise a disintegrant as an excipient. In some instances, a pharmaceutical composition as described herein can comprise a chelator.

Also contemplated are combination products that include one or more recombinant zika virus or one or more zika-virus-like particle described herein.

Under ordinary conditions of storage and use, the pharmaceutical compositions as described herein can comprise a preservative to prevent the growth of microorganisms. In certain examples, the pharmaceutical compositions as described herein may not comprise a preservative. The pharmaceutical forms suitable for injectable use can include sterile aqueous solutions or dispersions and sterile powders for the extemporaneous preparation of sterile injectable solutions or dispersions. The prevention of the action of microorganisms can be brought about by various antibacterial and antifungal agents.

For parenteral administration in an aqueous solution, for example, the liquid dosage form can be suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage may be dissolved in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions can be prepared by incorporating the recombinant zika virus or the zika-virus-like particle according to the present disclosure, the recombinant zika virus or the zika-virus-like particle as described herein or a pharmaceutical composition containing the same, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein may be formulated in a neutral or salt form. Upon formulation, the pharmaceutical compositions can be administered in a manner compatible with the dosage formulation and in such amount as is therapeutically effective.

In certain embodiments, a pharmaceutical composition of this disclosure can comprise an effective amount of a recombinant virus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the recombinant zika virus or the zika-virus-like particle or any other suitable vehicle, delivery or dispensing means or material. Such carriers can be formulated by conventional methods and can be administered to the subject at an effective amount.

### Kits of the Recombinant Zika Virus or the Zika-Virus-Like Particle

In one aspect of the disclosure, provided herein are kits which include one or more reagents or devices for the performance of the methods disclosed herein. In some embodiments, the kit comprises the recombinant zika virus or the zika-virus-like particle provided herein. In some embodiments, the kit comprises a means to administrate the recombinant zika virus or the zika-virus-like particle provided herein.

In some embodiments, the kit comprises suitable instructions in order to perform the methods of the kit. The instructions may provide information of performing any of the methods disclosed herein, whether or not the methods may be performed using only the reagents provided in the kit. The kit and instructions may require additional reagents or systems.

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of the recombinant zika virus or the zika-virus-like particle described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

In some embodiments, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a pharmaceutical composition comprising the recombinant zika virus or the zika-virus-like particle provided herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing the recombinant zika virus or the zika-virus-like particle described herein formulated in a compatible pharmaceutical carrier can also be prepared, placed in an appropriate container, and labeled for treatment of an indicated condition.

### Nucleic Acid Composition Comprising Regulatory Regions of Zika Genome

A wild type zika genome comprises several regulatory regions, such as 5′ UTR and 3′ UTR. UTRs are found to be important in viral replication and immune modulation. Elements in UTRs are found to be essential for genome cyclization, resulting in initiation of RNA synthesis.

Ribozymes are self-cleaving RNAs. Small ribozyme motifs mainly fall within four types: hammerhead, hairpin, Varkud satellite (VS), and hepatitis delta virus (HDV).

In some embodiments, a nucleic acid composition described herein comprises a polynucleotide encoding a ribozyme. In some embodiments, a zika virus or zika-virus-like particle described herein comprises a ribozyme.

In one aspect, provided herein is a nucleic acid composition comprising (i) a polynucleotide encoding a ribozyme (ii) 5′untranslated region (UTR) of a zika virus; (iii) 3′UTR of the zika virus; and (iv) a polynucleotide exogenous to the zika virus. In some embodiments, the ribozyme is a hammerhead ribozyme or a HDV ribozyme. In some embodiments, the nucleic acid further comprises a polynucleotide encoding a second ribozyme. In some specific embodiments, the second ribozyme is a hammerhead ribozyme or a hepatitis delta virus (HDV) ribozyme. In some embodiments, the polynucleotide encoding the ribozyme encodes a hammerhead ribozyme and the polynucleotide encoding the second ribozyme encodes an HDV ribozyme. In some embodiments, the nucleic acid composition further comprises a C-partial.

In some embodiments, the zika virus is an African zika virus, an Asian zika virus, a Brazilian zika virus component, or a combination of one or two thereof. In specific embodiments, the zika virus is African MR766 strain.

### Exogenous Polynucleotides

In some embodiments, the exogeneous polynucleotide is an antigen or an antigenic epitope thereof. In some embodiments, the antigen or an antigenic epitope thereof is disease associated. In specific embodiments, the antigen or an antigenic epitope thereof is from a pathogen.

In some embodiments, the pathogen is a virus. In specific embodiments, the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

In some embodiments, the pathogen is a bacteria. In specific embodiments, the bacteria is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

In some embodiments, the pathogen is a fungus, an amoeba, or a parasite. In specific embodiments, the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

In some embodiments, the exogeneous polynucleotide encodes a gene editing tool. In specific embodiments, the gene editing tool is selected from a group consisting of meganuclease associated agents, CRISPR associated agents, TALEN associated agents, and zinc finger associated agents.

In some embodiments, the exogeneous polynucleotide is a small interfering RNA (siRNA), antisense RNA, micro RNA (miRNA), small or short hairpin RNA (shRNA), guide RNA (gRNA), clustered regularly interspaced short palindromic repeat RNA (crRNA), trans-activating clustered regularly interspaced short palindromic repeat RNA (tracrRNA), immune-stimulating oligonucleotide, antisense nucleic acid, or ribozyme. In specific embodiments, the exogeneous polynucleotide targets beta-secretase 1 (BACE1) and/or amyloid precursor protein (APP).

In some embodiments, the exogeneous polynucleotide encodes a polypeptide associated with a genetic disorder. In specific embodiments, the polypeptide is brain-derived neurotrophin factor (BDNF), nerve growth factor (NGF), Neprilysin inhibitors (NEP), endothelin converting enzyme (ECE), cathepsin B (CTSB), apolipoprotein E 2 (APOE2), SH3 and multiple ankyrin repeat domains protein (SHANK), neurturin (NRTN), glial cell-derived neurotrophic factor (GDNF), cerebral dopamine neurotrophic factor (CDNF), vascular endothelial growth factor A (VEGF-A), or aromatic L-amino acid decarboxylase (AADC).

In some embodiments, the exogeneous polynucleotide encodes a therapeutic agent or a diagnostic agent. In specific embodiments, the therapeutic agent is an antibody-based therapeutic agent, a hormone, a cytokine, an inhibitor or antagonist of an immune checkpoint regulator, an immune stimulatory molecule, or an agonist of an immune co-stimulatory molecule. In further embodiments, the inhibitor or antagonist of an immune checkpoint regulator is an anti-PD1 antibody. In another embodiments, the antibody-based therapeutic agent is an antibody, a functional fragment of an antibody, a chimeric antigen receptor (CAR), or a T cell receptor (TCR). In still other embodiments, the cytokine comprises lymphokine, monokine, polypeptide hormone, growth hormone, parathyroid hormone, thyroxine, insulin, proinsulin, relaxin, prorelaxin, glycoprotein hormone, follicle stimulating hormone (FSH), thyroid stimulating hormone (TSH), luteinizing hormone (LH), hepatic growth factor, fibroblast growth factor, prolactin, placental lactogen, tumor necrosis factor-a and -β, mullerian-inhibiting substance, mouse gonadotropin-associated peptide, inhibin, activin, vascular endothelial growth factor, integrin, thrombopoietin (TPO), nerve growth factor, NGF-β, platelet growth factor, transforming growth factor (TGF), TGF-a, TGF-β, insulin-like growth factor-I and -II, erythropoietin (EPO), osteoinductive factor, interferon, such as interferon-a, -β and -γ, colony stimulating factor (CSF), macrophage-CSF (M-CSF), granulocyte-macrophage-CSF (GM-CSF), granulocyte-CSF (GCSF), interleukin (IL), tumor necrosis factor, TNF-a, TNF-β, LIF, or kit ligand (KL), or a combination of two or more thereof.

In some embodiments, the exogeneous polynucleotide is about or up to about 10000, 9000, 8000, 7000, 6000, or 5000 nucleotides long. For example, the exogenous polynucleotide is about 50 to about 10000, 9000, 8000, 7000, 6000, or 5000 nucleotides long. The exogenous polynucleotide may be about 100 to about 10000, 9000, 8000, 7000, 6000, or 5000 nucleotides long. The exogenous polynucleotide may be about 50 to about 6000 nucleotides long. The exogenous polynucleotide may be about 100 to about 6000 nucleotides long. The exogenous polynucleotide may be about 50 to about 5000 nucleotides long. The exogenous polynucleotide may be about 100 to about 5000 nucleotides long.

In some embodiments, the exogeneous polynucleotide is about 0.1 kb nucleotides long. In other embodiments, the exogenous sequence is about 0.2 kb long. In other embodiments, the exogenous sequence is about 0.3 kb long. In other embodiments, the exogenous sequence is about 0.4 kb long. In other embodiments, the exogenous sequence is about 0.5 kb long. In other embodiments, the exogenous sequence is about 0.6 kb long. In other embodiments, the exogenous sequence is about 0.7 kb long. In other embodiments, the exogenous sequence is about 0.8 kb long. In other embodiments, the exogenous sequence is about 0.9 kb long. In other embodiments, the exogenous sequence is about 1 kb nucleotides long. In other embodiments, the exogeneous polynucleotide is about 2 kb nucleotides long. In some embodiments, the exogeneous polynucleotide is about 3 kb nucleotides long. In some embodiments, the exogeneous polynucleotide is about 4 kb nucleotides long. In some embodiments, the exogeneous polynucleotide is about 5 kb nucleotides long. In some embodiments, the exogeneous polynucleotide is about 6 kb nucleotides long. In some embodiments, the exogeneous polynucleotide is about 7 kb nucleotides long. In some embodiments, the exogeneous polynucleotide is about 8 kb nucleotides long. In some embodiments, the exogeneous polynucleotide is about 9 kb nucleotides long. In some embodiments, the exogeneous polynucleotide is about 10 kb nucleotides long. In some embodiments, the exogeneous polynucleotide is about 11 kb nucleotides long. In some embodiments, the exogeneous polynucleotide is about 12 kb nucleotides long. In some embodiments, the exogeneous polynucleotide is about 13 kb nucleotides long. In some embodiments, the exogeneous polynucleotide is about 14 kb nucleotides long. In some embodiments, the exogeneous polynucleotide is about 15 kb nucleotides long. In some embodiments, the exogeneous polynucleotide is about 16 kb nucleotides long. In some embodiments, the exogeneous polynucleotide is about 17 kb nucleotides long. In some embodiments, the exogeneous polynucleotide is about 18 kb nucleotides long. In some embodiments, the exogeneous polynucleotide is about 19 kb nucleotides long. In some embodiments, the exogeneous polynucleotide is about 20 kb nucleotides long.

### Other Features of Nucleic Acid Composition Comprising Regulatory Regions of Zika Genome

In some embodiments, the nucleic acid composition further comprises one or more expression control elements in operable linkage that confers expression of the nucleic acid composition in vitro. In some embodiments, the expression control element is a promoter that drives expression of the nucleic acid complex in vitro. In specific embodiments, the promoter is T7, T3, SP6 or any phage promoter.

In another aspect, provided herein is a pharmaceutical composition comprising the nucleic acid composition described herein with a pharmaceutically acceptable salt or derivative thereof.

### Method of Uses of Nucleic Acid Composition

In another aspect, provided herein is method of delivering an exogeneous polynucleotide to a target cell, the method comprising administering to the target cell a compound produced from a nucleic acid composition as described herein comprising the exogeneous polynucleotide.

In some embodiments, the compound is produced by encapsulating a transcript produced from the nucleic acid composition described herein with a lipid-based agent. In specific embodiments, the transcript is capless. In another specific embodiments, the transcript is produced by in vitro transcribing the nucleic acid composition.

In some embodiments, the lipid-based agent is a lipofectamine-related reagent, a liposome, or a lipid nanoparticle.

In some embodiments, the lipid-based agent described herein is a cationic lipid, a neutral lipid, and a polyethyleneglycol conjugate, such as a PEG-diacylglycerol, PEG-diacylglycamide, PEG-cholesterol, or PEG-DMB conjugate, cholesterol, or a cholesterol derivative.

Suitable cationic lipids include those cationic lipids which carry a net negative charge at a selected pH, such as physiological pH. Particularly useful cationic lipids include those having a relatively small head group, such as a tertiary amine, quaternary amine or guanidine head group, and sterically hindered asymmetric lipid chains. In any of the embodiments described herein, the cationic lipid can be N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxy) propyl)-N,N,N-trimethylammonium chloride (DOTMA), N,N-dimethyl-2,3-dioleyloxy) propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), Dioleoyloxy-N-[2-sperminecarboxamido)ethyl}-N,N-dimethyl-1-propanaminiumtrifluoroacetate (DOSPA), Dioctadecylamidoglycyl spermine (DOGS), DC-Chol, 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-octadecadienoxy) propane (CLinDMA), 2-[5′-(cholest-5-en-3β-oxy)-3′-oxapentoxy)-3-dimethyl-1-(cis, cis-9′,12′-octadecadienoxy) propane (CpLinDMA), N,N-Dimethyl-3,4-dioleyloxybenzylamine (DMOBA), 1,2-N,N′-Dioleylcarbamyl-3-dimethylaminopropane (DOcarbDAP), and/or a mixture thereof, as well as other cationic lipids sharing similar properties. The above cationic lipids can include various differing salts as are known in the art.

In some embodiments, the head group of the cationic lipid can be attached to the lipid chain via a cleavable or non-cleavable linker, such as a linker described herein or otherwise known in the art. Non-limiting examples of suitable linkers include those comprising a C1 to C10 alkyl, alkyl ether, polyether, polyethylene glycol, acetal, amide, carbonyl, carbamide, carbamate, carbonate, ester (i.e., monoester, diester), or succinyl.

Suitable neutral lipids include those comprising any of a variety of neutral uncharged, zwitterionic or anionic lipids capable of producing a stable complex. They are preferably neutral, although they can alternatively be positively or negatively charged. In any of the embodiments described herein, suitable neutral lipids include those selected from compounds having formulae NLI-NL VII, dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG),-phosphatidylet-hanolamine (POPE) and dioleoyl-phosphatidylethariolamine 4-(N-maleimidomethyl)-cyclohexane-1-carboxylate (DOPE-mal), cholesterol, as well as other neutral lipids described herein below, and/or a mixture thereof.

Suitable polyethyleneglycol-diacylglycerol or polyethyleneglycol-diacylglycamide (PEG-DAG) conjugates include those comprising a dialkylglycerol or dialkylglycamide group having alkyl chain length independently comprising from about C4 to about C40 saturated or unsaturated carbon atoms. The dialkylglycerol or dialkylglycamide group can further comprise one or more substituted alkyl groups. In any of the embodiments described herein, the PEG conjugate can be selected from PEG-dilaurylglycerol (C12), PEG-dimyristylglycerol (C14), PEG-dipalmitoylglycerol (C16), PEG-disterylglycerol (C18), PEG-dilaurylglycamide (C12), PEG-dimyristylglycamide (C14), PEG-dipalmitoylglycamide (C16), and PEG-disterylglycamide (C18), PEG-cholesterol (1-[8′-(Cholest-5-en-3β-oxy) carboxamido-3′,6′-dioxaoctanyl]carbamoyl-ω-methyl-poly(ethylene glycol), and PEG-DMB (3,4-Ditetradecoxylbenzyl-ω-methyl-poly(ethylene glycol) ether).

In another aspect, provided herein is a method of triggering or boosting an immune response in a subject, the method comprising administering to the subject an effective amount of a compound produced from the nucleic acid composition as described herein comprising an antigen or an antigenic epitope thereof.

In some embodiments, the compound is produced by encapsulating a transcript produced from the nucleic acid composition described herein with a lipid-based agent. In specific embodiments, the transcript is capless. In another specific embodiments, the transcript is produced by in vitro transcribing the nucleic acid composition.

In some embodiments, the lipid-based agent is a lipofectamine-related reagent, a liposome, or a lipid nanoparticle.

In some embodiments, the administrating is performed intramuscularly.

In some embodiments, the antigen or an antigenic epitope thereof is from a pathogen.

In some embodiments, the pathogen is a virus. In specific embodiments, the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

In some embodiments, the pathogen is a bacteria. In specific embodiments, the bacteria is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

In some embodiments, the pathogen is a fungus, an amoeba, or a parasite. In specific embodiments, the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

### Non-Limiting Example Embodiments

1. A nucleic acid composition comprising (i) a polynucleotide encoding a zika virus 5′UTR or a derivative of the zika virus 5′ UTR, a polynucleotide encoding a zika virus 3′UTR or a derivative of the zika virus 3′ UTR, a polynucleotide encoding a zika virus capsid protein (C) or the derivative of the zika virus C, a polynucleotide encoding a zika virus viral membrane protein (prM/M) or the derivative of the zika virus prM/M, a polynucleotide encoding a zika virus viral envelope protein (E) or the derivative of the zika virus E, or any combination thereof; and (ii) a polynucleotide exogenous to the zika virus.

2. The nucleic acid composition of embodiment 1, wherein the nucleic acid composition comprises the polynucleotide encoding the zika virus 5′UTR, the polynucleotide encoding the zika virus 3′UTR, the polynucleotide encoding the zika virus C or the derivative of the zika virus C, the polynucleotide encoding the zika virus prM/M or the derivative of the zika virus prM/M, and the polynucleotide encoding the zika virus E or the derivative of the zika virus E. 3. The nucleic acid composition of embodiment 1 or embodiment 2, wherein the polynucleotide encoding the zika virus 5′UTR or a derivative of the zika virus 5′UTR, the polynucleotide encoding the zika virus 3′UTR or a derivative of zika virus 3′UTR, the polynucleotide encoding the zika virus C or the derivative of the zika virus C, the polynucleotide encoding the zika virus prM/M or the derivative of the zika virus prM/M, and the polynucleotide encoding the zika virus E or the derivative of the zika virus E are expressed on one or two or more separate nucleic acids.

4. The nucleic acid composition of the preceding embodiments, wherein the polynucleotide encoding the derivative of the zika virus 5′UTR comprises at least one substitution, at least one deletion, and/or at least one insertion as compared to a wild-type zika virus 5′UTR.

5. The nucleic acid composition of the proceeding embodiments, wherein the zika virus 5′UTR or the derivative of the zika virus 5′UTR comprises an amino acid sequence at least 90%, 91%, 92%, 93%, 94%, 95%, 96%, 97%, 98%, 99%, or 100% identical to a sequence of Table 1A, Table 1C, Table 1H, or Table 2.

6. The nucleic acid composition of the preceding embodiments, wherein the polynucleotide encoding the derivative of the zika virus 3′UTR comprises at least one substitution, at least one deletion, and/or at least one insertion as compared to a wild-type zika virus 3′UTR.

7. The nucleic acid composition of the preceding embodiments, wherein the zika virus 3′UTR or the derivative of the zika virus 3′UTR comprises an amino acid sequence at least 90%, 91%, 92%, 93%, 94%, 95%, 96%, 97%, 98%, 99%, or 100% identical to a sequence of Table 1A, Table 1B, Table 1G, or Table 2.

8 The nucleic acid composition of the preceding embodiments, wherein the polynucleotide encoding the derivative of the zika virus C comprises at least one substitution, at least one deletion, and/or at least one insertion as compared to a wild-type zika virus C.

9. The nucleic acid composition of the preceding embodiments, wherein the zika virus C or the derivative of the zika virus C comprises an amino acid sequence at least 90%, 91%, 92%, 93%, 94%, 95%, 96%, 97%, 98%, 99%, or 100% identical to a sequence of Table 1A, Table 1D, Table 1I, Table 2, or Table 3.

10. The nucleic acid composition of the preceding embodiments, wherein the polynucleotide encoding the derivative of the zika virus prM/M comprises at least one substitution, at least one deletion, and/or at least one insertion as compared to a wild-type zika virus prM/M.

11. The nucleic acid composition of the preceding embodiments, wherein the zika virus prM/M or the derivative of the zika virus prM/M comprises a sequence at least 90%, 91%, 92%, 93%, 94%, 95%, 96%, 97%, 98%, 99%, or 100% identical to a sequence of Table 1A, Table 1F, or Table 1K.

12. The nucleic acid composition of the preceding embodiments, wherein the polynucleotide encoding the derivative of the zika virus E comprises at least one substitution, at least one deletion, and/or at least one insertion as compared to a wild-type zika virus E.

13. The nucleic acid composition of the preceding embodiments, wherein the polynucleotide encoding E is translated into a wild zika virus type E.

14. The nucleic acid composition of the preceding embodiments, wherein the zika virus E or the derivative of the zika virus E comprises a sequence at least 90%, 91%, 92%, 93%, 94%, 95%, 96%, 97%, 98%, 99%, or 100% identical to sequence of Table 1A, Table IE, or Table 1J.

15. The nucleic acid composition of the preceding embodiments, wherein the nucleic acid composition comprises the 5′ untranslated region (5′ UTR) of the zika virus.

16. The nucleic acid composition of the preceding embodiments, wherein the nucleic acid composition comprises the 3′ untranslated region (3′ UTR) of the zika virus.

17. The nucleic acid composition of the preceding embodiments, wherein the nucleic acid composition does not comprise a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii).

18. The nucleic acid composition of the preceding embodiments, wherein nucleic acid composition further comprises a polynucleotide encoding one or more non-structural (NS) proteins selected from (i) a NS1, (ii) a NS2A, (iii) a NS2B, (iv) a NS3, (v) a NS4A, (vi) a NS4B, (vii) a NS5, or (viii) two or more of (i)-(vii).

19. The nucleic acid composition of embodiment 17 or embodiment 18, wherein the NS1 is a zika virus NS1, the NS2A is a zika virus NS2A, the NS2B is a zika virus NS2B, the NS3 is a zika virus NS3, the NS4A is a zika virus NS4A, the NS4B is a zika virus NS4B, or the NS5 is a zika virus NS5, or any combination of two or more thereof.

20. The nucleic acid composition of the preceding embodiments, wherein the components of the nucleic acid composition comprise African zika virus components, Asian zika virus components, or Brazilian zika virus components, or a combination thereof.

21. The nucleic acid composition of embodiment 20, wherein the zika virus is African MR766 strain.

22. The nucleic acid composition of the preceding embodiments, wherein the exogeneous polynucleotide is an antigen or an antigenic epitope thereof.

23. The nucleic acid composition of embodiment 22, wherein the antigen or an antigenic epitope thereof is disease associated.

24. The nucleic acid composition of embodiment 23, wherein the antigen or an antigenic epitope thereof is from a pathogen.

25. The nucleic acid composition of embodiment 24, wherein the pathogen is a virus.

26. The nucleic acid composition of embodiment 25, wherein the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

27. The nucleic acid composition of embodiment 24, wherein the pathogen is a bacteria

28. The nucleic acid composition of embodiment 27, wherein the bacteria is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

29. The nucleic acid composition of embodiment 24, wherein the pathogen is a fungus, an amoeba, or a parasite.

30. The nucleic acid composition of embodiment 29, wherein the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

31. The nucleic acid composition of any one of embodiments 1-30, wherein the exogeneous polynucleotide encodes a gene editing tool.

32 The nucleic acid composition of embodiment 31, wherein the gene editing tool is selected from a group consisting of meganuclease associated agents, CRISPR associated agents, TALEN associated agents, and zinc finger associated agents.

33. The nucleic acid composition of any one of embodiments 1-30, wherein the exogeneous polynucleotide is a small interfering RNA (siRNA), antisense RNA, micro RNA (miRNA), small or short hairpin RNA (shRNA), guide RNA (gRNA), clustered regularly interspaced short palindromic repeat RNA (crRNA), trans-activating clustered regularly interspaced short palindromic repeat RNA (tracrRNA), immune-stimulating oligonucleotide, antisense nucleic acid, or ribozyme.

34. The nucleic acid composition of embodiment 33, wherein the exogeneous polynucleotide targets beta-secretase 1 (BACE1) and/or amyloid precursor protein (APP).

35. The nucleic acid composition of any one of embodiments 1-30, wherein the exogeneous polynucleotide encodes a polypeptide associated with a genetic disorder.

36. The nucleic acid composition of embodiment 35, wherein the polypeptide is brain-derived neurotrophin factor (BDNF), nerve growth factor (NGF), Neprilysin inhibitors (NEP), endothelin converting enzyme (ECE), cathepsin B (CTSB), apolipoprotein E 2 (APOE2), SH3 and multiple ankyrin repeat domains protein (SHANK), neurturin (NRTN), glial cell-derived neurotrophic factor (GDNF), cerebral dopamine neurotrophic factor (CDNF), vascular endothelial growth factor A (VEGF-A), or aromatic L-amino acid decarboxylase (AADC).

37. The nucleic acid composition of any one of embodiments 1-30, wherein the exogeneous polynucleotide encodes a therapeutic agent or a diagnostic agent.

38. The nucleic acid composition of embodiment 37, wherein the therapeutic agent is an antibody-based therapeutic agent, a hormone, a cytokine, an inhibitor or antagonist of an immune checkpoint regulator, an immune stimulatory molecule, or an agonist of an immune co-stimulatory molecule.

39. The nucleic acid composition of embodiment 38, wherein the inhibitor or antagonist of an immune checkpoint regulator is an anti-PD1 antibody.

40. The nucleic acid composition of embodiment 38, wherein the antibody-based therapeutic agent is an antibody, a functional fragment of an antibody, a chimeric antigen receptor (CAR), or a T cell receptor (TCR).

41. The nucleic acid composition of embodiment 38, wherein the cytokine comprises lymphokine, monokine, polypeptide hormone, growth hormone, parathyroid hormone, thyroxine, insulin, proinsulin, relaxin, prorelaxin, glycoprotein hormone, follicle stimulating hormone (FSH), thyroid stimulating hormone (TSH), luteinizing hormone (LH), hepatic growth factor, fibroblast growth factor, prolactin, placental lactogen, tumor necrosis factor-a and -β, mullerian-inhibiting substance, mouse gonadotropin-associated peptide, inhibin, activin, vascular endothelial growth factor, integrin, thrombopoietin (TPO), nerve growth factor, NGF-β, platelet growth factor, transforming growth factor (TGF), TGF-a, TGF-β, insulin-like growth factor-I and -II, erythropoietin (EPO), osteoinductive factor, interferon, such as interferon-a, -β and -γ, colony stimulating factor (CSF), macrophage-CSF (M-CSF), granulocyte-macrophage-CSF (GM-CSF), granulocyte-CSF (GCSF), interleukin (IL), tumor necrosis factor, TNF-a, TNF-β, LIF, or kit ligand (KL), or a combination of two or more thereof.

42. The nucleic acid composition of the preceding embodiments, wherein the exogeneous polynucleotide is up to or about 10,000 nucleotides long.

43. The nucleic acid composition of the preceding embodiments, wherein components of the nucleic acid composition are expressed on one or more separate nucleic acids.

44. The nucleic acid composition of the preceding embodiments, further comprising one or more expression control elements in operable linkage that confers expression of the nucleic acid composition in vitro or in vivo.

45. The nucleic acid composition of embodiment 44, wherein the expression control element is a promoter that drives expression of the nucleic acid complex in vitro.

46. The nucleic acid composition of embodiment 45, wherein the promoter is T7, T3, SP6 or any phage promoter.

47. The nucleic acid composition of embodiment 44, wherein the expression control element is a promoter that drives expression of the nucleic acid complex in a target cell.

48. The nucleic acid composition of embodiment 47, wherein the promoter is CMV, SV40 or any eukaryotic promoter.

49. The nucleic acid composition of embodiment 47 or embodiment 48, wherein the target cell is a neuron, or a non-neuron cell, VERO, COS, CHO, C6/36, HeLa, HEK, or HepG2.

50. The nucleic acid composition of embodiment 47 or embodiment 48, wherein the target cell is an oligodendrocyte, microglia, or astrocyte.

51. A pharmaceutical composition comprising the nucleic acid composition of the preceding embodiments with a pharmaceutically acceptable salt or derivative thereof.

52. A recombinant zika virus or a zika-virus-like particle that is generated from expressing the nucleic acid of one of embodiments 1-51 in a producer cell, wherein the producer cell is further infected by a second zika virus, such that the zika virus C or the derivative of the zika virus C, the zika virus prM/M or the derivative of the zika virus prM/M, and the zika virus E or the derivative of the zika virus E are present in the recombinant zika virus or the zika-virus-like particle.

53. The recombinant zika virus or the zika-virus-like particle of embodiment 52, wherein the second zika virus is a wild-type zika virus.

54. The recombinant zika virus or the zika-virus-like particle of embodiment 53, wherein the wild-type zika virus is an African, an Asian and a Brazilian strain.

55. The recombinant zika virus or the zika-virus-like particle of embodiment 53, wherein the second zika virus is a modified zika virus.

56. The recombinant zika virus or the zika-virus-like particle of embodiment 55, wherein the modified zika virus comprises one or more microRNA-based gene-silencing machineries.

57. The recombinant zika virus or the zika-virus-like particle of embodiment 56, wherein the one or more microRNA-based gene-silencing machineries control viral replication.

58. A recombinant zika virus or a zika-virus-like particle that is generated from expressing the nucleic acid of embodiments 1-51 in a producer cell, without an infection of a second zika virus, wherein the zika virus C or the derivative of the zika virus C, the zika virus prM/M or the derivative of the zika virus prM/M, and the zika virus E or the derivative of the zika virus E are present in the recombinant zika virus or the zika-virus-like particle.

59. The recombinant zika virus or the zika-virus-like particle of one of embodiments 52-58, wherein the producer cell is a Vero E6, HEK, HEK 293T, HEK 293TT, FreeStyle™ 293-F Cell, HEK-293.2sus or C6/36 cell.

60. The recombinant zika virus or the zika-virus-like particle of one of embodiments 52-59, wherein the recombinant zika virus is replication competent.

61. The recombinant zika virus or the zika-virus-like particle of one of embodiments 52-60, wherein the recombinant zika virus is replication incompetent without lowering the vector titer or impairing expression of the exogeneous polynucleotide.

62. The recombinant zika virus or the zika-virus-like particle of one of embodiments 52-61, wherein the recombinant zika virus or the zika-virus-like particle has decreased insertional mutagenesis.

63. The recombinant zika virus or the zika-virus-like particle of one of embodiments 52-62, wherein the recombinant zika virus or the zika-virus-like particle has decreased immune response.

64. A pharmaceutical composition comprising the recombinant zika virus or the zika-virus-like particle of one of one of embodiments 52-63, with a pharmaceutically acceptable salt or derivative thereof.

65. A method of delivering an exogeneous polynucleotide to a target cell, the method comprising administering to the target cell with the recombinant zika virus or the zika-virus-like particle of one of embodiments 52-64 comprising the exogeneous polynucleotide.

66 The method of embodiment 65, wherein the target cell is a neuron or a non-neuron cell.

67. The method of embodiment 66, wherein the neuron is an oligodendrocyte, microglia, or astrocyte.

68. The method of embodiment 66, wherein the non-neuron cell is a prostate epithelial cell, a urethra epithelial cell, a Sertoli cell, a Leydig cell, a spermatogonium cell, or a retinal cell.

69. The method of one of embodiments 65-68, wherein the method is carried out in vitro, ex vivo, or in vivo.

70. The method of one of embodiments 65-69, wherein the target cell transiently expresses the exogeneous polynucleotide after delivery.

71. The method of one of embodiments 65-69, wherein the target cell persistently expresses the exogeneous polynucleotide after delivery.

72. A method of treating Alzheimer's disease in a subject in need thereof, the method comprising administering to the subject an effective amount of the recombinant zika virus or the zika-virus-like particle of one of embodiments 52-71 comprising a polynucleotide encoding a brain-derived neurotrophin factor (BDNF).

73. A method of treating autism in a subject in need thereof, the method comprising administering to the subject an effective amount of the recombinant zika virus or the zika-virus-like particle of one of embodiments 52-71 comprising a polynucleotide encoding SH3 and multiple ankyrin repeat domains protein (SHANK).

74. A method of treating Parkinson's disease in a subject in need thereof, the method comprising administering the subject an effective amount of the recombinant zika virus or the zika-virus-like particle one of embodiments 52-71 comprising a polynucleotide encoding aromatic L-amino acid decarboxylase (AADC).

75. The method of one of embodiments 65-74, wherein the administrating is via systemic delivery.

76. The method of one of embodiments 65-74, wherein the administrating is performed intravenously and/or intratumorly.

77. The method of embodiment one of embodiments 65-76, wherein the administrating targets the cerebral spinal fluid in intracerebroventricular, cisterna magna, subpial, and/or intrathecal.

78. The method of embodiment one of embodiments 65-77, wherein the administrating is not performed intraparenchymally.

79. A nucleic acid composition comprising (i) a polynucleotide encoding a ribozyme (ii) 5′untranslated region (UTR) of a zika virus; (iii) 3′UTR of the zika virus; and (iv) a polynucleotide exogenous to the zika virus;
optionally wherein the polynucleotide encoding the ribozyme is positioned before the 5′ UTR or after the 3′ UTR;
further optionally wherein the polynucleotide encoding a ribozyme is a first polynucleotide encoding a ribozyme and the nucleic acid composition comprises a second polynucleotide encoding a ribozyme, and the first polynucleotide is positioned before the 5′ UTR and the second polynucleotide is positioned after the 3′ UTR.

80. The nucleic acid composition of embodiment 79, wherein the ribozyme is a hammerhead ribozyme or a hepatitis delta virus (HDV) ribozyme.

81. The nucleic acid composition of embodiment 79 or embodiment 80, comprising a polynucleotide encoding a second ribozyme.

82. The nucleic acid composition of embodiment 81, wherein the second ribozyme is a hammerhead ribozyme or a hepatitis delta virus (HDV) ribozyme.

83. The nucleic acid composition of embodiment 81 or embodiment 82, wherein the polynucleotide encoding the ribozyme encodes a hammerhead ribozyme and the polynucleotide encoding the second ribozyme encodes a HDV ribozyme.

84. The nucleic acid composition of any one of embodiments 79-83, comprising a C-partial (e.g., a polynucleotide encoding a truncation of C, such as less than or about 30, 25, 20, 15, 10, or 5 amino acids of C).

85. The nucleic acid composition of any one of embodiments 79-84, wherein the zika virus is an African zika virus, an Asian zika virus, a Brazilian zika virus components, or a combination of one or two thereof.

86 The nucleic acid composition of any one of embodiments 79-85, wherein the zika virus is African MR766 strain.

87. The nucleic acid composition of any one of embodiments 79-86, wherein the exogeneous polynucleotide is an antigen or an antigenic epitope thereof.

88. The nucleic acid composition of embodiment 87, wherein the antigen or an antigenic epitope thereof is disease associated.

89. The nucleic acid composition of embodiment 88, wherein the antigen or an antigenic epitope thereof is from a pathogen.

90. The nucleic acid composition of embodiment 89, wherein the pathogen is a virus.

91. The nucleic acid composition of embodiment 90, wherein the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

92. The nucleic acid composition of embodiment 89, wherein the pathogen is a bacteria.

93. The nucleic acid composition of embodiment 92, wherein the bacteria is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

94. The nucleic acid composition of embodiment 89, wherein the pathogen is a fungus, an amoeba, or a parasite.

95. The nucleic acid composition of embodiment 94, wherein the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

96. The nucleic acid composition of any one of embodiments 79-95, wherein the exogeneous polynucleotide encodes a gene editing tool.

97. The nucleic acid composition of embodiment 96, wherein the gene editing tool is selected from a group consisting of meganuclease associated agents, CRISPR associated agents, TALEN associated agents, and zinc finger associated agents.

98. The nucleic acid composition of any one of embodiments 79-97, wherein the exogeneous polynucleotide is a small interfering RNA (siRNA), antisense RNA, micro RNA (miRNA), small or short hairpin RNA (shRNA), guide RNA (gRNA), clustered regularly interspaced short palindromic repeat RNA (crRNA), trans-activating clustered regularly interspaced short palindromic repeat RNA (tracrRNA), immune-stimulating oligonucleotide, antisense nucleic acid, or ribozyme.

99. The nucleic acid composition of embodiment 98, wherein the exogeneous polynucleotide targets BACE1 or APP.

100. The nucleic acid composition of any one of embodiments 79-99, wherein the exogeneous polynucleotide encodes a polypeptide associated with a genetic disorder.

101. The nucleic acid composition of embodiment 100, wherein the polypeptide is brain-derived neurotrophin factor (BDNF), nerve growth factor (NGF), Neprilysin inhibitors (NEP), endothelin converting enzyme (ECE), cathepsin B (CTSB), apolipoprotein E 2 (APOE2), SH3 and multiple ankyrin repeat domains protein (SHANK), neurturin (NRTN), glial cell-derived neurotrophic factor (GDNF), cerebral dopamine neurotrophic factor (CDNF), vascular endothelial growth factor A (VEGF-A), or aromatic L-amino acid decarboxylase (AADC).

102. The nucleic acid composition of any one of embodiments 79-101, wherein the exogeneous polynucleotide encodes a therapeutic agent or a diagnostic agent.

103. The nucleic acid composition of embodiment 102, wherein the therapeutic agent is an antibody-based therapeutic agent, a hormone, a cytokine, an inhibitor or antagonist of an immune checkpoint regulator, an immune stimulatory molecule, or an agonist of an immune co-stimulatory molecule.

104. The nucleic acid composition of embodiment 103, wherein the inhibitor or antagonist of an immune checkpoint regulator is an anti-PD1 antibody.

105. The nucleic acid composition of embodiment 103, wherein the antibody-based therapeutic agent is an antibody, a functional fragment of an antibody, a chimeric antigen receptor (CAR), or a T cell receptor (TCR).

106. The nucleic acid composition of embodiment 103, wherein the cytokine comprises lymphokine, monokine, polypeptide hormone, growth hormone, parathyroid hormone, thyroxine, insulin, proinsulin, relaxin, prorelaxin, glycoprotein hormone, follicle stimulating hormone (FSH), thyroid stimulating hormone (TSH), luteinizing hormone (LH), hepatic growth factor, fibroblast growth factor, prolactin, placental lactogen, tumor necrosis factor-a and -β, mullerian-inhibiting substance, mouse gonadotropin-associated peptide, inhibin, activin, vascular endothelial growth factor, integrin, thrombopoietin (TPO), nerve growth factor, NGF-β, platelet growth factor, transforming growth factor (TGF), TGF-a, TGF-β, insulin-like growth factor-I and -II, erythropoietin (EPO), osteoinductive factor, interferon, such as interferon-a, -β and -γ, colony stimulating factor (CSF), macrophage-CSF (M-CSF), granulocyte-macrophage-CSF (GM-CSF), granulocyte-CSF (GCSF), interleukin (IL), tumor necrosis factor, TNF-a, TNF-β, LIF, or kit ligand (KL), or a combination of two or more thereof.

107. The nucleic acid composition of one of embodiments 79-106, wherein the exogeneous polynucleotide is up to or about 10,000 nucleotides long.

108. The nucleic acid composition of one of embodiments 79-107, comprising one or more expression control elements in operable linkage that confers expression of the nucleic acid composition in vitro.

109. The nucleic acid composition of embodiment 108, wherein the expression control element is a promoter that drives expression of the nucleic acid complex in vitro.

110. The nucleic acid composition of embodiment 109, wherein the promoter is T7, T3, SP6 or any phage promoter.

111. A pharmaceutical composition comprising the nucleic acid composition of one of embodiments 79-110 with a pharmaceutically acceptable salt or derivative thereof.

112. A method of delivering an exogeneous polynucleotide to a target cell, the method comprising applying to the target cell a compound produced from the nucleic acid composition of one of embodiments 79-111 comprising the exogeneous polynucleotide; optionally wherein the compound has a tropism for the target cell; further optionally wherein the target cell is a cell of the central nervous system; further optionally wherein the target cell is present in a subject and applying to the target cell comprises administering the compound to the subject; further optionally wherein the subject is a human or non-human animal.

113. The method of embodiment 112, wherein the compound is produced by encapsulating a transcript produced from the nucleic acid composition with a lipid-based agent.

114. The method of embodiment 113, wherein the transcript is capless.

115. The method of embodiment 113 or embodiment 114, wherein the transcript is produced by in vitro transcribing the nucleic acid composition.

116. The method of any one of embodiments 112-115, wherein the lipid-based agent is a lipofectamine-related reagent, a liposome, or a lipid nanoparticle.

117. A method of triggering or boosting an immune response in a subject, the method comprising administering to the subject an effective amount of a compound produced from the nucleic acid composition of one of embodiments 79-110 or the composition of embodiment 11 comprising an antigen or an antigenic epitope thereof; optionally wherein the subject is a human or non-human animal.

118. The method of embodiment 117, wherein the compound is produced by encapsulating a transcript produced from the nucleic acid composition with a lipid-based agent.

119. The method of embodiment 118, wherein the transcript is capless.

120. The method of embodiment 188 or embodiment 119, wherein the transcript is produced by in vitro transcribing the nucleic acid composition.

121. The method of any one of embodiments 117-120, wherein the lipid-based agent is a lipofectamine related reagent, a liposome, or a lipid nanoparticle.

122. The method of any one of embodiments 112-121, wherein the administrating is performed intramuscularly.

123. The method of any one of embodiments 112-122, wherein the antigen or an antigenic epitope thereof is from a pathogen.

124. The method of embodiment 123, wherein the pathogen is a virus.

125. The method of embodiment 124, wherein the virus is a human SARS coronavirus, an influenza A virus, an influenza B virus, an influenza C virus an ebolavirus, a hepatitis B virus, a hepatitis C virus, a herpes simplex virus, a human immunodeficiency virus (HIV), a human papillomavirus (HPV-6, HPV-11), a measles virus, a rabies virus, a poliovirus, or a yellow fever virus.

126. The method of embodiment 123, wherein the pathogen is a bacterium.

127. The method of embodiment 126, wherein the bacteria is Acinetobacter baumanii, Aggregatobacter actinomycetemcomitans, Bartonella bacilliformis, Bartonella. henselae, Bartonella quintana, Bifidobacterium Borrelia, Bortadella pertussis, Brucella sp, Burkholderia cepacis, Burkholderia psedomallei, Campylobacter jejuni, Cardiobacterium hominis, Campylobacter fetus, Chlamydia pneumonia, Chlymydia trahomatis, Clostridium difficile, Cyanobacteria, Eikennella corrodens, Enterobacter, Enterococcus faccium, Escherichia coli, Escherichia coli 0157, Franceilla tularensis, Fusobacterium nucleatum, Haemophilus influenza, Haemophilus aphrophilus, Haemophilus ducreyi, Haemophilus parainfluenzae, Helicobacter pylori, Kingella kingae, Klebsiella pneumonia, Legionella bacteria, Legionella pneumophila serogroup 1, Leptospria, Morganella morganii, Neisseria gonorrhoeae, Neisseria meningitidis, Proteus mirabilis, Proteus vulgaris, Proteus myxofaciens, Providencia rettgeri, Providencia alcalifaciens, Providencia stuartii, Pseudomonas aeruginosa, Pseudomonas paucimobilis, Pseudomonas putida, Pseudomonas fluorescens, Pseudomonas acidovorans, Rickettsiae, Salmonella enterica, Salmonella typhi, Salmonella paratyphi types A, B typhus, Salmonella. dublin, Salmonella arizonae, Salmonella choleraesuis, Serratia marcescens, Schigella dysenteriae, Schigella flexneri, Schigella boydii, Schigella sonnei, Treponema, Stenotrophomonas maltophilia, Vibrio cholerae, Vibrio mimicus, Vibrio alginolyticus, Vibrio hollisae, Vibrio parahaemolyticus, Vibrio vulnificus, Yersinia pestitis, Actinomycetes, Bacillus anthracis, Bacillus subtilis, Clostridium tetani, Clostridium. perfingens, Clostridium botulinum, Clostridium tetani. Corynebacterium diphtheriae, Enterococcus faecalis, Enterococcus faecium, Erysipelothrix ruhsiopathiae, Listeria monocytogenes, Mycobacterium leprae, Mycobacterium tuberculosis, Mycoplasma, Nocardia, Propionibacerium, Pseudomonas aeruginosa, Pneumococci, Staphylococcus aureus, Staphylococcus epidermidis, methicillin resistant Staphylococcus aureus (MRSA), vancomycin resistant Staphylococcus aureus (VRSA), Staphylococcus lugdunensis, Staphylococcus saprophyticus, Streptococcus pneumonia, Streptococcus pyogenes, or Streptococcus mutants.

128. The method of embodiment 123, wherein the pathogen is a fungus, an amoeba, or a parasite.

129. The method of embodiment 128, wherein the fungus, the amoeba, or the parasite is Acanthamoeba spp, American tryppanosomiasis, Balamuthia mandnillanis, Babesia divergenes, Babesia bigemina, Babesia equi, Babesia microfti, Babesia duncani, Balantidium coli, Blastocystis spp Cryptosporidium spp, Cyclospora cayetanensis, dientamoeba fragilis, Diphyllobothrium latum, Leishmania amazonesis, Naegleria fowderi, Plasmodium falciparum, Plasmodium vivax, Plasmodium ovale curtisi, Plasmodium malariae, Rhinosporidium seeberi, Sarcocystis bovihominis, Sarcocystiss suihominis, Toxoplasma gondii, Trichmonas vaginalis, Trypanosoma brucei, Trypanosoma cruzi, and Taenia multiceps.

TABLE 1A
Non-limiting example Zika sequences

| | Example Zika |
|---|---|
| SEQ ID NO | virus components |
| 1 | 5′UTR |
| 2 | 3′UTR |
| 3 | C |
| 4 | prM/M |
| 5 | E |
| 6 | NS1 |
| 7 | NS2A |
| 8 | NS2B |
| 9 | NS3 |
| 10 | NS4A |
| 11 | 2k |
| 12 | NS4B |
| 13 | NS5 |

TABLE 1B
Non-limiting example Zika 3′ UTR sequences

| SEQ | |
|---|---|
| ID NO | Zika viruses with example 3′ UTRs |
| 14 | NC_035889.1 Zika virus isolate ZIKV/H. sapiens/Brazil/Natal/2015, complete genome |
| 15 | NC_012532.1 Zika virus, complete genome |
| 16 | OL450364.1 Zika virus isolate ZIKV/Haiti/1238/2015, complete genome |
| 17 | OL450365.1 Zika virus isolate ZIKV/Haiti/0478/2015, complete genome |
| 18 | OL450366.1 Zika virus isolate ZIKV/Haiti/1332/2015, complete genome |
| 19 | OL414716.1 Zika virus isolate 15555, complete genome |
| 20 | OK571913.1 Zika virus isolate Homo sapiens/Haiti/0728/2014, complete genome |
| 21 | OK054351.1 Zika virus isolate 211784, complete genome |
| 22 | MT377491.1 Zika virus isolate MU-DMSC-1/2017, complete genome |
| 23 | MT377492.1 Zika virus isolate MU-DMSC-1/2016, complete genome |
| 24 | MT377493.1 Zika virus isolate MU-DMSC-2/2017, complete genome |
| 25 | MT377494.1 Zika virus isolate MU-DMSC-3/2017, complete genome |
| 26 | MT377495.1 Zika virus isolate MU-DMSC-4/2017, complete genome |
| 27 | MT377496.1 Zika virus isolate MU-DMSC-2/2016, complete genome |
| 28 | MT377497.1 Zika virus isolate MU-DMSC-3/2016, complete genome |
| 29 | MT377498.1 Zika virus isolate MU-DMSC-4/2016, complete genome |
| 30 | MT377499.1 Zika virus isolate MU-DMSC-5/2016, complete genome |
| 31 | MT377500.1 Zika virus isolate MU-DMSC-6/2016, complete genome |
| 32 | MT377501.1 Zika virus isolate MU-DMSC-5/2017, complete genome |
| 33 | MT377502.1 Zika virus isolate NS-274/2006, complete genome |
| 34 | MT377503.1 Zika virus isolate 15-1144, complete genome |
| 35 | MT377504.1 Zika virus isolate NS-110/2006, complete genome |
| 36 | MZ008356.1 Zika virus isolate ZIKV/Homo_{—}sapiens/Cambodia/2019/SZ1901, complete |
| | genome |
| 37 | MW143022.1 Zika virus strain MR766, complete genome |
| 38 | MW680969.1 Zika virus isolate rGZ02a/2018, complete genome |
| 39 | MW680970.1 Zika virus isolate rGZ02p/2018, complete genome |
| 40 | MW015936.1 Zika virus isolate Zika virus/H. sapiens-tc/THA/2006/CVD_06-020, complete |
| | genome |
| 41 | MN101548.1 Zika virus isolate Salvador, complete genome |
| 42 | MT505349.1 Mutant Zika virus isolate DK, complete genome |
| 43 | MT505350.1 Mutant Zika virus isolate DK23, complete genome |
| 44 | MT636065.1 Zika virus isolate ARCB116141, complete genome |
| 45 | MT483911.1 Zika virus isolate 800/16, complete genome |
| 46 | MT507047.1 Zika virus isolate S-542/Yucatan/2017, complete genome |
| 47 | MT507048.1 Zika virus isolate S-542/Yucatan/2017 clone 6C, complete genome |
| 48 | MT507049.1 Zika virus isolate S-542/Yucatan/2017 clone 6V, complete genome |
| 49 | MT507050.1 Zika virus isolate S-542/Yucatan/2017 clone 6A, complete genome |
| 50 | MK566202.1 Zika virus isolate 15098, complete genome |
| 51 | MK241415.1 Zika virus isolate ZIKV/Homo_{—}sapiens/CPV/2015/CV448c, complete genome |
| 52 | MK241416.1 Zika virus isolate ZIKV/Homo_{—}sapiens/CPV/2015/CV487, complete genome |
| 53 | MK241417.1 Zika virus isolate ZIKV/Homo_{—}sapiens/CPV/2016/CV907u, complete genome |
| 54 | MN611472.1 Zika virus isolate 2019YNZIKV02, complete genome |
| 55 | MN566104.1 Zika virus isolate Haiti/0011/2016, complete genome |
| 56 | MN566105.1 Zika virus isolate Haiti/0375/2016, complete genome |
| 57 | MN566106.1 Zika virus isolate Haiti/0395/2016, complete genome |
| 58 | MN566107.1 Zika virus isolate Haiti/0165/2016, complete genome |
| 59 | MN566108.1 Zika virus isolate Haiti/0866/2016, complete genome |
| 60 | MN577543.1 Zika virus isolate Haiti/1327/2014, complete genome |
| 61 | MN577544.1 Zika virus isolate Haiti/0148/2016, complete genome |
| 62 | MN577550.1 Zika virus isolate ZIKV-Nicaragua/2016-UCB7420, complete genome |
| 63 | MN100039.1 Zika virus isolate ZIKV/Homo sapiens/PAN/CDC-259249_V1-V3/2015, complete |
| | genome |
| 64 | MN124090.1 Zika virus isolate ZIKV/Homo sapiens/PAN/CDC-259249_V1-V3/2015, complete |
| | genome |
| 65 | MN124091.1 Zika virus isolate ZIKV/Homo sapiens/PAN/CDC-259249_V1-V3/2015, complete |
| | genome |
| 66 | MH675619.1 Zika virus isolate CTS-30-16p, complete genome |
| 67 | MH675620.1 Zika virus isolate CTS-36-16p, complete genome |
| 68 | MH675621.1 Zika virus isolate CTS-47-16p, complete genome |
| 69 | MH675622.1 Zika virus isolate CTS-50-16p, complete genome |
| 70 | MH675623.1 Zika virus isolate CTS-56-16p, complete genome |
| 71 | MH675624.1 Zika virus isolate CTS-61-16p, complete genome |
| 72 | MH675625.1 Zika virus isolate CTS-178-16p, complete genome |
| 73 | MH675626.1 Zika virus isolate CTS-183-16p, complete genome |
| 74 | MH675627.1 Zika virus isolate CTS-193-16p, complete genome |
| 75 | MH675628.1 Zika virus isolate CTS-223-16p, complete genome |
| 76 | MH675629.1 Zika virus isolate QTX-02, complete genome |
| 77 | MH675630.1 Zika virus isolate QTX-04, complete genome |
| 78 | MN025403.1 Zika virus isolate 15555, complete genome |
| 79 | MK713748.1 Zika virus isolate PRVABC59, complete genome |
| 80 | MH900227.1 Zika virus isolate 31N, complete genome |
| 81 | MH882527.1 Zika virus isolate Zika virus/H. sapiens-wt/BRA/2016/ZKV17Asem_2016-04-26, |
| | complete genome |
| 82 | MH882528.1 Zika virus isolate Zika virus/H. sapiens-wt/BRA/2016/ZKV17Auri_2016-04-26, |
| | complete genome |
| 83 | MH882529.1 Zika virus isolate Zika virus/H. sapiens-wt/BRA/2016/ZKV17Bsem_2016-05-03, |
| | complete genome |
| 84 | MH882530.1 Zika virus isolate Zika virus/H. sapiens-wt/BRA/2016/ZKV17Csem_2016-05-10, |
| | complete genome |
| 85 | MH882531.1 Zika virus isolate Zika virus/H. sapiens-wt/BRA/2016/ZKV17Dsem_2016-05-17, |
| | complete genome |
| 86 | MH882532.1 Zika virus isolate Zika virus/H. sapiens-wt/BRA/2016/ZKV17Esem_2016-05-24, |
| | complete genome |
| 87 | MH882533.1 Zika virus isolate Zika virus/H. sapiens-wt/BRA/2016/ZKV17Fsem_2016-05-31, |
| | complete genome |
| 88 | MH882534.1 Zika virus isolate Zika virus/H. sapiens-wt/BRA/2016/ZKV17Gsem_2016-06-08, |
| | complete genome |
| 89 | MH882535.1 Zika virus isolate Zika virus/H. sapiens-wt/BRA/2016/ZKV17Hsem_2016-06-16, |
| | complete genome |
| 90 | MH882536.1 Zika virus isolate Zika virus/H. sapiens-wt/BRA/2016/ZKV17Jsem_2016-06-28, |
| | complete genome |
| 91 | MH882538.1 Zika virus isolate Zika virus/H. sapiens-wt/BRA/2016/ZKV17Lsem_2016-07-12, |
| | complete genome |
| 92 | MH882539.1 Zika virus isolate Zika virus/H. sapiens-wt/BRA/2016/ZKV17Msem_2016-07-19, |
| | complete genome |
| 93 | MH882540.1 Zika virus isolate Zika virus/H. sapiens-wt/BRA/2016/ZKV17Nsem_2016-07-27, |
| | complete genome |
| 94 | MH882541.1 Zika virus isolate Zika virus/H. sapiens-wt/BRA/2016/ZKV17sem_2016-04-19, |
| | complete genome |
| 95 | MH882542.1 Zika virus isolate Zika virus/H. sapiens-wt/BRA/2016/ZKV17uri_2016-04-19, |
| | complete genome |
| 96 | MH882543.1 Zika virus isolate Zika virus/H. sapiens-wt/BRA/2016/ZKV19Auri_2016-05-05, |
| | complete genome |
| 97 | MH882544.1 Zika virus isolate Zika virus/H. sapiens-wt/BRA/2016/ZKV19Bsem_2016-05-12, |
| | complete genome |
| 98 | MH882545.1 Zika virus isolate Zika virus/H. sapiens-wt/BRA/2016/ZKV19Csem_2016-05-19, |
| | complete genome |
| 99 | MH882546.1 Zika virus isolate Zika virus/H. sapiens-wt/BRA/2016/ZKV19Dsem_2016-05-24, |
| | complete genome |
| 100 | MH882547.1 Zika virus isolate Zika virus/H. sapiens-wt/BRA/2016/ZKV19Esem_2016-06-02, |
| | complete genome |
| 101 | MH882548.1 Zika virus isolate Zika virus/H. sapiens-wt/BRA/2016/ZKV19Fsem_2016-06-09, |
| | complete genome |
| 102 | MK105975.1 Zika virus strain MR766, complete genome |
| 103 | MF783072.1 Zika virus strain Zika virus/mosquito/Haiti/1855/2016, complete genome |
| 104 | MF783073.1 Zika virus strain Zika virus/mosquito/Haiti/1919/2016, complete genome |
| 105 | MG770187.1 Zika virus isolate SV0127-14, complete genome |
| 106 | MG770188.1 Zika virus isolate SV0127-14, complete genome |
| 107 | MH916802.1 Zika virus strain ZIKV/Homo sapiens/PRI/PRVABC59_17/2015, complete genome |
| 108 | MH916803.1 Zika virus strain ZIKV/Homo sapiens/PRI/PRVABC59_1/2015, complete genome |
| 109 | MH916806.1 Zika virus strain ZIKV/Homo sapiens/PRI/PRVABC59_8/2015, complete genome |
| 110 | MF510857.1 Zika virus isolate Zika virus/Aedes taylori/Senegal/1984/DakAr41667, complete |
| | genome |
| 111 | MH513598.1 Zika virus isolate BR/Sinop/H355/2015, complete genome |
| 112 | MH513599.1 Zika virus isolate BR/Sinop/H366/2015, complete genome |
| 113 | MH513600.1 Zika virus isolate BR/Sinop/H366 2P/2015, complete genome |
| 114 | MH544701.2 Zika virus isolate 459148_Meta_Colombia_2016, complete genome |
| 115 | MF996804.1 Zika virus isolate SI-BKK02, complete genome |
| 116 | MG548660.1 Zika virus isolate BKK03, complete genome |
| 117 | MG548661.1 Zika virus isolate BKK04, complete genome |
| 118 | MG807646.1 Zika virus isolate SI-BKK05, complete genome |
| 119 | MG807647.1 Zika virus isolate SI-BKK06, complete genome |
| 120 | MH013290.1 Zika virus isolate BKK07, complete genome |
| 121 | MF073357.1 Zika virus isolate 16288, complete genome |
| 122 | MF073358.1 Zika virus isolate 15261, complete genome |
| 123 | MF073359.1 Zika virus isolate 15098, complete genome |
| 124 | MG758785.1 Zika virus isolate 41525, complete genome |
| 125 | MG758786.1 Zika virus isolate 41525, complete genome |
| 126 | MH158236.1 Zika virus isolate FSS13025, complete genome |
| 127 | MH158237.1 Zika virus isolate PRVABC59, complete genome |
| 128 | MH130094.1 Zika virus strain ZIKV/Macaca mulatta/UGA/MR-766-VEROE6-AC4-P12_05/1947, |
| | complete genome |
| 129 | MH130095.1 Zika virus strain ZIKV/Macaca mulatta/UGA/MR-766-VEROE6-AC4-P12_07/1947, |
| | complete genome |
| 130 | MH130096.1 Zika virus strain ZIKV/Macaca mulatta/UGA/MR-766-VEROE6-AC10- |
| | P12_05/1947, complete genome |
| 131 | MH130097.1 Zika virus strain ZIKV/Macaca mulatta/UGA/MR-766-VEROE6-AC10- |
| | P12_03/1947, complete genome |
| 132 | MH130098.1 Zika virus strain ZIKV/Macaca mulatta/UGA/MR-766-VEROE6-AC10- |
| | P12_01/1947, complete genome |
| 133 | MH130099.1 Zika virus strain ZIKV/Macaca mulatta/UGA/MR-766-VEROE6-AC4-P12_02/1947, |
| | complete genome |
| 134 | MH130100.1 Zika virus strain ZIKV/Macaca mulatta/UGA/MR-766-VEROE6-AC4-P12_01/1947, |
| | complete genome |
| 135 | MH130101.1 Zika virus strain ZIKV/Macaca mulatta/UGA/MR-766-VEROE6-AC4-P12_08/1947, |
| | complete genome |
| 136 | MH130102.1 Zika virus strain ZIKV/Macaca mulatta/UGA/MR-766-VEROE6-AC10- |
| | P12_02/1947, complete genome |
| 137 | MH130103.1 Zika virus strain ZIKV/Macaca mulatta/UGA/MR-766-VEROE6-AC10- |
| | P12_06/1947, complete genome |
| 138 | MH130104.1 Zika virus strain ZIKV/Macaca mulatta/UGA/MR-766-VEROE6-AC10- |
| | P12_07/1947, complete genome |
| 139 | MH130105.1 Zika virus strain ZIKV/Macaca mulatta/UGA/MR-766-VEROE6-AC10- |
| | P12_04/1947, complete genome |
| 140 | MH130106.1 Zika virus strain ZIKV/Macaca mulatta/UGA/MR-766-VEROE6-AC10- |
| | P12_08/1947, complete genome |
| 141 | MH130107.1 Zika virus strain ZIKV/Macaca mulatta/UGA/MR-766-VEROE6-AC4-P12_03/1947, |
| | complete genome |
| 142 | MH130108.1 Zika virus strain ZIKV/Macaca mulatta/UGA/MR-766-VEROE6-AC4-P12_04/1947, |
| | complete genome |
| 143 | MH130109.1 Zika virus strain ZIKV/Macaca mulatta/UGA/MR-766-VEROE6-AC4-P12_06/1947, |
| | complete genome |
| 144 | MH157202.1 Zika virus strain ZIKV/Homo sapiens/MEX/41-017-F_V3_O/2016, complete |
| | genome |
| 145 | MH157208.1 Zika virus strain ZIKV/Homo sapiens/MEX/41-022-F_V0_O/2016, complete |
| | genome |
| 146 | MH157213.1 Zika virus strain ZIKV/Homo sapiens/MEX/41-001-F_V3_O/2016, complete |
| | genome |
| 147 | MH119185.1 Zika virus isolate DMSc05684-16, complete genome |
| 148 | MH061852.1 Zika virus strain ZIKV/Macaca mulatta/UGA/MR-766-VEROE6-AC11-P5_08/1947, |
| | complete genome |
| 149 | MH061853.1 Zika virus strain ZIKV/Macaca mulatta/UGA/MR-766-VEROE6-GD12- |
| | P10_04/1947, complete genome |
| 150 | MH061854.1 Zika virus strain ZIKV/Macaca mulatta/UGA/MR-766-VEROE6-AC11-P5_06/1947, |
| | complete genome |
| 151 | MH061855.1 Zika virus strain ZIKV/Macaca mulatta/UGA/MR-766-VEROE6-FA12-P7_03/1947, |
| | complete genome |
| 152 | MH061856.1 Zika virus strain ZIKV/Macaca mulatta/UGA/MR-766-VEROE6-FC3-P8_03/1947, |
| | complete genome |
| 153 | MH061857.1 Zika virus strain ZIKV/Macaca mulatta/UGA/MR-766-VEROE6-AC11-P5_07/1947, |
| | complete genome |
| 154 | MH061858.1 Zika virus strain ZIKV/Macaca mulatta/UGA/MR-766-VEROE6-FC11-P7_05/1947, |
| | complete genome |
| 155 | MH061859.1 Zika virus strain ZIKV/Macaca mulatta/UGA/MR-766-VEROE6-AC11-P5_01/1947, |
| | complete genome |
| 156 | MH061860.1 Zika virus strain ZIKV/Macaca mulatta/UGA/MR-766-VEROE6-FC11-P7_06/1947, |
| | complete genome |
| 157 | MH061861.1 Zika virus strain ZIKV/Macaca mulatta/UGA/MR-766-VEROE6-GA3-P10_08/1947, |
| | complete genome |
| 158 | MH061862.1 Zika virus strain ZIKV/Macaca mulatta/UGA/MR-766-VEROE6-GD12- |
| | P10_01/1947, complete genome |
| 159 | MH061863.1 Zika virus strain ZIKV/Macaca mulatta/UGA/MR-766-VEROE6-FA12-P7_02/1947, |
| | complete genome |
| 160 | MH061864.1 Zika virus strain ZIKV/Macaca mulatta/UGA/MR-766-VEROE6-FA12-P7_07/1947, |
| | complete genome |
| 161 | MH061865.1 Zika virus strain ZIKV/Macaca mulatta/UGA/MR-766-VEROE6-AA1-P8_02/1947, |
| | complete genome |
| 162 | MH061866.1 Zika virus strain ZIKV/Macaca mulatta/UGA/MR-766-VEROE6-GD12- |
| | P10_07/1947, complete genome |
| 163 | MH061867.1 Zika virus strain ZIKV/Macaca mulatta/UGA/MR-766-VEROE6-GA3-P10_04/1947, |
| | complete genome |
| 164 | MH061868.1 Zika virus strain ZIKV/Macaca mulatta/UGA/MR-766-VEROE6-AA1-P8_07/1947, |
| | complete genome |
| 165 | MH061869.1 Zika virus strain ZIKV/Macaca mulatta/UGA/MR-766-VEROE6-AA1-P8_06/1947, |
| | complete genome |
| 166 | MH061870.1 Zika virus strain ZIKV/Macaca mulatta/UGA/MR-766-VEROE6-AC11-P5_05/1947, |
| | complete genome |
| 167 | MH061871.1 Zika virus strain ZIKV/Macaca mulatta/UGA/MR-766-VEROE6-FC11-P7_04/1947, |
| | complete genome |
| 168 | MH061872.1 Zika virus strain ZIKV/Macaca mulatta/UGA/MR-766-VEROE6-AA1-P8_05/1947, |
| | complete genome |
| 169 | MH061873.1 Zika virus strain ZIKV/Macaca mulatta/UGA/MR-766-VEROE6-FA12-P7_01/1947, |
| | complete genome |
| 170 | MH061874.1 Zika virus strain ZIKV/Macaca mulatta/UGA/MR-766-VEROE6-AA1-P8_03/1947, |
| | complete genome |
| 171 | MH061875.1 Zika virus strain ZIKV/Macaca mulatta/UGA/MR-766-VEROE6-AC11-P5_03/1947, |
| | complete genome |
| 172 | MH061876.1 Zika virus strain ZIKV/Macaca mulatta/UGA/MR-766-VEROE6-P12_07/1947, |
| | complete genome |
| 173 | MH061877.1 Zika virus strain ZIKV/Macaca mulatta/UGA/MR-766-VEROE6-FC11-P7_07/1947, |
| | complete genome |
| 174 | MH061878.1 Zika virus strain ZIKV/Macaca mulatta/UGA/MR-766-VEROE6-FC3-P8_05/1947, |
| | complete genome |
| 175 | MH061879.1 Zika virus strain ZIKV/Macaca mulatta/UGA/MR-766-VEROE6-GD12- |
| | P10_05/1947, complete genome |
| 176 | MH061880.1 Zika virus strain ZIKV/Macaca mulatta/UGA/MR-766-VEROE6-P12_08/1947, |
| | complete genome |
| 177 | MH061881.1 Zika virus strain ZIKV/Macaca mulatta/UGA/MR-766-VEROE6-GD12- |
| | P10_02/1947, complete genome |
| 178 | MH061882.1 Zika virus strain ZIKV/Macaca mulatta/UGA/MR-766-VEROE6-GD12- |
| | P10_08/1947, complete genome |
| 179 | MH061883.1 Zika virus strain ZIKV/Macaca mulatta/UGA/MR-766-VEROE6-FC11-P7_08/1947, |
| | complete genome |
| 180 | MH061884.1 Zika virus strain ZIKV/Macaca mulatta/UGA/MR-766-VEROE6-GA3-P10_01/1947, |
| | complete genome |
| 181 | MH061885.1 Zika virus strain ZIKV/Macaca mulatta/UGA/MR-766-VEROE6-FC3-P8_06/1947, |
| | complete genome |
| 182 | MH061886.1 Zika virus strain ZIKV/Macaca mulatta/UGA/MR-766-VEROE6-FA12-P7_05/1947, |
| | complete genome |
| 183 | MH061887.1 Zika virus strain ZIKV/Macaca mulatta/UGA/MR-766-VEROE6-P12_05/1947, |
| | complete genome |
| 184 | MH061888.1 Zika virus strain ZIKV/Macaca mulatta/UGA/MR-766-VEROE6-AC11-P5_02/1947, |
| | complete genome |
| 185 | MH061889.1 Zika virus strain ZIKV/Macaca mulatta/UGA/MR-766-VEROE6-FC3-P8_07/1947, |
| | complete genome |
| 186 | MH061890.1 Zika virus strain ZIKV/Macaca mulatta/UGA/MR-766-VEROE6-FC3-P8_08/1947, |
| | complete genome |
| 187 | MH061891.1 Zika virus strain ZIKV/Macaca mulatta/UGA/MR-766-VEROE6-FA12-P7_06/1947, |
| | complete genome |
| 188 | MH061892.1 Zika virus strain ZIKV/Macaca mulatta/UGA/MR-766-VEROE6-P12_03/1947, |
| | complete genome |
| 189 | MH061893.1 Zika virus strain ZIKV/Macaca mulatta/UGA/MR-766-VEROE6-GD12- |
| | P10_03/1947, complete genome |
| 190 | MH061894.1 Zika virus strain ZIKV/Macaca mulatta/UGA/MR-766-VEROE6-FC11-P7_03/1947, |
| | complete genome |
| 191 | MH061895.1 Zika virus strain ZIKV/Macaca mulatta/UGA/MR-766-VEROE6-P12_01/1947, |
| | complete genome |
| 192 | MH061896.1 Zika virus strain ZIKV/Macaca mulatta/UGA/MR-766-VEROE6-FC3-P8_04/1947, |
| | complete genome |
| 193 | MH061897.1 Zika virus strain ZIKV/Macaca mulatta/UGA/MR-766-VEROE6-FC3-P8_01/1947, |
| | complete genome |
| 194 | MH061898.1 Zika virus strain ZIKV/Macaca mulatta/UGA/MR-766-VEROE6-FC11-P7_02/1947, |
| | complete genome |
| 195 | MH061899.1 Zika virus strain ZIKV/Macaca mulatta/UGA/MR-766-VEROE6-AA1-P8_08/1947, |
| | complete genome |
| 196 | MH061900.1 Zika virus strain ZIKV/Macaca mulatta/UGA/MR-766-VEROE6-P12_04/1947, |
| | complete genome |
| 197 | MH061901.1 Zika virus strain ZIKV/Macaca mulatta/UGA/MR-766-VEROE6-GA3-P10_07/1947, |
| | complete genome |
| 198 | MH061902.1 Zika virus strain ZIKV/Macaca mulatta/UGA/MR-766-VEROE6-FA12-P7_08/1947, |
| | complete genome |
| 199 | MH061903.1 Zika virus strain ZIKV/Macaca mulatta/UGA/MR-766-VEROE6-AA1-P8_04/1947, |
| | complete genome |
| 200 | MH061904.1 Zika virus strain ZIKV/Macaca mulatta/UGA/MR-766-VEROE6-FC3-P8_02/1947, |
| | complete genome |
| 201 | MH061905.1 Zika virus strain ZIKV/Macaca mulatta/UGA/MR-766-VEROE6-AA1-P8_01/1947, |
| | complete genome |
| 202 | MH061906.1 Zika virus strain ZIKV/Macaca mulatta/UGA/MR-766-VEROE6-FC11-P7_01/1947, |
| | complete genome |
| 203 | MH061907.1 Zika virus strain ZIKV/Macaca mulatta/UGA/MR-766-VEROE6-GA3-P10_06/1947, |
| | complete genome |
| 204 | MH061908.1 Zika virus strain ZIKV/Macaca mulatta/UGA/MR-766-VEROE6-P12_06/1947, |
| | complete genome |
| 205 | MH061909.1 Zika virus strain ZIKV/Macaca mulatta/UGA/MR-766-VEROE6-AC11-P5_04/1947, |
| | complete genome |
| 206 | MH061910.1 Zika virus strain ZIKV/Macaca mulatta/UGA/MR-766-VEROE6-FA12-P7_04/1947, |
| | complete genome |
| 207 | MH061911.1 Zika virus strain ZIKV/Macaca mulatta/UGA/MR-766-VEROE6-GD12- |
| | P10_06/1947, complete genome |
| 208 | MH061912.1 Zika virus strain ZIKV/Macaca mulatta/UGA/MR-766-VEROE6-GA3-P10_03/1947, |
| | complete genome |
| 209 | MH061913.1 Zika virus strain ZIKV/Macaca mulatta/UGA/MR-766-VEROE6-GA3-P10_05/1947, |
| | complete genome |
| 210 | MH061914.1 Zika virus strain ZIKV/Macaca mulatta/UGA/MR-766-VEROE6-GA3-P10_02/1947, |
| | complete genome |
| 211 | MH061915.1 Zika virus strain ZIKV/Macaca mulatta/UGA/MR-766-VEROE6-P12_02/1947, |
| | complete genome |
| 212 | LC369584.1 Zika virus ZIKV/Hu/Thai/KngSG/17-D501 genomic RNA, nearly complete genome |
| 213 | MG827392.1 Zika virus strain PF13/251013-18, complete genome |
| 214 | MG674718.1 Zika virus isolate ZKC2P4, complete genome |
| 215 | MG674719.1 Zika virus isolate ZKC2P6, complete genome |
| 216 | KY766069.1 Zika virus isolate Pf13/251013-18, complete genome |
| 217 | MF794971.1 Zika virus strain ZIKV/EC/Esmeraldas/121/2016, complete genome |
| 218 | KY553111.1 Zika virus isolate AFMC-U, complete genome |
| 219 | MF384325.1 Zika virus isolate mosquito/Haiti/1682/2016, complete genome |
| 220 | MF352141.1 Zika virus isolate PE243, complete genome |
| 221 | KY415986.1 Zika virus isolate Haiti/0029/2014, complete genome |
| 222 | KY415987.1 Zika virus isolate Haiti/0033/2014, complete genome |
| 223 | KY415988.1 Zika virus isolate Haiti/0054/2014, complete genome |
| 224 | KY415989.1 Zika virus isolate Haiti/0074/2014, complete genome |
| 225 | KY415990.1 Zika virus isolate Haiti/0036/2014, complete genome |
| 226 | KY415991.1 Zika virus isolate Haiti/0097/2014, complete genome |
| 227 | KY989511.1 Zika virus strain MR 766, complete genome |
| 228 | KY989971.1 Zika virus isolate FLA, complete genome |
| 229 | KX051560.1 Zika virus isolate SK364/13AS, complete genome |
| 230 | KX051561.1 Zika virus isolate SK403/13AS, complete genome |
| 231 | KX051562.1 Zika virus isolate SV0010/15, complete genome |
| 232 | KY765317.1 Zika virus strain ZIKV/Homo sapiens/NIC/7252_12A1/2016, complete genome |
| 233 | KY765318.1 Zika virus strain ZIKV/Homo sapiens/NIC/4886_12A1/2016, complete genome |
| 234 | KY765320.1 Zika virus strain ZIKV/Homo sapiens/NIC/6406_13A1_SP/2016, complete genome |
| 235 | KY765321.1 Zika virus strain ZIKV/Homo sapiens/NIC/4886_12A1_SP/2016, complete genome |
| 236 | KY765322.1 Zika virus strain ZIKV/Homo sapiens/NIC/7252_12A1_SP/2016, complete genome |
| 237 | KY765323.1 Zika virus strain ZIKV/Homo sapiens/NIC/6188_13A1/2016, complete genome |
| 238 | KY765324.1 Zika virus strain ZIKV/Homo sapiens/NIC/8610_13A1/2016, complete genome |
| 239 | KY765325.1 Zika virus strain ZIKV/Homo sapiens/NIC/5005_13A1/2016, complete genome |
| 240 | KY765326.1 Zika virus strain ZIKV/Homo sapiens/NIC/6188_13A1_SP/2016, complete genome |
| 241 | KY765327.1 Zika virus strain ZIKV/Homo sapiens/NIC/5005_13A1_SP/2016, complete genome |
| 242 | KY631493.1 Zika virus isolate MEX_ENCB165, complete genome |
| 243 | KY631494.1 Zika virus isolate MEX_ENCB165P4, complete genome |
| 244 | LC219720.1 Zika virus genomic RNA, complete genome, strain: ZIKV/Hu/NIID123/2016 |
| 245 | KY648934.1 Zika virus strain ZIKV/Aedes aegypti/MEX/MEX_I-44/2016, complete genome |
| 246 | KX827268.1 Zika virus strain ZIKV/Homo sapiens/USA/UT-1/2016, complete genome |
| 247 | KY348640.1 Zika virus strain SL1602, complete genome |
| 248 | KU761560.1 Zika virus isolate ZJ03, complete genome |
| 249 | KU761561.1 Zika virus isolate ZJ02, complete genome |
| 250 | KY272991.1 Zika virus isolate RIO-BM1, complete genome |
| 251 | KY288905.1 Zika virus strain MP1751, complete genome |
| 252 | KY328289.1 Zika virus isolate HN16, complete genome |
| 253 | KY272987.1 Zika virus isolate SI-BKK01, complete genome |
| 254 | KY120348.1 Zika virus strain MEX_CIENI551P4, complete genome |
| 255 | KY120349.2 Zika virus strain MEX_CIENI551, complete genome |
| 256 | KX269878.1 Zika virus isolate Haiti/2016/PD, complete genome |
| 257 | LC191864.1 Zika virus genomic RNA, complete genome, strain: ZIKV/Hu/Chiba/S36/2016 |
| 258 | LC190723.1 Zika virus genomic RNA, complete genome, strain: ZIKV/Hu/Yokohama/1/2016 |
| 259 | KX893855.1 Zika virus strain Zika virus/Homo sapiens/VEN/UF-2/2016, complete genome |
| 260 | KX879603.1 Zika virus strain ZIKV/EC/Esmeraldas/062/2016, complete genome |
| 261 | KX879604.1 Zika virus strain ZIKV/EC/Esmeraldas/089/2016, complete genome |
| 262 | KX830960.1 Zika virus culture ATCC: VR-84 isolate MR776, complete genome |
| 263 | KX811222.1 Zika virus isolate Brazil_2015_MG, complete genome |
| 264 | KX856011.1 Zika virus strain ZIKV/Aedes sp./MEX_I-44/2016, complete genome |
| 265 | KX197205.1 Zika virus isolate 9, complete genome |
| 266 | KX806557.3 Zika virus isolate TS17-2016, complete genome |
| 267 | KX766028.1 Zika virus isolate R114916, complete genome |
| 268 | KX766029.1 Zika virus isolate R116265, complete genome |
| 269 | KX702400.1 Zika virus strain Zika virus/Homo sapiens/VEN/UF-1/2016, complete genome |
| 270 | KX694532.2 Zika virus strain ZIKV/Homo sapiens/THA/PLCal_ZV/2013, complete genome |
| 271 | KX694533.2 Zika virus strain ZIKV/Aedes aegypti/MYS/P6-740/1966, complete genome |
| 272 | KX694534.2 Zika virus strain ZIKV/Homo sapiens/HND/R103451/2015, complete genome |
| 273 | KX673530.1 Zika virus isolate PHE_semen_Guadeloupe, complete genome |
| 274 | KX266255.1 Zika virus isolate ZIKV_SMGC-1, complete genome |
| 275 | KX601166.2 Zika virus strain ZIKV/Aedes africanus/SEN/DakAr41524/1984, complete genome |
| 276 | KX601167.1 Zika virus strain ZIKV/Aedes sp./MYS/P6-740/1966, complete genome |
| 277 | KX601168.1 Zika virus strain ZIKV/Homo sapiens/PRI/PRVABC59/2015, complete genome |
| 278 | KX601169.1 Zika virus strain ZIKV/Macaca mulatta/UGA/MR-766/1947, complete genome |
| 279 | KX369547.1 Zika virus strain PF13/251013-18, complete genome |
| 280 | KX446950.2 Zika virus strain ZIKV/Aedes. sp/MEX/MEX_2-81/2016, complete genome |
| 281 | KX446951.2 Zika virus strain ZIKV/Aedes. sp/MEX/MEX_I-7/2016, complete genome |
| 282 | KX377335.1 Zika virus strain MR-766, complete genome |
| 283 | KX377336.1 Zika virus strain P6-740, complete genome |
| 284 | KX377337.1 Zika virus strain PRVABC-59, complete genome |
| 285 | KX280026.1 Zika virus isolate Paraiba_01, complete genome |
| 286 | KX262887.1 Zika virus isolate 103451, complete genome |
| 287 | KX253996.1 Zika virus isolate ZKC2/2016, complete genome |
| 288 | KX247646.1 Zika virus isolate Zika virus/Homo sapiens/COL/UF-1/2016, complete genome |
| 289 | KX197192.1 Zika virus isolate ZIKV/H. sapiens/Brazil/PE243/2015, complete genome |
| 290 | KX198134.2 Zika virus strain ZIKV/Aedes africanus/SEN/DAK-AR-41524_A1C1-V2/1984, |
| | complete genome |
| 291 | KX198135.2 Zika virus strain ZIKV/Homo sapiens/PAN/BEI-259634_V4/2016, complete |
| | genome |
| 292 | KX156774.2 Zika virus strain ZIKV/Homo sapiens/PAN/CDC-259359_V1-V3/2015, complete |
| | genome |
| 293 | KX156775.2 Zika virus strain ZIKV/Homo sapiens/PAN/CDC-259249_V1-V3/2015, complete |
| | genome |
| 294 | KX156776.2 Zika virus strain ZIKV/Homo sapiens/PAN/CDC-259364_V1-V2/2015, complete |
| | genome |
| 295 | KX117076.1 Zika virus isolate Zhejiang04, complete genome |
| 296 | KX087101.3 Zika virus strain ZIKV/Homo sapiens/PRI/PRVABC59/2015, complete genome |
| 297 | KX087102.2 Zika virus strain ZIKV/Homo sapiens/COL/FLR/2015, complete genome |
| 298 | KX051563.1 Zika virus isolate Haiti/1/2016, complete genome |
| 299 | KU955591.1 Zika virus isolate Zika virus/A. africanus-tc/SEN/1984/41525-DAK, complete |
| | genome |
| 300 | KU955592.1 Zika virus isolate Zika virus/A. taylori-tc/SEN/1984/41662-DAK, complete genome |
| 301 | KU955593.1 Zika virus isolate Zika virus/H. sapiens-tc/KHM/2010/FSS13025, complete genome |
| 302 | KU955594.1 Zika virus isolate Zika virus/M. mulatta-tc/UGA/1947/MR-766, complete genome |
| 303 | KU955595.1 Zika virus isolate Zika virus/A. taylori-tc/SEN/1984/41671-DAK, complete genome |
| 304 | KU870645.1 Zika virus isolate FB-GWUH-2016, complete genome |
| 305 | KU926309.2 Zika virus isolate Rio-U1, complete genome |
| 306 | KU926310.2 Zika virus isolate Rio-S1, complete genome |
| 307 | KU922923.1 Zika virus isolate MEX/InDRE/Lm/2016, complete genome |
| 308 | KU922960.1 Zika virus isolate MEX/InDRE/Sm/2016, complete genome |
| 309 | KU853012.1 Zika virus isolate Dominican Republic/2016/PD1, complete genome |
| 310 | KU853013.1 Zika virus isolate Dominican Republic/2016/PD2, complete genome |
| 311 | KU820899.2 Zika virus isolate ZJ03, complete genome |
| 312 | KU744693.1 Zika virus isolate VE_Ganxian, complete genome |
| 313 | KU497555.1 Zika virus isolate Brazil-ZKV2015, complete genome |
| 314 | KU707826.1 Zika virus isolate SSABR1, complete genome |
| 315 | KU527068.1 Zika virus strain Natal RGN, complete genome |
| 316 | KU681081.3 Zika virus isolate Zika virus/H. sapiens-tc/THA/2014/SV0127- 14, complete |
| | genome |
| 317 | KU681082.3 Zika virus isolate Zika virus/H. sapiens-tc/PHL/2012/CPC-0740, complete genome |
| 318 | KU509998.3 Zika virus strain Haiti/1225/2014, complete genome |
| 319 | KU501215.1 Zika virus strain PRVABC59, complete genome |
| 320 | KU321639.1 Zika virus strain ZikaSPH2015, complete genome |
| 321 | LC002520.1 Zika virus genomic RNA, complete genome, strain: MR766-NIID |
| 322 | KJ776791.2 Zika virus strain H/PF/2013, complete genome |
| 323 | AY632535.2 Zika virus strain MR 766, complete genome |

TABLE 1C
Non-limiting example Zika 5′ UTR sequences

| SEQ | |
|---|---|
| ID NO | Zika viruses with example 5′ UTRs |
| 324 | >NC_035889.1 Zika virus isolate ZIKV/H. sapiens/Brazil/Natal/2015, complete genome |
| 325 | >NC_012532.1 Zika virus, complete genome |
| 326 | >OL450364.1 Zika virus isolate ZIKV/Haiti/1238/2015, complete genome |
| 327 | >OL450365.1 Zika virus isolate ZIKV/Haiti/0478/2015, complete genome |
| 328 | >OL450366.1 Zika virus isolate ZIKV/Haiti/1332/2015, complete genome |
| 329 | >OL414716.1 Zika virus isolate 15555, complete genome |
| 330 | >OK571913.1 Zika virus isolate Homo sapiens/Haiti/0728/2014, complete genome |
| 331 | >OK054351.1 Zika virus isolate 211784, complete genome |
| 332 | >MT377491.1 Zika virus isolate MU-DMSC-1/2017, complete genome |
| 333 | >MT377492.1 Zika virus isolate MU-DMSC-1/2016, complete genome |
| 334 | >MT377493.1 Zika virus isolate MU-DMSC-2/2017, complete genome |
| 335 | >MT377494.1 Zika virus isolate MU-DMSC-3/2017, complete genome |
| 336 | >MT377495.1 Zika virus isolate MU-DMSC-4/2017, complete genome |
| 337 | >MT377496.1 Zika virus isolate MU-DMSC-2/2016, complete genome |
| 338 | >MT377497.1 Zika virus isolate MU-DMSC-3/2016, complete genome |
| 339 | >MT377498.1 Zika virus isolate MU-DMSC-4/2016, complete genome |
| 340 | >MT377499.1 Zika virus isolate MU-DMSC-5/2016, complete genome |
| 341 | >MT377500.1 Zika virus isolate MU-DMSC-6/2016, complete genome |
| 342 | >MT377501.1 Zika virus isolate MU-DMSC-5/2017, complete genome |
| 343 | >MT377502.1 Zika virus isolate NS-274/2006, complete genome |
| 344 | >MT377503.1 Zika virus isolate 15-1144, complete genome |
| 345 | >MT377504.1 Zika virus isolate NS-110/2006, complete genome |
| 346 | >MZ008356.1 Zika virus isolate ZIKV/Homo_{—}sapiens/Cambodia/2019/SZ1901, complete genome |
| 347 | >MW143022.1 Zika virus strain MR766, complete genome |
| 348 | >MW680969.1 Zika virus isolate rGZ02a/2018, complete genome |
| 349 | >MW680970.1 Zika virus isolate rGZ02p/2018, complete genome |
| 350 | >MW015936.1 Zika virus isolate Zika virus/H. sapiens-tc/THA/2006/CVD_06-020, complete genome |
| 351 | >MN101548.1 Zika virus isolate Salvador, complete genome |
| 352 | >MT505349.1 Mutant Zika virus isolate DK, complete genome |
| 353 | >MT505350.1 Mutant Zika virus isolate DK23, complete genome |
| 354 | >MT636065.1 Zika virus isolate ARCB116141, complete genome |
| 355 | >MT483911.1 Zika virus isolate 800/16, complete genome |
| 356 | >MT507047.1 Zika virus isolate S-542/Yucatan/2017, complete genome |
| 357 | >MT507048.1 Zika virus isolate S-542/Yucatan/2017 clone 6C, complete genome |
| 358 | >MT507049.1 Zika virus isolate S-542/Yucatan/2017 clone 6V, complete genome |
| 359 | >MT507050.1 Zika virus isolate S-542/Yucatan/2017 clone 6A, complete genome |
| 360 | >MK566202.1 Zika virus isolate 15098, complete genome |
| 361 | >MK241415.1 Zika virus isolate ZIKV/Homo_{—}sapiens/CPV/2015/CV448c, complete genome |
| 362 | >MK241416.1 Zika virus isolate ZIKV/Homo_{—}sapiens/CPV/2015/CV487, complete genome |
| 363 | >MK241417.1 Zika virus isolate ZIKV/Homo_{—}sapiens/CPV/2016/CV907u, complete genome |
| 364 | >MN611472.1 Zika virus isolate 2019YNZIKV02, complete genome |
| 365 | >MN566104.1 Zika virus isolate Haiti/0011/2016, complete genome |
| 366 | >MN566105.1 Zika virus isolate Haiti/0375/2016, complete genome |
| 367 | >MN566106.1 Zika virus isolate Haiti/0395/2016, complete genome |
| 368 | >MN566107.1 Zika virus isolate Haiti/0165/2016, complete genome |
| 369 | >MN566108.1 Zika virus isolate Haiti/0866/2016, complete genome |
| 370 | >MN577543.1 Zika virus isolate Haiti/1327/2014, complete genome |
| 371 | >MN577544.1 Zika virus isolate Haiti/0148/2016, complete genome |
| 372 | >MN577550.1 Zika virus isolate ZIKV-Nicaragua/2016-UCB7420, complete genome |
| 373 | >MN100039.1 Zika virus isolate ZIKV/Homo sapiens/PAN/CDC-259249_V1-V3/2015, complete |
| | genome |
| 374 | >MN124090.1 Zika virus isolate ZIKV/Homo sapiens/PAN/CDC-259249_V1-V3/2015, complete |
| | genome |
| 375 | >MN124091.1 Zika virus isolate ZIKV/Homo sapiens/PAN/CDC-259249_V1-V3/2015, complete |
| | genome |
| 376 | >MH675619.1 Zika virus isolate CTS-30-16p, complete genome |
| 377 | >MH675620.1 Zika virus isolate CTS-36-16p, complete genome |
| 378 | >MH675621.1 Zika virus isolate CTS-47-16p, complete genome |
| 379 | >MH675622.1 Zika virus isolate CTS-50-16p, complete genome |
| 380 | >MH675623.1 Zika virus isolate CTS-56-16p, complete genome |
| 381 | >MH675624.1 Zika virus isolate CTS-61-16p, complete genome |
| 382 | >MH675625.1 Zika virus isolate CTS-178-16p, complete genome |
| 383 | >MH675626.1 Zika virus isolate CTS-183-16p, complete genome |
| 384 | >MH675627.1 Zika virus isolate CTS-193-16p, complete genome |
| 385 | >MH675628.1 Zika virus isolate CTS-223-16p, complete genome |
| 386 | >MH675629.1 Zika virus isolate QTX-02, complete genome |
| 387 | >MH675630.1 Zika virus isolate QTX-04, complete genome |
| 388 | >MN025403.1 Zika virus isolate 15555, complete genome |
| 389 | >MK713748.1 Zika virus isolate PRVABC59, complete genome |
| 390 | >MH900227.1 Zika virus isolate 31N, complete genome |
| 391 | >MH882527.1 Zika virus isolate Zika virus/H. sapiens-wt/BRA/2016/ZKV17Asem_2016-04-26, |
| | complete genome |
| 392 | >MH882528.1 Zika virus isolate Zika virus/H. sapiens-wt/BRA/2016/ZKV17Auri_2016-04-26, |
| | complete genome |
| 393 | >MH882529.1 Zika virus isolate Zika virus/H. sapiens-wt/BRA/2016/ZKV17Bsem_2016-05-03, |
| | complete genome |
| 394 | >MH882530.1 Zika virus isolate Zika virus/H. sapiens-wt/BRA/2016/ZKV17Csem_2016-05-10, |
| | complete genome |
| 395 | >MH882531.1 Zika virus isolate Zika virus/H. sapiens-wt/BRA/2016/ZKV17Dsem_2016-05-17, |
| | complete genome |
| 396 | >MH882532.1 Zika virus isolate Zika virus/H. sapiens-wt/BRA/2016/ZKV17Esem_2016-05-24, |
| | complete genome |
| 397 | >MH882533.1 Zika virus isolate Zika virus/H. sapiens-wt/BRA/2016/ZKV17Fsem_2016-05-31, |
| | complete genome |
| 398 | >MH882534.1 Zika virus isolate Zika virus/H. sapiens-wt/BRA/2016/ZKV17Gsem_2016-06-08, |
| | complete genome |
| 399 | >MH882535.1 Zika virus isolate Zika virus/H. sapiens-wt/BRA/2016/ZKV17Hsem_2016-06-16, |
| | complete genome |
| 400 | >MH882536.1 Zika virus isolate Zika virus/H. sapiens-wt/BRA/2016/ZKV17Jsem_2016-06-28, |
| | complete genome |
| 401 | >MH882538.1 Zika virus isolate Zika virus/H. sapiens-wt/BRA/2016/ZKV17Lsem_2016-07-12, |
| | complete genome |
| 402 | >MH882539.1 Zika virus isolate Zika virus/H. sapiens-wt/BRA/2016/ZKV17Msem_2016-07-19, |
| | complete genome |
| 403 | >MH882540.1 Zika virus isolate Zika virus/H. sapiens-wt/BRA/2016/ZKV17Nsem_2016-07-27, |
| | complete genome |
| 404 | >MH882541.1 Zika virus isolate Zika virus/H. sapiens-wt/BRA/2016/ZKV17sem_2016-04-19, |
| | complete genome |
| 405 | >MH882542.1 Zika virus isolate Zika virus/H. sapiens-wt/BRA/2016/ZKV17uri_2016-04-19, |
| | complete genome |
| 406 | >MH882543.1 Zika virus isolate Zika virus/H. sapiens-wt/BRA/2016/ZKV19Auri_2016-05-05, |
| | complete genome |
| 407 | >MH882544.1 Zika virus isolate Zika virus/H. sapiens-wt/BRA/2016/ZKV19Bsem_2016-05-12, |
| | complete genome |
| 408 | >MH882545.1 Zika virus isolate Zika virus/H. sapiens-wt/BRA/2016/ZKV19Csem_2016-05-19, |
| | complete genome |
| 409 | >MH882546.1 Zika virus isolate Zika virus/H. sapiens-wt/BRA/2016/ZKV19Dsem_2016-05-24, |
| | complete genome |
| 410 | >MH882547.1 Zika virus isolate Zika virus/H. sapiens-wt/BRA/2016/ZKV19Esem_2016-06-02, |
| | complete genome |
| 411 | >MH882548.1 Zika virus isolate Zika virus/H. sapiens-wt/BRA/2016/ZKV19Fsem_2016-06-09, |
| | complete genome |
| 412 | >MK105975.1 Zika virus strain MR766, complete genome |
| 413 | >MF783072.1 Zika virus strain Zika virus/mosquito/Haiti/1855/2016, complete genome |
| 414 | >MF783073.1 Zika virus strain Zika virus/mosquito/Haiti/1919/2016, complete genome |
| 415 | >MG770187.1 Zika virus isolate SV0127-14, complete genome |
| 416 | >MG770188.1 Zika virus isolate SV0127-14, complete genome |
| 417 | >MH916802.1 Zika virus strain ZIKV/Homo sapiens/PRI/PRVABC59_17/2015, complete genome |
| 418 | >MH916803.1 Zika virus strain ZIKV/Homo sapiens/PRI/PRVABC59_1/2015, complete genome |
| 419 | >MH916806.1 Zika virus strain ZIKV/Homo sapiens/PRI/PRVABC59_8/2015, complete genome |
| 420 | >MF510857.1 Zika virus isolate Zika virus/Aedes taylori/Senegal/1984/DakAr41667, complete |
| | genome |
| 421 | >MH513598.1 Zika virus isolate BR/Sinop/H355/2015, complete genome |
| 422 | >MH513599.1 Zika virus isolate BR/Sinop/H366/2015, complete genome |
| 423 | >MH513600.1 Zika virus isolate BR/Sinop/H366 2P/2015, complete genome |
| 424 | >MH544701.2 Zika virus isolate 459148_Meta_Colombia_2016, complete genome |
| 425 | >MF996804.1 Zika virus isolate SI-BKK02, complete genome |
| 426 | >MG548660.1 Zika virus isolate BKK03, complete genome |
| 427 | >MG548661.1 Zika virus isolate BKK04, complete genome |
| 428 | >MG807646.1 Zika virus isolate SI-BKK05, complete genome |
| 429 | >MG807647.1 Zika virus isolate SI-BKK06, complete genome |
| 430 | >MH013290.1 Zika virus isolate BKK07, complete genome |
| 431 | >MF073357.1 Zika virus isolate 16288, complete genome |
| 432 | >MF073358.1 Zika virus isolate 15261, complete genome |
| 433 | >MF073359.1 Zika virus isolate 15098, complete genome |
| 434 | >MG758785.1 Zika virus isolate 41525, complete genome |
| 435 | >MG758786.1 Zika virus isolate 41525, complete genome |
| 436 | >MH158236.1 Zika virus isolate FSS13025, complete genome |
| 437 | >MH158237.1 Zika virus isolate PRVABC59, complete genome |
| 438 | >MH130094.1 Zika virus strain ZIKV/Macaca mulatta/UGA/MR-766-VEROE6-AC4-P12_05/1947, |
| | complete genome |
| 439 | >MH130095.1 Zika virus strain ZIKV/Macaca mulatta/UGA/MR-766-VEROE6-AC4-P12_07/1947, |
| | complete genome |
| 440 | >MH130096.1 Zika virus strain ZIKV/Macaca mulatta/UGA/MR-766-VEROE6-AC10-P12_05/1947, |
| | complete genome |
| 441 | >MH130097.1 Zika virus strain ZIKV/Macaca mulatta/UGA/MR-766-VEROE6-AC10-P12_03/1947, |
| | complete genome |
| 442 | >MH130098.1 Zika virus strain ZIKV/Macaca mulatta/UGA/MR-766-VEROE6-AC10-P12_01/1947, |
| | complete genome |
| 443 | >MH130099.1 Zika virus strain ZIKV/Macaca mulatta/UGA/MR-766-VEROE6-AC4-P12_02/1947, |
| | complete genome |
| 444 | >MH130100.1 Zika virus strain ZIKV/Macaca mulatta/UGA/MR-766-VEROE6-AC4-P12_01/1947, |
| | complete genome |
| 445 | >MH130101.1 Zika virus strain ZIKV/Macaca mulatta/UGA/MR-766-VEROE6-AC4-P12_08/1947, |
| | complete genome |
| 446 | >MH130102.1 Zika virus strain ZIKV/Macaca mulatta/UGA/MR-766-VEROE6-AC10-P12_02/1947, |
| | complete genome |
| 447 | >MH130103.1 Zika virus strain ZIKV/Macaca mulatta/UGA/MR-766-VEROE6-AC10-P12_06/1947, |
| | complete genome |
| 448 | >MH130104.1 Zika virus strain ZIKV/Macaca mulatta/UGA/MR-766-VEROE6-AC10-P12_07/1947, |
| | complete genome |
| 449 | >MH130105.1 Zika virus strain ZIKV/Macaca mulatta/UGA/MR-766-VEROE6-AC10-P12_04/1947, |
| | complete genome |
| 450 | >MH130106.1 Zika virus strain ZIKV/Macaca mulatta/UGA/MR-766-VEROE6-AC10-P12_08/1947, |
| | complete genome |
| 451 | >MH130107.1 Zika virus strain ZIKV/Macaca mulatta/UGA/MR-766-VEROE6-AC4-P12_03/1947, |
| | complete genome |
| 452 | >MH130108.1 Zika virus strain ZIKV/Macaca mulatta/UGA/MR-766-VEROE6-AC4-P12_04/1947, |
| | complete genome |
| 453 | >MH130109.1 Zika virus strain ZIKV/Macaca mulatta/UGA/MR-766-VEROE6-AC4-P12_06/1947, |
| | complete genome |
| 454 | >MH157202.1 Zika virus strain ZIKV/Homo sapiens/MEX/41-017-F_V3_O/2016, complete genome |
| 455 | >MH157208.1 Zika virus strain ZIKV/Homo sapiens/MEX/41-022-F_V0_O/2016, complete genome |
| 456 | >MH157213.1 Zika virus strain ZIKV/Homo sapiens/MEX/41-001-F_V3_O/2016, complete genome |
| 457 | >MH119185.1 Zika virus isolate DMSc05684-16, complete genome |
| 458 | >MH061852.1 Zika virus strain ZIKV/Macaca mulatta/UGA/MR-766-VEROE6-AC11-P5_08/1947, |
| | complete genome |
| 459 | >MH061853.1 Zika virus strain ZIKV/Macaca mulatta/UGA/MR-766-VEROE6-GD12-P10_04/1947, |
| | complete genome |
| 460 | >MH061854.1 Zika virus strain ZIKV/Macaca mulatta/UGA/MR-766-VEROE6-AC11-P5_06/1947, |
| | complete genome |
| 461 | >MH061855.1 Zika virus strain ZIKV/Macaca mulatta/UGA/MR-766-VEROE6-FA12-P7_03/1947, |
| | complete genome |
| 462 | >MH061856.1 Zika virus strain ZIKV/Macaca mulatta/UGA/MR-766-VEROE6-FC3-P8_03/1947, |
| | complete genome |
| 463 | >MH061857.1 Zika virus strain ZIKV/Macaca mulatta/UGA/MR-766-VEROE6-AC11-P5_07/1947, |
| | complete genome |
| 464 | >MH061858.1 Zika virus strain ZIKV/Macaca mulatta/UGA/MR-766-VEROE6-FC11-P7_05/1947, |
| | complete genome |
| 465 | >MH061859.1 Zika virus strain ZIKV/Macaca mulatta/UGA/MR-766-VEROE6-AC11-P5_01/1947, |
| | complete genome |
| 466 | >MH061860.1 Zika virus strain ZIKV/Macaca mulatta/UGA/MR-766-VEROE6-FC11-P7_06/1947, |
| | complete genome |
| 467 | >MH061861.1 Zika virus strain ZIKV/Macaca mulatta/UGA/MR-766-VEROE6-GA3-P10_08/1947, |
| | complete genome |
| 468 | >MH061862.1 Zika virus strain ZIKV/Macaca mulatta/UGA/MR-766-VEROE6-GD12- |
| | P10_01/1947, complete genome |
| 469 | >MH061863.1 Zika virus strain ZIKV/Macaca mulatta/UGA/MR-766-VEROE6-FA12-P7_02/1947, |
| | complete genome |
| 470 | >MH061864.1 Zika virus strain ZIKV/Macaca mulatta/UGA/MR-766-VEROE6-FA12-P7_07/1947, |
| | complete genome |
| 471 | >MH061865.1 Zika virus strain ZIKV/Macaca mulatta/UGA/MR-766-VEROE6-AA1-P8_02/1947, |
| | complete genome |
| 472 | >MH061866.1 Zika virus strain ZIKV/Macaca mulatta/UGA/MR-766-VEROE6-GD12- |
| | P10_07/1947, complete genome |
| 473 | >MH061867.1 Zika virus strain ZIKV/Macaca mulatta/UGA/MR-766-VEROE6-GA3-P10_04/1947, |
| | complete genome |
| 474 | >MH061868.1 Zika virus strain ZIKV/Macaca mulatta/UGA/MR-766-VEROE6-AA1-P8_07/1947, |
| | complete genome |
| 475 | >MH061869.1 Zika virus strain ZIKV/Macaca mulatta/UGA/MR-766-VEROE6-AA1-P8_06/1947, |
| | complete genome |
| 476 | >MH061870.1 Zika virus strain ZIKV/Macaca mulatta/UGA/MR-766-VEROE6-AC11-P5_05/1947, |
| | complete genome |
| 477 | >MH061871.1 Zika virus strain ZIKV/Macaca mulatta/UGA/MR-766-VEROE6-FC11-P7_04/1947, |
| | complete genome |
| 478 | >MH061872.1 Zika virus strain ZIKV/Macaca mulatta/UGA/MR-766-VEROE6-AA1-P8_05/1947, |
| | complete genome |
| 479 | >MH061873.1 Zika virus strain ZIKV/Macaca mulatta/UGA/MR-766-VEROE6-FA12-P7_01/1947, |
| | complete genome |
| 480 | >MH061874.1 Zika virus strain ZIKV/Macaca mulatta/UGA/MR-766-VEROE6-AA1-P8_03/1947, |
| | complete genome |
| 481 | >MH061875.1 Zika virus strain ZIKV/Macaca mulatta/UGA/MR-766-VEROE6-AC11-P5_03/1947, |
| | complete genome |
| 482 | >MH061876.1 Zika virus strain ZIKV/Macaca mulatta/UGA/MR-766-VEROE6-P12_07/1947, |
| | complete genome |
| 483 | >MH061877.1 Zika virus strain ZIKV/Macaca mulatta/UGA/MR-766-VEROE6-FC11-P7_07/1947, |
| | complete genome |
| 484 | >MH061878.1 Zika virus strain ZIKV/Macaca mulatta/UGA/MR-766-VEROE6-FC3-P8_05/1947, |
| | complete genome |
| 485 | >MH061879.1 Zika virus strain ZIKV/Macaca mulatta/UGA/MR-766-VEROE6-GD12- |
| | P10_05/1947, complete genome |
| 486 | >MH061880.1 Zika virus strain ZIKV/Macaca mulatta/UGA/MR-766-VEROE6-P12_08/1947, |
| | complete genome |
| 487 | >MH061881.1 Zika virus strain ZIKV/Macaca mulatta/UGA/MR-766-VEROE6-GD12- |
| | P10_02/1947, complete genome |
| 488 | >MH061882.1 Zika virus strain ZIKV/Macaca mulatta/UGA/MR-766-VEROE6-GD12- |
| | P10_08/1947, complete genome |
| 489 | >MH061883.1 Zika virus strain ZIKV/Macaca mulatta/UGA/MR-766-VEROE6-FC11-P7_08/1947, |
| | complete genome |
| 490 | >MH061884.1 Zika virus strain ZIKV/Macaca mulatta/UGA/MR-766-VEROE6-GA3-P10_01/1947, |
| | complete genome |
| 491 | >MH061885.1 Zika virus strain ZIKV/Macaca mulatta/UGA/MR-766-VEROE6-FC3-P8_06/1947, |
| | complete genome |
| 492 | >MH061886.1 Zika virus strain ZIKV/Macaca mulatta/UGA/MR-766-VEROE6-FA12-P7_05/1947, |
| | complete genome |
| 493 | >MH061887.1 Zika virus strain ZIKV/Macaca mulatta/UGA/MR-766-VEROE6-P12_05/1947, |
| | complete genome |
| 494 | >MH061888.1 Zika virus strain ZIKV/Macaca mulatta/UGA/MR-766-VEROE6-AC11-P5_02/1947, |
| | complete genome |
| 495 | >MH061889.1 Zika virus strain ZIKV/Macaca mulatta/UGA/MR-766-VEROE6-FC3-P8_07/1947, |
| | complete genome |
| 496 | >MH061890.1 Zika virus strain ZIKV/Macaca mulatta/UGA/MR-766-VEROE6-FC3-P8_08/1947, |
| | complete genome |
| 497 | >MH061891.1 Zika virus strain ZIKV/Macaca mulatta/UGA/MR-766-VEROE6-FA12-P7_06/1947, |
| | complete genome |
| 498 | >MH061892.1 Zika virus strain ZIKV/Macaca mulatta/UGA/MR-766-VEROE6-P12_03/1947, |
| | complete genome |
| 499 | >MH061893.1 Zika virus strain ZIKV/Macaca mulatta/UGA/MR-766-VEROE6-GD12- |
| | P10_03/1947, complete genome |
| 500 | >MH061894.1 Zika virus strain ZIKV/Macaca mulatta/UGA/MR-766-VEROE6-FC11-P7_03/1947, |
| | complete genome |
| 501 | >MH061895.1 Zika virus strain ZIKV/Macaca mulatta/UGA/MR-766-VEROE6-P12_01/1947, |
| | complete genome |
| 502 | >MH061896.1 Zika virus strain ZIKV/Macaca mulatta/UGA/MR-766-VEROE6-FC3-P8_04/1947, |
| | complete genome |
| 503 | >MH061897.1 Zika virus strain ZIKV/Macaca mulatta/UGA/MR-766-VEROE6-FC3-P8_01/1947, |
| | complete genome |
| 504 | >MH061898.1 Zika virus strain ZIKV/Macaca mulatta/UGA/MR-766-VEROE6-FC11-P7_02/1947, |
| | complete genome |
| 505 | >MH061899.1 Zika virus strain ZIKV/Macaca mulatta/UGA/MR-766-VEROE6-AA1-P8_08/1947, |
| | complete genome |
| 506 | >MH061900.1 Zika virus strain ZIKV/Macaca mulatta/UGA/MR-766-VEROE6-P12_04/1947, |
| | complete genome |
| 507 | >MH061901.1 Zika virus strain ZIKV/Macaca mulatta/UGA/MR-766-VEROE6-GA3-P10_07/1947, |
| | complete genome |
| 508 | >MH061902.1 Zika virus strain ZIKV/Macaca mulatta/UGA/MR-766-VEROE6-FA12-P7_08/1947, |
| | complete genome |
| 509 | >MH061903.1 Zika virus strain ZIKV/Macaca mulatta/UGA/MR-766-VEROE6-AA1-P8_04/1947, |
| | complete genome |
| 510 | >MH061904.1 Zika virus strain ZIKV/Macaca mulatta/UGA/MR-766-VEROE6-FC3-P8_02/1947, |
| | complete genome |
| 511 | >MH061905.1 Zika virus strain ZIKV/Macaca mulatta/UGA/MR-766-VEROE6-AA1-P8_01/1947, |
| | complete genome |
| 512 | >MH061906.1 Zika virus strain ZIKV/Macaca mulatta/UGA/MR-766-VEROE6-FC11-P7_01/1947, |
| | complete genome |
| 513 | >MH061907.1 Zika virus strain ZIKV/Macaca mulatta/UGA/MR-766-VEROE6-GA3-P10_06/1947, |
| | complete genome |
| 514 | >MH061908.1 Zika virus strain ZIKV/Macaca mulatta/UGA/MR-766-VEROE6-P12_06/1947, |
| | complete genome |
| 515 | >MH061909.1 Zika virus strain ZIKV/Macaca mulatta/UGA/MR-766-VEROE6-AC11-P5_04/1947, |
| | complete genome |
| 516 | >MH061910.1 Zika virus strain ZIKV/Macaca mulatta/UGA/MR-766-VEROE6-FA12-P7_04/1947, |
| | complete genome |
| 517 | >MH061911.1 Zika virus strain ZIKV/Macaca mulatta/UGA/MR-766-VEROE6-GD12- |
| | P10_06/1947, complete genome |
| 518 | >MH061912.1 Zika virus strain ZIKV/Macaca mulatta/UGA/MR-766-VEROE6-GA3-P10_03/1947, |
| | complete genome |
| 519 | >MH061913.1 Zika virus strain ZIKV/Macaca mulatta/UGA/MR-766-VEROE6-GA3-P10_05/1947, |
| | complete genome |
| 520 | >MH061914.1 Zika virus strain ZIKV/Macaca mulatta/UGA/MR-766-VEROE6-GA3-P10_02/1947, |
| | complete genome |
| 521 | >MH061915.1 Zika virus strain ZIKV/Macaca mulatta/UGA/MR-766-VEROE6-P12_02/1947, |
| | complete genome |
| 522 | >LC369584.1 Zika virus ZIKV/Hu/Thai/KngSG/17-D501 genomic RNA, nearly complete genome |
| 523 | >MG827392.1 Zika virus strain PF13/251013-18, complete genome |
| 524 | >MG674718.1 Zika virus isolate ZKC2P4, complete genome |
| 525 | >MG674719.1 Zika virus isolate ZKC2P6, complete genome |
| 526 | >KY766069.1 Zika virus isolate Pf13/251013-18, complete genome |
| 527 | >MF794971.1 Zika virus strain ZIKV/EC/Esmeraldas/121/2016, complete genome |
| 528 | >KY553111.1 Zika virus isolate AFMC-U, complete genome |
| 529 | >MF384325.1 Zika virus isolate mosquito/Haiti/1682/2016, complete genome |
| 530 | >MF352141.1 Zika virus isolate PE243, complete genome |
| 531 | >KY415986.1 Zika virus isolate Haiti/0029/2014, complete genome |
| 532 | >KY415987.1 Zika virus isolate Haiti/0033/2014, complete genome |
| 533 | >KY415988.1 Zika virus isolate Haiti/0054/2014, complete genome |
| 534 | >KY415989.1 Zika virus isolate Haiti/0074/2014, complete genome |
| 535 | >KY415990.1 Zika virus isolate Haiti/0036/2014, complete genome |
| 536 | >KY415991.1 Zika virus isolate Haiti/0097/2014, complete genome |
| 537 | >KY989511.1 Zika virus strain MR 766, complete genome |
| 538 | >KY989971.1 Zika virus isolate FLA, complete genome |
| 539 | >KX051560.1 Zika virus isolate SK364/13AS, complete genome |
| 540 | >KX051561.1 Zika virus isolate SK403/13AS, complete genome |
| 541 | >KX051562.1 Zika virus isolate SV0010/15, complete genome |
| 542 | >KY765317.1 Zika virus strain ZIKV/Homo sapiens/NIC/7252_12A1/2016, complete genome |
| 543 | >KY765318.1 Zika virus strain ZIKV/Homo sapiens/NIC/4886_12A1/2016, complete genome |
| 544 | >KY765320.1 Zika virus strain ZIKV/Homo sapiens/NIC/6406_13A1_SP/2016, complete genome |
| 545 | >KY765321.1 Zika virus strain ZIKV/Homo sapiens/NIC/4886_12A1_SP/2016, complete genome |
| 546 | >KY765322.1 Zika virus strain ZIKV/Homo sapiens/NIC/7252_12A1_SP/2016, complete genome |
| 547 | >KY765323.1 Zika virus strain ZIKV/Homo sapiens/NIC/6188_13A1/2016, complete genome |
| 548 | >KY765324.1 Zika virus strain ZIKV/Homo sapiens/NIC/8610_13A1/2016, complete genome |
| 549 | >KY765325.1 Zika virus strain ZIKV/Homo sapiens/NIC/5005_13A1/2016, complete genome |
| 550 | >KY765326.1 Zika virus strain ZIKV/Homo sapiens/NIC/6188_13A1_SP/2016, complete genome |
| 551 | >KY765327.1 Zika virus strain ZIKV/Homo sapiens/NIC/5005_13A1_SP/2016, complete genome |
| 552 | >KY631493.1 Zika virus isolate MEX_ENCB165, complete genome |
| 553 | >KY631494.1 Zika virus isolate MEX_ENCB165P4, complete genome |
| 554 | >LC219720.1 Zika virus genomic RNA, complete genome, strain: ZIKV/Hu/NIID123/2016 |
| 555 | >KY648934.1 Zika virus strain ZIKV/Aedes aegypti/MEX/MEX_I-44/2016, complete genome |
| 556 | >KX827268.1 Zika virus strain ZIKV/Homo sapiens/USA/UT-1/2016, complete genome |
| 557 | >KY348640.1 Zika virus strain SL1602, complete genome |
| 558 | >KU761560.1 Zika virus isolate ZJ03, complete genome |
| 559 | >KU761561.1 Zika virus isolate ZJ02, complete genome |
| 560 | >KY272991.1 Zika virus isolate RIO-BM1, complete genome |
| 561 | >KY288905.1 Zika virus strain MP1751, complete genome |
| 562 | >KY328289.1 Zika virus isolate HN16, complete genome |
| 563 | >KY272987.1 Zika virus isolate SI-BKK01, complete genome |
| 564 | >KY120348.1 Zika virus strain MEX_CIENI551P4, complete genome |
| 565 | >KY120349.2 Zika virus strain MEX_CIENI551, complete genome |
| 566 | >KX269878.1 Zika virus isolate Haiti/2016/PD, complete genome |
| 567 | >LC191864.1 Zika virus genomic RNA, complete genome, strain: ZIKV/Hu/Chiba/S36/2016 |
| 568 | >LC190723.1 Zika virus genomic RNA, complete genome, strain: ZIKV/Hu/Yokohama/1/2016 |
| 569 | >KX893855.1 Zika virus strain Zika virus/Homo sapiens/VEN/UF-2/2016, complete genome |
| 570 | >KX879603.1 Zika virus strain ZIKV/EC/Esmeraldas/062/2016, complete genome |
| 571 | >KX879604.1 Zika virus strain ZIKV/EC/Esmeraldas/089/2016, complete genome |
| 572 | >KX830960.1 Zika virus culture ATCC: VR-84 isolate MR776, complete genome |
| 573 | >KX811222.1 Zika virus isolate Brazil_2015_MG, complete genome |
| 574 | >KX856011.1 Zika virus strain ZIKV/Aedes sp./MEX_I-44/2016, complete genome |
| 575 | >KX197205.1 Zika virus isolate 9, complete genome |
| 576 | >KX806557.3 Zika virus isolate TS17-2016, complete genome |
| 577 | >KX766028.1 Zika virus isolate R114916, complete genome |
| 578 | >KX766029.1 Zika virus isolate R116265, complete genome |
| 579 | >KX702400.1 Zika virus strain Zika virus/Homo sapiens/VEN/UF-1/2016, complete genome |
| 580 | >KX694532.2 Zika virus strain ZIKV/Homo sapiens/THA/PLCal_ZV/2013, complete genome |
| 581 | >KX694533.2 Zika virus strain ZIKV/Aedes aegypti/MYS/P6-740/1966, complete genome |
| 582 | >KX694534.2 Zika virus strain ZIKV/Homo sapiens/HND/R103451/2015, complete genome |
| 583 | >KX673530.1 Zika virus isolate PHE_semen_Guadeloupe, complete genome |
| 584 | >KX266255.1 Zika virus isolate ZIKV_SMGC-1, complete genome |
| 585 | >KX601166.2 Zika virus strain ZIKV/Aedes africanus/SEN/DakAr41524/1984, complete genome |
| 586 | >KX601167.1 Zika virus strain ZIKV/Aedes sp./MYS/P6-740/1966, complete genome |
| 587 | >KX601168.1 Zika virus strain ZIKV/Homo Sapiens/PRI/PRVABC59/2015, complete genome |
| 588 | >KX601169.1 Zika virus strain ZIKV/Macaca mulatta/UGA/MR-766/1947, complete genome |
| 589 | >KX369547.1 Zika virus strain PF13/251013-18, complete genome |
| 590 | >KX446950.2 Zika virus strain ZIKV/Aedes. sp/MEX/MEX_2-81/2016, complete genome |
| 591 | >KX446951.2 Zika virus strain ZIKV/Aedes. sp/MEX/MEX_I-7/2016, complete genome |
| 592 | >KX377335.1 Zika virus strain MR-766, complete genome |
| 593 | >KX377336.1 Zika virus strain P6-740, complete genome |
| 594 | >KX377337.1 Zika virus strain PRVABC-59, complete genome |
| 595 | >KX280026.1 Zika virus isolate Paraiba_01, complete genome |
| 596 | >KX262887.1 Zika virus isolate 103451, complete genome |
| 597 | >KX253996.1 Zika virus isolate ZKC2/2016, complete genome |
| 598 | >KX247646.1 Zika virus isolate Zika virus/Homo sapiens/COL/UF-1/2016, complete genome |
| 599 | >KX197192.1 Zika virus isolate ZIKV/H. sapiens/Brazil/PE243/2015, complete genome |
| 600 | >KX198134.2 Zika virus strain ZIKV/Aedes africanus/SEN/DAK-AR-41524_A1C1-V2/1984, |
| | complete genome |
| 601 | >KX198135.2 Zika virus strain ZIKV/Homo sapiens/PAN/BEI-259634_V4/2016, complete genome |
| 602 | >KX156774.2 Zika virus strain ZIKV/Homo sapiens/PAN/CDC-259359_V1-V3/2015, complete |
| | genome |
| 603 | >KX156775.2 Zika virus strain ZIKV/Homo sapiens/PAN/CDC-259249_V1-V3/2015, complete |
| | genome |
| 604 | >KX156776.2 Zika virus strain ZIKV/Homo sapiens/PAN/CDC-259364_V1-V2/2015, complete |
| | genome |
| 605 | >KX117076.1 Zika virus isolate Zhejiang04, complete genome |
| 606 | >KX087101.3 Zika virus strain ZIKV/Homo sapiens/PRI/PRVABC59/2015, complete genome |
| 607 | >KX087102.2 Zika virus strain ZIKV/Homo sapiens/COL/FLR/2015, complete genome |
| 608 | >KX051563.1 Zika virus isolate Haiti/1/2016, complete genome |
| 609 | >KU955591.1 Zika virus isolate Zika virus/A. africanus-tc/SEN/1984/41525-DAK, complete genome |
| 610 | >KU955592.1 Zika virus isolate Zika virus/A. taylori-tc/SEN/1984/41662-DAK, complete genome |
| 611 | >KU955593.1 Zika virus isolate Zika virus/H. sapiens-tc/KHM/2010/FSS13025, complete genome |
| 612 | >KU955594.1 Zika virus isolate Zika virus/M. mulatta-tc/UGA/1947/MR-766, complete genome |
| 613 | >KU955595.1 Zika virus isolate Zika virus/A. taylori-tc/SEN/1984/41671-DAK, complete genome |
| 614 | >KU870645.1 Zika virus isolate FB-GWUH-2016, complete genome |
| 615 | >KU926309.2 Zika virus isolate Rio-U1, complete genome |
| 616 | >KU926310.2 Zika virus isolate Rio-S1, complete genome |
| 617 | >KU922923.1 Zika virus isolate MEX/InDRE/Lm/2016, complete genome |
| 618 | >KU922960.1 Zika virus isolate MEX/InDRE/Sm/2016, complete genome |
| 619 | >KU853012.1 Zika virus isolate Dominican Republic/2016/PD1, complete genome |
| 620 | >KU853013.1 Zika virus isolate Dominican Republic/2016/PD2, complete genome |
| 621 | >KU820899.2 Zika virus isolate ZJ03, complete genome |
| 622 | >KU744693.1 Zika virus isolate VE_Ganxian, complete genome |
| 623 | >KU497555.1 Zika virus isolate Brazil-ZKV2015, complete genome |
| 624 | >KU707826.1 Zika virus isolate SSABR1, complete genome |
| 625 | >KU527068.1 Zika virus strain Natal RGN, complete genome |
| 626 | >KU681081.3 Zika virus isolate Zika virus/H. sapiens-tc/THA/2014/SV0127- 14, complete genome |
| 627 | >KU681082.3 Zika virus isolate Zika virus/H. sapiens-tc/PHL/2012/CPC-0740, complete genome |
| 628 | >KU509998.3 Zika virus strain Haiti/1225/2014, complete genome |
| 629 | >KU501215.1 Zika virus strain PRVABC59, complete genome |
| 630 | >KU321639.1 Zika virus strain ZikaSPH2015, complete genome |
| 631 | >LC002520.1 Zika virus genomic RNA, complete genome, strain: MR766-NIID |
| 632 | >KJ776791.2 Zika virus strain H/PF/2013, complete genome |
| 633 | >AY632535.2 Zika virus strain MR 766, complete genome |

TABLE 1D
Non-limiting example Zika Capsid (C) sequences

| SEQ | Zika viruses with example Capsid | SEQ | Zika viruses having with example Capsid |
|---|---|---|---|
| ID NO | proteins | ID NO | proteins |
| 634 | >YP_009430295.1 anchored capsid protein C | 891 | >AWF93634.1 Flavivirus capsid protein C |
| 635 | >YP_009430295.1 Flavivirus capsid protein C | 892 | >AWF93635.1 capsid protein C |
| 636 | >YP_009430296.1 capsid protein C | 893 | >AWF93635.1 Flavivirus capsid protein C |
| 637 | >YP_009430296.1 Flavivirus capsid protein C | 894 | >AVZ47169.1 Flavivirus capsid protein C |
| 638 | >YP_009428568.1 anchored capsid protein C | 895 | >AVW85800.1 capsid protein C |
| 639 | >YP_009428568.1 capsid protein C | 896 | >AVW85800.1 Flavivirus capsid protein C |
| 640 | >YP_009428568.1 Flavivirus capsid protein C | 897 | >AVW85801.1 capsid protein C |
| 641 | >YP_009227196.1 capsid protein C | 898 | >AVW85801.1 Flavivirus capsid protein C |
| 642 | >YP_009227196.1 Flavivirus capsid protein C | 899 | >AVW85802.1 capsid protein C |
| 643 | >YP_009227206.1 anchored capsid protein C | 900 | >AVW85802.1 Flavivirus capsid protein C |
| 644 | >YP_009227206.1 Flavivirus capsid protein C | 901 | >AVW85803.1 capsid protein C |
| 645 | >YP_002790881.1 capsid protein C | 902 | >AVW85803.1 Flavivirus capsid protein C |
| 646 | >YP_002790881.1 Flavivirus capsid protein C | 903 | >AVW85804.1 capsid protein C |
| 647 | >UHI99865.1 anchored capsid protein C | 904 | >AVW85804.1 Flavivirus capsid protein C |
| 648 | >UHI99865.1 capsid protein C | 905 | >AVW85805.1 capsid protein C |
| 649 | >UHI99865.1 Flavivirus capsid protein C | 906 | >AVW85805.1 Flavivirus capsid protein C |
| 650 | >UHI99866.1 anchored capsid protein C | 907 | >AVW85806.1 capsid protein C |
| 651 | >UHI99866.1 capsid protein C | 908 | >AVW85806.1 Flavivirus capsid protein C |
| 652 | >UHI99866.1 Flavivirus capsid protein C | 909 | >AVW85807.1 capsid protein C |
| 653 | >UHI99867.1 anchored capsid protein C | 910 | >AVW85807.1 Flavivirus capsid protein C |
| 654 | >UHI99867.1 capsid protein C | 911 | >AVW85808.1 capsid protein C |
| 655 | >UED15620.1 anchored capsid protein C | 912 | >AVW85808.1 Flavivirus capsid protein C |
| 656 | >UED15620.1 capsid protein C | 913 | >AVW85809.1 capsid protein C |
| 657 | >UED15620.1 Flavivirus capsid protein C | 914 | >AVW85809.1 Flavivirus capsid protein C |
| 658 | >UBI73854.1 anchored capsid protein C | 915 | >AVW85810.1 capsid protein C |
| 659 | >UBI73854.1 capsid protein C | 916 | >AVW85810.1 Flavivirus capsid protein C |
| 660 | >UBI73854.1 Flavivirus capsid protein C | 917 | >AVW85811.1 capsid protein C |
| 661 | >QKF93428.1 anchored capsid protein C | 918 | >AVW85811.1 Flavivirus capsid protein C |
| 662 | >QKF93428.1 capsid protein C | 919 | >AVW85812.1 capsid protein C |
| 663 | >QKF93428.1 Flavivirus capsid protein C | 920 | >AVW85812.1 Flavivirus capsid protein C |
| 664 | >QKF93429.1 anchored capsid protein C | 921 | >AVW85813.1 capsid protein C |
| 665 | >QKF93429.1 capsid protein C | 922 | >AVW85813.1 Flavivirus capsid protein C |
| 666 | >QKF93429.1 Flavivirus capsid protein C | 923 | >AVW85814.1 capsid protein C |
| 667 | >QKF93430.1 anchored capsid protein C | 924 | >AVW85814.1 Flavivirus capsid protein C |
| 668 | >QKF93430.1 capsid protein C | 925 | >AVW85815.1 capsid protein C |
| 669 | >QKF93430.1 Flavivirus capsid protein C | 926 | >AVW85815.1 Flavivirus capsid protein C |
| 670 | >QKF93431.1 anchored capsid protein C | 927 | >AVW85816.1 capsid protein C |
| 671 | >QKF93431.1 capsid protein C | 928 | >AVW85816.1 Flavivirus capsid protein C |
| 672 | >QKF93431.1 Flavivirus capsid protein C | 929 | >AVW85817.1 capsid protein C |
| 673 | >QKF93432.1 anchored capsid protein C | 930 | >AVW85817.1 Flavivirus capsid protein C |
| 674 | >QKF93432.1 capsid protein C | 931 | >AVW85818.1 capsid protein C |
| 675 | >QKF93432.1 Flavivirus capsid protein C | 932 | >AVW85818.1 Flavivirus capsid protein C |
| 676 | >QKF93433.1 anchored capsid protein C | 933 | >AVW85819.1 capsid protein C |
| 677 | >QKF93433.1 capsid protein C | 934 | >AVW85819.1 Flavivirus capsid protein C |
| 678 | >QKF93433.1 Flavivirus capsid protein C | 935 | >AVW85820.1 capsid protein C |
| 679 | >QKF93434.1 anchored capsid protein C | 936 | >AVW85820.1 Flavivirus capsid protein C |
| 680 | >QKF93434.1 capsid protein C | 937 | >AVW85821.1 capsid protein C |
| 681 | >QKF93434.1 Flavivirus capsid protein C | 938 | >AVW85821.1 Flavivirus capsid protein C |
| 682 | >QKF93435.1 anchored capsid protein C | 939 | >AVW85822.1 capsid protein C |
| 683 | >QKF93435.1 capsid protein C | 940 | >AVW85822.1 Flavivirus capsid protein C |
| 684 | >QKF93435.1 Flavivirus capsid protein C | 941 | >AVW85823.1 capsid protein C |
| 685 | >QKF93436.1 anchored capsid protein C | 942 | >AVW85823.1 Flavivirus capsid protein C |
| 686 | >QKF93436.1 capsid protein C | 943 | >AVW85824.1 capsid protein C |
| 687 | >QKF93436.1 Flavivirus capsid protein C | 944 | >AVW85824.1 Flavivirus capsid protein C |
| 688 | >QKF93437.1 anchored capsid protein C | 945 | >AVW85825.1 capsid protein C |
| 689 | >QKF93437.1 capsid protein C | 946 | >AVW85825.1 Flavivirus capsid protein C |
| 690 | >QKF93437.1 Flavivirus capsid protein C | 947 | >AVW85826.1 capsid protein C |
| 691 | >QKF93438.1 anchored capsid protein C | 948 | >AVW85826.1 Flavivirus capsid protein C |
| 692 | >QKF93438.1 capsid protein C | 949 | >AVW85827.1 capsid protein C |
| 693 | >QKF93438.1 Flavivirus capsid protein C | 950 | >AVW85827.1 Flavivirus capsid protein C |
| 694 | >QKF93439.1 anchored capsid protein C | 951 | >AVW85828.1 capsid protein C |
| 695 | >QKF93439.1 capsid protein C | 952 | >AVW85828.1 Flavivirus capsid protein C |
| 696 | >QKF93439.1 Flavivirus capsid protein C | 953 | >AVW85829.1 capsid protein C |
| 697 | >QKF93440.1 anchored capsid protein C | 954 | >AVW85829.1 Flavivirus capsid protein C |
| 698 | >QKF93440.1 capsid protein C | 955 | >AVW85830.1 capsid protein C |
| 699 | >QKF93440.1 Flavivirus capsid protein C | 956 | >AVW85830.1 Flavivirus capsid protein C |
| 700 | >QKF93441.1 anchored capsid protein C | 957 | >AVW85831.1 capsid protein C |
| 701 | >QKF93441.1 capsid protein C | 958 | >AVW85831.1 Flavivirus capsid protein C |
| 702 | >QKF93441.1 Flavivirus capsid protein C | 959 | >AVW85832.1 capsid protein C |
| 703 | >QVL61761.1 Flavivirus capsid protein C | 960 | >AVW85832.1 Flavivirus capsid protein C |
| 704 | >QPC45464.1 Flavivirus capsid protein C | 961 | >AVW85833.1 capsid protein C |
| 705 | >QTA38993.1 anchored capsid protein C | 962 | >AVW85833.1 Flavivirus capsid protein C |
| 706 | >QTA38993.1 Flavivirus capsid protein C | 963 | >AVW85834.1 capsid protein C |
| 707 | >QTA38994.1 anchored capsid protein C | 964 | >AVW85834.1 Flavivirus capsid protein C |
| 708 | >QTA38994.1 Flavivirus capsid protein C | 965 | >AVW85835.1 capsid protein C |
| 709 | >QOF88708.1 anchored capsid protein C | 966 | >AVW85835.1 Flavivirus capsid protein C |
| 710 | >QOF88708.1 capsid protein C | 967 | >AVW85836.1 capsid protein C |
| 711 | >QOF88708.1 Flavivirus capsid protein C | 968 | >AVW85836.1 Flavivirus capsid protein C |
| 712 | >QLI34051.1 Flavivirus capsid protein C | 969 | >AVW85837.1 capsid protein C |
| 713 | >QKX95861.1 capsid protein C | 970 | >AVW85837.1 Flavivirus capsid protein C |
| 714 | >QKX95861.1 Flavivirus capsid protein C | 971 | >AVW85838.1 capsid protein C |
| 715 | >QKU35968.1 anchored capsid protein C | 972 | >AVW85838.1 Flavivirus capsid protein C |
| 716 | >QKU35968.1 capsid protein C | 973 | >AVW85839.1 capsid protein C |
| 717 | >QKU35968.1 Flavivirus capsid protein C | 974 | >AVW85839.1 Flavivirus capsid protein C |
| 718 | >QKM77298.1 anchored capsid protein C | 975 | >AVW85840.1 capsid protein C |
| 719 | >QKM77298.1 capsid protein C | 976 | >AVW85840.1 Flavivirus capsid protein C |
| 720 | >OKM77298. 1 Flavivirus capsid protein C | 977 | >AVW85841.1 capsid protein C |
| 721 | >QKM77299.1 anchored capsid protein C | 978 | >AVW85841.1 Flavivirus capsid protein C |
| 722 | >QKM77299.1 capsid protein C | 979 | >AVW85842.1 capsid protein C |
| 723 | >QKM77299.1 Flavivirus capsid protein C | 980 | >AVW85842.1 Flavivirus capsid protein C |
| 724 | >QKM77300.1 anchored capsid protein C | 981 | >AVW85843.1 capsid protein C |
| 725 | >QKM77300.1 capsid protein C | 982 | >AVW85843.1 Flavivirus capsid protein C |
| 726 | >QKM77300.1 Flavivirus capsid protein C | 983 | >AVW85844.1 capsid protein C |
| 727 | >QKM77301.1 anchored capsid protein C | 984 | >AVW85844.1 Flavivirus capsid protein C |
| 728 | >QKM77301.1 capsid protein C | 985 | >AVW85845.1 capsid protein C |
| 729 | >QKM77301.1 Flavivirus capsid protein C | 986 | >AVW85845.1 Flavivirus capsid protein C |
| 730 | >QBM11757.1 capsid | 987 | >AVW85846.1 capsid protein C |
| 731 | >QBM11757.1 Flavivirus capsid protein C | 988 | >AVW85846.1 Flavivirus capsid protein C |
| 732 | >AZS35403.1 Flavivirus capsid protein C | 989 | >AVW85847.1 capsid protein C |
| 733 | >AZS35404.1 Flavivirus capsid protein C | 990 | >AVW85847.1 Flavivirus capsid protein C |
| 734 | >AZS35405.1 Flavivirus capsid protein C | 991 | >AVW85848.1 capsid protein C |
| 735 | >QGA72951.1 anchored capsid protein C | 992 | >AVW85848.1 Flavivirus capsid protein C |
| 736 | >QGA72951.1 capsid protein C | 993 | >AVW85849.1 capsid protein C |
| 737 | >QGA72951.1 Flavivirus capsid protein C | 994 | >AVW85849.1 Flavivirus capsid protein C |
| 738 | >QGA67050.1 anchored capsid protein C | 995 | >AVW85850.1 capsid protein C |
| 739 | >QGA67050.1 capsid protein C | 996 | >AVW85850.1 Flavivirus capsid protein C |
| 740 | >QGA67050.1 Flavivirus capsid protein C | 997 | >AVW85851.1 capsid protein C |
| 741 | >QGA67051.1 anchored capsid protein C | 998 | >AVW85851.1 Flavivirus capsid protein C |
| 742 | >QGA67051.1 capsid protein C | 999 | >AVW85852.1 capsid protein C |
| 743 | >QGA67051.1 Flavivirus capsid protein C | 1000 | >AVW85852.1 Flavivirus capsid protein C |
| 744 | >QGA67052.1 anchored capsid protein C | 1001 | >AVW85853.1 capsid protein C |
| 745 | >QGA67052.1 capsid protein C | 1002 | >AVW85853.1 Flavivirus capsid protein C |
| 746 | >QGA67052.1 Flavivirus capsid protein C | 1003 | >AVW85854.1 capsid protein C |
| 747 | >QGA67053.1 anchored capsid protein C | 1004 | >AVW85854.1 Flavivirus capsid protein C |
| 748 | >QGA67053.1 capsid protein C | 1005 | >AVW85855.1 capsid protein C |
| 749 | >QGA67053.1 Flavivirus capsid protein C | 1006 | >AVW85855.1 Flavivirus capsid protein C |
| 750 | >QGA67054.1 anchored capsid protein C | 1007 | >AVW85856.1 capsid protein C |
| 751 | >QGA67054.1 capsid protein C | 1008 | >AVW85856.1 Flavivirus capsid protein C |
| 752 | >QGA67054.1 Flavivirus capsid protein C | 1009 | >AVW85857.1 capsid protein C |
| 753 | >QGA67055.1 anchored capsid protein C | 1010 | >AVW85857.1 Flavivirus capsid protein C |
| 754 | >QGA67055.1 capsid protein C | 1011 | >AVW85858.1 capsid protein C |
| 755 | >QGA67055.1 Flavivirus capsid protein C | 1012 | >AVW85858.1 Flavivirus capsid protein C |
| 756 | >QGA67056.1 anchored capsid protein C | 1013 | >AVW85859.1 capsid protein C |
| 757 | >QGA67056.1 capsid protein C | 1014 | >AVW85859.1 Flavivirus capsid protein C |
| 758 | >QGA67056.1 Flavivirus capsid protein C | 1015 | >AVW85860.1 capsid protein C |
| 759 | >QFZ95661.1 anchored capsid protein C | 1016 | >AVW85860.1 Flavivirus capsid protein C |
| 760 | >QFZ95661.1 capsid protein C | 1017 | >AVW85861.1 capsid protein C |
| 761 | >QFZ95661.1 Flavivirus capsid protein C | 1018 | >AVW85861.1 Flavivirus capsid protein C |
| 762 | >QDZ58854.1 Flavivirus capsid protein C | 1019 | >AVW85862.1 capsid protein C |
| 763 | >QDZ58855.1 Flavivirus capsid protein C | 1020 | >AVW85862.1 Flavivirus capsid protein C |
| 764 | >QDZ58856.1 Flavivirus capsid protein C | 1021 | >AVW85863.1 capsid protein C |
| 765 | >AXQ39996.1 anchored capsid protein C | 1022 | >AVW85863.1 Flavivirus capsid protein C |
| 766 | >AXQ39996.1 capsid protein C | 1023 | >BBC70847.1 Flavivirus capsid protein C |
| 767 | >AXQ39996.1 Flavivirus capsid protein C | 1024 | >AVG19275.1 Flavivirus capsid protein C |
| 768 | >AXQ39997.1 anchored capsid protein C | 1025 | >AUI42329.1 Flavivirus capsid protein C |
| 769 | >AXQ39997.1 capsid protein C | 1026 | >AUI42330.1 Flavivirus capsid protein C |
| 770 | >AXQ39997.1 Flavivirus capsid protein C | 1027 | >ARB08102.1 Flavivirus capsid protein C |
| 771 | >AXQ39998.1 anchored capsid protein C | 1028 | >ATB53752.1 Flavivirus capsid protein C |
| 772 | >AXQ39998.1 capsid protein C | 1029 | >AQS26698.1 Flavivirus capsid protein C |
| 773 | >AXQ39998.1 Flavivirus capsid protein C | 1030 | >ASF57880.1 Flavivirus capsid protein C |
| 774 | >AXQ39999.1 anchored capsid protein C | 1031 | >ASB32509.1 capsid protein C |
| 775 | >AXQ39999.1 capsid protein C | 1032 | >ASB32509.1 Flavivirus capsid protein C |
| 776 | >AXQ39999.1 Flavivirus capsid protein C | 1033 | >APW84872.1 Flavivirus capsid protein C |
| 777 | >AXQ40000.1 anchored capsid protein C | 1034 | >APW84873.1 Flavivirus capsid protein C |
| 778 | >AXQ40000.1 capsid protein C | 1035 | >APW84874.1 Flavivirus capsid protein C |
| 779 | >AXQ40000.1 Flavivirus capsid protein C | 1036 | >APW84875.1 Flavivirus capsid protein C |
| 780 | >AXQ40001.1 anchored capsid protein C | 1037 | >APW84876.1 Flavivirus capsid protein C |
| 781 | >AXQ40001.1 capsid protein C | 1038 | >APW84877.1 Flavivirus capsid protein C |
| 782 | >AXQ40001.1 Flavivirus capsid protein C | 1039 | >ARM59239.1 Flavivirus capsid protein C |
| 783 | >AXQ40002.1 anchored capsid protein C | 1040 | >ARM59240.1 capsid protein C |
| 784 | >AXQ40002.1 capsid protein C | 1041 | >ARM59240.1 Flavivirus capsid protein C |
| 785 | >AXQ40002.1 Flavivirus capsid protein C | 1042 | >AMX81915.1 Flavivirus capsid protein C |
| 786 | >AXQ40003.1 anchored capsid protein C | 1043 | >AMX81916.1 Flavivirus capsid protein C |
| 787 | >AXQ40003.1 capsid protein C | 1044 | >AMX81917.1 Flavivirus capsid protein C |
| 788 | >AXQ40003.1 Flavivirus capsid protein C | 1045 | >AQZ41947.1 capsid protein C |
| 789 | >AXQ40004.1 anchored capsid protein C | 1046 | >AQZ41947.1 Flavivirus capsid protein C |
| 790 | >AXQ40004.1 capsid protein C | 1047 | >AQZ41948.1 capsid protein C |
| 791 | >AXQ40004.1 Flavivirus capsid protein C | 1048 | >AQZ41948.1 Flavivirus capsid protein C |
| 792 | >AXQ40005.1 anchored capsid protein C | 1049 | >AQZ41949.1 capsid protein C |
| 793 | >AXQ40005.1 capsid protein C | 1050 | >AQZ41949.1 Flavivirus capsid protein C |
| 794 | >AXQ40005.1 Flavivirus capsid protein C | 1051 | >AQZ41950.1 capsid protein C |
| 795 | >AXQ40006.1 anchored capsid protein C | 1052 | >AQZ41950.1 Flavivirus capsid protein C |
| 796 | >AXQ40006.1 capsid protein C | 1053 | >AQZ41951.1 capsid protein C |
| 797 | >AXQ40006.1 Flavivirus capsid protein C | 1054 | >AQZ41951.1 Flavivirus capsid protein C |
| 798 | >AXQ40007.1 anchored capsid protein C | 1055 | >AQZ41952.1 capsid protein C |
| 799 | >AXQ40007.1 capsid protein C | 1056 | >AQZ41952.1 Flavivirus capsid protein C |
| 800 | >AXQ40007.1 Flavivirus capsid protein C | 1057 | >AQZ41953.1 capsid protein C |
| 801 | >QDH45910.1 Flavivirus capsid protein C | 1058 | >AQZ41953.1 Flavivirus capsid protein C |
| 802 | >QCG74166.1 Flavivirus capsid protein C | 1059 | >AQZ41954.1 capsid protein C |
| 803 | >AYV74916.1 Flavivirus capsid protein C | 1060 | >AQZ41954.1 Flavivirus capsid protein C |
| 804 | >AYV74917.1 Flavivirus capsid protein C | 1061 | >AQZ41955.1 capsid protein C |
| 805 | >AYV74918.1 Flavivirus capsid protein C | 1062 | >AQZ41955.1 Flavivirus capsid protein C |
| 806 | >AYV74919.1 Flavivirus capsid protein C | 1063 | >AQZ41956.1 capsid protein C |
| 807 | >AYV74920.1 Flavivirus capsid protein C | 1064 | >AQZ41956.1 Flavivirus capsid protein C |
| 808 | >AYV74921.1 Flavivirus capsid protein C | 1065 | >AQW43707.1 Flavivirus capsid protein C |
| 809 | >AYV74922.1 Flavivirus capsid protein C | 1066 | >AQW43708.1 Flavivirus capsid protein C |
| 810 | >AYV74923.1 Flavivirus capsid protein C | 1067 | >BAX00477.1 Flavivirus capsid protein C |
| 811 | >AYV74924.1 Flavivirus capsid protein C | 1068 | >AQV07165.1 capsid protein C |
| 812 | >AYV74925.1 Flavivirus capsid protein C | 1069 | >AQV07165.1 Flavivirus capsid protein C |
| 813 | >AYV74927.1 Flavivirus capsid protein C | 1070 | >AOO19564.1 Flavivirus capsid protein C |
| 814 | >AYV74928.1 Flavivirus capsid protein C | 1071 | >APO36913.1 Flavivirus capsid protein C |
| 815 | >AYV74929.1 Flavivirus capsid protein C | 1072 | >AMM43325.1 Flavivirus capsid protein C |
| 816 | >AYV74930.1 Flavivirus capsid protein C | 1073 | >AMM43326.1 Flavivirus capsid protein C |
| 817 | >AYV74931.1 Flavivirus capsid protein C | 1074 | >APH11492.1 anchored capsid protein C |
| 818 | >AYV74932.1 Flavivirus capsid protein C | 1075 | >APH11492.1 capsid protein C |
| 819 | >AYV74933.1 Flavivirus capsid protein C | 1076 | >APH11492.1 Flavivirus capsid protein C |
| 820 | >AYV74934.1 Flavivirus capsid protein C | 1077 | >APO08503.1 Flavivirus capsid protein C |
| 821 | >AYV74935.1 Flavivirus capsid protein C | 1078 | >APC60216.2 Flavivirus capsid protein C |
| 822 | >AYV74936.1 Flavivirus capsid protein C | 1079 | >ANH22038.1 Flavivirus capsid protein C |
| 823 | >AYV74937.1 Flavivirus capsid protein C | 1080 | >BAV82373.1 Flavivirus capsid protein C |
| 824 | >AYU74865.1 Flavivirus capsid protein C | 1081 | >AOR82892.1 Flavivirus capsid protein C |
| 825 | >ATB53753.1 Flavivirus capsid protein C | 1082 | >AOR82893.1 Flavivirus capsid protein C |
| 826 | >ATB53754.1 Flavivirus capsid protein C | 1083 | >AOR51315.1 Flavivirus capsid protein C |
| 827 | >AVC63697.1 Flavivirus capsid protein C | 1084 | >AND01116.2 Flavivirus capsid protein C |
| 828 | >AVC63698.1 Flavivirus capsid protein C | 1085 | >AOG18296.1 Flavivirus capsid protein C |
| 829 | >AYI50273.1 capsid protein C | 1086 | >AOC50652.1 capsid protein C |
| 830 | >AYI50273.1 Flavivirus capsid protein C | 1087 | >AOC50653.1 capsid protein C |
| 831 | >AYI50274.1 capsid protein C | 1088 | >AOC50654.1 capsid protein C |
| 832 | >AYI50274.1 Flavivirus capsid protein C | 1089 | >ANZ46736.1 Flavivirus capsid protein C |
| 833 | >AYI50275.1 capsid protein C | 1090 | >ANW07474.2 capsid protein C |
| 834 | >AYI50275.1 Flavivirus capsid protein C | 1091 | >ANW07474.2 Flavivirus capsid protein C |
| 835 | >ASR91936.1 Flavivirus capsid protein C | 1092 | >ANW07475.1 capsid protein C |
| 836 | >AXF50014.1 capsid | 1093 | >ANW07476.1 capsid protein C |
| 837 | >AXF50014.1 Flavivirus capsid protein C | 1094 | >ANW07476.1 Flavivirus capsid protein C |
| 838 | >AXF50015.1 capsid | 1095 | >ANW07477.1 capsid protein C |
| 839 | >AXF50015.1 Flavivirus capsid protein C | 1096 | >ANW07477.1 Flavivirus capsid protein C |
| 840 | >AXF50016.1 capsid | 1097 | >ANN44857.1 Flavivirus capsid protein C |
| 841 | >AXF50016.1 Flavivirus capsid protein C | 1098 | >ANN83272.1 capsid protein C |
| 842 | >AXF50052.1 Flavivirus capsid protein C | 1099 | >ANN83273.1 capsid protein C |
| 843 | >ATL14618.1 Flavivirus capsid protein C | 1100 | >ANK57897.1 Flavivirus capsid protein C |
| 844 | >AUF35021.1 Flavivirus capsid protein C | 1101 | >ANH10698.1 Flavivirus capsid protein C |
| 845 | >AUF35022.1 Flavivirus capsid protein C | 1102 | >ANG09399.1 Flavivirus capsid protein C |
| 846 | >AVG19202.1 Flavivirus capsid protein C | 1103 | >ANF16414.1 Flavivirus capsid protein C |
| 847 | >AVG19203.1 Flavivirus capsid protein C | 1104 | >ANC90426.1 capsid |
| 848 | >AVV62004.1 Flavivirus capsid protein C | 1105 | >ANC90427.1 capsid protein C |
| 849 | >ARU07074.1 Flavivirus capsid protein C | 1106 | >ANC90428.1 capsid protein C |
| 850 | >ARU07075.1 Flavivirus capsid protein C | 1107 | >ANC90428.1 Flavivirus capsid protein C |
| 851 | >ARU07076.1 Flavivirus capsid protein C | 1108 | >ANB66182.1 capsid protein C |
| 852 | >AUY62552.1 Flavivirus capsid protein C | 1109 | >ANB66182.1 Flavivirus capsid protein C |
| 853 | >AUY62553.1 Flavivirus capsid protein C | 1110 | >ANB66183.1 capsid protein C |
| 854 | >AWH65848.1 Flavivirus capsid protein C | 1111 | >ANB66183.1 Flavivirus capsid protein C |
| 855 | >AWH65849.1 Flavivirus capsid protein C | 1112 | >ANB66184.1 capsid protein C |
| 856 | >AWF93617.1 capsid protein C | 1113 | >ANA12599.1 Flavivirus capsid protein C |
| 857 | >AWF93617.1 Flavivirus capsid protein C | 1114 | >AMX81919.1 Flavivirus capsid protein C |
| 858 | >AWF93618.1 capsid protein C | 1115 | >AMR39832.1 Flavivirus capsid protein C |
| 859 | >AWF93618.1 Flavivirus capsid protein C | 1116 | >AMR39833.1 Flavivirus capsid protein C |
| 860 | >AWF93619.1 capsid protein C | 1117 | >AMR39834.1 Flavivirus capsid protein C |
| 861 | >AWF93619.1 Flavivirus capsid protein C | 1118 | >AMR39835.1 Flavivirus capsid protein C |
| 862 | >AWF93620.1 capsid protein C | 1119 | >AMR39836.1 Flavivirus capsid protein C |
| 863 | >AWF93620.1 Flavivirus capsid protein C | 1120 | >AMQ48986.1 Flavivirus capsid protein C |
| 864 | >AWF93621.1 capsid protein C | 1121 | >AMQ48981.1 anchored capsid protein C |
| 865 | >AWF93621.1 Flavivirus capsid protein C | 1122 | >AMQ48981.1 capsid protein C |
| 866 | >AWF93622.1 capsid protein C | 1123 | >AMQ48981.1 Flavivirus capsid protein C |
| 867 | >AWF93622.1 Flavivirus capsid protein C | 1124 | >AMQ48982.1 anchored capsid protein C |
| 868 | >AWF93623.1 capsid protein C | 1125 | >AMQ48982.1 capsid protein C |
| 869 | >AWF93623.1 Flavivirus capsid protein C | 1126 | >AMQ48982.1 Flavivirus capsid protein C |
| 870 | >AWF93624.1 capsid protein C | 1127 | >AMQ34003.1 capsid |
| 871 | >AWF93624.1 Flavivirus capsid protein C | 1128 | >AMQ34003.1 Flavivirus capsid protein C |
| 872 | >AWF93625.1 capsid protein C | 1129 | >AMQ34004.1 capsid |
| 873 | >AWF93625.1 Flavivirus capsid protein C | 1130 | >AMQ34004.1 Flavivirus capsid protein C |
| 874 | >AWF93626.1 capsid protein C | 1131 | >AMN14619.1 Flavivirus capsid protein C |
| 875 | >AWF93626.1 Flavivirus capsid protein C | 1132 | >AMN14620.1 Flavivirus capsid protein C |
| 876 | >AWF93627.1 capsid protein C | 1133 | >AMM39806.1 Flavivirus capsid protein C |
| 877 | >AWF93627.1 Flavivirus capsid protein C | 1134 | >AMK79469.1 Flavivirus capsid protein C |
| 878 | >AWF93628.1 capsid protein C | 1135 | >AMD16557.1 Flavivirus capsid protein C |
| 879 | >AWF93628.1 Flavivirus capsid protein C | 1136 | >AMH87239.1 Flavivirus capsid protein C |
| 880 | >AWF93629.1 capsid protein C | 1137 | >AMB18850.1 Flavivirus capsid protein C |
| 881 | >AWF93629.1 Flavivirus capsid protein C | 1138 | >AMD61710.1 Flavivirus capsid protein C |
| 882 | >AWF93630.1 capsid protein C | 1139 | >AMD61711.1 Flavivirus capsid protein C |
| 883 | >AWF93630.1 Flavivirus capsid protein C | 1140 | >AMB37295.1 Flavivirus capsid protein C |
| 884 | >AWF93631.1 capsid protein C | 1141 | >AMC13911.1 Flavivirus capsid protein C |
| 885 | >AWF93631.1 Flavivirus capsid protein C | 1142 | >ALU33341.1 capsid |
| 886 | >AWF93632.1 capsid protein C | 1143 | >ALU33341.1 Flavivirus capsid protein C |
| 887 | >AWF93632.1 Flavivirus capsid protein C | 1144 | >BAP47441.1 Flavivirus capsid protein C |
| 888 | >AWF93633.1 capsid protein C | 1145 | >AHZ13508.1 Flavivirus capsid protein C |
| 889 | >AWF93633.1 Flavivirus capsid protein C | 1146 | >AAV34151.1 Flavivirus capsid protein C |
| 890 | >AWF93634.1 capsid protein C | | |

TABLE 1E
Non-limiting example Envelope protein (E) sequences

| SEQ | Zika viruses with example Envelope |
|---|---|
| ID NO | protein (E) sequences |
| 1147 | >YP_009430300.1 envelope protein E |
| 1148 | >YP_009430300.1 Flavi_E_C |
| 1149 | >YP_009430300.1 flavi_E_stem |
| 1150 | >YP_009428568.1 envelope protein E |
| 1151 | >YP_009428568.1 Flavi_glycoprot |
| 1152 | >YP_009428568.1 Flavi_E_C |
| 1153 | >YP_009428568.1 flavi_E_stem |
| 1154 | >YP_009227198.1 envelope protein E |
| 1155 | >YP_009227198.1 Flavi_E_C |
| 1156 | >YP_009227198.1 flavi_E_stem |
| 1157 | >YP_002790881.1 envelope protein E |
| 1158 | >YP_002790881.1 Flavi_E_C |
| 1159 | >YP_002790881.1 flavi_E_stem |
| 1160 | >UHI99865.1 envelope protein E |
| 1161 | >UHI99865.1 Flavi_E_C |
| 1162 | >UHI99865.1 flavi_E_stem |
| 1163 | >UHI99866.1 envelope protein E |
| 1164 | >UHI99866.1 Flavi_E_C |
| 1165 | >UHI99866.1 flavi_E_stem |
| 1166 | >UHI99867.1 envelope protein E |
| 1167 | >UED15620.1 envelope protein E |
| 1168 | >UED15620.1 Flavi_E_C |
| 1169 | >UED15620.1 flavi_E_stem |
| 1170 | >UBI73854.1 envelope protein E |
| 1171 | >UBI73854.1 Flavi_E_C |
| 1172 | >UBI73854.1 flavi_E_stem |
| 1173 | >QKF93428.1 envelope protein E |
| 1174 | >QKF93428.1 Flavi_E_C |
| 1175 | >QKF93428.1 flavi_E_stem |
| 1176 | >QKF93429.1 envelope protein E |
| 1177 | >QKF93429.1 Flavi_E_C |
| 1178 | >QKF93429.1 flavi_E_stem |
| 1179 | >QKF93430.1 envelope protein E |
| 1180 | >QKF93430.1 Flavi_E_C |
| 1181 | >QKF93430.1 flavi_E_stem |
| 1182 | >QKF93431.1 envelope protein E |
| 1183 | >QKF93431.1 Flavi_E_C |
| 1184 | >QKF93431.1 flavi_E_stem |
| 1185 | >QKF93432.1 envelope protein E |
| 1186 | >QKF93432.1 Flavi_E_C |
| 1187 | >QKF93432.1 flavi_E_stem |
| 1188 | >QKF93433.1 envelope protein E |
| 1189 | >QKF93433.1 Flavi_E_C |
| 1190 | >QKF93433.1 flavi_E_stem |
| 1191 | >QKF93434.1 envelope protein E |
| 1192 | >QKF93434.1 Flavi_E_C |
| 1193 | >QKF93434.1 flavi_E_stem |
| 1194 | >QKF93435.1 envelope protein E |
| 1195 | >QKF93435.1 Flavi_E_C |
| 1196 | >QKF93435.1 flavi_E_stem |
| 1197 | >QKF93436.1 envelope protein E |
| 1198 | >QKF93436.1 Flavi_E_C |
| 1199 | >QKF93436.1 flavi_E_stem |
| 1200 | >QKF93437.1 envelope protein E |
| 1201 | >QKF93437.1 Flavi_E_C |
| 1202 | >QKF93437.1 flavi_E_stem |
| 1203 | >QKF93438.1 envelope protein E |
| 1204 | >QKF93438.1 Flavi_E_C |
| 1205 | >QKF93438.1 flavi_E_stem |
| 1206 | >QKF93439.1 envelope protein E |
| 1207 | >QKF93439.1 Flavi_E_C |
| 1208 | >QKF93439.1 flavi_E_stem |
| 1209 | >QKF93440.1 envelope protein E |
| 1210 | >QKF93440.1 Flavi_E_C |
| 1211 | >QKF93440.1 flavi_E_stem |
| 1212 | >QKF93441.1 envelope protein E |
| 1213 | >QKF93441.1 Flavi_E_C |
| 1214 | >QKF93441.1 flavi_E_stem |
| 1215 | >QVL61761.1 Flavi_E_C |
| 1216 | >QVL61761.1 flavi_E_stem |
| 1217 | >QPC45464.1 Flavi_E_C |
| 1218 | >QPC45464.1 flavi_E_stem |
| 1219 | >QTA38993.1 envelope protein E |
| 1220 | >QTA38993.1 Flavi_E_C |
| 1221 | >QTA38993.1 flavi_E_stem |
| 1222 | >QTA38994.1 envelope protein E |
| 1223 | >QTA38994.1 Flavi_E_C |
| 1224 | >QTA38994.1 flavi_E_stem |
| 1225 | >QOF88708.1 envelope protein E |
| 1226 | >QOF88708.1 Flavi_E_C |
| 1227 | >QOF88708.1 flavi_E_stem |
| 1228 | >QLI34051.1 Flavi_E_C |
| 1229 | >QLI34051.1 flavi_E_stem |
| 1230 | >QKX95861.1 envelope protein E |
| 1231 | >QKX95861.1 Flavi_E_C |
| 1232 | >QKX95861.1 flavi_E_stem |
| 1233 | >QKU35968.1 envelope protein E |
| 1234 | >QKU35968.1 Flavi_E_C |
| 1235 | >QKU35968.1 flavi_E_stem |
| 1236 | >QKM77298.1 envelope protein E |
| 1237 | >QKM77298.1 Flavi_E_C |
| 1238 | >QKM77298.1 flavi_E_stem |
| 1239 | >QKM77299.1 envelope protein E |
| 1240 | >QKM77299.1 Flavi_E_C |
| 1241 | >QKM77299.1 flavi_E_stem |
| 1242 | >QKM77300.1 envelope protein E |
| 1243 | >QKM77300.1 Flavi_E_C |
| 1244 | >QKM77300.1 flavi_E_stem |
| 1245 | >QKM77301.1 envelope protein E |
| 1246 | >QKM77301.1 Flavi_E_C |
| 1247 | >QKM77301.1 flavi_E_stem |
| 1248 | >QBM11757.1 Flavi_E_C |
| 1249 | >QBM11757.1 flavi_E_stem |
| 1250 | >AZS35403.1 Flavi_E_C |
| 1251 | >AZS35403.1 flavi_E_stem |
| 1252 | >AZS35404.1 Flavi_E_C |
| 1253 | >AZS35404.1 flavi_E_stem |
| 1254 | >AZS35405.1 Flavi_E_C |
| 1255 | >AZS35405.1 flavi_E_stem |
| 1256 | >QGA72951.1 envelope protein E |
| 1257 | >QGA72951.1 Flavi_E_C |
| 1258 | >QGA72951.1 flavi_E_stem |
| 1259 | >QGA67050.1 envelope protein E |
| 1260 | >QGA67050.1 Flavi_E_C |
| 1261 | >QGA67050.1 flavi_E_stem |
| 1262 | >QGA67051.1 envelope protein E |
| 1263 | >QGA67051.1 Flavi_E_C |
| 1264 | >QGA67051.1 flavi_E_stem |
| 1265 | >QGA67052.1 envelope protein E |
| 1266 | >QGA67052.1 Flavi_E_C |
| 1267 | >QGA67052.1 flavi_E_stem |
| 1268 | >QGA67053.1 envelope protein E |
| 1269 | >QGA67053.1 Flavi_E_C |
| 1270 | >QGA67053.1 flavi_E_stem |
| 1271 | >QGA67054.1 envelope protein E |
| 1272 | >QGA67054.1 Flavi_E_C |
| 1273 | >QGA67054.1 flavi_E_stem |
| 1274 | >QGA67055.1 envelope protein E |
| 1275 | >QGA67055.1 Flavi_E_C |
| 1276 | >QGA67055.1 flavi_E_stem |
| 1277 | >QGA67056.1 envelope protein E |
| 1278 | >QGA67056.1 Flavi_E_C |
| 1279 | >QGA67056.1 flavi_E_stem |
| 1280 | >QFZ95661.1 envelope protein E |
| 1281 | >QFZ95661.1 Flavi_E_C |
| 1282 | >QFZ95661.1 flavi_E_stem |
| 1283 | >QDZ58854.1 Flavi_E_C |
| 1284 | >QDZ58854.1 flavi_E_stem |
| 1285 | >QDZ58855.1 Flavi_E_C |
| 1286 | >QDZ58855.1 flavi_E_stem |
| 1287 | >QDZ58856.1 Flavi_E_C |
| 1288 | >QDZ58856.1 flavi_E_stem |
| 1289 | >AXQ39996.1 envelope protein E |
| 1290 | >AXQ39996.1 Flavi_E_C |
| 1291 | >AXQ39996.1 flavi_E_stem |
| 1292 | >AXQ39997.1 envelope protein E |
| 1293 | >AXQ39997.1 Flavi_E_C |
| 1294 | >AXQ39997.1 flavi_E_stem |
| 1295 | >AXQ39998.1 envelope protein E |
| 1296 | >AXQ39998.1 Flavi_E_C |
| 1297 | >AXQ39998.1 flavi_E_stem |
| 1298 | >AXQ39999.1 envelope protein E |
| 1299 | >AXQ39999.1 Flavi_E_C |
| 1300 | >AXQ39999.1 flavi_E_stem |
| 1301 | >AXQ40000.1 envelope protein E |
| 1302 | >AXQ40000.1 Flavi_E_C |
| 1303 | >AXQ40000.1 flavi_E_stem |
| 1304 | >AXQ40001.1 envelope protein E |
| 1305 | >AXQ40001.1 Flavi_E_C |
| 1306 | >AXQ40001.1 flavi_E_stem |
| 1307 | >AXQ40002.1 envelope protein E |
| 1308 | >AXQ40002.1 Flavi_E_C |
| 1309 | >AXQ40002.1 flavi_E_stem |
| 1310 | >AXQ40003.1 envelope protein E |
| 1311 | >AXQ40003.1 Flavi_E_C |
| 1312 | >AXQ40003.1 flavi_E_stem |
| 1313 | >AXQ40004.1 envelope protein E |
| 1314 | >AXQ40004.1 Flavi_E_C |
| 1315 | >AXQ40004.1 flavi_E_stem |
| 1316 | >AXQ40005.1 envelope protein E |
| 1317 | >AXQ40005.1 Flavi_E_C |
| 1318 | >AXQ40005.1 flavi_E_stem |
| 1319 | >AXQ40006.1 envelope protein E |
| 1320 | >AXQ40006.1 Flavi_E_C |
| 1321 | >AXQ40006.1 flavi_E_stem |
| 1322 | >AXQ40007.1 envelope protein E |
| 1323 | >AXQ40007.1 Flavi_E_C |
| 1324 | >AXQ40007.1 flavi_E_stem |
| 1325 | >QDH45910.1 Flavi_E_C |
| 1326 | >QDH45910.1 flavi_E_stem |
| 1327 | >QCG74166.1 Flavi_E_C |
| 1328 | >QCG74166.1 flavi_E_stem |
| 1329 | >AYV74916.1 Flavi_glycoprot |
| 1330 | >AYV74916.1 Flavi_E_C |
| 1331 | >AYV74916.1 flavi_E_stem |
| 1332 | >AYV74917.1 Flavi_glycoprot |
| 1333 | >AYV74917.1 Flavi_E_C |
| 1334 | >AYV74917.1 flavi_E_stem |
| 1335 | >AYV74918.1 Flavi_glycoprot |
| 1336 | >AYV74918.1 Flavi_E_C |
| 1337 | >AYV74918.1 flavi_E_stem |
| 1338 | >AYV74919.1 Flavi_glycoprot |
| 1339 | >AYV74919.1 Flavi_E_C |
| 1340 | >AYV74919.1 flavi_E_stem |
| 1341 | >AYV74920.1 Flavi_glycoprot |
| 1342 | >AYV74920.1 Flavi_E_C |
| 1343 | >AYV74920.1 flavi_E_stem |
| 1344 | >AYV74921.1 Flavi_glycoprot |
| 1345 | >AYV74921.1 Flavi_E_C |
| 1346 | >AYV74921.1 flavi_E_stem |
| 1347 | >AYV74922.1 Flavi_glycoprot |
| 1348 | >AYV74922.1 Flavi_E_C |
| 1349 | >AYV74922.1 flavi_E_stem |
| 1350 | >AYV74923.1 Flavi_glycoprot |
| 1351 | >AYV74923.1 Flavi_E_C |
| 1352 | >AYV74923.1 flavi_E_stem |
| 1353 | >AYV74924.1 Flavi_glycoprot |
| 1354 | >AYV74924.1 Flavi_E_C |
| 1355 | >AYV74924.1 flavi_E_stem |
| 1356 | >AYV74925.1 Flavi_glycoprot |
| 1357 | >AYV74925.1 Flavi_E_C |
| 1358 | >AYV74925.1 flavi_E_stem |
| 1359 | >AYV74927.1 Flavi_glycoprot |
| 1360 | >AYV74927.1 Flavi_E_C |
| 1361 | >AYV74927.1 flavi_E_stem |
| 1362 | >AYV74928.1 Flavi_glycoprot |
| 1363 | >AYV74928.1 Flavi_E_C |
| 1364 | >AYV74928.1 flavi_E_stem |
| 1365 | >AYV74929.1 Flavi_glycoprot |
| 1366 | >AYV74929.1 Flavi_E_C |
| 1367 | >AYV74929.1 flavi_E_stem |
| 1368 | >AYV74930.1 Flavi_glycoprot |
| 1369 | >AYV74930.1 Flavi_E_C |
| 1370 | >AYV74930.1 flavi_E_stem |
| 1371 | >AYV74931.1 Flavi_glycoprot |
| 1372 | >AYV74931.1 Flavi_E_C |
| 1373 | >AYV74931.1 flavi_E_stem |
| 1374 | >AYV74932.1 Flavi_glycoprot |
| 1375 | >AYV74932.1 Flavi_E_C |
| 1376 | >AYV74932.1 flavi_E_stem |
| 1377 | >AYV74933.1 Flavi_glycoprot |
| 1378 | >AYV74933.1 Flavi_E_C |
| 1379 | >AYV74933.1 flavi_E_stem |
| 1380 | >AYV74934.1 Flavi_glycoprot |
| 1381 | >AYV74934.1 Flavi_E_C |
| 1382 | >AYV74934.1 flavi_E_stem |
| 1383 | >AYV74935.1 Flavi_glycoprot |
| 1384 | >AYV74935.1 Flavi_E_C |
| 1385 | >AYV74935.1 flavi_E_stem |
| 1386 | >AYV74936.1 Flavi_glycoprot |
| 1387 | >AYV74936.1 Flavi_E_C |
| 1388 | >AYV74936.1 flavi_E_stem |
| 1389 | >AYV74937.1 Flavi_glycoprot |
| 1390 | >AYV74937.1 Flavi_E_C |
| 1391 | >AYV74937.1 flavi_E_stem |
| 1392 | >AYU74865.1 Flavi_glycoprot |
| 1393 | >AYU74865.1 Flavi_E_C |
| 1394 | >AYU74865.1 flavi_E_stem |
| 1395 | >ATB53753.1 Flavi_E_C |
| 1396 | >ATB53753.1 flavi_E_stem |
| 1397 | >ATB53754.1 Flavi_glycoprot |
| 1398 | >ATB53754.1 Flavi_E_C |
| 1399 | >ATB53754.1 flavi_E_stem |
| 1400 | >AVC63697.1 Flavi_glycoprot |
| 1401 | >AVC63697.1 Flavi_E_C |
| 1402 | >AVC63697.1 flavi_E_stem |
| 1403 | >AVC63698.1 Flavi_glycoprot |
| 1404 | >AVC63698.1 Flavi_E_C |
| 1405 | >AVC63698.1 flavi_E_stem |
| 1406 | >AYI50273.1 envelope protein E |
| 1407 | >AYI50273.1 Flavi_glycoprot |
| 1408 | >AYI50273.1 Flavi_E_C |
| 1409 | >AYI50273.1 flavi_E_stem |
| 1410 | >AYI50274.1 envelope protein E |
| 1411 | >AYI50274.1 Flavi_glycoprot |
| 1412 | >AYI50274.1 Flavi_E_C |
| 1413 | >AYI50274.1 flavi_E_stem |
| 1414 | >AYI50275.1 envelope protein E |
| 1415 | >AYI50275.1 Flavi_glycoprot |
| 1416 | >AYI50275.1 Flavi_E_C |
| 1417 | >AYI50275.1 flavi_E_stem |
| 1418 | >ASR91936.1 Flavi_glycoprot |
| 1419 | >ASR91936.1 Flavi_E_C |
| 1420 | >ASR91936.1 flavi_E_stem |
| 1421 | >AXF50014.1 Flavi_glycoprot |
| 1422 | >AXF50014.1 Flavi_E_C |
| 1423 | >AXF50014.1 flavi_E_stem |
| 1424 | >AXF50015.1 Flavi_E_C |
| 1425 | >AXF50015.1 flavi_E_stem |
| 1426 | >AXF50016.1 Flavi_glycoprot |
| 1427 | >AXF50016.1 Flavi_E_C |
| 1428 | >AXF50016.1 flavi_E_stem |
| 1429 | >AXF50052.1 Flavi_glycoprot |
| 1430 | >AXF50052.1 Flavi_E_C |
| 1431 | >AXF50052.1 flavi_E_stem |
| 1432 | >ATL14618.1 Flavi_E_C |
| 1433 | >ATL14618.1 flavi_E_stem |
| 1434 | >AUF35021.1 Flavi_glycoprot |
| 1435 | >AUF35021.1 Flavi_E_C |
| 1436 | >AUF35021.1 flavi_E_stem |
| 1437 | >AUF35022.1 Flavi_glycoprot |
| 1438 | >AUF35022.1 Flavi_E_C |
| 1439 | >AUF35022.1 flavi_E_stem |
| 1440 | >AVG19202.1 Flavi_glycoprot |
| 1441 | >AVG19202.1 Flavi_E_C |
| 1442 | >AVG19202.1 flavi_E_stem |
| 1443 | >AVG19203.1 Flavi_glycoprot |
| 1444 | >AVG19203.1 Flavi_E_C |
| 1445 | >AVG19203.1 flavi_E_stem |
| 1446 | >AVV62004.1 Flavi_glycoprot |
| 1447 | >AVV62004.1 Flavi_E_C |
| 1448 | >AVV62004.1 flavi_E_stem |
| 1449 | >ARU07074.1 Flavi_glycoprot |
| 1450 | >ARU07074.1 Flavi_E_C |
| 1451 | >ARU07074.1 flavi_E_stem |
| 1452 | >ARU07075.1 Flavi_glycoprot |
| 1453 | >ARU07075.1 Flavi_E_C |
| 1454 | >ARU07075.1 flavi_E_stem |
| 1455 | >ARU07076.1 Flavi_glycoprot |
| 1456 | >ARU07076.1 Flavi_E_C |
| 1457 | >ARU07076.1 flavi_E_stem |
| 1458 | >AUY62552.1 Flavi_E_C |
| 1459 | >AUY62552.1 flavi_E_stem |
| 1460 | >AUY62553.1 Flavi_glycoprot |
| 1461 | >AUY62553.1 Flavi_E_C |
| 1462 | >AUY62553.1 flavi_E_stem |
| 1463 | >AWH65848.1 Flavi_E_C |
| 1464 | >AWH65848.1 flavi_E_stem |
| 1465 | >AWH65849.1 Flavi_glycoprot |
| 1466 | >AWH65849.1 Flavi_E_C |
| 1467 | >AWH65849.1 flavi_E_stem |
| 1468 | >AWF93617.1 envelope protein E |
| 1469 | >AWF93617.1 Flavi_glycoprot |
| 1470 | >AWF93617.1 Flavi_E_C |
| 1471 | >AWF93617.1 flavi_E_stem |
| 1472 | >AWF93618.1 envelope protein E |
| 1473 | >AWF93618.1 Flavi_glycoprot |
| 1474 | >AWF93618.1 Flavi_E_C |
| 1475 | >AWF93618.1 flavi_E_stem |
| 1476 | >AWF93619.1 envelope protein E |
| 1477 | >AWF93619.1 Flavi_glycoprot |
| 1478 | >AWF93619.1 Flavi_E_C |
| 1479 | >AWF93619.1 flavi_E_stem |
| 1480 | >AWF93620.1 envelope protein E |
| 1481 | >AWF93620.1 Flavi_glycoprot |
| 1482 | >AWF93620.1 Flavi_E_C |
| 1483 | >AWF93620.1 flavi_E_stem |
| 1484 | >AWF93621.1 envelope protein E |
| 1485 | >AWF93621.1 Flavi_glycoprot |
| 1486 | >AWF93621.1 Flavi_E_C |
| 1487 | >AWF93621.1 flavi_E_stem |
| 1488 | >AWF93622.1 envelope protein E |
| 1489 | >AWF93622.1 Flavi_glycoprot |
| 1490 | >AWF93622.1 Flavi_E_C |
| 1491 | >AWF93622.1 flavi_E_stem |
| 1492 | >AWF93623.1 envelope protein E |
| 1493 | >AWF93623.1 Flavi_glycoprot |
| 1494 | >AWF93623.1 Flavi_E_C |
| 1495 | >AWF93623.1 flavi_E_stem |
| 1496 | >AWF93624.1 envelope protein E |
| 1497 | >AWF93624.1 Flavi_glycoprot |
| 1498 | >AWF93624.1 Flavi_E_C |
| 1499 | >AWF93624.1 flavi_E_stem |
| 1500 | >AWF93625.1 envelope protein E |
| 1501 | >AWF93625.1 Flavi_glycoprot |
| 1502 | >AWF93625.1 Flavi_E_C |
| 1503 | >AWF93625.1 flavi_E_stem |
| 1504 | >AWF93626.1 envelope protein E |
| 1505 | >AWF93626.1 Flavi_glycoprot |
| 1506 | >AWF93626.1 Flavi_E_C |
| 1507 | >AWF93626.1 flavi_E_stem |
| 1508 | >AWF93627.1 envelope protein E |
| 1509 | >AWF93627.1 Flavi_glycoprot |
| 1510 | >AWF93627.1 Flavi_E_C |
| 1511 | >AWF93627.1 flavi_E_stem |
| 1512 | >AWF93628.1 envelope protein E |
| 1513 | >AWF93628.1 Flavi_glycoprot |
| 1514 | >AWF93628.1 Flavi_E_C |
| 1515 | >AWF93628.1 flavi_E_stem |
| 1516 | >AWF93629.1 envelope protein E |
| 1517 | >AWF93629.1 Flavi_glycoprot |
| 1518 | >AWF93629.1 Flavi_E_C |
| 1519 | >AWF93629.1 flavi_E_stem |
| 1520 | >AWF93630.1 envelope protein E |
| 1521 | >AWF93630.1 Flavi_glycoprot |
| 1522 | >AWF93630.1 Flavi_E_C |
| 1523 | >AWF93630.1 flavi_E_stem |
| 1524 | >AWF93631.1 envelope protein E |
| 1525 | >AWF93631.1 Flavi_glycoprot |
| 1526 | >AWF93631.1 Flavi_E_C |
| 1527 | >AWF93631.1 flavi_E_stem |
| 1528 | >AWF93632.1 envelope protein E |
| 1529 | >AWF93632.1 Flavi_glycoprot |
| 1530 | >AWF93632.1 Flavi_E_C |
| 1531 | >AWF93632.1 flavi_E stem |
| 1532 | >AWF93633.1 envelope protein E |
| 1533 | >AWF93633.1 Flavi_glycoprot |
| 1534 | >AWF93633.1 Flavi_E_C |
| 1535 | >AWF93633.1 flavi_E_stem |
| 1536 | >AWF93634.1 envelope protein E |
| 1537 | >AWF93634.1 Flavi_glycoprot |
| 1538 | >AWF93634.1 Flavi_E_C |
| 1539 | >AWF93634.1 flavi_E_stem |
| 1540 | >AWF93635.1 envelope protein E |
| 1541 | >AWF93635.1 Flavi_glycoprot |
| 1542 | >AWF93635.1 Flavi_E_C |
| 1543 | >AWF93635.1 flavi_E_stem |
| 1544 | >AVZ47169.1 Flavi_glycoprot |
| 1545 | >AVZ47169.1 Flavi_E_C |
| 1546 | >AVZ47169.1 flavi_E_stem |
| 1547 | >AVW85800.1 envelope protein E |
| 1548 | >AVW85800.1 Flavi_glycoprot |
| 1549 | >AVW85800.1 Flavi_E_C |
| 1550 | >AVW85800.1 flavi_E_stem |
| 1551 | >AVW85801.1 envelope protein E |
| 1552 | >AVW85801.1 Flavi_glycoprot |
| 1553 | >AVW85801.1 Flavi_E_C |
| 1554 | >AVW85801.1 flavi_E_stem |
| 1555 | >AVW85802.1 envelope protein E |
| 1556 | >AVW85802.1 Flavi_glycoprot |
| 1557 | >AVW85802.1 Flavi_E_C |
| 1558 | >AVW85802.1 flavi_E_stem |
| 1559 | >AVW85803.1 envelope protein E |
| 1560 | >AVW85803.1 Flavi_glycoprot |
| 1561 | >AVW85803.1 Flavi_E_C |
| 1562 | >AVW85803.1 flavi_E_stem |
| 1563 | >AVW85804.1 envelope protein E |
| 1564 | >AVW85804.1 Flavi_glycoprot |
| 1565 | >AVW85804.1 Flavi_E_C |
| 1566 | >AVW85804.1 flavi_E_stem |
| 1567 | >AVW85805.1 envelope protein E |
| 1568 | >AVW85805.1 Flavi_glycoprot |
| 1569 | >AVW85805.1 Flavi_E_C |
| 1570 | >AVW85805.1 flavi_E_stem |
| 1571 | >AVW85806.1 envelope protein E |
| 1572 | >AVW85806.1 Flavi_glycoprot |
| 1573 | >AVW85806.1 Flavi_E_C |
| 1574 | >AVW85806.1 flavi_E_stem |
| 1575 | >AVW85807.1 envelope protein E |
| 1576 | >AVW85807.1 Flavi_glycoprot |
| 1577 | >AVW85807.1 Flavi_E_C |
| 1578 | >AVW85807.1 flavi_E_stem |
| 1579 | >AVW85808.1 envelope protein E |
| 1580 | >AVW85808.1 Flavi_glycoprot |
| 1581 | >AVW85808.1 Flavi_E_C |
| 1582 | >AVW85808.1 flavi_E_stem |
| 1583 | >AVW85809.1 envelope protein E |
| 1584 | >AVW85809.1 Flavi_glycoprot |
| 1585 | >AVW85809.1 Flavi_E_C |
| 1586 | >AVW85809.1 flavi_E_stem |
| 1587 | >AVW85810.1 envelope protein E |
| 1588 | >AVW85810.1 Flavi_glycoprot |
| 1589 | >AVW85810.1 Flavi_E_C |
| 1590 | >AVW85810.1 flavi_E_stem |
| 1591 | >AVW85811.1 envelope protein E |
| 1592 | >AVW85811.1 Flavi_glycoprot |
| 1593 | >AVW85811.1 Flavi_E_C |
| 1594 | >AVW85811.1 flavi_E_stem |
| 1595 | >AVW85812.1 envelope protein E |
| 1596 | >AVW85812.1 Flavi_glycoprot |
| 1597 | >AVW85812.1 Flavi_E_C |
| 1598 | >AVW85812.1 flavi_E_stem |
| 1599 | >AVW85813.1 envelope protein E |
| 1600 | >AVW85813.1 Flavi_glycoprot |
| 1601 | >AVW85813.1 Flavi_E_C |
| 1602 | >AVW85813.1 flavi_E_stem |
| 1603 | >AVW85814.1 envelope protein E |
| 1604 | >AVW85814.1 Flavi_glycoprot |
| 1605 | >AVW85814.1 Flavi_E_C |
| 1606 | >AVW85814.1 flavi_E_stem |
| 1607 | >AVW85815.1 envelope protein E |
| 1608 | >AVW85815.1 Flavi_glycoprot |
| 1609 | >AVW85815.1 Flavi_E_C |
| 1610 | >AVW85815.1 flavi_E_stem |
| 1611 | >AVW85816.1 envelope protein E |
| 1612 | >AVW85816.1 Flavi_glycoprot |
| 1613 | >AVW85816.1 Flavi_E_C |
| 1614 | >AVW85816.1 flavi_E_stem |
| 1615 | >AVW85817.1 envelope protein E |
| 1616 | >AVW85817.1 Flavi_glycoprot |
| 1617 | >AVW85817.1 Flavi_E_C |
| 1618 | >AVW85817.1 flavi_E_stem |
| 1619 | >AVW85818.1 envelope protein E |
| 1620 | >AVW85818.1 Flavi_glycoprot |
| 1621 | >AVW85818.1 Flavi_E_C |
| 1622 | >AVW85818.1 flavi_E_stem |
| 1623 | >AVW85819.1 envelope protein E |
| 1624 | >AVW85819.1 Flavi_glycoprot |
| 1625 | >AVW85819.1 Flavi_E_C |
| 1626 | >AVW85819.1 flavi_E_stem |
| 1627 | >AVW85820.1 envelope protein E |
| 1628 | >AVW85820.1 Flavi_glycoprot |
| 1629 | >AVW85820.1 Flavi_E_C |
| 1630 | >AVW85820.1 flavi_E_stem |
| 1631 | >AVW85821.1 envelope protein E |
| 1632 | >AVW85821.1 Flavi_glycoprot |
| 1633 | >AVW85821.1 Flavi_E_C |
| 1634 | >AVW85821.1 flavi_E_stem |
| 1635 | >AVW85822.1 envelope protein E |
| 1636 | >AVW85822.1 Flavi_glycoprot |
| 1637 | >AVW85822.1 Flavi_E_C |
| 1638 | >AVW85822.1 flavi_E_stem |
| 1639 | >AVW85823.1 envelope protein E |
| 1640 | >AVW85823.1 Flavi_glycoprot |
| 1641 | >AVW85823.1 Flavi_E_C |
| 1642 | >AVW85823.1 flavi_E_stem |
| 1643 | >AVW85824.1 envelope protein E |
| 1644 | >AVW85824.1 Flavi_glycoprot |
| 1645 | >AVW85824.1 Flavi_E_C |
| 1646 | >AVW85824.1 flavi_E_stem |
| 1647 | >AVW85825.1 envelope protein E |
| 1648 | >AVW85825.1 Flavi_glycoprot |
| 1649 | >AVW85825.1 Flavi_E_C |
| 1650 | >AVW85825.1 flavi_E_stem |
| 1651 | >AVW85826.1 envelope protein E |
| 1652 | >AVW85826.1 Flavi_glycoprot |
| 1653 | >AVW85826.1 Flavi_E_C |
| 1654 | >AVW85826.1 flavi_E_stem |
| 1655 | >AVW85827.1 envelope protein E |
| 1656 | >AVW85827.1 Flavi_glycoprot |
| 1657 | >AVW85827.1 Flavi_E_C |
| 1658 | >AVW85827.1 flavi_E_stem |
| 1659 | >AVW85828.1 envelope protein E |
| 1660 | >AVW85828.1 Flavi_glycoprot |
| 1661 | >AVW85828.1 Flavi_E_C |
| 1662 | >AVW85828.1 flavi_E_stem |
| 1663 | >AVW85829.1 envelope protein E |
| 1664 | >AVW85829.1 Flavi_glycoprot |
| 1665 | >AVW85829.1 Flavi_E_C |
| 1666 | >AVW85829.1 flavi_E_stem |
| 1667 | >AVW85830.1 envelope protein E |
| 1668 | >AVW85830.1 Flavi_glycoprot |
| 1669 | >AVW85830.1 Flavi_E_C |
| 1670 | >AVW85830.1 flavi_E_stem |
| 1671 | >AVW85831.1 envelope protein E |
| 1672 | >AVW85831.1 Flavi_glycoprot |
| 1673 | >AVW85831.1 Flavi_E_C |
| 1674 | >AVW85831.1 flavi_E_stem |
| 1675 | >AVW85832.1 envelope protein E |
| 1676 | >AVW85832.1 Flavi_glycoprot |
| 1677 | >AVW85832.1 Flavi_E_C |
| 1678 | >AVW85832.1 flavi_E_stem |
| 1679 | >AVW85833.1 envelope protein E |
| 1680 | >AVW85833.1 Flavi_glycoprot |
| 1681 | >AVW85833.1 Flavi_E_C |
| 1682 | >AVW85833.1 flavi_E_stem |
| 1683 | >AVW85834.1 envelope protein E |
| 1684 | >AVW85834.1 Flavi_glycoprot |
| 1685 | >AVW85834.1 Flavi_E_C |
| 1686 | >AVW85834.1 flavi_E_stem |
| 1687 | >AVW85835.1 envelope protein E |
| 1688 | >AVW85835.1 Flavi_glycoprot |
| 1689 | >AVW85835.1 Flavi_E_C |
| 1690 | >AVW85835.1 flavi_E_stem |
| 1691 | >AVW85836.1 envelope protein E |
| 1692 | >AVW85836.1 Flavi_glycoprot |
| 1693 | >AVW85836.1 Flavi_E_C |
| 1694 | >AVW85836.1 flavi_E_stem |
| 1695 | >AVW85837.1 envelope protein E |
| 1696 | >AVW85837.1 Flavi_glycoprot |
| 1697 | >AVW85837.1 Flavi_E_C |
| 1698 | >AVW85837.1 flavi_E_stem |
| 1699 | >AVW85838.1 envelope protein E |
| 1700 | >AVW85838.1 Flavi_glycoprot |
| 1701 | >AVW85838.1 Flavi_E_C |
| 1702 | >AVW85838.1 flavi_E_stem |
| 1703 | >AVW85839.1 envelope protein E |
| 1704 | >AVW85839.1 Flavi_glycoprot |
| 1705 | >AVW85839.1 Flavi_E_C |
| 1706 | >AVW85839.1 flavi_E_stem |
| 1707 | >AVW85840.1 envelope protein E |
| 1708 | >AVW85840.1 Flavi_glycoprot |
| 1709 | >AVW85840.1 Flavi_E_C |
| 1710 | >AVW85840.1 flavi_E_stem |
| 1711 | >AVW85841.1 envelope protein E |
| 1712 | >AVW85841.1 Flavi_glycoprot |
| 1713 | >AVW85841.1 Flavi_E_C |
| 1714 | >AVW85841.1 flavi_E_stem |
| 1715 | >AVW85842.1 envelope protein E |
| 1716 | >AVW85842.1 Flavi_glycoprot |
| 1717 | >AVW85842.1 Flavi_E_C |
| 1718 | >AVW85842.1 flavi_E_stem |
| 1719 | >AVW85843.1 envelope protein E |
| 1720 | >AVW85843.1 Flavi_glycoprot |
| 1721 | >AVW85843.1 Flavi_E_C |
| 1722 | >AVW85843.1 flavi_E_stem |
| 1723 | >AVW85844.1 envelope protein E |
| 1724 | >AVW85844.1 Flavi_glycoprot |
| 1725 | >AVW85844.1 Flavi_E_C |
| 1726 | >AVW85844.1 flavi_E_stem |
| 1727 | >AVW85845.1 envelope protein E |
| 1728 | >AVW85845.1 Flavi_glycoprot |
| 1729 | >AVW85845.1 Flavi_E_C |
| 1730 | >AVW85845.1 flavi_E_stem |
| 1731 | >AVW85846.1 envelope protein E |
| 1732 | >AVW85846.1 Flavi_glycoprot |
| 1733 | >AVW85846.1 Flavi_E_C |
| 1734 | >AVW85846.1 flavi_E_stem |
| 1735 | >AVW85847.1 envelope protein E |
| 1736 | >AVW85847.1 Flavi_glycoprot |
| 1737 | >AVW85847.1 Flavi_E_C |
| 1738 | >AVW85847.1 flavi_E_stem |
| 1739 | >AVW85848.1 envelope protein E |
| 1740 | >AVW85848.1 Flavi_glycoprot |
| 1741 | >AVW85848.1 Flavi_E_C |
| 1742 | >AVW85848.1 flavi_E_stem |
| 1743 | >AVW85849.1 envelope protein E |
| 1744 | >AVW85849.1 Flavi_glycoprot |
| 1745 | >AVW85849.1 Flavi_E_C |
| 1746 | >AVW85849.1 flavi_E_stem |
| 1747 | >AVW85850.1 envelope protein E |
| 1748 | >AVW85850.1 Flavi_glycoprot |
| 1749 | >AVW85850.1 Flavi_E_C |
| 1750 | >AVW85850.1 flavi_E_stem |
| 1751 | >AVW85851.1 envelope protein E |
| 1752 | >AVW85851.1 Flavi_glycoprot |
| 1753 | >AVW85851.1 Flavi_E_C |
| 1754 | >AVW85851.1 flavi_E_stem |
| 1755 | >AVW85852.1 envelope protein E |
| 1756 | >AVW85852.1 Flavi_glycoprot |
| 1757 | >AVW85852.1 Flavi_E_C |
| 1758 | >AVW85852.1 flavi_E_stem |
| 1759 | >AVW85853.1 envelope protein E |
| 1760 | >AVW85853.1 Flavi_glycoprot |
| 1761 | >AVW85853.1 Flavi_E_C |
| 1762 | >AVW85853.1 flavi_E_stem |
| 1763 | >AVW85854.1 envelope protein E |
| 1764 | >AVW85854.1 Flavi_glycoprot |
| 1765 | >AVW85854.1 Flavi_E_C |
| 1766 | >AVW85854.1 flavi_E_stem |
| 1767 | >AVW85855.1 envelope protein E |
| 1768 | >AVW85855.1 Flavi_glycoprot |
| 1769 | >AVW85855.1 Flavi_E_C |
| 1770 | >AVW85855.1 flavi_E_stem |
| 1771 | >AVW85856.1 envelope protein E |
| 1772 | >AVW85856.1 Flavi_glycoprot |
| 1773 | >AVW85856.1 Flavi_E_C |
| 1774 | >AVW85856.1 flavi_E_stem |
| 1775 | >AVW85857.1 envelope protein E |
| 1776 | >AVW85857.1 Flavi_glycoprot |
| 1777 | >AVW85857.1 Flavi_E_C |
| 1778 | >AVW85857.1 flavi_E_stem |
| 1779 | >AVW85858.1 envelope protein E |
| 1780 | >AVW85858.1 Flavi_glycoprot |
| 1781 | >AVW85858.1 Flavi_E_C |
| 1782 | >AVW85858.1 flavi_E_stem |
| 1783 | >AVW85859.1 envelope protein E |
| 1784 | >AVW85859.1 Flavi_glycoprot |
| 1785 | >AVW85859.1 Flavi_E_C |
| 1786 | >AVW85859.1 flavi_E_stem |
| 1787 | >AVW85860.1 envelope protein E |
| 1788 | >AVW85860.1 Flavi_glycoprot |
| 1789 | >AVW85860.1 Flavi_E_C |
| 1790 | >AVW85860.1 flavi_E_stem |
| 1791 | >AVW85861.1 envelope protein E |
| 1792 | >AVW85861.1 Flavi_glycoprot |
| 1793 | >AVW85861.1 Flavi_E_C |
| 1794 | >AVW85861.1 flavi_E_stem |
| 1795 | >AVW85862.1 envelope protein E |
| 1796 | >AVW85862.1 Flavi_glycoprot |
| 1797 | >AVW85862.1 Flavi_E_C |
| 1798 | >AVW85862.1 flavi_E_stem |
| 1799 | >AVW85863.1 envelope protein E |
| 1800 | >AVW85863.1 Flavi_glycoprot |
| 1801 | >AVW85863.1 Flavi_E_C |
| 1802 | >AVW85863.1 flavi_E_stem |
| 1803 | >BBC70847.1 Flavi_glycoprot |
| 1804 | >BBC70847.1 Flavi_E_C |
| 1805 | >BBC70847.1 flavi_E_stem |
| 1806 | >AVG19275.1 Flavi_glycoprot |
| 1807 | >AVG19275.1 Flavi_E_C |
| 1808 | >AVG19275.1 flavi_E_stem |
| 1809 | >AUI42329.1 Flavi_glycoprot |
| 1810 | >AUI42329.1 Flavi_E_C |
| 1811 | >AUI42329.1 flavi_E_stem |
| 1812 | >AUI42330.1 Flavi_glycoprot |
| 1813 | >AUI42330.1 Flavi_E_C |
| 1814 | >AUI42330.1 flavi_E_stem |
| 1815 | >ARB08102.1 Flavi_E_C |
| 1816 | >ARB08102.1 flavi_E_stem |
| 1817 | >ATB53752.1 Flavi_E_C |
| 1818 | >ATB53752.1 flavi_E_stem |
| 1819 | >AQS26698.1 Flavi_glycoprot |
| 1820 | >AQS26698.1 Flavi_E_C |
| 1821 | >AQS26698.1 flavi_E_stem |
| 1822 | >ASF57880.1 Flavi_E_C |
| 1823 | >ASF57880.1 flavi_E_stem |
| 1824 | >ASB32509.1 envelope protein E |
| 1825 | >ASB32509.1 Flavi_E_C |
| 1826 | >ASB32509.1 flavi_E_stem |
| 1827 | >APW84872.1 Flavi_E_C |
| 1828 | >APW84872.1 flavi_E_stem |
| 1829 | >APW84873.1 Flavi_E_C |
| 1830 | >APW84873.1 flavi_E_stem |
| 1831 | >APW84874.1 Flavi_E_C |
| 1832 | >APW84874.1 flavi_E_stem |
| 1833 | >APW84875.1 Flavi_E_C |
| 1834 | >APW84875.1 flavi_E_stem |
| 1835 | >APW84876.1 Flavi_E_C |
| 1836 | >APW84876.1 flavi_E_stem |
| 1837 | >APW84877.1 Flavi_E_C |
| 1838 | >APW84877.1 flavi_E_stem |
| 1839 | >ARM59239.1 Flavi_glycoprot |
| 1840 | >ARM59239.1 Flavi_E_C |
| 1841 | >ARM59239.1 flavi_E_stem |
| 1842 | >ARM59240.1 envelope protein E |
| 1843 | >ARM59240.1 Flavi_glycoprot |
| 1844 | >ARM59240.1 Flavi_E_C |
| 1845 | >ARM59240.1 flavi_E_stem |
| 1846 | >AMX81915.1 Flavi_glycoprot |
| 1847 | >AMX81915.1 Flavi_E_C |
| 1848 | >AMX81915.1 flavi_E_stem |
| 1849 | >AMX81916.1 Flavi_glycoprot |
| 1850 | >AMX81916.1 Flavi_E_C |
| 1851 | >AMX81916.1 flavi_E_stem |
| 1852 | >AMX81917.1 Flavi_glycoprot |
| 1853 | >AMX81917.1 Flavi_E_C |
| 1854 | >AMX81917.1 flavi_E_stem |
| 1855 | >AQZ41947.1 envelope protein E |
| 1856 | >AQZ41947.1 Flavi_E_C |
| 1857 | >AQZ41947.1 flavi_E_stem |
| 1858 | >AQZ41948.1 envelope protein E |
| 1859 | >AQZ41948.1 Flavi_E_C |
| 1860 | >AQZ41948.1 flavi_E_stem |
| 1861 | >AQZ41949.1 envelope protein E |
| 1862 | >AQZ41949.1 Flavi_glycoprot |
| 1863 | >AQZ41949.1 Flavi_E_C |
| 1864 | >AQZ41949.1 flavi_E_stem |
| 1865 | >AQZ41950.1 envelope protein E |
| 1866 | >AQZ41950.1 Flavi_E_C |
| 1867 | >AQZ41950.1 flavi_E_stem |
| 1868 | >AQZ41951.1 envelope protein E |
| 1869 | >AQZ41951.1 Flavi_E_C |
| 1870 | >AQZ41951.1 flavi_E_stem |
| 1871 | >AQZ41952.1 envelope protein E |
| 1872 | >AQZ41952.1 Flavi_glycoprot |
| 1873 | >AQZ41952.1 Flavi_E_C |
| 1874 | >AQZ41952.1 flavi_E_stem |
| 1875 | >AQZ41953.1 envelope protein E |
| 1876 | >AQZ41953.1 Flavi_glycoprot |
| 1877 | >AQZ41953.1 Flavi_E_C |
| 1878 | >AQZ41953.1 flavi_E_stem |
| 1879 | >AQZ41954.1 envelope protein E |
| 1880 | >AQZ41954.1 Flavi_E_C |
| 1881 | >AQZ41954.1 flavi_E_stem |
| 1882 | >AQZ41955.1 envelope protein E |
| 1883 | >AQZ41955.1 Flavi_glycoprot |
| 1884 | >AQZ41955.1 Flavi_E_C |
| 1885 | >AQZ41955.1 flavi_E_stem |
| 1886 | >AQZ41956.1 envelope protein E |
| 1887 | >AQZ41956.1 Flavi_E_C |
| 1888 | >AQZ41956.1 flavi_E_stem |
| 1889 | >AQW43707.1 Flavi_glycoprot |
| 1890 | >AQW43707.1 Flavi_E_C |
| 1891 | >AQW43707.1 flavi_E_stem |
| 1892 | >AQW43708.1 Flavi_glycoprot |
| 1893 | >AQW43708.1 Flavi_E_C |
| 1894 | >AQW43708.1 flavi_E_stem |
| 1895 | >BAX00477.1 Flavi_glycoprot |
| 1896 | >BAX00477.1 Flavi_E_C |
| 1897 | >BAX00477.1 flavi_E_stem |
| 1898 | >AQV07165.1 envelope protein E |
| 1899 | >AQV07165.1 Flavi_glycoprot |
| 1900 | >AQV07165.1 Flavi_E_C |
| 1901 | >AQV07165.1 flavi_E_stem |
| 1902 | >AOO19564.1 Flavi_glycoprot |
| 1903 | >AOO19564.1 Flavi_E_C |
| 1904 | >AOO19564.1 flavi_E_stem |
| 1905 | >APO36913.1 Flavi_glycoprot |
| 1906 | >APO36913.1 Flavi_E_C |
| 1907 | >APO36913.1 flavi_E_stem |
| 1908 | >AMM43325.1 Flavi_glycoprot |
| 1909 | >AMM43325.1 Flavi_E_C |
| 1910 | >AMM43325.1 flavi_E_stem |
| 1911 | >AMM43326.1 Flavi_glycoprot |
| 1912 | >AMM43326.1 Flavi_E_C |
| 1913 | >AMM43326.1 flavi_E_stem |
| 1914 | >APH11492.1 envelope protein E |
| 1915 | >APH11492.1 Flavi_glycoprot |
| 1916 | >APH11492.1 Flavi_E_C |
| 1917 | >APH11492.1 flavi_E_stem |
| 1918 | >APO08503.1 Flavi_E_C |
| 1919 | >APO08503.1 flavi_E_stem |
| 1920 | >APC60216.2 Flavi_glycoprot |
| 1921 | >APC60216.2 Flavi_E_C |
| 1922 | >APC60216.2 flavi_E_stem |
| 1923 | >ANH22038.1 Flavi_E_C |
| 1924 | >ANH22038.1 flavi_E_stem |
| 1925 | >BAV82373.1 Flavi_E_C |
| 1926 | >BAV82373.1 flavi_E_stem |
| 1927 | >AOR82892.1 Flavi_E_C |
| 1928 | >AOR82892.1 flavi_E_stem |
| 1929 | >AOR82893.1 Flavi_E_C |
| 1930 | >AOR82893.1 flavi_E_stem |
| 1931 | >AOR51315.1 Flavi_glycoprot |
| 1932 | >AOR51315.1 Flavi_E_C |
| 1933 | >AOR51315.1 flavi_E_stem |
| 1934 | >AND01116.2 Flavi_glycoprot |
| 1935 | >AND01116.2 Flavi_E_C |
| 1936 | >AND01116.2 flavi_E_stem |
| 1937 | >AOG18296.1 Flavi_glycoprot |
| 1938 | >AOG18296.1 Flavi_E_C |
| 1939 | >AOG18296.1 flavi_E_stem |
| 1940 | >AOC50652.1 envelope protein E |
| 1941 | >AOC50653.1 envelope protein E |
| 1942 | >AOC50654.1 envelope protein E |
| 1943 | >ANZ46736.1 Flavi_E_C |
| 1944 | >ANZ46736.1 flavi_E_stem |
| 1945 | >ANW07474.2 envelope protein E |
| 1946 | >ANW07474.2 Flavi_E_C |
| 1947 | >ANW07474.2 flavi_E_stem |
| 1948 | >ANW07475.1 envelope protein E |
| 1949 | >ANW07476.1 envelope protein E |
| 1950 | >ANW07476.1 Flavi_E_C |
| 1951 | >ANW07476.1 flavi_E_stem |
| 1952 | >ANW07477.1 envelope protein E |
| 1953 | >ANW07477.1 Flavi_E_C |
| 1954 | >ANW07477.1 flavi_E_stem |
| 1955 | >ANN44857.1 Flavi_glycoprot |
| 1956 | >ANN44857.1 Flavi_E_C |
| 1957 | >ANN44857.1 flavi_E_stem |
| 1958 | >ANN83272.1 envelope protein E |
| 1959 | >ANN83273.1 envelope protein E |
| 1960 | >ANK57897.1 Flavi_glycoprot |
| 1961 | >ANK57897.1 Flavi_E_C |
| 1962 | >ANK57897.1 flavi_E_stem |
| 1963 | >ANH10698.1 Flavi_E_C |
| 1964 | >ANH10698.1 flavi_E_stem |
| 1965 | >ANG09399.1 Flavi_E_C |
| 1966 | >ANG09399.1 flavi_E_stem |
| 1967 | >ANF16414.1 Flavi_glycoprot |
| 1968 | >ANF16414.1 Flavi_E_C |
| 1969 | >ANF16414.1 flavi_E_stem |
| 1970 | >ANC90427.1 envelope protein E |
| 1971 | >ANC90428.1 envelope protein E |
| 1972 | >ANC90428.1 Flavi_glycoprot |
| 1973 | >ANC90428.1 Flavi_E_C |
| 1974 | >ANC90428.1 flavi_E_stem |
| 1975 | >ANB66182.1 envelope protein E |
| 1976 | >ANB66182.1 Flavi_glycoprot |
| 1977 | >ANB66182.1 Flavi_E_C |
| 1978 | >ANB66182.1 flavi_E_stem |
| 1979 | >ANB66183.1 envelope protein E |
| 1980 | >ANB66183.1 Flavi_E_C |
| 1981 | >ANB66183.1 flavi_E_stem |
| 1982 | >ANB66184.1 envelope protein E |
| 1983 | >ANA12599.1 Flavi_glycoprot |
| 1984 | >ANA12599.1 Flavi_E_C |
| 1985 | >ANA12599.1 flavi_E_stem |
| 1986 | >AMX81919.1 Flavi_E_C |
| 1987 | >AMX81919.1 flavi_E_stem |
| 1988 | >AMR39832.1 Flavi_E_C |
| 1989 | >AMR39832.1 flavi_E_stem |
| 1990 | >AMR39833.1 Flavi_E_C |
| 1991 | >AMR39833.1 flavi_E_stem |
| 1992 | >AMR39834.1 Flavi_E_C |
| 1993 | >AMR39834.1 flavi_E_stem |
| 1994 | >AMR39835.1 Flavi_E_C |
| 1995 | >AMR39835.1 flavi_E_stem |
| 1996 | >AMR39836.1 Flavi_E_C |
| 1997 | >AMR39836.1 flavi_E_stem |
| 1998 | >AMQ48986.1 Flavi_E_C |
| 1999 | >AMQ48986.1 flavi_E_stem |
| 2000 | >AMQ48981.1 envelope protein E |
| 2001 | >AMQ48981.1 Flavi_E_C |
| 2002 | >AMQ48981.1 flavi_E_stem |
| 2003 | >AMQ48982.1 envelope protein E |
| 2004 | >AMQ48982.1 Flavi_E_C |
| 2005 | >AMQ48982.1 flavi_E_stem |
| 2006 | >AMQ34003.1 Env |
| 2007 | >AMQ34003.1 Flavi_E_C |
| 2008 | >AMQ34003.1 flavi_E_stem |
| 2009 | >AMQ34004.1 Env |
| 2010 | >AMQ34004.1 Flavi_E_C |
| 2011 | >AMQ34004.1 flavi_E_stem |
| 2012 | >AMN14619.1 Flavi_E_C |
| 2013 | >AMN14619.1 flavi_E_stem |
| 2014 | >AMN14620.1 Flavi_E_C |
| 2015 | >AMN14620.1 flavi_E_stem |
| 2016 | >AMM39806.1 Flavi_glycoprot |
| 2017 | >AMM39806.1 Flavi_E_C |
| 2018 | >AMM39806.1 flavi_E_stem |
| 2019 | >AMK79469.1 Flavi_E_C |
| 2020 | >AMK79469.1 flavi_E_stem |
| 2021 | >AMD16557.1 Flavi_E_C |
| 2022 | >AMD16557.1 flavi_E_stem |
| 2023 | >AMH87239.1 Flavi_E_C |
| 2024 | >AMH87239.1 flavi_E_stem |
| 2025 | >AMB18850.1 Flavi_glycoprot |
| 2026 | >AMB18850.1 Flavi_E_C |
| 2027 | >AMB18850.1 flavi_E_stem |
| 2028 | >AMD61710.1 Flavi_glycoprot |
| 2029 | >AMD61710.1 Flavi_E_C |
| 2030 | >AMD61710.1 flavi_E_stem |
| 2031 | >AMD61711.1 Flavi_E_C |
| 2032 | >AMD61711.1 flavi_E_stem |
| 2033 | >AMB37295.1 Flavi_E_C |
| 2034 | >AMB37295.1 flavi_E_stem |
| 2035 | >AMC13911.1 Flavi_E_C |
| 2036 | >AMC13911.1 flavi_E_stem |
| 2037 | >ALU33341.1 Flavi_glycoprot |
| 2038 | >ALU33341.1 Flavi_E_C |
| 2039 | >ALU33341.1 flavi_E_stem |
| 2040 | >BAP47441.1 Flavi_E_C |
| 2041 | >BAP47441.1 flavi_E_stem |
| 2042 | >AHZ13508.1 Flavi_E_C |
| 2043 | >AHZ13508.1 flavi_E_stem |
| 2044 | >AAV34151.1 Flavi_E_C |
| 2045 | >AAV34151.1 flavi_E_stem |

TABLE 1F
Non-limiting example Zika Protein M sequences

| SEQ | Zika viruses with example |
|---|---|
| ID NO | Protein M sequences |
| 2046 | YP_009430297.1 membrane glycoprotein |
| | precursor M |
| 2047 | YP_009430297.1 Flavivirus polyprotein |
| | propeptide |
| 2048 | YP_009430297.1 Flavivirus envelope |
| | glycoprotein M |
| 2049 | YP_009430299.1 membrane glycoprotein M |
| 2050 | YP_009428568.1 membrane glycoprotein |
| | precursor M |
| 2051 | YP_009428568.1 protein pr |
| 2052 | YP_009428568.1 Flavivirus polyprotein |
| | propeptide |
| 2053 | YP_009428568.1 membrane glycoprotein M |
| 2054 | YP_009428568.1 Flavivirus envelope |
| | glycoprotein M |
| 2055 | YP_009227197.1 membrane glycoprotein |
| | precursor M |
| 2056 | YP_009227197.1 Flavivirus polyprotein |
| | propeptide |
| 2057 | YP_009227197.1 Flavivirus envelope |
| | glycoprotein M |
| 2058 | YP_009227208.1 membrane glycoprotein M |
| 2059 | YP_009227208.1 Flavivirus envelope |
| | glycoprotein M |
| 2060 | YP_002790881.1 protein pr |
| 2061 | YP_002790881.1 Flavivirus polyprotein |
| | propeptide |
| 2062 | YP_002790881.1 membrane glycoprotein M |
| 2063 | UHI99865.1 membrane glycoprotein |
| | precursor M |
| 2064 | UHI99865.1 protein pr |
| 2065 | UHI99865.1 Flavivirus polyprotein |
| | propeptide |
| 2066 | UHI99865.1 membrane glycoprotein M |
| 2067 | UHI99866.1 membrane glycoprotein |
| | precursor M |
| 2068 | UHI99866.1 protein pr |
| 2069 | UHI99866.1 Flavivirus polyprotein |
| | propeptide |
| 2070 | UHI99866.1 membrane glycoprotein M |
| 2071 | UHI99867.1 membrane glycoprotein |
| | precursor M |
| 2072 | UHI99867.1 protein pr |
| 2073 | UHI99867.1 membrane glycoprotein M |
| 2074 | UED15620.1 membrane glycoprotein |
| | precursor M |
| 2075 | UED15620.1 protein pr |
| 2076 | UED15620.1 Flavivirus polyprotein |
| | propeptide |
| 2077 | UED15620.1 membrane glycoprotein M |
| 2078 | UBI73854.1 membrane glycoprotein |
| | precursor M |
| 2079 | UBI73854.1 protein pr |
| 2080 | UBI73854.1 Flavivirus polyprotein |
| | propeptide |
| 2081 | UBI73854.1 membrane glycoprotein M |
| 2082 | QKF93428.1 membrane glycoprotein |
| | precursor M |
| 2083 | QKF93428.1 protein pr |
| 2084 | QKF93428.1 Flavivirus polyprotein |
| | propeptide |
| 2085 | QKF93428.1 membrane glycoprotein M |
| 2086 | QKF93429.1 membrane glycoprotein |
| | precursor M |
| 2087 | QKF93429.1 protein pr |
| 2088 | QKF93429.1 Flavivirus polyprotein |
| | propeptide |
| 2089 | QKF93429.1 membrane glycoprotein M |
| 2090 | QKF93430.1 membrane glycoprotein |
| | precursor M |
| 2091 | QKF93430.1 protein pr |
| 2092 | QKF93430.1 Flavivirus polyprotein |
| | propeptide |
| 2093 | QKF93430.1 membrane glycoprotein M |
| 2094 | QKF93431.1 membrane glycoprotein |
| | precursor M |
| 2095 | QKF93431.1 protein pr |
| 2096 | QKF93431.1 Flavivirus polyprotein |
| | propeptide |
| 2097 | QKF93431.1 membrane glycoprotein M |
| 2098 | QKF93432.1 membrane glycoprotein |
| | precursor M |
| 2099 | QKF93432.1 protein pr |
| 2100 | QKF93432.1 Flavivirus polyprotein |
| | propeptide |
| 2101 | QKF93432.1 membrane glycoprotein M |
| 2102 | QKF93433.1 membrane glycoprotein |
| | precursor M |
| 2103 | QKF93433.1 protein pr |
| 2104 | QKF93433.1 Flavivirus polyprotein |
| | propeptide |
| 2105 | QKF93433.1 membrane glycoprotein M |
| 2106 | QKF93434.1 membrane glycoprotein |
| | precursor M |
| 2107 | QKF93434.1 protein pr |
| 2108 | QKF93434.1 Flavivirus polyprotein |
| | propeptide |
| 2109 | QKF93434.1 membrane glycoprotein M |
| 2110 | QKF93435.1 membrane glycoprotein |
| | precursor M |
| 2111 | QKF93435.1 protein pr |
| 2112 | QKF93435.1 Flavivirus polyprotein |
| | propeptide |
| 2113 | QKF93435.1 membrane glycoprotein M |
| 2114 | QKF93436.1 membrane glycoprotein |
| | precursor M |
| 2115 | QKF93436.1 protein pr |
| 2116 | QKF93436.1 Flavivirus polyprotein |
| | propeptide |
| 2117 | QKF93436.1 membrane glycoprotein M |
| 2118 | QKF93437.1 membrane glycoprotein |
| | precursor M |
| 2119 | QKF93437.1 protein pr |
| 2120 | QKF93437.1 Flavivirus polyprotein |
| | propeptide |
| 2121 | QKF93437.1 membrane glycoprotein M |
| 2122 | QKF93438.1 membrane glycoprotein |
| | precursor M |
| 2123 | QKF93438.1 protein pr |
| 2124 | QKF93438.1 Flavivirus polyprotein |
| | propeptide |
| 2125 | QKF93438.1 membrane glycoprotein M |
| 2126 | QKF93439.1 membrane glycoprotein |
| | precursor M |
| 2127 | QKF93439.1 protein pr |
| 2128 | QKF93439.1 Flavivirus polyprotein |
| | propeptide |
| 2129 | QKF93439.1 membrane glycoprotein M |
| 2130 | QKF93440.1 membrane glycoprotein |
| | precursor M |
| 2131 | QKF93440.1 protein pr |
| 2132 | QKF93440.1 Flavivirus polyprotein |
| | propeptide |
| 2133 | QKF93440.1 membrane glycoprotein M |
| 2134 | QKF93441.1 membrane glycoprotein |
| | precursor M |
| 2135 | QKF93441.1 protein pr |
| 2136 | QKF93441.1 Flavivirus polyprotein |
| | propeptide |
| 2137 | QKF93441.1 membrane glycoprotein M |
| 2138 | QVL61761.1 Flavivirus polyprotein |
| | propeptide |
| 2139 | QPC45464.1 Flavivirus polyprotein |
| | propeptide |
| 2140 | QTA38993.1 Flavivirus polyprotein |
| | propeptide |
| 2141 | QTA38994.1 Flavivirus polyprotein |
| | propeptide |
| 2142 | QOF88708.1 membrane glycoprotein |
| | precursor M |
| 2143 | QOF88708.1 protein pr |
| 2144 | QOF88708.1 Flavivirus polyprotein |
| | propeptide |
| 2145 | QOF88708.1 membrane glycoprotein M |
| 2146 | QLI34051.1 Flavivirus polyprotein |
| | propeptide |
| 2147 | QKX95861.1 Flavivirus polyprotein |
| | propeptide |
| 2148 | QKU35968.1 membrane glycoprotein |
| | precursor M |
| 2149 | QKU35968.1 protein pr |
| 2150 | QKU35968.1 Flavivirus polyprotein |
| | propeptide |
| 2151 | QKU35968.1 membrane glycoprotein M |
| 2152 | QKM77298.1 membrane glycoprotein |
| | precursor M |
| 2153 | QKM77298.1 protein pr |
| 2154 | QKM77298.1 Flavivirus polyprotein |
| | propeptide |
| 2155 | QKM77298.1 membrane glycoprotein M |
| 2156 | QKM77299.1 membrane glycoprotein |
| | precursor M |
| 2157 | QKM77299.1 protein pr |
| 2158 | QKM77299.1 Flavivirus polyprotein |
| | propeptide |
| 2159 | QKM77299.1 membrane glycoprotein M |
| 2160 | QKM77300.1 membrane glycoprotein |
| | precursor M |
| 2161 | QKM77300.1 protein pr |
| 2162 | QKM77300.1 Flavivirus polyprotein |
| | propeptide |
| 2163 | QKM77300.1 membrane glycoprotein M |
| 2164 | QKM77301.1 membrane glycoprotein |
| | precursor M |
| 2165 | QKM77301.1 protein pr |
| 2166 | QKM77301.1 Flavivirus polyprotein |
| | propeptide |
| 2167 | QKM77301.1 membrane glycoprotein M |
| 2168 | QBM11757.1 Flavivirus polyprotein |
| | propeptide |
| 2169 | AZS35403.1 Flavivirus polyprotein |
| | propeptide |
| 2170 | AZS35404.1 Flavivirus polyprotein |
| | propeptide |
| 2171 | AZS35405.1 Flavivirus polyprotein |
| | propeptide |
| 2172 | QGA72951.1 membrane glycoprotein |
| | precursor M |
| 2173 | QGA72951.1 protein pr |
| 2174 | QGA72951.1 Flavivirus polyprotein |
| | propeptide |
| 2175 | QGA72951.1 membrane glycoprotein M |
| 2176 | QGA67050.1 membrane glycoprotein |
| | precursor M |
| 2177 | QGA67050.1 protein pr |
| 2178 | QGA67050.1 Flavivirus polyprotein |
| | propeptide |
| 2179 | QGA67050.1 membrane glycoprotein M |
| 2180 | QGA67051.1 membrane glycoprotein |
| | precursor M |
| 2181 | QGA67051.1 protein pr |
| 2182 | QGA67051.1 Flavivirus polyprotein |
| | propeptide |
| 2183 | QGA67051.1 membrane glycoprotein M |
| 2184 | QGA67052.1 membrane glycoprotein |
| | precursor M |
| 2185 | QGA67052.1 protein pr |
| 2186 | QGA67052.1 Flavivirus polyprotein |
| | propeptide |
| 2187 | QGA67052.1 membrane glycoprotein M |
| 2188 | QGA67053.1 membrane glycoprotein |
| | precursor M |
| 2189 | QGA67053.1 protein pr |
| 2190 | QGA67053.1 Flavivirus polyprotein |
| | propeptide |
| 2191 | QGA67053.1 membrane glycoprotein M |
| 2192 | QGA67054.1 membrane glycoprotein |
| | precursor M |
| 2193 | QGA67054.1 protein pr |
| 2194 | QGA67054.1 Flavivirus polyprotein |
| | propeptide |
| 2195 | QGA67054.1 membrane glycoprotein M |
| 2196 | QGA67055.1 membrane glycoprotein |
| | precursor M |
| 2197 | QGA67055.1 protein pr |
| 2198 | QGA67055.1 Flavivirus polyprotein |
| | propeptide |
| 2199 | QGA67055.1 membrane glycoprotein M |
| 2200 | QGA67056.1 membrane glycoprotein |
| | precursor M |
| 2201 | QGA67056.1 protein pr |
| 2202 | QGA67056.1 Flavivirus polyprotein |
| | propeptide |
| 2203 | QGA67056.1 membrane glycoprotein M |
| 2204 | QFZ95661.1 membrane glycoprotein |
| | precursor M |
| 2205 | QFZ95661.1 protein pr |
| 2206 | QFZ95661.1 Flavivirus polyprotein |
| | propeptide |
| 2207 | QFZ95661.1 membrane glycoprotein M |
| 2208 | QDZ58854.1 Flavivirus polyprotein |
| | propeptide |
| 2209 | QDZ58855.1 Flavivirus polyprotein |
| | propeptide |
| 2210 | QDZ58856.1 Flavivirus polyprotein |
| | propeptide |
| 2211 | AXQ39996.1 membrane glycoprotein |
| | precursor M |
| 2212 | AXQ39996.1 protein pr |
| 2213 | AXQ39996.1 Flavivirus polyprotein |
| | propeptide |
| 2214 | AXQ39996.1 membrane glycoprotein M |
| 2215 | AXQ39997.1 membrane glycoprotein |
| | precursor M |
| 2216 | AXQ39997.1 protein pr |
| 2217 | AXQ39997.1 Flavivirus polyprotein |
| | propeptide |
| 2218 | AXQ39997.1 membrane glycoprotein M |
| 2219 | AXQ39998.1 membrane glycoprotein |
| | precursor M |
| 2220 | AXQ39998.1 protein pr |
| 2221 | AXQ39998.1 Flavivirus polyprotein |
| | propeptide |
| 2222 | AXQ39998.1 membrane glycoprotein M |
| 2223 | AXQ39999.1 membrane glycoprotein |
| | precursor M |
| 2224 | AXQ39999.1 protein pr |
| 2225 | AXQ39999.1 Flavivirus polyprotein |
| | propeptide |
| 2226 | AXQ39999.1 membrane glycoprotein M |
| 2227 | AXQ40000.1 membrane glycoprotein |
| | precursor M |
| 2228 | AXQ40000.1 protein pr |
| 2229 | AXQ40000.1 Flavivirus polyprotein |
| | propeptide |
| 2230 | AXQ40000.1 membrane glycoprotein M |
| 2231 | AXQ40001.1 membrane glycoprotein |
| | precursor M |
| 2232 | AXQ40001.1 protein pr |
| 2233 | AXQ40001.1 Flavivirus polyprotein |
| | propeptide |
| 2234 | AXQ40001.1 membrane glycoprotein M |
| 2235 | AXQ40002.1 membrane glycoprotein |
| | precursor M |
| 2236 | AXQ40002.1 protein pr |
| 2237 | AXQ40002.1 Flavivirus polyprotein |
| | propeptide |
| 2238 | AXQ40002.1 membrane glycoprotein M |
| 2239 | AXQ40003.1 membrane glycoprotein |
| | precursor M |
| 2240 | AXQ40003.1 protein pr |
| 2241 | AXQ40003.1 Flavivirus polyprotein |
| | propeptide |
| 2242 | AXQ40003.1 membrane glycoprotein M |
| 2243 | AXQ40004.1 membrane glycoprotein |
| | precursor M |
| 2244 | AXQ40004.1 protein pr |
| 2245 | AXQ40004.1 Flavivirus polyprotein |
| | propeptide |
| 2246 | AXQ40004.1 membrane glycoprotein M |
| 2247 | AXQ40005.1 membrane glycoprotein |
| | precursor M |
| 2248 | AXQ40005.1 protein pr |
| 2249 | AXQ40005.1 Flavivirus polyprotein |
| | propeptide |
| 2250 | AXQ40005.1 membrane glycoprotein M |
| 2251 | AXQ40006.1 membrane glycoprotein |
| | precursor M |
| 2252 | AXQ40006.1 protein pr |
| 2253 | AXQ40006.1 Flavivirus polyprotein |
| | propeptide |
| 2254 | AXQ40006.1 membrane glycoprotein M |
| 2255 | AXQ40007.1 membrane glycoprotein |
| | precursor M |
| 2256 | AXQ40007.1 protein pr |
| 2257 | AXQ40007.1 Flavivirus polyprotein |
| | propeptide |
| 2258 | AXQ40007.1 membrane glycoprotein M |
| 2259 | QDH45910.1 Flavivirus polyprotein |
| | propeptide |
| 2260 | QCG74166.1 Flavivirus polyprotein |
| | propeptide |
| 2261 | AYV74916.1 Flavivirus polyprotein |
| | propeptide |
| 2262 | AYV74916.1 Flavivirus envelope |
| | glycoprotein M |
| 2263 | AYV74917.1 Flavivirus polyprotein |
| | propeptide |
| 2264 | AYV74917.1 Flavivirus envelope |
| | glycoprotein M |
| 2265 | AYV74918.1 Flavivirus polyprotein |
| | propeptide |
| 2266 | AYV74918.1 Flavivirus envelope |
| | glycoprotein M |
| 2267 | AYV74919.1 Flavivirus polyprotein |
| | propeptide |
| 2268 | AYV74919.1 Flavivirus envelope |
| | glycoprotein M |
| 2269 | AYV74920.1 Flavivirus polyprotein |
| | propeptide |
| 2270 | AYV74920.1 Flavivirus envelope |
| | glycoprotein M |
| 2271 | AYV74921.1 Flavivirus polyprotein |
| | propeptide |
| 2272 | AYV74921.1 Flavivirus envelope |
| | glycoprotein M |
| 2273 | AYV74922.1 Flavivirus polyprotein |
| | propeptide |
| 2274 | AYV74922.1 Flavivirus envelope |
| | glycoprotein M |
| 2275 | AYV74923.1 Flavivirus polyprotein |
| | propeptide |
| 2276 | AYV74923.1 Flavivirus envelope |
| | glycoprotein M |
| 2277 | AYV74924.1 Flavivirus polyprotein |
| | propeptide |
| 2278 | AYV74924.1 Flavivirus envelope |
| | glycoprotein M |
| 2279 | AYV74925.1 Flavivirus polyprotein |
| | propeptide |
| 2280 | AYV74925.1 Flavivirus envelope |
| | glycoprotein M |
| 2281 | AYV74927.1 Flavivirus polyprotein |
| | propeptide |
| 2282 | AYV74927.1 Flavivirus envelope |
| | glycoprotein M |
| 2283 | AYV74928.1 Flavivirus polyprotein |
| | propeptide |
| 2284 | AYV74928.1 Flavivirus envelope |
| | glycoprotein M |
| 2285 | AYV74929.1 Flavivirus polyprotein |
| | propeptide |
| 2286 | AYV74929.1 Flavivirus envelope |
| | glycoprotein M |
| 2287 | AYV74930.1 Flavivirus polyprotein |
| | propeptide |
| 2288 | AYV74930.1 Flavivirus envelope |
| | glycoprotein M |
| 2289 | AYV74931.1 Flavivirus polyprotein |
| | propeptide |
| 2290 | AYV74931.1 Flavivirus envelope |
| | glycoprotein M |
| 2291 | AYV74932.1 Flavivirus polyprotein |
| | propeptide |
| 2292 | AYV74932.1 Flavivirus envelope |
| | glycoprotein M |
| 2293 | AYV74933.1 Flavivirus polyprotein |
| | propeptide |
| 2294 | AYV74933.1 Flavivirus envelope |
| | glycoprotein M |
| 2295 | AYV74934.1 Flavivirus polyprotein |
| | propeptide |
| 2296 | AYV74934.1 Flavivirus envelope |
| | glycoprotein M |
| 2297 | AYV74935.1 Flavivirus polyprotein |
| | propeptide |
| 2298 | AYV74935.1 Flavivirus envelope |
| | glycoprotein M |
| 2299 | AYV74936.1 Flavivirus polyprotein |
| | propeptide |
| 2300 | AYV74936.1 Flavivirus envelope |
| | glycoprotein M |
| 2301 | AYV74937.1 Flavivirus polyprotein |
| | propeptide |
| 2302 | AYV74937.1 Flavivirus envelope |
| | glycoprotein M |
| 2303 | AYU74865.1 Flavivirus polyprotein |
| | propeptide |
| 2304 | AYU74865.1 Flavivirus envelope |
| | glycoprotein M |
| 2305 | ATB53753.1 Flavivirus polyprotein |
| | propeptide |
| 2306 | ATB53754.1 Flavivirus polyprotein |
| | propeptide |
| 2307 | ATB53754.1 Flavivirus envelope |
| | glycoprotein M |
| 2308 | AVC63697.1 Flavivirus polyprotein |
| | propeptide |
| 2309 | AVC63697.1 Flavivirus envelope |
| | glycoprotein M |
| 2310 | AVC63698.1 Flavivirus polyprotein |
| | propeptide |
| 2311 | AVC63698.1 Flavivirus envelope |
| | glycoprotein M |
| 2312 | AYI50273.1 protein pr |
| 2313 | AYI50273.1 Flavivirus polyprotein |
| | propeptide |
| 2314 | AYI50273.1 small envelope protein M |
| 2315 | AYI50273.1 Flavivirus envelope |
| | glycoprotein M |
| 2316 | AYI50274.1 protein pr |
| 2317 | AYI50274.1 Flavivirus polyprotein |
| | propeptide |
| 2318 | AYI50274.1 small envelope protein M |
| 2319 | AYI50274.1 Flavivirus envelope |
| | glycoprotein M |
| 2320 | AYI50275.1 protein pr |
| 2321 | AYI50275.1 Flavivirus polyprotein |
| | propeptide |
| 2322 | AYI50275.1 small envelope protein M |
| 2323 | AYI50275.1 Flavivirus envelope |
| | glycoprotein M |
| 2324 | ASR91936.1 Flavivirus polyprotein |
| | propeptide |
| 2325 | ASR91936.1 Flavivirus envelope |
| | glycoprotein M |
| 2326 | AXF50014.1 Flavivirus polyprotein |
| | propeptide |
| 2327 | AXF50014.1 Flavivirus envelope |
| | glycoprotein M |
| 2328 | AXF50015.1 Flavivirus polyprotein |
| | propeptide |
| 2329 | AXF50016.1 Flavivirus polyprotein |
| | propeptide |
| 2330 | AXF50016.1 Flavivirus envelope |
| | glycoprotein M |
| 2331 | AXF50052.1 Flavivirus polyprotein |
| | propeptide |
| 2332 | AXF50052.1 Flavivirus envelope |
| | glycoprotein M |
| 2333 | ATL14618.1 Flavivirus polyprotein |
| | propeptide |
| 2334 | AUF35021.1 Flavivirus polyprotein |
| | propeptide |
| 2335 | AUF35021.1 Flavivirus envelope |
| | glycoprotein M |
| 2336 | AUF35022.1 Flavivirus polyprotein |
| | propeptide |
| 2337 | AUF35022.1 Flavivirus envelope |
| | glycoprotein M |
| 2338 | AVG19202.1 Flavivirus polyprotein |
| | propeptide |
| 2339 | AVG19202.1 Flavivirus envelope |
| | glycoprotein M |
| 2340 | AVG19203.1 Flavivirus polyprotein |
| | propeptide |
| 2341 | AVG19203.1 Flavivirus envelope |
| | glycoprotein M |
| 2342 | AVV62004.1 Flavivirus polyprotein |
| | propeptide |
| 2343 | AVV62004.1 Flavivirus envelope |
| | glycoprotein M |
| 2344 | ARU07074.1 Flavivirus polyprotein |
| | propeptide |
| 2345 | ARU07074.1 Flavivirus envelope |
| | glycoprotein M |
| 2346 | ARU07075.1 Flavivirus polyprotein |
| | propeptide |
| 2347 | ARU07075.1 Flavivirus envelope |
| | glycoprotein M |
| 2348 | ARU07076.1 Flavivirus polyprotein |
| | propeptide |
| 2349 | ARU07076.1 Flavivirus envelope |
| | glycoprotein M |
| 2350 | AUY62552.1 Flavivirus polyprotein |
| | propeptide |
| 2351 | AUY62553.1 Flavivirus polyprotein |
| | propeptide |
| 2352 | AUY62553.1 Flavivirus envelope |
| | glycoprotein M |
| 2353 | AWH65848.1 Flavivirus polyprotein |
| | propeptide |
| 2354 | AWH65849.1 Flavivirus polyprotein |
| | propeptide |
| 2355 | AWH65849.1 Flavivirus envelope |
| | glycoprotein M |
| 2356 | AWF93617.1 protein pr |
| 2357 | AWF93617.1 Flavivirus polyprotein |
| | propeptide |
| 2358 | AWF93617.1 small envelope protein M |
| 2359 | AWF93617.1 Flavivirus envelope |
| | glycoprotein M |
| 2360 | AWF93618.1 protein pr |
| 2361 | AWF93618.1 Flavivirus polyprotein |
| | propeptide |
| 2362 | AWF93618.1 small envelope protein M |
| 2363 | AWF93618.1 Flavivirus envelope |
| | glycoprotein M |
| 2364 | AWF93619.1 protein pr |
| 2365 | AWF93619.1 Flavivirus polyprotein |
| | propeptide |
| 2366 | AWF93619.1 small envelope protein M |
| 2367 | AWF93619.1 Flavivirus envelope |
| | glycoprotein M |
| 2368 | AWF93620.1 protein pr |
| 2369 | AWF93620.1 Flavivirus polyprotein |
| | propeptide |
| 2370 | AWF93620.1 small envelope protein M |
| 2371 | AWF93620.1 Flavivirus envelope |
| | glycoprotein M |
| 2372 | AWF93621.1 protein pr |
| 2373 | AWF93621.1 Flavivirus polyprotein |
| | propeptide |
| 2374 | AWF93621.1 small envelope protein M |
| 2375 | AWF93621.1 Flavivirus envelope |
| | glycoprotein M |
| 2376 | AWF93622.1 protein pr |
| 2377 | AWF93622.1 Flavivirus polyprotein |
| | propeptide |
| 2378 | AWF93622.1 small envelope protein M |
| 2379 | AWF93622.1 Flavivirus envelope |
| | glycoprotein M |
| 2380 | AWF93623.1 protein pr |
| 2381 | AWF93623.1 Flavivirus polyprotein |
| | propeptide |
| 2382 | AWF93623.1 small envelope protein M |
| 2383 | AWF93623.1 Flavivirus envelope |
| | glycoprotein M |
| 2384 | AWF93624.1 protein pr |
| 2385 | AWF93624.1 Flavivirus polyprotein |
| | propeptide |
| 2386 | AWF93624.1 small envelope protein M |
| 2387 | AWF93624.1 Flavivirus envelope |
| | glycoprotein M |
| 2388 | AWF93625.1 protein pr |
| 2389 | AWF93625.1 Flavivirus polyprotein |
| | propeptide |
| 2390 | AWF93625.1 small envelope protein M |
| 2391 | AWF93625.1 Flavivirus envelope |
| | glycoprotein M |
| 2392 | AWF93626.1 protein pr |
| 2393 | AWF93626.1 Flavivirus polyprotein |
| | propeptide |
| 2394 | AWF93626.1 small envelope protein M |
| 2395 | AWF93626.1 Flavivirus envelope |
| | glycoprotein M |
| 2396 | AWF93627.1 protein pr |
| 2397 | AWF93627.1 Flavivirus polyprotein |
| | propeptide |
| 2398 | AWF93627.1 small envelope protein M |
| 2399 | AWF93627.1 Flavivirus envelope |
| | glycoprotein M |
| 2400 | AWF93628.1 protein pr |
| 2401 | AWF93628.1 Flavivirus polyprotein |
| | propeptide |
| 2402 | AWF93628.1 small envelope protein M |
| 2403 | AWF93628.1 Flavivirus envelope |
| | glycoprotein M |
| 2404 | AWF93629.1 protein pr |
| 2405 | AWF93629.1 Flavivirus polyprotein |
| | propeptide |
| 2406 | AWF93629.1 small envelope protein M |
| 2407 | AWF93629.1 Flavivirus envelope |
| | glycoprotein M |
| 2408 | AWF93630.1 protein pr |
| 2409 | AWF93630.1 Flavivirus polyprotein |
| | propeptide |
| 2410 | AWF93630.1 small envelope protein M |
| 2411 | AWF93630.1 Flavivirus envelope |
| | glycoprotein M |
| 2412 | AWF93631.1 protein pr |
| 2413 | AWF93631.1 Flavivirus polyprotein |
| | propeptide |
| 2414 | AWF93631.1 small envelope protein M |
| 2415 | AWF93631.1 Flavivirus envelope |
| | glycoprotein M |
| 2416 | AWF93632.1 protein pr |
| 2417 | AWF93632.1 Flavivirus polyprotein |
| | propeptide |
| 2418 | AWF93632.1 small envelope protein M |
| 2419 | AWF93632.1 Flavivirus envelope |
| | glycoprotein M |
| 2420 | AWF93633.1 protein pr |
| 2421 | AWF93633.1 Flavivirus polyprotein |
| | propeptide |
| 2422 | AWF93633.1 small envelope protein M |
| 2423 | AWF93633.1 Flavivirus envelope |
| | glycoprotein M |
| 2424 | AWF93634.1 protein pr |
| 2425 | AWF93634.1 Flavivirus polyprotein |
| | propeptide |
| 2426 | AWF93634.1 small envelope protein M |
| 2427 | AWF93634.1 Flavivirus envelope |
| | glycoprotein M |
| 2428 | AWF93635.1 protein pr |
| 2429 | AWF93635.1 Flavivirus polyprotein |
| | propeptide |
| 2430 | AWF93635.1 small envelope protein M |
| 2431 | AWF93635.1 Flavivirus envelope |
| | glycoprotein M |
| 2432 | AVZ47169.1 Flavivirus polyprotein |
| | propeptide |
| 2433 | AVZ47169.1 Flavivirus envelope |
| | glycoprotein M |
| 2434 | AVW85800.1 protein pr |
| 2435 | AVW85800.1 Flavivirus polyprotein |
| | propeptide |
| 2436 | AVW85800.1 small envelope protein M |
| 2437 | AVW85800.1 Flavivirus envelope |
| | glycoprotein M |
| 2438 | AVW85801.1 protein pr |
| 2439 | AVW85801.1 Flavivirus polyprotein |
| | propeptide |
| 2440 | AVW85801.1 small envelope protein M |
| 2441 | AVW85801.1 Flavivirus envelope |
| | glycoprotein M |
| 2442 | AVW85802.1 protein pr |
| 2443 | AVW85802.1 Flavivirus polyprotein |
| | propeptide |
| 2444 | AVW85802.1 small envelope protein M |
| 2445 | AVW85802.1 Flavivirus envelope |
| | glycoprotein M |
| 2446 | AVW85803.1 protein pr |
| 2447 | AVW85803.1 Flavivirus polyprotein |
| | propeptide |
| 2448 | AVW85803.1 small envelope protein M |
| 2449 | AVW85803.1 Flavivirus envelope |
| | glycoprotein M |
| 2450 | AVW85804.1 protein pr |
| 2451 | AVW85804.1 Flavivirus polyprotein |
| | propeptide |
| 2452 | AVW85804.1 small envelope protein M |
| 2453 | AVW85804.1 Flavivirus envelope |
| | glycoprotein M |
| 2454 | AVW85805.1 protein pr |
| 2455 | AVW85805.1 Flavivirus polyprotein |
| | propeptide |
| 2456 | AVW85805.1 small envelope protein M |
| 2457 | AVW85805.1 Flavivirus envelope |
| | glycoprotein M |
| 2458 | AVW85806.1 protein pr |
| 2459 | AVW85806.1 Flavivirus polyprotein |
| | propeptide |
| 2460 | AVW85806.1 small envelope protein M |
| 2461 | AVW85806.1 Flavivirus envelope |
| | glycoprotein M |
| 2462 | AVW85807.1 protein pr |
| 2463 | AVW85807.1 Flavivirus polyprotein |
| | propeptide |
| 2464 | AVW85807.1 small envelope protein M |
| 2465 | AVW85807.1 Flavivirus envelope |
| | glycoprotein M |
| 2466 | AVW85808.1 protein pr |
| 2467 | AVW85808.1 Flavivirus polyprotein |
| | propeptide |
| 2468 | AVW85808.1 small envelope protein M |
| 2469 | AVW85808.1 Flavivirus envelope |
| | glycoprotein M |
| 2470 | AVW85809.1 protein pr |
| 2471 | AVW85809.1 Flavivirus polyprotein |
| | propeptide |
| 2472 | AVW85809.1 small envelope protein M |
| 2473 | AVW85809.1 Flavivirus envelope |
| | glycoprotein M |
| 2474 | AVW85810.1 protein pr |
| 2475 | AVW85810.1 Flavivirus polyprotein |
| | propeptide |
| 2476 | AVW85810.1 small envelope protein M |
| 2477 | AVW85810.1 Flavivirus envelope |
| | glycoprotein M |
| 2478 | AVW85811.1 protein pr |
| 2479 | AVW85811.1 Flavivirus polyprotein |
| | propeptide |
| 2480 | AVW85811.1 small envelope protein M |
| 2481 | AVW85811.1 Flavivirus envelope |
| | glycoprotein M |
| 2482 | AVW85812.1 protein pr |
| 2483 | AVW85812.1 Flavivirus polyprotein |
| | propeptide |
| 2484 | AVW85812.1 small envelope protein M |
| 2485 | AVW85812.1 Flavivirus envelope |
| | glycoprotein M |
| 2486 | AVW85813.1 protein pr |
| 2487 | AVW85813.1 Flavivirus polyprotein |
| | propeptide |
| 2488 | AVW85813.1 small envelope protein M |
| 2489 | AVW85813.1 Flavivirus envelope |
| | glycoprotein M |
| 2490 | AVW85814.1 protein pr |
| 2491 | AVW85814.1 Flavivirus polyprotein |
| | propeptide |
| 2492 | AVW85814.1 small envelope protein M |
| 2493 | AVW85814.1 Flavivirus envelope |
| | glycoprotein M |
| 2494 | AVW85815.1 protein pr |
| 2495 | AVW85815.1 Flavivirus polyprotein |
| | propeptide |
| 2496 | AVW85815.1 small envelope protein M |
| 2497 | AVW85815.1 Flavivirus envelope |
| | glycoprotein M |
| 2498 | AVW85816.1 protein pr |
| 2499 | AVW85816.1 Flavivirus polyprotein |
| | propeptide |
| 2500 | AVW85816.1 small envelope protein M |
| 2501 | AVW85816.1 Flavivirus envelope |
| | glycoprotein M |
| 2502 | AVW85817.1 protein pr |
| 2503 | AVW85817.1 Flavivirus polyprotein |
| | propeptide |
| 2504 | AVW85817.1 small envelope protein M |
| 2505 | AVW85817.1 Flavivirus envelope |
| | glycoprotein M |
| 2506 | AVW85818.1 protein pr |
| 2507 | AVW85818.1 Flavivirus polyprotein |
| | propeptide |
| 2508 | AVW85818.1 small envelope protein M |
| 2509 | AVW85818.1 Flavivirus envelope |
| | glycoprotein M |
| 2510 | AVW85819.1 protein pr |
| 2511 | AVW85819.1 Flavivirus polyprotein |
| | propeptide |
| 2512 | AVW85819.1 small envelope protein M |
| 2513 | AVW85819.1 Flavivirus envelope |
| | glycoprotein M |
| 2514 | AVW85820.1 protein pr |
| 2515 | AVW85820.1 Flavivirus polyprotein |
| | propeptide |
| 2516 | AVW85820.1 small envelope protein M |
| 2517 | AVW85820.1 Flavivirus envelope |
| | glycoprotein M |
| 2518 | AVW85821.1 protein pr |
| 2519 | AVW85821.1 Flavivirus polyprotein |
| | propeptide |
| 2520 | AVW85821.1 small envelope protein M |
| 2521 | AVW85821.1 Flavivirus envelope |
| | glycoprotein M |
| 2522 | AVW85822.1 protein pr |
| 2523 | AVW85822.1 Flavivirus polyprotein |
| | propeptide |
| 2524 | AVW85822.1 small envelope protein M |
| 2525 | AVW85822.1 Flavivirus envelope |
| | glycoprotein M |
| 2526 | AVW85823.1 protein pr |
| 2527 | AVW85823.1 Flavivirus polyprotein |
| | propeptide |
| 2528 | AVW85823.1 small envelope protein M |
| 2529 | AVW85823.1 Flavivirus envelope |
| | glycoprotein M |
| 2530 | AVW85824.1 protein pr |
| 2531 | AVW85824.1 Flavivirus polyprotein |
| | propeptide |
| 2532 | AVW85824.1 small envelope protein M |
| 2533 | AVW85824.1 Flavivirus envelope |
| | glycoprotein M |
| 2534 | AVW85825.1 protein pr |
| 2535 | AVW85825.1 Flavivirus polyprotein |
| | propeptide |
| 2536 | AVW85825.1 small envelope protein M |
| 2537 | AVW85825.1 Flavivirus envelope |
| | glycoprotein M |
| 2538 | AVW85826.1 protein pr |
| 2539 | AVW85826.1 Flavivirus polyprotein |
| | propeptide |
| 2540 | AVW85826.1 small envelope protein M |
| 2541 | AVW85826.1 Flavivirus envelope |
| | glycoprotein M |
| 2542 | AVW85827.1 protein pr |
| 2543 | AVW85827.1 Flavivirus polyprotein |
| | propeptide |
| 2544 | AVW85827.1 small envelope protein M |
| 2545 | AVW85827.1 Flavivirus envelope |
| | glycoprotein M |
| 2546 | AVW85828.1 protein pr |
| 2547 | AVW85828.1 Flavivirus polyprotein |
| | propeptide |
| 2548 | AVW85828.1 small envelope protein M |
| 2549 | AVW85828.1 Flavivirus envelope |
| | glycoprotein M |
| 2550 | AVW85829.1 protein pr |
| 2551 | AVW85829.1 Flavivirus polyprotein |
| | propeptide |
| 2552 | AVW85829.1 small envelope protein M |
| 2553 | AVW85829.1 Flavivirus envelope |
| | glycoprotein M |
| 2554 | AVW85830.1 protein pr |
| 2555 | AVW85830.1 Flavivirus polyprotein |
| | propeptide |
| 2556 | AVW85830.1 small envelope protein M |
| 2557 | AVW85830.1 Flavivirus envelope |
| | glycoprotein M |
| 2558 | AVW85831.1 protein pr |
| 2559 | AVW85831.1 Flavivirus polyprotein |
| | propeptide |
| 2560 | AVW85831.1 small envelope protein M |
| 2561 | AVW85831.1 Flavivirus envelope |
| | glycoprotein M |
| 2562 | AVW85832.1 protein pr |
| 2563 | AVW85832.1 Flavivirus polyprotein |
| | propeptide |
| 2564 | AVW85832.1 small envelope protein M |
| 2565 | AVW85832.1 Flavivirus envelope |
| | glycoprotein M |
| 2566 | AVW85833.1 protein pr |
| 2567 | AVW85833.1 Flavivirus polyprotein |
| | propeptide |
| 2568 | AVW85833.1 small envelope protein M |
| 2569 | AVW85833.1 Flavivirus envelope |
| | glycoprotein M |
| 2570 | AVW85834.1 protein pr |
| 2571 | AVW85834.1 Flavivirus polyprotein |
| | propeptide |
| 2572 | AVW85834.1 small envelope protein M |
| 2573 | AVW85834.1 Flavivirus envelope |
| | glycoprotein M |
| 2574 | AVW85835.1 protein pr |
| 2575 | AVW85835.1 Flavivirus polyprotein |
| | propeptide |
| 2576 | AVW85835.1 small envelope protein M |
| 2577 | AVW85835.1 Flavivirus envelope |
| | glycoprotein M |
| 2578 | AVW85836.1 protein pr |
| 2579 | AVW85836.1 Flavivirus polyprotein |
| | propeptide |
| 2580 | AVW85836.1 small envelope protein M |
| 2581 | AVW85836.1 Flavivirus envelope |
| | glycoprotein M |
| 2582 | AVW85837.1 protein pr |
| 2583 | AVW85837.1 Flavivirus polyprotein |
| | propeptide |
| 2584 | AVW85837.1 small envelope protein M |
| 2585 | AVW85837.1 Flavivirus envelope |
| | glycoprotein M |
| 2586 | AVW85838.1 protein pr |
| 2587 | AVW85838.1 Flavivirus polyprotein |
| | propeptide |
| 2588 | AVW85838.1 small envelope protein M |
| 2589 | AVW85838.1 Flavivirus envelope |
| | glycoprotein M |
| 2590 | AVW85839.1 protein pr |
| 2591 | AVW85839.1 Flavivirus polyprotein |
| | propeptide |
| 2592 | AVW85839.1 small envelope protein M |
| 2593 | AVW85839.1 Flavivirus envelope |
| | glycoprotein M |
| 2594 | AVW85840.1 protein pr |
| 2595 | AVW85840.1 Flavivirus polyprotein |
| | propeptide |
| 2596 | AVW85840.1 small envelope protein M |
| 2597 | AVW85840.1 Flavivirus envelope |
| | glycoprotein M |
| 2598 | AVW85841.1 protein pr |
| 2599 | AVW85841.1 Flavivirus polyprotein |
| | propeptide |
| 2600 | AVW85841.1 small envelope protein M |
| 2601 | AVW85841.1 Flavivirus envelope |
| | glycoprotein M |
| 2602 | AVW85842.1 protein pr |
| 2603 | AVW85842.1 Flavivirus polyprotein |
| | propeptide |
| 2604 | AVW85842.1 small envelope protein M |
| 2605 | AVW85842.1 Flavivirus envelope |
| | glycoprotein M |
| 2606 | AVW85843.1 protein pr |
| 2607 | AVW85843.1 Flavivirus polyprotein |
| | propeptide |
| 2608 | AVW85843.1 small envelope protein M |
| 2609 | AVW85843.1 Flavivirus envelope |
| | glycoprotein M |
| 2610 | AVW85844.1 protein pr |
| 2611 | AVW85844.1 Flavivirus polyprotein |
| | propeptide |
| 2612 | AVW85844.1 small envelope protein M |
| 2613 | AVW85844.1 Flavivirus envelope |
| | glycoprotein M |
| 2614 | AVW85845.1 protein pr |
| 2615 | AVW85845.1 Flavivirus polyprotein |
| | propeptide |
| 2616 | AVW85845.1 small envelope protein M |
| 2617 | AVW85845.1 Flavivirus envelope |
| | glycoprotein M |
| 2618 | AVW85846.1 protein pr |
| 2619 | AVW85846.1 Flavivirus polyprotein |
| | propeptide |
| 2620 | AVW85846.1 small envelope protein M |
| 2621 | AVW85846.1 Flavivirus envelope |
| | glycoprotein M |
| 2622 | AVW85847.1 protein pr |
| 2623 | AVW85847.1 Flavivirus polyprotein |
| | propeptide |
| 2624 | AVW85847.1 small envelope protein M |
| 2625 | AVW85847.1 Flavivirus envelope |
| | glycoprotein M |
| 2626 | AVW85848.1 protein pr |
| 2627 | AVW85848.1 Flavivirus polyprotein |
| | propeptide |
| 2628 | AVW85848.1 small envelope protein M |
| 2629 | AVW85848.1 Flavivirus envelope |
| | glycoprotein M |
| 2630 | AVW85849.1 protein pr |
| 2631 | AVW85849.1 Flavivirus polyprotein |
| | propeptide |
| 2632 | AVW85849.1 small envelope protein M |
| 2633 | AVW85849.1 Flavivirus envelope |
| | glycoprotein M |
| 2634 | AVW85850.1 protein pr |
| 2635 | AVW85850.1 Flavivirus polyprotein |
| | propeptide |
| 2636 | AVW85850.1 small envelope protein M |
| 2637 | AVW85850.1 Flavivirus envelope |
| | glycoprotein M |
| 2638 | AVW85851.1 protein pr |
| 2639 | AVW85851.1 Flavivirus polyprotein |
| | propeptide |
| 2640 | AVW85851.1 small envelope protein M |
| 2641 | AVW85851.1 Flavivirus envelope |
| | glycoprotein M |
| 2642 | AVW85852.1 protein pr |
| 2643 | AVW85852.1 Flavivirus polyprotein |
| | propeptide |
| 2644 | AVW85852.1 small envelope protein M |
| 2645 | AVW85852.1 Flavivirus envelope |
| | glycoprotein M |
| 2646 | AVW85853.1 protein pr |
| 2647 | AVW85853.1 Flavivirus polyprotein |
| | propeptide |
| 2648 | AVW85853.1 small envelope protein M |
| 2649 | AVW85853.1 Flavivirus envelope |
| | glycoprotein M |
| 2650 | AVW85854.1 protein pr |
| 2651 | AVW85854.1 Flavivirus polyprotein |
| | propeptide |
| 2652 | AVW85854.1 small envelope protein M |
| 2653 | AVW85854.1 Flavivirus envelope |
| | glycoprotein M |
| 2654 | AVW85855.1 protein pr |
| 2655 | AVW85855.1 Flavivirus polyprotein |
| | propeptide |
| 2656 | AVW85855.1 small envelope protein M |
| 2657 | AVW85855.1 Flavivirus envelope |
| | glycoprotein M |
| 2658 | AVW85856.1 protein pr |
| 2659 | AVW85856.1 Flavivirus polyprotein |
| | propeptide |
| 2660 | AVW85856.1 small envelope protein M |
| 2661 | AVW85856.1 Flavivirus envelope |
| | glycoprotein M |
| 2662 | AVW85857.1 protein pr |
| 2663 | AVW85857.1 Flavivirus polyprotein |
| | propeptide |
| 2664 | AVW85857.1 small envelope protein M |
| 2665 | AVW85857.1 Flavivirus envelope |
| | glycoprotein M |
| 2666 | AVW85858.1 protein pr |
| 2667 | AVW85858.1 Flavivirus polyprotein |
| | propeptide |
| 2668 | AVW85858.1 small envelope protein M |
| 2669 | AVW85858.1 Flavivirus envelope |
| | glycoprotein M |
| 2670 | AVW85859.1 protein pr |
| 2671 | AVW85859.1 Flavivirus polyprotein |
| | propeptide |
| 2672 | AVW85859.1 small envelope protein M |
| 2673 | AVW85859.1 Flavivirus envelope |
| | glycoprotein M |
| 2674 | AVW85860.1 protein pr |
| 2675 | AVW85860.1 Flavivirus polyprotein |
| | propeptide |
| 2676 | AVW85860.1 small envelope protein M |
| 2677 | AVW85860.1 Flavivirus envelope |
| | glycoprotein M |
| 2678 | AVW85861.1 protein pr |
| 2679 | AVW85861.1 Flavivirus polyprotein |
| | propeptide |
| 2680 | AVW85861.1 small envelope protein M |
| 2681 | AVW85861.1 Flavivirus envelope |
| | glycoprotein M |
| 2682 | AVW85862.1 protein pr |
| 2683 | AVW85862.1 Flavivirus polyprotein |
| | propeptide |
| 2684 | AVW85862.1 small envelope protein M |
| 2685 | AVW85862.1 Flavivirus envelope |
| | glycoprotein M |
| 2686 | AVW85863.1 protein pr |
| 2687 | AVW85863.1 Flavivirus polyprotein |
| | propeptide |
| 2688 | AVW85863.1 small envelope protein M |
| 2689 | AVW85863.1 Flavivirus envelope |
| | glycoprotein M |
| 2690 | BBC70847.1 Flavivirus polyprotein |
| | propeptide |
| 2691 | BBC70847.1 Flavivirus envelope |
| | glycoprotein M |
| 2692 | AVG19275.1 Flavivirus polyprotein |
| | propeptide |
| 2693 | AVG19275.1 Flavivirus envelope |
| | glycoprotein M |
| 2694 | AUI42329.1 Flavivirus polyprotein |
| | propeptide |
| 2695 | AUI42329.1 Flavivirus envelope |
| | glycoprotein M |
| 2696 | AUI42330.1 Flavivirus polyprotein |
| | propeptide |
| 2697 | AUI42330.1 Flavivirus envelope |
| | glycoprotein M |
| 2698 | ARB08102.1 Flavivirus polyprotein |
| | propeptide |
| 2699 | ATB53752.1 Flavivirus polyprotein |
| | propeptide |
| 2700 | AQS26698.1 Flavivirus polyprotein |
| | propeptide |
| 2701 | AQS26698.1 Flavivirus envelope |
| | glycoprotein M |
| 2702 | ASF57880.1 Flavivirus polyprotein |
| | propeptide |
| 2703 | ASB32509.1 Flavivirus polyprotein |
| | propeptide |
| 2704 | APW84872.1 Flavivirus polyprotein |
| | propeptide |
| 2705 | APW84873.1 Flavivirus polyprotein |
| | propeptide |
| 2706 | APW84874.1 Flavivirus polyprotein |
| | propeptide |
| 2707 | APW84875.1 Flavivirus polyprotein |
| | propeptide |
| 2708 | APW84876.1 Flavivirus polyprotein |
| | propeptide |
| 2709 | APW84877.1 Flavivirus polyprotein |
| | propeptide |
| 2710 | ARM59239.1 Flavivirus polyprotein |
| | propeptide |
| 2711 | ARM59239.1 Flavivirus envelope |
| | glycoprotein M |
| 2712 | ARM59240.1 protein pr |
| 2713 | ARM59240.1 Flavivirus polyprotein |
| | propeptide |
| 2714 | ARM59240.1 small envelope protein M |
| 2715 | ARM59240.1 Flavivirus envelope |
| | glycoprotein M |
| 2716 | AMX81915.1 Flavivirus polyprotein |
| | propeptide |
| 2717 | AMX81915.1 Flavivirus envelope |
| | glycoprotein M |
| 2718 | AMX81916.1 Flavivirus polyprotein |
| | propeptide |
| 2719 | AMX81916.1 Flavivirus envelope |
| | glycoprotein M |
| 2720 | AMX81917.1 Flavivirus polyprotein |
| | propeptide |
| 2721 | AMX81917.1 Flavivirus envelope |
| | glycoprotein M |
| 2722 | AQZ41947.1 protein pr |
| 2723 | AQZ41947.1 Flavivirus polyprotein |
| | propeptide |
| 2724 | AQZ41947.1 small envelope protein M |
| 2725 | AQZ41948.1 protein pr |
| 2726 | AQZ41948.1 Flavivirus polyprotein |
| | propeptide |
| 2727 | AQZ41948.1 small envelope protein M |
| 2728 | AQZ41949.1 protein pr |
| 2729 | AQZ41949.1 Flavivirus polyprotein |
| | propeptide |
| 2730 | AQZ41949.1 small envelope protein M |
| 2731 | AQZ41949.1 Flavivirus envelope |
| | glycoprotein M |
| 2732 | AQZ41950.1 protein pr |
| 2733 | AQZ41950.1 Flavivirus polyprotein |
| | propeptide |
| 2734 | AQZ41950.1 small envelope protein M |
| 2735 | AQZ41951.1 protein pr |
| 2736 | AQZ41951.1 Flavivirus polyprotein |
| | propeptide |
| 2737 | AQZ41951.1 small envelope protein M |
| 2738 | AQZ41952.1 protein pr |
| 2739 | AQZ41952.1 Flavivirus polyprotein |
| | propeptide |
| 2740 | AQZ41952.1 small envelope protein M |
| 2741 | AQZ41952.1 Flavivirus envelope |
| | glycoprotein M |
| 2742 | AQZ41953.1 protein pr |
| 2743 | AQZ41953.1 Flavivirus polyprotein |
| | propeptide |
| 2744 | AQZ41953.1 small envelope protein M |
| 2745 | AQZ41953.1 Flavivirus envelope |
| | glycoprotein M |
| 2746 | AQZ41954.1 protein pr |
| 2747 | AQZ41954.1 Flavivirus polyprotein |
| | propeptide |
| 2748 | AQZ41954.1 small envelope protein M |
| 2749 | AQZ41955.1 protein pr |
| 2750 | AQZ41955.1 Flavivirus polyprotein |
| | propeptide |
| 2751 | AQZ41955.1 small envelope protein M |
| 2752 | AQZ41955.1 Flavivirus envelope |
| | glycoprotein M |
| 2753 | AQZ41956.1 protein pr |
| 2754 | AQZ41956.1 Flavivirus polyprotein |
| | propeptide |
| 2755 | AQZ41956.1 small envelope protein M |
| 2756 | AQW43707.1 Flavivirus polyprotein |
| | propeptide |
| 2757 | AQW43707.1 Flavivirus envelope |
| | glycoprotein M |
| 2758 | AQW43708.1 Flavivirus polyprotein |
| | propeptide |
| 2759 | AQW43708.1 Flavivirus envelope |
| | glycoprotein M |
| 2760 | BAX00477.1 Flavivirus polyprotein |
| | propeptide |
| 2761 | BAX00477.1 Flavivirus envelope |
| | glycoprotein M |
| 2762 | AQV07165.1 protein pr |
| 2763 | AQV07165.1 Flavivirus polyprotein |
| | propeptide |
| 2764 | AQV07165.1 small envelope protein M |
| 2765 | AQV07165.1 Flavivirus envelope |
| | glycoprotein M |
| 2766 | AOO19564.1 protein pr |
| 2767 | AOO19564.1 Flavivirus polyprotein |
| | propeptide |
| 2768 | AOO19564.1 membrane glycoprotein M |
| 2769 | AOO19564.1 Flavivirus envelope |
| | glycoprotein M |
| 2770 | APO36913.1 Flavivirus polyprotein |
| | propeptide |
| 2771 | APO36913.1 Flavivirus envelope |
| | glycoprotein M |
| 2772 | AMM43325.1 Flavivirus polyprotein |
| | propeptide |
| 2773 | AMM43325.1 Flavivirus envelope |
| | glycoprotein M |
| 2774 | AMM43326.1 Flavivirus polyprotein |
| | propeptide |
| 2775 | AMM43326.1 Flavivirus envelope |
| | glycoprotein M |
| 2776 | APH11492.1 membrane glycoprotein |
| | precursor M |
| 2777 | APH11492.1 Flavivirus polyprotein |
| | propeptide |
| 2778 | APH11492.1 Flavivirus envelope |
| | glycoprotein M |
| 2779 | APO08503.1 Flavivirus polyprotein |
| | propeptide |
| 2780 | APC60216.2 Flavivirus polyprotein |
| | propeptide |
| 2781 | APC60216.2 Flavivirus envelope |
| | glycoprotein M |
| 2782 | ANH22038.1 Flavivirus polyprotein |
| | propeptide |
| 2783 | BAV82373.1 Flavivirus polyprotein |
| | propeptide |
| 2784 | AOR82892.1 Flavivirus polyprotein |
| | propeptide |
| 2785 | AOR82893.1 Flavivirus polyprotein |
| | propeptide |
| 2786 | AOR51315.1 Flavivirus polyprotein |
| | propeptide |
| 2787 | AOR51315.1 Flavivirus envelope |
| | glycoprotein M |
| 2788 | AND01116.2 Flavivirus polyprotein |
| | propeptide |
| 2789 | AND01116.2 Flavivirus envelope |
| | glycoprotein M |
| 2790 | AOG18296.1 Flavivirus polyprotein |
| | propeptide |
| 2791 | AOG18296.1 Flavivirus envelope |
| | glycoprotein M |
| 2792 | AOC50652.1 protein pr |
| 2793 | AOC50652.1 small envelope protein M |
| 2794 | AOC50653.1 protein pr |
| 2795 | AOC50653.1 small envelope protein M |
| 2796 | AOC50654.1 protein pr |
| 2797 | AOC50654.1 small envelope protein M |
| 2798 | ANZ46736.1 Flavivirus polyprotein |
| | propeptide |
| 2799 | ANW07474.2 protein pr |
| 2800 | ANW07474.2 Flavivirus polyprotein |
| | propeptide |
| 2801 | ANW07474.2 small envelope protein M |
| 2802 | ANW07475.1 protein pr |
| 2803 | ANW07475.1 small envelope protein M |
| 2804 | ANW07476.1 protein pr |
| 2805 | ANW07476.1 Flavivirus polyprotein |
| | propeptide |
| 2806 | ANW07476.1 small envelope protein M |
| 2807 | ANW07477.1 protein pr |
| 2808 | ANW07477.1 Flavivirus polyprotein |
| | propeptide |
| 2809 | ANW07477.1 small envelope protein M |
| 2810 | ANW07477.1 Flavivirus envelope |
| | glycoprotein M |
| 2811 | ANN44857.1 Flavivirus polyprotein |
| | propeptide |
| 2812 | ANN44857.1 Flavivirus envelope |
| | glycoprotein M |
| 2813 | ANN83272.1 protein pr |
| 2814 | ANN83272.1 small envelope protein M |
| 2815 | ANN83273.1 protein pr |
| 2816 | ANN83273.1 small envelope protein M |
| 2817 | ANK57897.1 Flavivirus polyprotein |
| | propeptide |
| 2818 | ANK57897.1 Flavivirus envelope |
| | glycoprotein M |
| 2819 | ANH10698.1 Flavivirus polyprotein |
| | propeptide |
| 2820 | ANG09399.1 Flavivirus polyprotein |
| | propeptide |
| 2821 | ANF16414.1 Flavivirus polyprotein |
| | propeptide |
| 2822 | ANF16414.1 Flavivirus envelope |
| | glycoprotein M |
| 2823 | ANC90427.1 protein pr |
| 2824 | ANC90427.1 small envelope protein M |
| 2825 | ANC90428.1 protein pr |
| 2826 | ANC90428.1 Flavivirus polyprotein |
| | propeptide |
| 2827 | ANC90428.1 small envelope protein M |
| 2828 | ANC90428.1 Flavivirus envelope |
| | glycoprotein M |
| 2829 | ANB66182.1 protein pr |
| 2830 | ANB66182.1 Flavivirus polyprotein |
| | propeptide |
| 2831 | ANB66182.1 small envelope protein M |
| 2832 | ANB66182.1 Flavivirus envelope |
| | glycoprotein M |
| 2833 | ANB66183.1 protein pr |
| 2834 | ANB66183.1 Flavivirus polyprotein |
| | propeptide |
| 2835 | ANB66183.1 small envelope protein M |
| 2836 | ANB66184.1 protein pr |
| 2837 | ANB66184.1 small envelope protein M |
| 2838 | ANA12599.1 Flavivirus polyprotein |
| | propeptide |
| 2839 | ANA12599.1 Flavivirus envelope |
| | glycoprotein M |
| 2840 | AMX81919.1 Flavivirus polyprotein |
| | propeptide |
| 2841 | AMX81919.1 Flavivirus envelope |
| | glycoprotein M |
| 2842 | AMR39832.1 Flavivirus polyprotein |
| | propeptide |
| 2843 | AMR39833.1 Flavivirus polyprotein |
| | propeptide |
| 2844 | AMR39833.1 Flavivirus envelope |
| | glycoprotein M |
| 2845 | AMR39834.1 Flavivirus polyprotein |
| | propeptide |
| 2846 | AMR39835.1 Flavivirus polyprotein |
| | propeptide |
| 2847 | AMR39835.1 Flavivirus envelope |
| | glycoprotein M |
| 2848 | AMR39836.1 Flavivirus polyprotein |
| | propeptide |
| 2849 | AMR39836.1 Flavivirus envelope |
| | glycoprotein M |
| 2850 | AMQ48986.1 Flavivirus polyprotein |
| | propeptide |
| 2851 | AMQ48986.1 Flavivirus envelope |
| | glycoprotein M |
| 2852 | AMQ48981.1 membrane glycoprotein |
| | precursor M |
| 2853 | AMQ48981.1 protein pr |
| 2854 | AMQ48981.1 Flavivirus polyprotein |
| | propeptide |
| 2855 | AMQ48981.1 membrane glycoprotein M |
| 2856 | AMQ48981.1 Flavivirus envelope |
| | glycoprotein M |
| 2857 | AMQ48982.1 membrane glycoprotein |
| | precursor M |
| 2858 | AMQ48982.1 protein pr |
| 2859 | AMQ48982.1 Flavivirus polyprotein |
| | propeptide |
| 2860 | AMQ48982.1 membrane glycoprotein M |
| 2861 | AMQ48982.1 Flavivirus envelope |
| | glycoprotein M |
| 2862 | AMQ34003.1 protein pr |
| 2863 | AMQ34003.1 Flavivirus polyprotein |
| | propeptide |
| 2864 | AMQ34003.1 membrane glycoprotein M |
| 2865 | AMQ34003.1 Flavivirus envelope |
| | glycoprotein M |
| 2866 | AMQ34004.1 protein pr |
| 2867 | AMQ34004.1 Flavivirus polyprotein |
| | propeptide |
| 2868 | AMQ34004.1 membrane glycoprotein M |
| 2869 | AMQ34004.1 Flavivirus envelope |
| | glycoprotein M |
| 2870 | AMN14619.1 Flavivirus polyprotein |
| | propeptide |
| 2871 | AMN14619.1 Flavivirus envelope |
| | glycoprotein M |
| 2872 | AMN14620.1 Flavivirus polyprotein |
| | propeptide |
| 2873 | AMM39806.1 Flavivirus polyprotein |
| | propeptide |
| 2874 | AMM39806.1 Flavivirus envelope |
| | glycoprotein M |
| 2875 | AMK79469.1 Flavivirus polyprotein |
| | propeptide |
| 2876 | AMK79469.1 Flavivirus envelope |
| | glycoprotein M |
| 2877 | AMD16557.1 Flavivirus polyprotein |
| | propeptide |
| 2878 | AMD16557.1 Flavivirus envelope |
| | glycoprotein M |
| 2879 | AMH87239.1 Flavivirus polyprotein |
| | propeptide |
| 2880 | AMB18850.1 Flavivirus polyprotein |
| | propeptide |
| 2881 | AMB18850.1 Flavivirus envelope |
| | glycoprotein M |
| 2882 | AMD61710.1 Flavivirus polyprotein |
| | propeptide |
| 2883 | AMD61710.1 Flavivirus envelope |
| | glycoprotein M |
| 2884 | AMD61711.1 Flavivirus polyprotein |
| | propeptide |
| 2885 | AMD61711.1 Flavivirus envelope |
| | glycoprotein M |
| 2886 | AMB37295.1 Flavivirus polyprotein |
| | propeptide |
| 2887 | AMB37295.1 Flavivirus envelope |
| | glycoprotein M |
| 2888 | AMC13911.1 Flavivirus polyprotein |
| | propeptide |
| 2889 | AMC13911.1 Flavivirus envelope |
| | glycoprotein M |
| 2890 | ALU33341.1 Flavivirus polyprotein |
| | propeptide |
| 2891 | ALU33341.1 Flavivirus envelope |
| | glycoprotein M |
| 2892 | BAP47441.1 Flavivirus polyprotein |
| | propeptide |
| 2893 | BAP47441.1 Flavivirus envelope |
| | glycoprotein M |
| 2894 | AHZ13508.1 Flavivirus polyprotein |
| | propeptide |
| 2895 | AAV34151.1 Flavivirus polyprotein |
| | propeptide |

TABLE 1G
Non-limiting example Zika 3′ UTR sequences by accession number

| | |
|---|---|
| KU527068.1 Zika virus strain Natal RGN, complete | KU761561.1 Zika virus isolate ZJ02, complete |
| genome | genome |
| NC_035889.1 Zika virus isolate ZIKV/H. | KU761560.1 Zika virus isolate ZJ03, complete |
| sapiens/Brazil/Natal/2015, complete genome | genome |
| KY765321.1 Zika virus strain ZIKV/Homo | MH013290.1 Zika virus isolate BKK07, complete |
| sapiens/NIC/4886_12A1_SP/2016, complete | genome |
| genome | |
| KU509998.3 Zika virus strain Haiti/1225/2014, | MK241416.1 Zika virus isolate |
| complete genome | ZIKV/Homo_sapiens/CPV/2015/CV487, complete |
| | genome |
| KX247646.1 Zika virus isolate Zika virus/Homo | MH882534.1 Zika virus isolate Zika virus/H. sapiens- |
| sapiens/COL/UF-1/2016, complete genome | wt/BRA/2016/ZKV17Gsem_2016-06-08, complete |
| | genome |
| KX702400.1 Zika virus strain Zika virus/Homo | MH882532.1 Zika virus isolate Zika virus/H. sapiens- |
| sapiens/VEN/UF-1/2016, complete genome | wt/BRA/2016/ZKV17Esem_2016-05-24, complete |
| | genome |
| KX893855.1 Zika virus strain Zika virus/Homo | MH882530.1 Zika virus isolate Zika virus/H. sapiens- |
| sapiens/VEN/UF-2/2016, complete genome | wt/BRA/2016/ZKV17Csem_2016-05-10, complete |
| | genome |
| KY415991.1 Zika virus isolate Haiti/0097/2014, | MH882531.1 Zika virus isolate Zika virus/H. sapiens- |
| complete genome | wt/BRA/2016/ZKV17Dsem_2016-05-17, complete |
| | genome |
| KY415990.1 Zika virus isolate Haiti/0036/2014, | MH882540.1 Zika virus isolate Zika virus/H. sapiens- |
| complete genome | wt/BRA/2016/ZKV17Nsem 2016-07-27, complete |
| | genome |
| KY415989.1 Zika virus isolate Haiti/0074/2014, | MT377494.1 Zika virus isolate MU-DMSC-3/2017, |
| complete genome | complete genome |
| KY415988.1 Zika virus isolate Haiti/0054/2014, | MH882533.1 Zika virus isolate Zika virus/H. sapiens- |
| complete genome | wt/BRA/2016/ZKV17Fsem_2016-05-31, complete |
| | genome |
| KY415987.1 Zika virus isolate Haiti/0033/2014, | MZ008356.1 Zika virus isolate |
| complete genome | ZIKV/Homo_sapiens/Cambodia/2019/SZ1901, |
| | complete genome |
| KY415986.1 Zika virus isolate Haiti/0029/2014, | MH882539.1 Zika virus isolate Zika virus/H. sapiens- |
| complete genome | wt/BRA/2016/ZKV17Msem_2016-07-19, complete |
| | genome |
| MF384325.1 Zika virus isolate | KU681081.3 Zika virus isolate Zika virus/H. sapiens- |
| mosquito/Haiti/1682/2016, complete genome | tc/THA/2014/SV0127- 14, complete genome |
| MF783073.1 Zika virus strain Zika | KU955593.1 Zika virus isolate Zika virus/H. sapiens- |
| virus/mosquito/Haiti/1919/2016, complete genome | tc/KHM/2010/FSS13025, complete genome |
| MF783072.1 Zika virus strain Zika | KX694532.2 Zika virus strain ZIKV/Homo |
| virus/mosquito/Haiti/1855/2016, complete genome | sapiens/THA/PLCal_ZV/2013, complete genome |
| MN577544.1 Zika virus isolate Haiti/0148/2016, | KY272987.1 Zika virus isolate SI-BKK01, complete |
| complete genome | genome |
| MN577543.1 Zika virus isolate Haiti/1327/2014, | MH158236.1 Zika virus isolate FSS13025, complete |
| complete genome | genome |
| MN566108.1 Zika virus isolate Haiti/0866/2016, | MH675629.1 Zika virus isolate QTX-02, complete |
| complete genome | genome |
| MN566107.1 Zika virus isolate Haiti/0165/2016, | MW015936.1 Zika virus isolate Zika |
| complete genome | virus/H. sapiens-tc/THA/2006/CVD_06-020, |
| | complete genome |
| MN566106.1 Zika virus isolate Haiti/0395/2016, | KX051560.1 Zika virus isolate SK364/13AS, |
| complete genome | complete genome |
| MN566105.1 Zika virus isolate Haiti/0375/2016, | MT377502.1 Zika virus isolate NS-274/2006, |
| complete genome | complete genome |
| MN566104.1 Zika virus isolate Haiti/0011/2016, | MT377492.1 Zika virus isolate MU-DMSC-1/2016, |
| complete genome | complete genome |
| OL450366.1 Zika virus isolate | MT377491.1 Zika virus isolate MU-DMSC-1/2017, |
| ZIKV/Haiti/1332/2015, complete genome | complete genome |
| OL450365.1 Zika virus isolate | MG770187.1 Zika virus isolate SV0127-14, |
| ZIKV/Haiti/0478/2015, complete genome | complete genome |
| OL450364.1 Zika virus isolate | MT377495.1 Zika virus isolate MU-DMSC-4/2017, |
| ZIKV/Haiti/1238/2015, complete genome | complete genome |
| KX051563.1 Zika virus isolate Haiti/1/2016, | MT377503.1 Zika virus isolate 15-1144, complete |
| complete genome | genome |
| KU926310.2 Zika virus isolate Rio-S1, complete | MG770188.1 Zika virus isolate SV0127-14, |
| genome | complete genome |
| KU926309.2 Zika virus isolate Rio-U1, complete | KX051562.1 Zika virus isolate SV0010/15, complete |
| genome | genome |
| KX087102.2 Zika virus strain ZIKV/Homo | LC369584.1 Zika virus ZIKV/Hu/Thai/KngSG/17- |
| sapiens/COL/FLR/2015, complete genome | D501 genomic RNA, nearly complete genome |
| KX156776.2 Zika virus strain ZIKV/Homo | MT377500.1 Zika virus isolate MU-DMSC-6/2016, |
| sapiens/PAN/CDC-259364_V1-V2/2015, complete | complete genome |
| genome | |
| KX156774.2 Zika virus strain ZIKV/Homo | MT377504.1 Zika virus isolate NS-110/2006, |
| sapiens/PAN/CDC-259359_V1-V3/2015, complete | complete genome |
| genome | |
| KX197192.1 Zika virus isolate | MT377493.1 Zika virus isolate MU-DMSC-2/2017, |
| ZIKV/H. sapiens/Brazil/PE243/2015, complete | complete genome |
| genome | |
| KX280026.1 Zika virus isolate Paraiba_01, | MN101548.1 Zika virus isolate Salvador, complete |
| complete genome | genome |
| KX377337.1 Zika virus strain PRVABC-59, | MT377501.1 Zika virus isolate MU-DMSC-5/2017, |
| complete genome | complete genome |
| KX446951.2 Zika virus strain | KX051561.1 Zika virus isolate SK403/13AS, |
| ZIKV/Aedes. sp/MEX/MEX_I-7/2016, complete | complete genome |
| genome | |
| KX446950.2 Zika virus strain | MH119185.1 Zika virus isolate DMSc05684-16, |
| ZIKV/Aedes. sp/MEX/MEX_2-81/2016, complete | complete genome |
| genome | |
| KY328289.1 Zika virus isolate HN16, complete | LC219720.1 Zika virus genomic RNA, complete |
| genome | genome, strain: ZIKV/Hu/NIID123/2016 |
| KY348640.1 Zika virus strain SL1602, complete | KX377336.1 Zika virus strain P6-740, complete |
| genome | genome |
| KY989971.1 Zika virus isolate FLA, complete | KX694533.2 Zika virus strain ZIKV/Aedes |
| genome | aegypti/MYS/P6-740/1966, complete genome |
| MH158237.1 Zika virus isolate PRVABC59, | MH675626.1 Zika virus isolate CTS-183-16p, |
| complete genome | complete genome |
| MF073358.1 Zika virus isolate 15261, complete | MH675621.1 Zika virus isolate CTS-47-16p, |
| genome | complete genome |
| MF073357.1 Zika virus isolate 16288, complete | MT377498.1 Zika virus isolate MU-DMSC-4/2016, |
| genome | complete genome |
| MH544701.2 Zika virus isolate | MN611472.1 Zika virus isolate 2019YNZIKV02, |
| 459148_Meta_Colombia_2016, complete genome | complete genome |
| MH675630.1 Zika virus isolate QTX-04, complete | KX601167.1 Zika virus strain ZIKV/Aedes |
| genome | sp./MYS/P6-740/1966, complete genome |
| MH675628.1 Zika virus isolate CTS-223-16p, | MT377499.1 Zika virus isolate MU-DMSC-5/2016, |
| complete genome | complete genome |
| MH675625.1 Zika virus isolate CTS-178-16p, | MT377497.1 Zika virus isolate MU-DMSC-3/2016, |
| complete genome | complete genome |
| MH675624.1 Zika virus isolate CTS-61-16p, | MT377496.1 Zika virus isolate MU-DMSC-2/2016, |
| complete genome | complete genome |
| MN577550.1 Zika virus isolate ZIKV- | MH061852.1 Zika virus strain ZIKV/Macaca |
| Nicaragua/2016-UCB7420, complete genome | mulatta/UGA/MR-766-VEROE6-AC11-P5_08/1947, |
| | complete genome |
| MT507050.1 Zika virus isolate S-542/Yucatan/2017 | MH061907.1 Zika virus strain ZIKV/Macaca |
| clone 6A, complete genome | mulatta/UGA/MR-766-VEROE6-GA3-P10_06/1947, |
| | complete genome |
| MT507049.1 Zika virus isolate S-542/Yucatan/2017 | MH061902.1 Zika virus strain ZIKV/Macaca |
| clone 6V, complete genome | mulatta/UGA/MR-766-VEROE6-FA12-P7_08/1947, |
| | complete genome |
| MT507048.1 Zika virus isolate S-542/Yucatan/2017 | MH061866.1 Zika virus strain ZIKV/Macaca |
| clone 6C, complete genome | mulatta/UGA/MR-766-VEROE6-GD12- |
| | P10_07/1947, complete genome |
| MT507047.1 Zika virus isolate S- | MH061914.1 Zika virus strain ZIKV/Macaca |
| 542/Yucatan/2017, complete genome | mulatta/UGA/MR-766-VEROE6-GA3-P10_02/1947, |
| | complete genome |
| OK571913.1 Zika virus isolate Homo | MH061915.1 Zika virus strain ZIKV/Macaca |
| sapiens/Haiti/0728/2014, complete genome | mulatta/UGA/MR-766-VEROE6-P12_02/1947, |
| | complete genome |
| KX087101.3 Zika virus strain ZIKV/Homo | MH061904.1 Zika virus strain ZIKV/Macaca |
| sapiens/PRI/PRVABC59/2015, complete genome | mulatta/UGA/MR-766-VEROE6-FC3-P8_02/1947, |
| | complete genome |
| KX156775.2 Zika virus strain ZIKV/Homo | MH061900.1 Zika virus strain ZIKV/Macaca |
| sapiens/PAN/CDC-259249_V1-V3/2015, complete | mulatta/UGA/MR-766-VEROE6-P12_04/1947, |
| genome | complete genome |
| LC190723.1 Zika virus genomic RNA, complete | MH061896.1 Zika virus strain ZIKV/Macaca |
| genome, strain: ZIKV/Hu/Yokohama/1/2016 | mulatta/UGA/MR-766-VEROE6-FC3-P8_04/1947, |
| | complete genome |
| MF352141.1 Zika virus isolate PE243, complete | MH061887.1 Zika virus strain ZIKV/Macaca |
| genome | mulatta/UGA/MR-766-VEROE6-P12_05/1947, |
| | complete genome |
| MH900227.1 Zika virus isolate 31N, complete | MH061870.1 Zika virus strain ZIKV/Macaca |
| genome | mulatta/UGA/MR-766-VEROE6-AC11-P5_05/1947, |
| | complete genome |
| KU870645.1 Zika virus isolate FB-GWUH-2016, | MH061857.1 Zika virus strain ZIKV/Macaca |
| complete genome | mulatta/UGA/MR-766-VEROE6-AC11-P5_07/1947, |
| | complete genome |
| KX827268.1 Zika virus strain ZIKV/Homo | MH061853.1 Zika virus strain ZIKV/Macaca |
| sapiens/USA/UT-1/2016, complete genome | mulatta/UGA/MR-766-VEROE6-GD12- |
| | P10_04/1947, complete genome |
| MN100039.1 Zika virus isolate ZIKV/Homo | MH061880.1 Zika virus strain ZIKV/Macaca |
| sapiens/PAN/CDC-259249_V1-V3/2015, complete | mulatta/UGA/MR-766-VEROE6-P12_08/1947, |
| genome | complete genome |
| MH882544.1 Zika virus isolate Zika | MH061913.1 Zika virus strain ZIKV/Macaca |
| virus/H. sapiens-wt/BRA/2016/ZKV19Bsem 2016- | mulatta/UGA/MR-766-VEROE6-GA3-P10_05/1947, |
| 05-12, complete genome | complete genome |
| MN124090.1 Zika virus isolate ZIKV/Homo | MH061912.1 Zika virus strain ZIKV/Macaca |
| sapiens/PAN/CDC-259249_V1-V3/2015, complete | mulatta/UGA/MR-766-VEROE6-GA3-P10_03/1947, |
| genome | complete genome |
| MN124091.1 Zika virus isolate ZIKV/Homo | MH061910.1 Zika virus strain ZIKV/Macaca |
| sapiens/PAN/CDC-259249_V1-V3/2015, complete | mulatta/UGA/MR-766-VEROE6-FA12-P7_04/1947, |
| genome | complete genome |
| MH882547.1 Zika virus isolate Zika | MH061909.1 Zika virus strain ZIKV/Macaca |
| virus/H. sapiens-wt/BRA/2016/ZKV19Esem_2016- | mulatta/UGA/MR-766-VEROE6-AC11-P5_04/1947, |
| 06-02, complete genome | complete genome |
| KX856011.1 Zika virus strain ZIKV/Aedes | MH061908.1 Zika virus strain ZIKV/Macaca |
| sp./MEX_I-44/2016, complete genome | mulatta/UGA/MR-766-VEROE6-P12_06/1947, |
| | complete genome |
| KY120348.1 Zika virus strain MEX_CIENI551P4, | MH061906.1 Zika virus strain ZIKV/Macaca |
| complete genome | mulatta/UGA/MR-766-VEROE6-FC11-P7_01/1947, |
| | complete genome |
| KY765327.1 Zika virus strain ZIKV/Homo | MH061905.1 Zika virus strain ZIKV/Macaca |
| sapiens/NIC/5005_13A1_SP/2016, complete | mulatta/UGA/MR-766-VEROE6-AA1-P8_01/1947, |
| genome | complete genome |
| KY765325.1 Zika virus strain ZIKV/Homo | MH061903.1 Zika virus strain ZIKV/Macaca |
| sapiens/NIC/5005_13A1/2016, complete genome | mulatta/UGA/MR-766-VEROE6-AA1-P8_04/1947, |
| | complete genome |
| KY765323.1 Zika virus strain ZIKV/Homo | MH061901.1 Zika virus strain ZIKV/Macaca |
| sapiens/NIC/6188_13A1/2016, complete genome | mulatta/UGA/MR-766-VEROE6-GA3-P10_07/1947, |
| | complete genome |
| KY765320.1 Zika virus strain ZIKV/Homo | MH061899.1 Zika virus strain ZIKV/Macaca |
| sapiens/NIC/6406_13A1_SP/2016, complete | mulatta/UGA/MR-766-VEROE6-AA1-P8_08/1947, |
| genome | complete genome |
| KY765317.1 Zika virus strain ZIKV/Homo | MH061898.1 Zika virus strain ZIKV/Macaca |
| sapiens/NIC/7252_12A1/2016, complete genome | mulatta/UGA/MR-766-VEROE6-FC11-P7_02/1947, |
| | complete genome |
| KX766029.1 Zika virus isolate R116265, complete | MH061897.1 Zika virus strain ZIKV/Macaca |
| genome | mulatta/UGA/MR-766-VEROE6-FC3-P8_01/1947, |
| | complete genome |
| KY631494.1 Zika virus isolate MEX_ENCB165P4, | MH061895.1 Zika virus strain ZIKV/Macaca |
| complete genome | mulatta/UGA/MR-766-VEROE6-P12_01/1947, |
| | complete genome |
| KY765318.1 Zika virus strain ZIKV/Homo | MH061894.1 Zika virus strain ZIKV/Macaca |
| sapiens/NIC/4886_12A1/2016, complete genome | mulatta/UGA/MR-766-VEROE6-FC11-P7_03/1947, |
| | complete genome |
| KX673530.1 Zika virus isolate | MH061893.1 Zika virus strain ZIKV/Macaca |
| PHE semen_Guadeloupe, complete genome | mulatta/UGA/MR-766-VEROE6-GD12- |
| | P10_03/1947, complete genome |
| KY765324.1 Zika virus strain ZIKV/Homo | MH061892.1 Zika virus strain ZIKV/Macaca |
| sapiens/NIC/8610_13A1/2016, complete genome | mulatta/UGA/MR-766-VEROE6-P12_03/1947, |
| | complete genome |
| KY631493.1 Zika virus isolate MEX_ENCB165, | MH061891.1 Zika virus strain ZIKV/Macaca |
| complete genome | mulatta/UGA/MR-766-VEROE6-FA12-P7_06/1947, |
| | complete genome |
| KX601168.1 Zika virus strain ZIKV/Homo | MH061890.1 Zika virus strain ZIKV/Macaca |
| Sapiens/PRI/PRVABC59/2015, complete genome | mulatta/UGA/MR-766-VEROE6-FC3-P8_08/1947, |
| | complete genome |
| KX766028.1 Zika virus isolate R114916, complete | MH061889.1 Zika virus strain ZIKV/Macaca |
| genome | mulatta/UGA/MR-766-VEROE6-FC3-P8_07/1947, |
| | complete genome |
| MH513600.1 Zika virus isolate BR/Sinop/H366 | MH061888.1 Zika virus strain ZIKV/Macaca |
| 2P/2015, complete genome | mulatta/UGA/MR-766-VEROE6-AC11-P5_02/1947, |
| | complete genome |
| MH513599.1 Zika virus isolate | MH061886.1 Zika virus strain ZIKV/Macaca |
| BR/Sinop/H366/2015, complete genome | mulatta/UGA/MR-766-VEROE6-FA12-P7_05/1947, |
| | complete genome |
| KU321639.1 Zika virus strain ZikaSPH2015, | MH061885.1 Zika virus strain ZIKV/Macaca |
| complete genome | mulatta/UGA/MR-766-VEROE6-FC3-P8_06/1947, |
| | complete genome |
| KU744693.1 Zika virus isolate VE_Ganxian, | MH061884.1 Zika virus strain ZIKV/Macaca |
| complete genome | mulatta/UGA/MR-766-VEROE6-GA3-P10_01/1947, |
| | complete genome |
| KY648934.1 Zika virus strain ZIKV/Aedes | MH061883.1 Zika virus strain ZIKV/Macaca |
| aegypti/MEX/MEX_I-44/2016, complete genome | mulatta/UGA/MR-766-VEROE6-FC11-P7_08/1947, |
| | complete genome |
| KY765322.1 Zika virus strain ZIKV/Homo | MH061882.1 Zika virus strain ZIKV/Macaca |
| sapiens/NIC/7252_12A1_SP/2016, complete | mulatta/UGA/MR-766-VEROE6-GD12- |
| genome | P10_08/1947, complete genome |
| MH157213.1 Zika virus strain ZIKV/Homo | MH061881.1 Zika virus strain ZIKV/Macaca |
| sapiens/MEX/41-001-F_V3_O/2016, complete | mulatta/UGA/MR-766-VEROE6-GD12- |
| genome | P10_02/1947, complete genome |
| MH157208.1 Zika virus strain ZIKV/Homo | MH061879.1 Zika virus strain ZIKV/Macaca |
| sapiens/MEX/41-022-F_V0_O/2016, complete | mulatta/UGA/MR-766-VEROE6-GD12- |
| genome | P10_05/1947, complete genome |
| MH157202.1 Zika virus strain ZIKV/Homo | MH061878.1 Zika virus strain ZIKV/Macaca |
| sapiens/MEX/41-017-F_V3_O/2016, complete | mulatta/UGA/MR-766-VEROE6-FC3-P8_05/1947, |
| genome | complete genome |
| MH916806.1 Zika virus strain ZIKV/Homo | MH061877.1 Zika virus strain ZIKV/Macaca |
| sapiens/PRI/PRVABC59_8/2015, complete genome | mulatta/UGA/MR-766-VEROE6-FC11-P7_07/1947, |
| | complete genome |
| MH916803.1 Zika virus strain ZIKV/Homo | MH061876.1 Zika virus strain ZIKV/Macaca |
| sapiens/PRI/PRVABC59_1/2015, complete genome | mulatta/UGA/MR-766-VEROE6-P12_07/1947, |
| | complete genome |
| MH916802.1 Zika virus strain ZIKV/Homo | MH061874.1 Zika virus strain ZIKV/Macaca |
| sapiens/PRI/PRVABC59_17/2015, complete | mulatta/UGA/MR-766-VEROE6-AA1-P8_03/1947, |
| genome | complete genome |
| MT483911.1 Zika virus isolate 800/16, complete | MH061873.1 Zika virus strain ZIKV/Macaca |
| genome | mulatta/UGA/MR-766-VEROE6-FA12-P7_01/1947, |
| | complete genome |
| KU922960.1 Zika virus isolate | MH061872.1 Zika virus strain ZIKV/Macaca |
| MEX/InDRE/Sm/2016, complete genome | mulatta/UGA/MR-766-VEROE6-AA1-P8_05/1947, |
| | complete genome |
| KU922923.1 Zika virus isolate | MH061871.1 Zika virus strain ZIKV/Macaca |
| MEX/InDRE/Lm/2016, complete genome | mulatta/UGA/MR-766-VEROE6-FC11-P7_04/1947, |
| | complete genome |
| KY765326.1 Zika virus strain ZIKV/Homo | MH061869.1 Zika virus strain ZIKV/Macaca |
| sapiens/NIC/6188_13A1_SP/2016, complete | mulatta/UGA/MR-766-VEROE6-AA1-P8_06/1947, |
| genome | complete genome |
| KU501215.1 Zika virus strain PRVABC59, | MH061868.1 Zika virus strain ZIKV/Macaca |
| complete genome | mulatta/UGA/MR-766-VEROE6-AA1-P8_07/1947, |
| | complete genome |
| MK713748.1 Zika virus isolate PRVABC59, | MH061867.1 Zika virus strain ZIKV/Macaca |
| complete genome | mulatta/UGA/MR-766-VEROE6-GA3-P10_04/1947, |
| | complete genome |
| MH513598.1 Zika virus isolate | MH061865.1 Zika virus strain ZIKV/Macaca |
| BR/Sinop/H355/2015, complete genome | mulatta/UGA/MR-766-VEROE6-AA1-P8_02/1947, |
| | complete genome |
| KY120349.2 Zika virus strain MEX_CIENI551, | MH061864.1 Zika virus strain ZIKV/Macaca |
| complete genome | mulatta/UGA/MR-766-VEROE6-FA12-P7_07/1947, |
| | complete genome |
| MK241417.1 Zika virus isolate | MH061863.1 Zika virus strain ZIKV/Macaca |
| ZIKV/Homo_sapiens/CPV/2016/CV907u, complete | mulatta/UGA/MR-766-VEROE6-FA12-P7_02/1947, |
| genome | complete genome |
| MK241415.1 Zika virus isolate | MH061862.1 Zika virus strain ZIKV/Macaca |
| ZIKV/Homo_sapiens/CPV/2015/CV448c, complete | mulatta/UGA/MR-766-VEROE6-GD12- |
| genome | P10_01/1947, complete genome |
| KX269878.1 Zika virus isolate Haiti/2016/PD, | MH061861.1 Zika virus strain ZIKV/Macaca |
| complete genome | mulatta/UGA/MR-766-VEROE6-GA3-P10_08/1947, |
| | complete genome |
| KU853013.1 Zika virus isolate Dominican | MH061860.1 Zika virus strain ZIKV/Macaca |
| Republic/2016/PD2, complete genome | mulatta/UGA/MR-766-VEROE6-FC11-P7_06/1947, |
| | complete genome |
| KU853012.1 Zika virus isolate Dominican | MH061859.1 Zika virus strain ZIKV/Macaca |
| Republic/2016/PD1, complete genome | mulatta/UGA/MR-766-VEROE6-AC11-P5_01/1947, |
| | complete genome |
| KU820899.2 Zika virus isolate ZJ03, complete | MH061858.1 Zika virus strain ZIKV/Macaca |
| genome | mulatta/UGA/MR-766-VEROE6-FC11-P7_05/1947, |
| | complete genome |
| KX117076.1 Zika virus isolate Zhejiang04, | MH061856.1 Zika virus strain ZIKV/Macaca |
| complete genome | mulatta/UGA/MR-766-VEROE6-FC3-P8_03/1947, |
| | complete genome |
| KJ776791.2 Zika virus strain H/PF/2013, complete | MH061855.1 Zika virus strain ZIKV/Macaca |
| genome | mulatta/UGA/MR-766-VEROE6-FA12-P7_03/1947, |
| | complete genome |
| KU681082.3 Zika virus isolate Zika | MH061854.1 Zika virus strain ZIKV/Macaca |
| virus/H. sapiens-tc/PHL/2012/CPC-0740, complete | mulatta/UGA/MR-766-VEROE6-AC11-P5_06/1947, |
| genome | complete genome |
| KX198135.2 Zika virus strain ZIKV/Homo | LC002520.1 Zika virus genomic RNA, complete |
| sapiens/PAN/BEI-259634_V4/2016, complete | genome, strain: MR766-NIID |
| genome | |
| KX262887.1 Zika virus isolate 103451, complete | KU955594.1 Zika virus isolate Zika virus/M. mulatta- |
| genome | tc/UGA/1947/MR-766, complete genome |
| KX694534.2 Zika virus strain ZIKV/Homo | KX377335.1 Zika virus strain MR-766, complete |
| sapiens/HND/R103451/2015, complete genome | genome |
| KX806557.3 Zika virus isolate TS17-2016, | MK105975.1 Zika virus strain MR766, complete |
| complete genome | genome |
| KY272991.1 Zika virus isolate RIO-BM1, complete | MW680970.1 Zika virus isolate rGZ02p/2018, |
| genome | complete genome |
| MG827392.1 Zika virus strain PF13/251013-18, | MW680969.1 Zika virus isolate rGZ02a/2018, |
| complete genome | complete genome |
| MF073359.1 Zika virus isolate 15098, complete | MH130109.1 Zika virus strain ZIKV/Macaca |
| genome | mulatta/UGA/MR-766-VEROE6-AC4-P12_06/1947, |
| | complete genome |
| MH675627.1 Zika virus isolate CTS-193-16p, | MH130103.1 Zika virus strain ZIKV/Macaca |
| complete genome | mulatta/UGA/MR-766-VEROE6-AC10- |
| | P12_06/1947, complete genome |
| MH675623.1 Zika virus isolate CTS-56-16p, | MH130100.1 Zika virus strain ZIKV/Macaca |
| complete genome | mulatta/UGA/MR-766-VEROE6-AC4-P12_01/1947, |
| | complete genome |
| MH675622.1 Zika virus isolate CTS-50-16p, | MH130096.1 Zika virus strain ZIKV/Macaca |
| complete genome | mulatta/UGA/MR-766-VEROE6-AC10- |
| | P12_05/1947, complete genome |
| MH675620.1 Zika virus isolate CTS-36-16p, | MH130105.1 Zika virus strain ZIKV/Macaca |
| complete genome | mulatta/UGA/MR-766-VEROE6-AC10- |
| | P12_04/1947, complete genome |
| MK566202.1 Zika virus isolate 15098, complete | MH130108.1 Zika virus strain ZIKV/Macaca |
| genome | mulatta/UGA/MR-766-VEROE6-AC4-P12_04/1947, |
| | complete genome |
| KX879604.1 Zika virus strain | MH130099.1 Zika virus strain ZIKV/Macaca |
| ZIKV/EC/Esmeraldas/089/2016, complete genome | mulatta/UGA/MR-766-VEROE6-AC4-P12_02/1947, |
| | complete genome |
| KX197205.1 Zika virus isolate 9, complete genome | MH130097.1 Zika virus strain ZIKV/Macaca |
| | mulatta/UGA/MR-766-VEROE6-AC10- |
| | P12_03/1947, complete genome |
| KX879603.1 Zika virus strain | MH130107.1 Zika virus strain ZIKV/Macaca |
| ZIKV/EC/Esmeraldas/062/2016, complete genome | mulatta/UGA/MR-766-VEROE6-AC4-P12_03/1947, |
| | complete genome |
| LC191864.1 Zika virus genomic RNA, complete | MH130106.1 Zika virus strain ZIKV/Macaca |
| genome, strain: ZIKV/Hu/Chiba/S36/2016 | mulatta/UGA/MR-766-VEROE6-AC10- |
| | P12_08/1947, complete genome |
| MH882546.1 Zika virus isolate Zika | MH130104.1 Zika virus strain ZIKV/Macaca |
| virus/H. sapiens-wt/BRA/2016/ZKV19Dsem_2016- | mulatta/UGA/MR-766-VEROE6-AC10- |
| 05-24, complete genome | P12_07/1947, complete genome |
| MH882545.1 Zika virus isolate Zika | MH130102.1 Zika virus strain ZIKV/Macaca |
| virus/H. sapiens-wt/BRA/2016/ZKV19Csem_2016- | mulatta/UGA/MR-766-VEROE6-AC10- |
| 05-19, complete genome | P12_02/1947, complete genome |
| MH882543.1 Zika virus isolate Zika | MH130101.1 Zika virus strain ZIKV/Macaca |
| virus/H. sapiens-wt/BRA/2016/ZKV19Auri_2016- | mulatta/UGA/MR-766-VEROE6-AC4-P12_08/1947, |
| 05-05, complete genome | complete genome |
| MH882548.1 Zika virus isolate Zika | MH130098.1 Zika virus strain ZIKV/Macaca |
| virus/H. sapiens-wt/BRA/2016/ZKV19Fsem_2016- | mulatta/UGA/MR-766-VEROE6-AC10- |
| 06-09, complete genome | P12_01/1947, complete genome |
| KX369547.1 Zika virus strain PF13/251013-18, | MH130095.1 Zika virus strain ZIKV/Macaca |
| complete genome | mulatta/UGA/MR-766-VEROE6-AC4-P12_07/1947, |
| | complete genome |
| MF794971.1 Zika virus strain | MH130094.1 Zika virus strain ZIKV/Macaca |
| ZIKV/EC/Esmeraldas/121/2016, complete genome | mulatta/UGA/MR-766-VEROE6-AC4-P12_05/1947, |
| | complete genome |
| KX811222.1 Zika virus isolate Brazil_2015_MG, | OL414716.1 Zika virus isolate 15555, complete |
| complete genome | genome |
| MH882542.1 Zika virus isolate Zika | MH061911.1 Zika virus strain ZIKV/Macaca |
| virus/H. sapiens-wt/BRA/2016/ZKV17uri_2016-04- | mulatta/UGA/MR-766-VEROE6-GD12- |
| 19, complete genome | P10_06/1947, complete genome |
| MH882541.1 Zika virus isolate Zika | MH061875.1 Zika virus strain ZIKV/Macaca |
| virus/H. sapiens-wt/BRA/2016/ZKV17sem_2016- | mulatta/UGA/MR-766-VEROE6-AC11-P5_03/1947, |
| 04-19, complete genome | complete genome |
| MH882529.1 Zika virus isolate Zika | KX830960.1 Zika virus culture ATCC: VR-84 isolate |
| virus/H. sapiens-wt/BRA/2016/ZKV17Bsem_2016- | MR776, complete genome |
| 05-03, complete genome | |
| MH882528.1 Zika virus isolate Zika | KY989511.1 Zika virus strain MR 766, complete |
| virus/H. sapiens-wt/BRA/2016/ZKV17Auri_2016- | genome |
| 04-26, complete genome | |
| MH882527.1 Zika virus isolate Zika | OK054351.1 Zika virus isolate 211784, complete |
| virus/H. sapiens-wt/BRA/2016/ZKV17Asem_2016- | genome |
| 04-26, complete genome | |
| KU707826.1 Zika virus isolate SSABR1, complete | KY288905.1 Zika virus strain MP1751, complete |
| genome | genome |
| KX253996.1 Zika virus isolate ZKC2/2016, | AY632535.2 Zika virus strain MR 766, complete |
| complete genome | genome |
| KY553111.1 Zika virus isolate AFMC-U, complete | NC_012532.1 Zika virus, complete genome |
| genome | |
| MG674719.1 Zika virus isolate ZKC2P6, complete | KX601169.1 Zika virus strain ZIKV/Macaca |
| genome | mulatta/UGA/MR-766/1947, complete genome |
| MG674718.1 Zika virus isolate ZKC2P4, complete | MW143022.1 Zika virus strain MR766, complete |
| genome | genome |
| MG807647.1 Zika virus isolate SI-BKK06, | KU955595.1 Zika virus isolate Zika virus/A. taylori- |
| complete genome | tc/SEN/1984/41671-DAK, complete genome |
| MG807646.1 Zika virus isolate SI-BKK05, | KU955592.1 Zika virus isolate Zika virus/A. taylori- |
| complete genome | tc/SEN/1984/41662-DAK, complete genome |
| MG548661.1 Zika virus isolate BKK04, complete | KX198134.2 Zika virus strain ZIKV/Aedes |
| genome | africanus/SEN/DAK-AR-41524 A1C1-V2/1984, |
| | complete genome |
| MG548660.1 Zika virus isolate BKK03, complete | KX601166.2 Zika virus strain ZIKV/Aedes |
| genome | africanus/SEN/DakAr41524/1984, complete genome |
| MF996804.1 Zika virus isolate SI-BKK02, | MN025403.1 Zika virus isolate 15555, complete |
| complete genome | genome |
| MH675619.1 Zika virus isolate CTS-30-16p, | KU955591.1 Zika virus isolate Zika |
| complete genome | virus/A. africanus-tc/SEN/1984/41525-DAK. |
| | complete genome |
| MT636065.1 Zika virus isolate ARCB116141, | MG758786.1 Zika virus isolate 41525, complete |
| complete genome | genome |
| KU497555.1 Zika virus isolate Brazil-ZKV2015, | MG758785.1 Zika virus isolate 41525, complete |
| complete genome | genome |
| MH882538.1 Zika virus isolate Zika | MT505350.1 Mutant Zika virus isolate DK23, |
| virus/H. sapiens-wt/BRA/2016/ZKV17Lsem_2016- | complete genome |
| 07-12, complete genome | |
| MH882536.1 Zika virus isolate Zika | MT505349.1 Mutant Zika virus isolate DK, complete |
| virus/H. sapiens-wt/BRA/2016/ZKV17Jsem_2016- | genome |
| 06-28, complete genome | |
| KX266255.1 Zika virus isolate ZIKV_SMGC-1, | MF510857.1 Zika virus isolate Zika virus/Aedes |
| complete genome | taylori/Senegal/1984/DakAr41667, complete genome |
| MH882535.1 Zika virus isolate Zika | KY766069.1 Zika virus isolate Pf13/251013-18, |
| virus/H. sapiens-wt/BRA/2016/ZKV17Hsem_2016- | complete genome |
| 06-16, complete genome | |

TABLE 1H
Non-limiting example Zika 5′ UTR sequences by accession number

KJ776791.2 Zika virus strain H/PF/2013, complete genome
KU681082.3 Zika virus isolate Zika virus/H. sapiens-tc/PHL/2012/CPC-0740, complete genome
KU681081.3 Zika virus isolate Zika virus/H. sapiens-tc/THA/2014/SV0127- 14, complete genome
KU527068.1 Zika virus strain Natal RGN, complete genome
KU853013.1 Zika virus isolate Dominican Republic/2016/PD2, complete genome
KU853012.1 Zika virus isolate Dominican Republic/2016/PD1, complete genome
KU926310.2 Zika virus isolate Rio-S1, complete genome
KU926309.2 Zika virus isolate Rio-U1, complete genome
KU955593.1 Zika virus isolate Zika virus/H. sapiens-tc/KHM/2010/FSS13025, complete genome
KX197192.1 Zika virus isolate ZIKV/H. sapiens/Brazil/PE243/2015, complete genome
KX253996.1 Zika virus isolate ZKC2/2016, complete genome
KX280026.1 Zika virus isolate Paraiba_01, complete genome
KX377337.1 Zika virus strain PRVABC-59, complete genome
KX377336.1 Zika virus strain P6-740, complete genome
KX806557.3 Zika virus isolate TS17-2016, complete genome
KX879604.1 Zika virus strain ZIKV/EC/Esmeraldas/089/2016, complete genome
KX269878.1 Zika virus isolate Haiti/2016/PD, complete genome
KY272987.1 Zika virus isolate SI-BKK01, complete genome
KY272991.1 Zika virus isolate RIO-BMI, complete genome
KY348640.1 Zika virus strain SL1602, complete genome
KY631494.1 Zika virus isolate MEX_ENCB165P4, complete genome
KY415991.1 Zika virus isolate Haiti/0097/2014, complete genome
KY415990.1 Zika virus isolate Haiti/0036/2014, complete genome
KY415989.1 Zika virus isolate Haiti/0074/2014, complete genome
KY415988.1 Zika virus isolate Haiti/0054/2014, complete genome
KY415987.1 Zika virus isolate Haiti/0033/2014, complete genome
KY415986.1 Zika virus isolate Haiti/0029/2014, complete genome
MF384325.1 Zika virus isolate mosquito/Haiti/1682/2016, complete genome
KY553111.1 Zika virus isolate AFMC-U, complete genome
MG674719.1 Zika virus isolate ZKC2P6, complete genome
MG674718.1 Zika virus isolate ZKC2P4, complete genome
MG827392.1 Zika virus strain PF13/251013-18, complete genome
MH158237.1 Zika virus isolate PRVABC59, complete genome
MH158236.1 Zika virus isolate FSS13025, complete genome
MF073359.1 Zika virus isolate 15098, complete genome
MF073358.1 Zika virus isolate 15261, complete genome
MF073357.1 Zika virus isolate 16288, complete genome
MG807646.1 Zika virus isolate SI-BKK05, complete genome
MG548661.1 Zika virus isolate BKK04, complete genome
MG548660.1 Zika virus isolate BKK03, complete genome
MF996804.1 Zika virus isolate SI-BKK02, complete genome
MH544701.2 Zika virus isolate 459148_Meta_Colombia_2016, complete genome
MF783073.1 Zika virus strain Zika virus/mosquito/Haiti/1919/2016, complete genome
MF783072.1 Zika virus strain Zika virus/mosquito/Haiti/1855/2016, complete genome
MH675630.1 Zika virus isolate QTX-04, complete genome
MH675629.1 Zika virus isolate QTX-02, complete genome
MH675628.1 Zika virus isolate CTS-223-16p, complete genome
MH675627.1 Zika virus isolate CTS-193-16p, complete genome
MH675626.1 Zika virus isolate CTS-183-16p, complete genome
MH675625.1 Zika virus isolate CTS-178-16p, complete genome
MH675624.1 Zika virus isolate CTS-61-16p, complete genome
MH675623.1 Zika virus isolate CTS-56-16p, complete genome
MH675622.1 Zika virus isolate CTS-50-16p, complete genome
MH675621.1 Zika virus isolate CTS-47-16p, complete genome
MH675620.1 Zika virus isolate CTS-36-16p, complete genome
MH675619.1 Zika virus isolate CTS-30-16p, complete genome
MN577550.1 Zika virus isolate ZIKV-Nicaragua/2016-UCB7420, complete genome
MN577544.1 Zika virus isolate Haiti/0148/2016, complete genome
MN577543.1 Zika virus isolate Haiti/1327/2014, complete genome
MN566108.1 Zika virus isolate Haiti/0866/2016, complete genome
MN566107.1 Zika virus isolate Haiti/0165/2016, complete genome
MN566106.1 Zika virus isolate Haiti/0395/2016, complete genome
MN566105.1 Zika virus isolate Haiti/0375/2016, complete genome
MN566104.1 Zika virus isolate Haiti/0011/2016, complete genome
MN611472.1 Zika virus isolate 2019YNZIKV02, complete genome
MK566202.1 Zika virus isolate 15098, complete genome
MT507050.1 Zika virus isolate S-542/Yucatan/2017 clone 6A, complete genome
MT507049.1 Zika virus isolate S-542/Yucatan/2017 clone 6V, complete genome
MT507048.1 Zika virus isolate S-542/Yucatan/2017 clone 6C, complete genome
MT507047.1 Zika virus isolate S-542/Yucatan/2017, complete genome
MT636065.1 Zika virus isolate ARCB116141, complete genome
MW015936.1 Zika virus isolate Zika virus/H. sapiens-tc/THA/2006/CVD_06-020, complete genome
OL450366.1 Zika virus isolate ZIKV/Haiti/1332/2015, complete genome
OL450365.1 Zika virus isolate ZIKV/Haiti/0478/2015, complete genome
OL450364.1 Zika virus isolate ZIKV/Haiti/1238/2015, complete genome
NC 035889.1 Zika virus isolate ZIKV/H. sapiens/Brazil/Natal/2015, complete genome
KU501215.1 Zika virus strain PRVABC59, complete genome
KX262887.1 Zika virus isolate 103451, complete genome
KY328289.1 Zika virus isolate HN16, complete genome
KY989971.1 Zika virus isolate FLA, complete genome
MK713748.1 Zika virus isolate PRVABC59, complete genome
OK571913.1 Zika virus isolate Homo sapiens/Haiti/0728/2014, complete genome
KX051561.1 Zika virus isolate SK403/13AS, complete genome
MH882543.1 Zika virus isolate Zika virus/H. sapiens-wt/BRA/2016/ZKV19Auri_2016-05-05, complete
genome
MH882546.1 Zika virus isolate Zika virus/H. sapiens-wt/BRA/2016/ZKV19Dsem_2016-05-24, complete
genome
MH882545.1 Zika virus isolate Zika virus/H. sapiens-wt/BRA/2016/ZKV19Csem_2016-05-19, complete
genome
KX811222.1 Zika virus isolate Brazil_2015_MG, complete genome
KX051560.1 Zika virus isolate SK364/13AS, complete genome
MF352141.1 Zika virus isolate PE243, complete genome
MH900227.1 Zika virus isolate 31N, complete genome
KX601167.1 Zika virus strain ZIKV/Aedes sp./MYS/P6-740/1966, complete genome
KX266255.1 Zika virus isolate ZIKV_SMGC-1, complete genome
KX197205.1 Zika virus isolate 9, complete genome
MH119185.1 Zika virus isolate DMSc05684-16, complete genome
MH882544.1 Zika virus isolate Zika virus/H. sapiens-wt/BRA/2016/ZKV19Bsem_2016-05-12, complete
genome
KU870645.1 Zika virus isolate FB-GWUH-2016, complete genome
KX446950.2 Zika virus strain ZIKV/Aedes. sp/MEX/MEX_2-81/2016, complete genome
KX827268.1 Zika virus strain ZIKV/Homo sapiens/USA/UT-1/2016, complete genome
MH882541.1 Zika virus isolate Zika virus/H. sapiens-wt/BRA/2016/ZKV17sem_2016-04-19, complete
genome
MH882538.1 Zika virus isolate Zika virus/H. sapiens-wt/BRA/2016/ZKV17Lsem_2016-07-12, complete
genome
MH882533.1 Zika virus isolate Zika virus/H. sapiens-wt/BRA/2016/ZKV17Fsem_2016-05-31, complete
genome
MH882531.1 Zika virus isolate Zika virus/H. sapiens-wt/BRA/2016/ZKV17Dsem_2016-05-17, complete
genome
MH882527.1 Zika virus isolate Zika virus/H. sapiens-wt/BRA/2016/ZKV17Asem_2016-04-26, complete
genome
MN100039.1 Zika virus isolate ZIKV/Homo sapiens/PAN/CDC-259249_V1-V3/2015, complete genome
KU497555.1 Zika virus isolate Brazil-ZKV2015, complete genome
KU761561.1 Zika virus isolate ZJ02, complete genome
KU761560.1 Zika virus isolate ZJ03, complete genome
MG770188.1 Zika virus isolate SV0127-14, complete genome
MG770187.1 Zika virus isolate SV0127-14, complete genome
MH882548.1 Zika virus isolate Zika virus/H. sapiens-wt/BRA/2016/ZKV19Fsem_2016-06-09, complete
genome
MH882547.1 Zika virus isolate Zika virus/H. sapiens-wt/BRA/2016/ZKV19Esem_2016-06-02, complete
genome
MH882542.1 Zika virus isolate Zika virus/H. sapiens-wt/BRA/2016/ZKV17uri_2016-04-19, complete
genome
MH882540.1 Zika virus isolate Zika virus/H. sapiens-wt/BRA/2016/ZKV17Nsem_2016-07-27, complete
genome
MH882539.1 Zika virus isolate Zika virus/H. sapiens-wt/BRA/2016/ZKV17Msem_2016-07-19, complete
genome
MH882536.1 Zika virus isolate Zika virus/H. sapiens-wt/BRA/2016/ZKV17Jsem_2016-06-28, complete
genome
MH882535.1 Zika virus isolate Zika virus/H. sapiens-wt/BRA/2016/ZKV17Hsem_2016-06-16, complete
genome
MH882534.1 Zika virus isolate Zika virus/H. sapiens-wt/BRA/2016/ZKV17Gsem_2016-06-08, complete
genome
MH882532.1 Zika virus isolate Zika virus/H. sapiens-wt/BRA/2016/ZKV17Esem_2016-05-24, complete
genome
MH882530.1 Zika virus isolate Zika virus/H. sapiens-wt/BRA/2016/ZKV17Csem_2016-05-10, complete
genome
MH882529.1 Zika virus isolate Zika virus/H. sapiens-wt/BRA/2016/ZKV17Bsem_2016-05-03, complete
genome
MH882528.1 Zika virus isolate Zika virus/H. sapiens-wt/BRA/2016/ZKV17Auri_2016-04-26, complete
genome
MN124091.1 Zika virus isolate ZIKV/Homo sapiens/PAN/CDC-259249_V1-V3/2015, complete genome
MN124090.1 Zika virus isolate ZIKV/Homo sapiens/PAN/CDC-259249_V1-V3/2015, complete genome
MN101548.1 Zika virus isolate Salvador, complete genome
MT377494.1 Zika virus isolate MU-DMSC-3/2017, complete genome
KX766028.1 Zika virus isolate R114916, complete genome
KX673530.1 Zika virus isolate PHE_semen_Guadeloupe, complete genome
MH013290.1 Zika virus isolate BKK07, complete genome
MT377502.1 Zika virus isolate NS-274/2006, complete genome
KX087102.2 Zika virus strain ZIKV/Homo sapiens/COL/FLR/2015, complete genome
KX446951.2 Zika virus strain ZIKV/Aedes. sp/MEX/MEX_I-7/2016, complete genome
KX856011.1 Zika virus strain ZIKV/Aedes sp./MEX_I-44/2016, complete genome
KY120348.1 Zika virus strain MEX_CIENI551P4, complete genome
KY631493.1 Zika virus isolate MEX_ENCB165, complete genome
KX601168.1 Zika virus strain ZIKV/Homo Sapiens/PRI/PRVABC59/2015, complete genome
LC190723.1 Zika virus genomic RNA, complete genome, strain: ZIKV/Hu/Yokohama/1/2016
KY765327.1 Zika virus strain ZIKV/Homo sapiens/NIC/5005_13A1_SP/2016, complete genome
KY765325.1 Zika virus strain ZIKV/Homo sapiens/NIC/5005_13A1/2016, complete genome
KY765324.1 Zika virus strain ZIKV/Homo sapiens/NIC/8610_13A1/2016, complete genome
KY765320.1 Zika virus strain ZIKV/Homo sapiens/NIC/6406_13A1_SP/2016, complete genome
MH157208.1 Zika virus strain ZIKV/Homo sapiens/MEX/41-022-F_V0_O/2016, complete genome
MH157202.1 Zika virus strain ZIKV/Homo sapiens/MEX/41-017-F_V3_O/2016, complete genome
KY765318.1 Zika virus strain ZIKV/Homo sapiens/NIC/4886_12A1/2016, complete genome
MH916806.1 Zika virus strain ZIKV/Homo sapiens/PRI/PRVABC59_8/2015, complete genome
MH916802.1 Zika virus strain ZIKV/Homo sapiens/PRI/PRVABC59_17/2015, complete genome
LC219720.1 Zika virus genomic RNA, complete genome, strain: ZIKV/Hu/NIID123/2016
KY120349.2 Zika virus strain MEX_CIENI551, complete genome
MH513599.1 Zika virus isolate BR/Sinop/H366/2015, complete genome
MH513598.1 Zika virus isolate BR/Sinop/H355/2015, complete genome
KX087101.3 Zika virus strain ZIKV/Homo sapiens/PRI/PRVABC59/2015, complete genome
MK241417.1 Zika virus isolate ZIKV/Homo_sapiens/CPV/2016/CV907u, complete genome
MK241416.1 Zika virus isolate ZIKV/Homo_sapiens/CPV/2015/CV487, complete genome
MK241415.1 Zika virus isolate ZIKV/Homo_sapiens/CPV/2015/CV448c, complete genome
KX198135.2 Zika virus strain ZIKV/Homo sapiens/PAN/BEI-259634_V4/2016, complete genome
KX694532.2 Zika virus strain ZIKV/Homo sapiens/THA/PLCal_ZV/2013, complete genome
KY648934.1 Zika virus strain ZIKV/Aedes aegypti/MEX/MEX_I-44/2016, complete genome
MH916803.1 Zika virus strain ZIKV/Homo sapiens/PRI/PRVABC59_1/2015, complete genome
KX694534.2 Zika virus strain ZIKV/Homo sapiens/HND/R103451/2015, complete genome
MZ008356.1 Zika virus isolate ZIKV/Homo_sapiens/Cambodia/2019/SZ1901, complete genome
LC369584.1 Zika virus ZIKV/Hu/Thai/KngSG/17-D501 genomic RNA, nearly complete genome
KU922960.1 Zika virus isolate MEX/InDRE/Sm/2016, complete genome
KU922923.1 Zika virus isolate MEX/InDRE/Lm/2016, complete genome
MT377503.1 Zika virus isolate 15-1144, complete genome
MT377500.1 Zika virus isolate MU-DMSC-6/2016, complete genome
MT377499.1 Zika virus isolate MU-DMSC-5/2016, complete genome
MT377493.1 Zika virus isolate MU-DMSC-2/2017, complete genome
MT377492.1 Zika virus isolate MU-DMSC-1/2016, complete genome
MT377497.1 Zika virus isolate MU-DMSC-3/2016, complete genome
MT377496.1 Zika virus isolate MU-DMSC-2/2016, complete genome
MT377495.1 Zika virus isolate MU-DMSC-4/2017, complete genome
MT377498.1 Zika virus isolate MU-DMSC-4/2016, complete genome
MT377491.1 Zika virus isolate MU-DMSC-1/2017, complete genome
MT377504.1 Zika virus isolate NS-110/2006, complete genome
KX879603.1 Zika virus strain ZIKV/EC/Esmeraldas/062/2016, complete genome
LC191864.1 Zika virus genomic RNA, complete genome, strain: ZIKV/Hu/Chiba/S36/2016
MF794971.1 Zika virus strain ZIKV/EC/Esmeraldas/121/2016, complete genome
MG807647.1 Zika virus isolate SI-BKK06, complete genome
KX766029.1 Zika virus isolate R116265, complete genome
KX051563.1 Zika virus isolate Haiti/1/2016, complete genome
KX247646.1 Zika virus isolate Zika virus/Homo sapiens/COL/UF-1/2016, complete genome
KX702400.1 Zika virus strain Zika virus/Homo sapiens/VEN/UF-1/2016, complete genome
KX893855.1 Zika virus strain Zika virus/Homo sapiens/VEN/UF-2/2016, complete genome
KY765317.1 Zika virus strain ZIKV/Homo sapiens/NIC/7252_12A1/2016, complete genome
KX369547.1 Zika virus strain PF13/251013-18, complete genome
MH513600.1 Zika virus isolate BR/Sinop/H366 2P/2015, complete genome
KX694533.2 Zika virus strain ZIKV/Aedes aegypti/MYS/P6-740/1966, complete genome
KU707826.1 Zika virus isolate SSABRI, complete genome
KY765323.1 Zika virus strain ZIKV/Homo sapiens/NIC/6188_13A1/2016, complete genome
KY765321.1 Zika virus strain ZIKV/Homo sapiens/NIC/4886_12A1_SP/2016, complete genome
KY765326.1 Zika virus strain ZIKV/Homo sapiens/NIC/6188_13A1_SP/2016, complete genome
KY765322.1 Zika virus strain ZIKV/Homo sapiens/NIC/7252_12A1_SP/2016, complete genome
KX156776.2 Zika virus strain ZIKV/Homo sapiens/PAN/CDC-259364_V1-V2/2015, complete genome
KX156775.2 Zika virus strain ZIKV/Homo sapiens/PAN/CDC-259249_V1-V3/2015, complete genome
KU820899.2 Zika virus isolate ZJ03, complete genome
KX117076.1 Zika virus isolate Zhejiang04, complete genome
KX051562.1 Zika virus isolate SV0010/15, complete genome
KY766069.1 Zika virus isolate Pf13/251013-18, complete genome
KU321639.1 Zika virus strain ZikaSPH2015, complete genome
KU509998.3 Zika virus strain Haiti/1225/2014, complete genome
KU744693.1 Zika virus isolate VE_Ganxian, complete genome
MT483911.1 Zika virus isolate 800/16, complete genome
MH157213.1 Zika virus strain ZIKV/Homo sapiens/MEX/41-001-F_V3_O/2016, complete genome
MT377501.1 Zika virus isolate MU-DMSC-5/2017, complete genome
MH061869.1 Zika virus strain ZIKV/Macaca mulatta/UGA/MR-766-VEROE6-AA1-P8_06/1947, complete
genome
MH061867.1 Zika virus strain ZIKV/Macaca mulatta/UGA/MR-766-VEROE6-GA3-P10_04/1947,
complete genome
MH061858.1 Zika virus strain ZIKV/Macaca mulatta/UGA/MR-766-VEROE6-FC11-P7_05/1947, complete
genome
OK054351.1 Zika virus isolate 211784, complete genome
KU955595.1 Zika virus isolate Zika virus/A. taylori-tc/SEN/1984/41671-DAK, complete genome
KU955592.1 Zika virus isolate Zika virus/A. taylori-tc/SEN/1984/41662-DAK, complete genome
KU955591.1 Zika virus isolate Zika virus/A. africanus-tc/SEN/1984/41525-DAK, complete genome
MG758786.1 Zika virus isolate 41525, complete genome
MG758785.1 Zika virus isolate 41525, complete genome
MT505350.1 Mutant Zika virus isolate DK23, complete genome
MT505349.1 Mutant Zika virus isolate DK, complete genome
KY288905.1 Zika virus strain MP1751, complete genome
MF510857.1 Zika virus isolate Zika virus/Aedes taylori/Senegal/1984/DakAr41667, complete genome
OL414716.1 Zika virus isolate 15555, complete genome
MW143022.1 Zika virus strain MR766, complete genome
KX156774.2 Zika virus strain ZIKV/Homo sapiens/PAN/CDC-259359_V1-V3/2015, complete genome
MH061868.1 Zika virus strain ZIKV/Macaca mulatta/UGA/MR-766-VEROE6-AA1-P8_07/1947, complete
genome
MN025403.1 Zika virus isolate 15555, complete genome
MH061906.1 Zika virus strain ZIKV/Macaca mulatta/UGA/MR-766-VEROE6-FC11-P7_01/1947, complete
genome
MH061878.1 Zika virus strain ZIKV/Macaca mulatta/UGA/MR-766-VEROE6-FC3-P8_05/1947, complete
genome
MH061891.1 Zika virus strain ZIKV/Macaca mulatta/UGA/MR-766-VEROE6-FA12-P7_06/1947,
complete genome
MH061877.1 Zika virus strain ZIKV/Macaca mulatta/UGA/MR-766-VEROE6-FC11-P7_07/1947, complete
genome
MH061860.1 Zika virus strain ZIKV/Macaca mulatta/UGA/MR-766-VEROE6-FC11-P7_06/1947, complete
genome
MH061904.1 Zika virus strain ZIKV/Macaca mulatta/UGA/MR-766-VEROE6-FC3-P8_02/1947, complete
genome
MH061886.1 Zika virus strain ZIKV/Macaca mulatta/UGA/MR-766-VEROE6-FA12-P7_05/1947,
complete genome
MH061859.1 Zika virus strain ZIKV/Macaca mulatta/UGA/MR-766-VEROE6-AC11-P5_01/1947,
complete genome
MH061894.1 Zika virus strain ZIKV/Macaca mulatta/UGA/MR-766-VEROE6-FC11-P7_03/1947, complete
genome
KX601166.2 Zika virus strain ZIKV/Aedes africanus/SEN/DakAr41524/1984, complete genome
KX198134.2 Zika virus strain ZIKV/Aedes africanus/SEN/DAK-AR-41524_A1C1-V2/1984, complete
genome
MH061881.1 Zika virus strain ZIKV/Macaca mulatta/UGA/MR-766-VEROE6-GD12-P10_02/1947,
complete genome
MH061913.1 Zika virus strain ZIKV/Macaca mulatta/UGA/MR-766-VEROE6-GA3-P10_05/1947,
complete genome
AY632535.2 Zika virus strain MR 766, complete genome
LC002520.1 Zika virus genomic RNA, complete genome, strain: MR766-NIID
KU955594.1 Zika virus isolate Zika virus/M. mulatta-tc/UGA/1947/MR-766, complete genome
KX377335.1 Zika virus strain MR-766, complete genome
KX830960.1 Zika virus culture ATCC: VR-84 isolate MR776, complete genome
KY989511.1 Zika virus strain MR 766, complete genome
NC 012532.1 Zika virus, complete genome
MH061905.1 Zika virus strain ZIKV/Macaca mulatta/UGA/MR-766-VEROE6-AA1-P8_01/1947, complete
genome
MH061903.1 Zika virus strain ZIKV/Macaca mulatta/UGA/MR-766-VEROE6-AA1-P8_04/1947, complete
genome
MH061901.1 Zika virus strain ZIKV/Macaca mulatta/UGA/MR-766-VEROE6-GA3-P10_07/1947,
complete genome
MH061893.1 Zika virus strain ZIKV/Macaca mulatta/UGA/MR-766-VEROE6-GD12-P10_03/1947,
complete genome
MH061884.1 Zika virus strain ZIKV/Macaca mulatta/UGA/MR-766-VEROE6-GA3-P10_01/1947,
complete genome
MH061882.1 Zika virus strain ZIKV/Macaca mulatta/UGA/MR-766-VEROE6-GD12-P10_08/1947,
complete genome
MH061879.1 Zika virus strain ZIKV/Macaca mulatta/UGA/MR-766-VEROE6-GD12-P10_05/1947,
complete genome
MH061874.1 Zika virus strain ZIKV/Macaca mulatta/UGA/MR-766-VEROE6-AA1-P8_03/1947, complete
genome
MH061865.1 Zika virus strain ZIKV/Macaca mulatta/UGA/MR-766-VEROE6-AA1-P8_02/1947, complete
genome
MH061863.1 Zika virus strain ZIKV/Macaca mulatta/UGA/MR-766-VEROE6-FA12-P7_02/1947,
complete genome
MH061915.1 Zika virus strain ZIKV/Macaca mulatta/UGA/MR-766-VEROE6-P12_02/1947, complete
genome
MH061911.1 Zika virus strain ZIKV/Macaca mulatta/UGA/MR-766-VEROE6-GD12-P10_06/1947,
complete genome
MH061909.1 Zika virus strain ZIKV/Macaca mulatta/UGA/MR-766-VEROE6-AC11-P5_04/1947,
complete genome
MH061908.1 Zika virus strain ZIKV/Macaca mulatta/UGA/MR-766-VEROE6-P12_06/1947, complete
genome
MH061902.1 Zika virus strain ZIKV/Macaca mulatta/UGA/MR-766-VEROE6-FA12-P7_08/1947,
complete genome
MH061900.1 Zika virus strain ZIKV/Macaca mulatta/UGA/MR-766-VEROE6-P12_04/1947, complete
genome
MH061897.1 Zika virus strain ZIKV/Macaca mulatta/UGA/MR-766-VEROE6-FC3-P8_01/1947, complete
genome
MH061895.1 Zika virus strain ZIKV/Macaca mulatta/UGA/MR-766-VEROE6-P12_01/1947, complete
genome
MH061892.1 Zika virus strain ZIKV/Macaca mulatta/UGA/MR-766-VEROE6-P12_03/1947, complete
genome
MH061890.1 Zika virus strain ZIKV/Macaca mulatta/UGA/MR-766-VEROE6-FC3-P8_08/1947, complete
genome
MH061889.1 Zika virus strain ZIKV/Macaca mulatta/UGA/MR-766-VEROE6-FC3-P8_07/1947, complete
genome
MH061888.1 Zika virus strain ZIKV/Macaca mulatta/UGA/MR-766-VEROE6-AC11-P5_02/1947,
complete genome
MH061880.1 Zika virus strain ZIKV/Macaca mulatta/UGA/MR-766-VEROE6-P12_08/1947, complete
genome
MH061875.1 Zika virus strain ZIKV/Macaca mulatta/UGA/MR-766-VEROE6-AC11-P5_03/1947,
complete genome
MH061873.1 Zika virus strain ZIKV/Macaca mulatta/UGA/MR-766-VEROE6-FA12-P7_01/1947,
complete genome
MH061870.1 Zika virus strain ZIKV/Macaca mulatta/UGA/MR-766-VEROE6-AC11-P5_05/1947,
complete genome
MH061862.1 Zika virus strain ZIKV/Macaca mulatta/UGA/MR-766-VEROE6-GD12-P10_01/1947,
complete genome
MH061857.1 Zika virus strain ZIKV/Macaca mulatta/UGA/MR-766-VEROE6-AC11-P5_07/1947,
complete genome
MH061855.1 Zika virus strain ZIKV/Macaca mulatta/UGA/MR-766-VEROE6-FA12-P7_03/1947,
complete genome
MH061852.1 Zika virus strain ZIKV/Macaca mulatta/UGA/MR-766-VEROE6-AC11-P5_08/1947,
complete genome
MH061856.1 Zika virus strain ZIKV/Macaca mulatta/UGA/MR-766-VEROE6-FC3-P8_03/1947, complete
genome
MH130096.1 Zika virus strain ZIKV/Macaca mulatta/UGA/MR-766-VEROE6-AC10-P12_05/1947,
complete genome
MH061914.1 Zika virus strain ZIKV/Macaca mulatta/UGA/MR-766-VEROE6-GA3-P10_02/1947,
complete genome
MH061898.1 Zika virus strain ZIKV/Macaca mulatta/UGA/MR-766-VEROE6-FC11-P7_02/1947, complete
genome
MH061899.1 Zika virus strain ZIKV/Macaca mulatta/UGA/MR-766-VEROE6-AA1-P8_08/1947, complete
genome
MH061871.1 Zika virus strain ZIKV/Macaca mulatta/UGA/MR-766-VEROE6-FC11-P7_04/1947, complete
genome
MK105975.1 Zika virus strain MR766, complete genome
MW680970.1 Zika virus isolate rGZ02p/2018, complete genome
MW680969.1 Zika virus isolate rGZ02a/2018, complete genome
MH061887.1 Zika virus strain ZIKV/Macaca mulatta/UGA/MR-766-VEROE6-P12_05/1947, complete
genome
MH061872.1 Zika virus strain ZIKV/Macaca mulatta/UGA/MR-766-VEROE6-AA1-P8_05/1947, complete
genome
MH061866.1 Zika virus strain ZIKV/Macaca mulatta/UGA/MR-766-VEROE6-GD12-P10_07/1947,
complete genome
MH061861.1 Zika virus strain ZIKV/Macaca mulatta/UGA/MR-766-VEROE6-GA3-P10_08/1947,
complete genome
MH061910.1 Zika virus strain ZIKV/Macaca mulatta/UGA/MR-766-VEROE6-FA12-P7_04/1947,
complete genome
MH130102.1 Zika virus strain ZIKV/Macaca mulatta/UGA/MR-766-VEROE6-AC10-P12_02/1947,
complete genome
MH061876.1 Zika virus strain ZIKV/Macaca mulatta/UGA/MR-766-VEROE6-P12_07/1947, complete
genome
MH061854.1 Zika virus strain ZIKV/Macaca mulatta/UGA/MR-766-VEROE6-AC11-P5_06/1947,
complete genome
MH061907.1 Zika virus strain ZIKV/Macaca mulatta/UGA/MR-766-VEROE6-GA3-P10_06/1947,
complete genome
MH130109.1 Zika virus strain ZIKV/Macaca mulatta/UGA/MR-766-VEROE6-AC4-P12_06/1947,
complete genome
MH061853.1 Zika virus strain ZIKV/Macaca mulatta/UGA/MR-766-VEROE6-GD12-P10_04/1947,
complete genome
KX601169.1 Zika virus strain ZIKV/Macaca mulatta/UGA/MR-766/1947, complete genome
MH061864.1 Zika virus strain ZIKV/Macaca mulatta/UGA/MR-766-VEROE6-FA12-P7_07/1947,
complete genome
MH130094.1 Zika virus strain ZIKV/Macaca mulatta/UGA/MR-766-VEROE6-AC4-P12_05/1947,
complete genome
MH061912.1 Zika virus strain ZIKV/Macaca mulatta/UGA/MR-766-VEROE6-GA3-P10_03/1947,
complete genome
MH061896.1 Zika virus strain ZIKV/Macaca mulatta/UGA/MR-766-VEROE6-FC3-P8_04/1947, complete
genome
MH061885.1 Zika virus strain ZIKV/Macaca mulatta/UGA/MR-766-VEROE6-FC3-P8_06/1947, complete
genome
MH130108.1 Zika virus strain ZIKV/Macaca mulatta/UGA/MR-766-VEROE6-AC4-P12_04/1947,
complete genome
MH130107.1 Zika virus strain ZIKV/Macaca mulatta/UGA/MR-766-VEROE6-AC4-P12_03/1947,
complete genome
MH130106.1 Zika virus strain ZIKV/Macaca mulatta/UGA/MR-766-VEROE6-AC10-P12_08/1947,
complete genome
MH130105.1 Zika virus strain ZIKV/Macaca mulatta/UGA/MR-766-VEROE6-AC10-P12_04/1947,
complete genome
MH130103.1 Zika virus strain ZIKV/Macaca mulatta/UGA/MR-766-VEROE6-AC10-P12_06/1947,
complete genome
MH130101.1 Zika virus strain ZIKV/Macaca mulatta/UGA/MR-766-VEROE6-AC4-P12_08/1947,
complete genome
MH130100.1 Zika virus strain ZIKV/Macaca mulatta/UGA/MR-766-VEROE6-AC4-P12_01/1947,
complete genome
MH130099.1 Zika virus strain ZIKV/Macaca mulatta/UGA/MR-766-VEROE6-AC4-P12_02/1947,
complete genome
MH130098.1 Zika virus strain ZIKV/Macaca mulatta/UGA/MR-766-VEROE6-AC10-P12_01/1947,
complete genome
MH130097.1 Zika virus strain ZIKV/Macaca mulatta/UGA/MR-766-VEROE6-AC10-P12_03/1947,
complete genome
MH130095.1 Zika virus strain ZIKV/Macaca mulatta/UGA/MR-766-VEROE6-AC4-P12_07/1947,
complete genome
MH061883.1 Zika virus strain ZIKV/Macaca mulatta/UGA/MR-766-VEROE6-FC11-P7_08/1947, complete
genome
MH130104.1 Zika virus strain ZIKV/Macaca mulatta/UGA/MR-766-VEROE6-AC10-P12_07/1947,
complete genome

TABLE 1I
Non-limiting example Zika capsid (C) sequences by accession number

| | |
|---|---|
| ASB32509.1 capsid protein C | AVC63698.1 Flavivirus capsid protein C |
| ALU33341.1 capsid | AVC63697.1 Flavivirus capsid protein C |
| AMQ48982.1 anchored capsid protein C | QOF88708.1 Flavivirus capsid protein C |
| AMQ48981.1 anchored capsid protein C | QKF93441.1 Flavivirus capsid protein C |
| ANC90426.1 capsid | QKF93440.1 Flavivirus capsid protein C |
| APH11492.1 anchored capsid protein C | QKF93439.1 Flavivirus capsid protein C |
| AXF50016.1 capsid | QKF93435.1 Flavivirus capsid protein C |
| AXF50015.1 capsid | QKF93430.1 Flavivirus capsid protein C |
| AXF50014.1 capsid | AMM39806.1 Flavivirus capsid protein C |
| AXQ40007.1 anchored capsid protein C | AMQ34004.1 Flavivirus capsid protein C |
| AXQ40006.1 anchored capsid protein C | AMQ34003.1 Flavivirus capsid protein C |
| QGA67054.1 anchored capsid protein C | ANA12599.1 Flavivirus capsid protein C |
| QGA67053.1 anchored capsid protein C | ANB66183.1 Flavivirus capsid protein C |
| QGA67052.1 anchored capsid protein C | ANB66182.1 Flavivirus capsid protein C |
| QGA67051.1 anchored capsid protein C | ANC90428.1 Flavivirus capsid protein C |
| QGA67050.1 anchored capsid protein C | ANF16414.1 Flavivirus capsid protein C |
| QGA72951.1 anchored capsid protein C | AMM43326.1 Flavivirus capsid protein C |
| QBM11757.1 capsid | ARM59239.1 Flavivirus capsid protein C |
| QKM77299.1 anchored capsid protein C | AUI42330.1 Flavivirus capsid protein C |
| QKM77298.1 anchored capsid protein C | AUI42329.1 Flavivirus capsid protein C |
| QKU35968.1 anchored capsid protein C | AXF50052.1 Flavivirus capsid protein C |
| QKX95861.1 capsid protein C | QDZ58856.1 Flavivirus capsid protein C |
| QTA38994.1 anchored capsid protein C | QDZ58855.1 Flavivirus capsid protein C |
| QTA38993.1 anchored capsid protein C | QDZ58854.1 Flavivirus capsid protein C |
| QKF93438.1 anchored capsid protein C | QGA67056.1 Flavivirus capsid protein C |
| QKF93437.1 anchored capsid protein C | QGA67055.1 Flavivirus capsid protein C |
| QKF93436.1 anchored capsid protein C | UED15620.1 Flavivirus capsid protein C |
| QKF93434.1 anchored capsid protein C | AMC13911.1 Flavivirus capsid protein C |
| QKF93433.1 anchored capsid protein C | ANK57897.1 Flavivirus capsid protein C |
| QKF93432.1 anchored capsid protein C | ANW07476.1 Flavivirus capsid protein C |
| QKF93431.1 anchored capsid protein C | AWH65849.1 Flavivirus capsid protein C |
| QKF93429.1 anchored capsid protein C | AYI50275.1 Flavivirus capsid protein C |
| QKF93428.1 anchored capsid protein C | AYI50274.1 Flavivirus capsid protein C |
| UHI99867.1 anchored capsid protein C | AYI50273.1 Flavivirus capsid protein C |
| UHI99866.1 anchored capsid protein C | QCG74166.1 Flavivirus capsid protein C |
| UHI99865.1 anchored capsid protein C | AXQ40005.1 Flavivirus capsid protein C |
| YP 009428568.1 anchored capsid protein C | AXQ40004.1 Flavivirus capsid protein C |
| YP_009430295.1 anchored capsid protein C | AXQ40003.1 Flavivirus capsid protein C |
| AHZ13508.1 Flavivirus capsid protein C | AXQ40002.1 Flavivirus capsid protein C |
| ALU33341.1 Flavivirus capsid protein C | AXQ40001.1 Flavivirus capsid protein C |
| AMB37295.1 Flavivirus capsid protein C | AXQ40000.1 Flavivirus capsid protein C |
| AMD61711.1 Flavivirus capsid protein C | AXQ39999.1 Flavivirus capsid protein C |
| AMB18850.1 Flavivirus capsid protein C | AXQ39998.1 Flavivirus capsid protein C |
| AMH87239.1 Flavivirus capsid protein C | AXQ39997.1 Flavivirus capsid protein C |
| AMD16557.1 Flavivirus capsid protein C | AXQ39996.1 Flavivirus capsid protein C |
| AMN14620.1 Flavivirus capsid protein C | QKM77301.1 Flavivirus capsid protein C |
| AMN14619.1 Flavivirus capsid protein C | QKM77300. 1 Flavivirus capsid protein C |
| AMQ48982.1 Flavivirus capsid protein C | AWF93618.1 Flavivirus capsid protein C |
| AMQ48981.1 Flavivirus capsid protein C | QFZ95661.1 Flavivirus capsid protein C |
| AMQ48986.1 Flavivirus capsid protein C | ANW07476.1 capsid protein C |
| AMX81919.1 Flavivirus capsid protein C | AYI50275.1 capsid protein C |
| ANG09399.1 Flavivirus capsid protein C | AYI50274.1 capsid protein C |
| ANH10698.1 Flavivirus capsid protein C | AYI50273.1 capsid protein C |
| ANN44857.1 Flavivirus capsid protein C | AXQ40005.1 capsid protein C |
| ANZ46736.1 Flavivirus capsid protein C | AXQ40004.1 capsid protein C |
| AOG18296.1 Flavivirus capsid protein C | AXQ40003.1 capsid protein C |
| AND01116.2 Flavivirus capsid protein C | AXQ40002.1 capsid protein C |
| AOR82893.1 Flavivirus capsid protein C | AXQ40001.1 capsid protein C |
| AOR82892.1 Flavivirus capsid protein C | AXQ40000.1 capsid protein C |
| BAV82373.1 Flavivirus capsid protein C | AXQ39999.1 capsid protein C |
| ANH22038.1 Flavivirus capsid protein C | AXQ39998.1 capsid protein C |
| APC60216.2 Flavivirus capsid protein C | AXQ39997.1 capsid protein C |
| APO08503. 1 Flavivirus capsid protein C | AXQ39996.1 capsid protein C |
| APH11492.1 Flavivirus capsid protein C | QKM77301.1 capsid protein C |
| APO36913. 1 Flavivirus capsid protein C | QKM77300.1 capsid protein C |
| AOO19564.1 Flavivirus capsid protein C | AWF93618.1 capsid protein C |
| AQV07165.1 Flavivirus capsid protein C | UBI73854.1 anchored capsid protein C |
| BAX00477.1 Flavivirus capsid protein C | AMX81915.1 Flavivirus capsid protein C |
| AQW43708.1 Flavivirus capsid protein C | UBI73854.1 Flavivirus capsid protein C |
| AQW43707.1 Flavivirus capsid protein C | AMM43325.1 Flavivirus capsid protein C |
| AQZ41956.1 Flavivirus capsid protein C | ANC90427.1 capsid protein C |
| AQZ41955.1 Flavivirus capsid protein C | ANW07477.1 capsid protein C |
| AQZ41954.1 Flavivirus capsid protein C | ANW07474.2 capsid protein C |
| AQZ41953.1 Flavivirus capsid protein C | AVW85863.1 capsid protein C |
| AQZ41952.1 Flavivirus capsid protein C | AVW85862.1 capsid protein C |
| AQZ41951.1 Flavivirus capsid protein C | AVW85861.1 capsid protein C |
| AQZ41950.1 Flavivirus capsid protein C | AVW85860.1 capsid protein C |
| AQZ41949.1 Flavivirus capsid protein C | AVW85859.1 capsid protein C |
| AQZ41948.1 Flavivirus capsid protein C | AVW85858.1 capsid protein C |
| AQZ41947.1 Flavivirus capsid protein C | AVW85857.1 capsid protein C |
| APW84877.1 Flavivirus capsid protein C | AVW85856.1 capsid protein C |
| APW84876.1 Flavivirus capsid protein C | AVW85855.1 capsid protein C |
| APW84875.1 Flavivirus capsid protein C | AVW85854.1 capsid protein C |
| APW84874.1 Flavivirus capsid protein C | AVW85853.1 capsid protein C |
| APW84873.1 Flavivirus capsid protein C | AVW85852.1 capsid protein C |
| APW84872.1 Flavivirus capsid protein C | AVW85851.1 capsid protein C |
| ASB32509.1 Flavivirus capsid protein C | AVW85850.1 capsid protein C |
| ASF57880.1 Flavivirus capsid protein C | AVW85849.1 capsid protein C |
| AQS26698.1 Flavivirus capsid protein C | AVW85848.1 capsid protein C |
| ATB53752.1 Flavivirus capsid protein C | AVW85847.1 capsid protein C |
| ARB08102.1 Flavivirus capsid protein C | AVW85846.1 capsid protein C |
| AVG19275.1 Flavivirus capsid protein C | AVW85845.1 capsid protein C |
| BBC70847.1 Flavivirus capsid protein C | AVW85844.1 capsid protein C |
| AWF93619.1 Flavivirus capsid protein C | AVW85843.1 capsid protein C |
| AWF93617.1 Flavivirus capsid protein C | AVW85842.1 capsid protein C |
| ARU07076.1 Flavivirus capsid protein C | AVW85841.1 capsid protein C |
| ARU07075.1 Flavivirus capsid protein C | AVW85840.1 capsid protein C |
| ARU07074.1 Flavivirus capsid protein C | AVW85839.1 capsid protein C |
| AVV62004.1 Flavivirus capsid protein C | AVW85838.1 capsid protein C |
| AVG19203.1 Flavivirus capsid protein C | AVW85837.1 capsid protein C |
| AVG19202.1 Flavivirus capsid protein C | AVW85836.1 capsid protein C |
| AUF35022.1 Flavivirus capsid protein C | AVW85835.1 capsid protein C |
| AUF35021.1 Flavivirus capsid protein C | AVW85834.1 capsid protein C |
| ATL14618.1 Flavivirus capsid protein C | AVW85833.1 capsid protein C |
| AXF50016.1 Flavivirus capsid protein C | AVW85832.1 capsid protein C |
| AXF50015.1 Flavivirus capsid protein C | AVW85831.1 capsid protein C |
| AXF50014.1 Flavivirus capsid protein C | AVW85830.1 capsid protein C |
| ATB53754.1 Flavivirus capsid protein C | AVW85829.1 capsid protein C |
| ATB53753.1 Flavivirus capsid protein C | AVW85828.1 capsid protein C |
| AYV74937.1 Flavivirus capsid protein C | AVW85827.1 capsid protein C |
| AYV74936.1 Flavivirus capsid protein C | AVW85826.1 capsid protein C |
| AYV74935.1 Flavivirus capsid protein C | AVW85825.1 capsid protein C |
| AYV74934.1 Flavivirus capsid protein C | AVW85824.1 capsid protein C |
| AYV74933.1 Flavivirus capsid protein C | AVW85823.1 capsid protein C |
| AYV74932.1 Flavivirus capsid protein C | AVW85822.1 capsid protein C |
| AYV74931.1 Flavivirus capsid protein C | AVW85821.1 capsid protein C |
| AYV74930.1 Flavivirus capsid protein C | AVW85820.1 capsid protein C |
| AYV74929.1 Flavivirus capsid protein C | AVW85819.1 capsid protein C |
| AYV74928.1 Flavivirus capsid protein C | AVW85818.1 capsid protein C |
| AYV74927.1 Flavivirus capsid protein C | AVW85817.1 capsid protein C |
| AYV74925.1 Flavivirus capsid protein C | AVW85816.1 capsid protein C |
| AYV74924.1 Flavivirus capsid protein C | AVW85815.1 capsid protein C |
| AYV74923.1 Flavivirus capsid protein C | AVW85814.1 capsid protein C |
| AYV74922.1 Flavivirus capsid protein C | AVW85813.1 capsid protein C |
| AYV74921.1 Flavivirus capsid protein C | AVW85812.1 capsid protein C |
| AYV74920.1 Flavivirus capsid protein C | AVW85811.1 capsid protein C |
| AYV74919.1 Flavivirus capsid protein C | AVW85810.1 capsid protein C |
| AYV74918.1 Flavivirus capsid protein C | AVW85809.1 capsid protein C |
| AYV74917.1 Flavivirus capsid protein C | AVW85808.1 capsid protein C |
| AYV74916.1 Flavivirus capsid protein C | AVW85807.1 capsid protein C |
| AXQ40007.1 Flavivirus capsid protein C | AVW85806.1 capsid protein C |
| AXQ40006.1 Flavivirus capsid protein C | AVW85805.1 capsid protein C |
| QGA67054.1 Flavivirus capsid protein C | AVW85804.1 capsid protein C |
| QGA67053.1 Flavivirus capsid protein C | AVW85803.1 capsid protein C |
| QGA67052.1 Flavivirus capsid protein C | AVW85802.1 capsid protein C |
| QGA67051.1 Flavivirus capsid protein C | AVW85801.1 capsid protein C |
| QGA67050.1 Flavivirus capsid protein C | AVW85800.1 capsid protein C |
| QGA72951.1 Flavivirus capsid protein C | AWF93635.1 capsid protein C |
| AZS35405.1 Flavivirus capsid protein C | AWF93634.1 capsid protein C |
| AZS35404.1 Flavivirus capsid protein C | AWF93633.1 capsid protein C |
| AZS35403.1 Flavivirus capsid protein C | AWF93632.1 capsid protein C |
| QBM11757.1 Flavivirus capsid protein C | AWF93631.1 capsid protein C |
| QKM77299.1 Flavivirus capsid protein C | AWF93630.1 capsid protein C |
| QKM77298.1 Flavivirus capsid protein C | AWF93629.1 capsid protein C |
| QKU35968.1 Flavivirus capsid protein C | AWF93628.1 capsid protein C |
| QKX95861.1 Flavivirus capsid protein C | AWF93627.1 capsid protein C |
| QLI34051.1 Flavivirus capsid protein C | AWF93626.1 capsid protein C |
| QTA38994.1 Flavivirus capsid protein C | AWF93625.1 capsid protein C |
| QTA38993.1 Flavivirus capsid protein C | AWF93624.1 capsid protein C |
| QVL61761.1 Flavivirus capsid protein C | AWF93623.1 capsid protein C |
| QKF93438.1 Flavivirus capsid protein C | AWF93622.1 capsid protein C |
| QKF93437.1 Flavivirus capsid protein C | AWF93621.1 capsid protein C |
| QKF93436.1 Flavivirus capsid protein C | AWF93620.1 capsid protein C |
| QKF93434.1 Flavivirus capsid protein C | YP 009227196.1 Flavivirus capsid protein C |
| QKF93433.1 Flavivirus capsid protein C | AMR39836.1 Flavivirus capsid protein C |
| QKF93432.1 Flavivirus capsid protein C | AMR39833.1 Flavivirus capsid protein C |
| QKF93431.1 Flavivirus capsid protein C | AMR39832.1 Flavivirus capsid protein C |
| QKF93429.1 Flavivirus capsid protein C | ANW07474.2 Flavivirus capsid protein C |
| QKF93428.1 Flavivirus capsid protein C | AUY62553.1 Flavivirus capsid protein C |
| UHI99866.1 Flavivirus capsid protein C | AUY62552.1 Flavivirus capsid protein C |
| UHI99865.1 Flavivirus capsid protein C | ARM59240.1 capsid protein C |
| YP_009428568.1 Flavivirus capsid protein C | BAP47441.1 Flavivirus capsid protein C |
| YP_009430295.1 Flavivirus capsid protein C | AMR39835.1 Flavivirus capsid protein C |
| AMQ48982.1 capsid protein C | ANW07477.1 Flavivirus capsid protein C |
| AMQ48981.1 capsid protein C | AOR51315.1 Flavivirus capsid protein C |
| ANB66184.1 capsid protein C | ARM59240.1 Flavivirus capsid protein C |
| ANB66183.1 capsid protein C | AVW85863.1 Flavivirus capsid protein C |
| ANB66182.1 capsid protein C | AVW85862.1 Flavivirus capsid protein C |
| ANC90428.1 capsid protein C | AVW85861.1 Flavivirus capsid protein C |
| ANN83273.1 capsid protein C | AVW85860.1 Flavivirus capsid protein C |
| ANN83272.1 capsid protein C | AVW85859.1 Flavivirus capsid protein C |
| ANW07475.1 capsid protein C | AVW85858.1 Flavivirus capsid protein C |
| AOC50654.1 capsid protein C | AVW85857.1 Flavivirus capsid protein C |
| AOC50653.1 capsid protein C | AVW85856.1 Flavivirus capsid protein C |
| AOC50652.1 capsid protein C | AVW85855.1 Flavivirus capsid protein C |
| APH11492.1 capsid protein C | AVW85854.1 Flavivirus capsid protein C |
| AQV07165.1 capsid protein C | AVW85853.1 Flavivirus capsid protein C |
| AQZ41956.1 capsid protein C | AVW85852.1 Flavivirus capsid protein C |
| AQZ41955.1 capsid protein C | AVW85851.1 Flavivirus capsid protein C |
| AQZ41954.1 capsid protein C | AVW85850.1 Flavivirus capsid protein C |
| AQZ41953.1 capsid protein C | AVW85849.1 Flavivirus capsid protein C |
| AQZ41952.1 capsid protein C | AVW85848.1 Flavivirus capsid protein C |
| AQZ41951.1 capsid protein C | AVW85847.1 Flavivirus capsid protein C |
| AQZ41950.1 capsid protein C | AVW85846.1 Flavivirus capsid protein C |
| AQZ41949.1 capsid protein C | AVW85845.1 Flavivirus capsid protein C |
| AQZ41948.1 capsid protein C | AVW85844.1 Flavivirus capsid protein C |
| AQZ41947.1 capsid protein C | AVW85843.1 Flavivirus capsid protein C |
| AWF93619.1 capsid protein C | AVW85842.1 Flavivirus capsid protein C |
| AWF93617.1 capsid protein C | AVW85841.1 Flavivirus capsid protein C |
| AXQ40007.1 capsid protein C | AVW85840.1 Flavivirus capsid protein C |
| AXQ40006.1 capsid protein C | AVW85839.1 Flavivirus capsid protein C |
| QFZ95661.1 capsid protein C | AVW85838.1 Flavivirus capsid protein C |
| QGA67056.1 capsid protein C | AVW85837.1 Flavivirus capsid protein C |
| QGA67055.1 capsid protein C | AVW85836.1 Flavivirus capsid protein C |
| QGA67054.1 capsid protein C | AVW85835.1 Flavivirus capsid protein C |
| QGA67053.1 capsid protein C | AVW85834.1 Flavivirus capsid protein C |
| QGA67052.1 capsid protein C | AVW85833.1 Flavivirus capsid protein C |
| QGA67051.1 capsid protein C | AVW85832.1 Flavivirus capsid protein C |
| QGA67050.1 capsid protein C | AVW85831.1 Flavivirus capsid protein C |
| QGA72951.1 capsid protein C | AVW85830.1 Flavivirus capsid protein C |
| QKM77299.1 capsid protein C | AVW85829.1 Flavivirus capsid protein C |
| QKM77298.1 capsid protein C | AVW85828.1 Flavivirus capsid protein C |
| QKU35968.1 capsid protein C | AVW85827.1 Flavivirus capsid protein C |
| QOF88708.1 capsid protein C | AVW85826.1 Flavivirus capsid protein C |
| QKF93441.1 capsid protein C | AVW85825.1 Flavivirus capsid protein C |
| QKF93440.1 capsid protein C | AVW85824.1 Flavivirus capsid protein C |
| QKF93439.1 capsid protein C | AVW85823.1 Flavivirus capsid protein C |
| QKF93438.1 capsid protein C | AVW85822.1 Flavivirus capsid protein C |
| QKF93437.1 capsid protein C | AVW85821.1 Flavivirus capsid protein C |
| QKF93436.1 capsid protein C | AVW85820.1 Flavivirus capsid protein C |
| QKF93435.1 capsid protein C | AVW85819.1 Flavivirus capsid protein C |
| QKF93434.1 capsid protein C | AVW85818.1 Flavivirus capsid protein C |
| QKF93433.1 capsid protein C | AVW85817.1 Flavivirus capsid protein C |
| QKF93432.1 capsid protein C | AVW85816.1 Flavivirus capsid protein C |
| QKF93431.1 capsid protein C | AVW85815.1 Flavivirus capsid protein C |
| QKF93430.1 capsid protein C | AVW85814.1 Flavivirus capsid protein C |
| QKF93429.1 capsid protein C | AVW85813.1 Flavivirus capsid protein C |
| QKF93428.1 capsid protein C | AVW85812.1 Flavivirus capsid protein C |
| UBI73854.1 capsid protein C | AVW85811.1 Flavivirus capsid protein C |
| UED15620.1 capsid protein C | AVW85810.1 Flavivirus capsid protein C |
| UHI99867.1 capsid protein C | AVW85809.1 Flavivirus capsid protein C |
| UHI99866.1 capsid protein C | AVW85808.1 Flavivirus capsid protein C |
| UHI99865.1 capsid protein C | AVW85807.1 Flavivirus capsid protein C |
| YP_009428568.1 capsid protein C | AVW85806.1 Flavivirus capsid protein C |
| YP_009430296.1 capsid protein C | AVW85805.1 Flavivirus capsid protein C |
| YP_009430296.1 Flavivirus capsid protein C | AVW85804.1 Flavivirus capsid protein C |
| QOF88708.1 anchored capsid protein C | AVW85803.1 Flavivirus capsid protein C |
| QKF93441.1 anchored capsid protein C | AVW85802.1 Flavivirus capsid protein C |
| QKF93440.1 anchored capsid protein C | AVW85801.1 Flavivirus capsid protein C |
| QKF93439.1 anchored capsid protein C | AVW85800.1 Flavivirus capsid protein C |
| QKF93435.1 anchored capsid protein C | AWF93635.1 Flavivirus capsid protein C |
| QKF93430.1 anchored capsid protein C | AWF93634.1 Flavivirus capsid protein C |
| AMQ34004.1 capsid | AWF93633.1 Flavivirus capsid protein C |
| AMQ34003.1 capsid | AWF93632.1 Flavivirus capsid protein C |
| QGA67056.1 anchored capsid protein C | AWF93631.1 Flavivirus capsid protein C |
| QGA67055.1 anchored capsid protein C | AWF93630.1 Flavivirus capsid protein C |
| UED15620.1 anchored capsid protein C | AWF93629.1 Flavivirus capsid protein C |
| AXQ40005.1 anchored capsid protein C | AWF93628.1 Flavivirus capsid protein C |
| AXQ40004.1 anchored capsid protein C | AWF93627.1 Flavivirus capsid protein C |
| AXQ40003.1 anchored capsid protein C | AWF93626.1 Flavivirus capsid protein C |
| AXQ40002.1 anchored capsid protein C | AWF93625.1 Flavivirus capsid protein C |
| AXQ40001.1 anchored capsid protein C | AWF93624.1 Flavivirus capsid protein C |
| AXQ40000.1 anchored capsid protein C | AWF93623.1 Flavivirus capsid protein C |
| AXQ39999.1 anchored capsid protein C | AWF93622.1 Flavivirus capsid protein C |
| AXQ39998.1 anchored capsid protein C | AWF93621.1 Flavivirus capsid protein C |
| AXQ39997.1 anchored capsid protein C | AWF93620.1 Flavivirus capsid protein C |
| AXQ39996.1 anchored capsid protein C | AYU74865.1 Flavivirus capsid protein C |
| QKM77301.1 anchored capsid protein C | QPC45464.1 Flavivirus capsid protein C |
| QKM77300.1 anchored capsid protein C | ASR91936.1 Flavivirus capsid protein C |
| QFZ95661.1 anchored capsid protein C | QDH45910.1 Flavivirus capsid protein C |
| AMK79469.1 Flavivirus capsid protein C | AAV34151.1 Flavivirus capsid protein C |
| AMD61710.1 Flavivirus capsid protein C | YP_002790881.1 Flavivirus capsid protein C |
| AMR39834.1 Flavivirus capsid protein C | YP_009227206.1 Flavivirus capsid protein C |
| AMX81917.1 Flavivirus capsid protein C | YP_002790881.1 capsid protein C |
| AMX81916.1 Flavivirus capsid protein C | YP_009227196.1 capsid protein C |
| AVZ47169.1 Flavivirus capsid protein C | YP_009227206.1 anchored capsid protein C |
| AWH65848.1 Flavivirus capsid protein C | |

TABLE 1J
Non-limiting example Zika Protein E sequences by accession number

| | |
|---|---|
| AMQ34004.1 Env | ARU07076.1 Flavi_glycoprot |
| AMQ34003.1 Env | AYV74937.1 Flavi_glycoprot |
| AMQ48981.1 envelope protein E | AYV74936.1 Flavi_glycoprot |
| ANB66184.1 envelope protein E | AYV74935.1 Flavi_glycoprot |
| ANB66183.1 envelope protein E | AYV74934.1 Flavi_glycoprot |
| ANB66182.1 envelope protein E | AYV74933.1 Flavi_glycoprot |
| ANC90428.1 envelope protein E | AYV74932.1 Flavi_glycoprot |
| ANN83273.1 envelope protein E | AWF93617.1 Flavi_glycoprot |
| ANN83272.1 envelope protein E | AMD61710.1 Flavi_glycoprot |
| AOC50652.1 envelope protein E | AVC63698.1 Flavi_glycoprot |
| APH11492.1 envelope protein E | AVC63697.1 Flavi_glycoprot |
| AQV07165.1 envelope protein E | AVZ47169.1 Flavi_glycoprot |
| AQZ41956.1 envelope protein E | QKF93435.1 envelope protein E |
| AQZ41955.1 envelope protein E | QFZ95661.1 envelope protein E |
| AQZ41954.1 envelope protein E | QKF93440.1 envelope protein E |
| AQZ41953.1 envelope protein E | QKF93433.1 envelope protein E |
| AQZ41952.1 envelope protein E | AXQ40006.1 envelope protein E |
| AQZ41951.1 envelope protein E | QOF88708.1 envelope protein E |
| AQZ41950.1 envelope protein E | QKF93439.1 envelope protein E |
| AQZ41949.1 envelope protein E | BAX00477.1 Flavi_glycoprot |
| AQZ41948.1 envelope protein E | QKF93441.1 envelope protein E |
| AQZ41947.1 envelope protein E | AOC50653.1 envelope protein E |
| AWF93619.1 envelope protein E | ANW07475.1 envelope protein E |
| AWF93618.1 envelope protein E | AUY62553.1 Flavi_glycoprot |
| QGA67055.1 envelope protein E | ASR91936.1 Flavi_glycoprot |
| QGA67054.1 envelope protein E | AZS35405.1 flavi_E_stem |
| QGA67053.1 envelope protein E | AZS35404.1 flavi_E_stem |
| QGA67052.1 envelope protein E | AZS35403.1 flavi_E_stem |
| QGA67051.1 envelope protein E | AMM39806.1 flavi_E_stem |
| QGA67050.1 envelope protein E | ANA12599.1 flavi_E_stem |
| QKM77301.1 envelope protein E | ANF16414.1 flavi_E_stem |
| QKM77300.1 envelope protein E | AMM43326.1 flavi_E_stem |
| QKM77299.1 envelope protein E | AMM43325.1 flavi_E_stem |
| QKM77298.1 envelope protein E | AUI42330.1 flavi_E_stem |
| QKU35968.1 envelope protein E | AUI42329.1 flavi_E_stem |
| QTA38994.1 envelope protein E | QKF93430.1 flavi_E_stem |
| QKF93438.1 envelope protein E | AMD61710.1 flavi_E_stem |
| QKF93436.1 envelope protein E | AVC63698.1 flavi_E_stem |
| QKF93434.1 envelope protein E | AVC63697.1 flavi_E_stem |
| QKF93432.1 envelope protein E | QDZ58856.1 flavi_E_stem |
| QKF93429.1 envelope protein E | AMR39834.1 flavi_E_stem |
| UED15620.1 envelope protein E | AWH65848.1 flavi_E_stem |
| UHI99867.1 envelope protein E | QOF88708.1 flavi_E_stem |
| UHI99866.1 envelope protein E | QKF93439.1 flavi_E_stem |
| UHI99865.1 envelope protein E | QKF93435.1 flavi_E_stem |
| YP_009428568.1 envelope protein E | QKF93437.1 flavi_E_stem |
| YP_009430300.1 envelope protein E | AXQ40007.1 flavi_E_stem |
| AXQ40005.1 envelope protein E | AXQ40006.1 flavi_E_stem |
| AXQ40004.1 envelope protein E | QKF93433.1 flavi_E_stem |
| AXQ40003.1 envelope protein E | APC60216.2 flavi_E_stem |
| AXQ40001.1 envelope protein E | AMK79469.1 flavi_E_stem |
| AXQ39999.1 envelope protein E | AMQ48986.1 Flavi_E_C |
| AXQ39998.1 envelope protein E | QKF93435.1 Flavi_E_C |
| AXQ39997.1 envelope protein E | QOF88708.1 Flavi_E_C |
| AXQ39996.1 envelope protein E | QKF93441.1 Flavi_E_C |
| AMB18850.1 Flavi_glycoprot | QKF93439.1 Flavi_E_C |
| AMM39806.1 Flavi_glycoprot | ANW07476.1 Flavi_E_C |
| ANA12599.1 Flavi_glycoprot | AYI50275.1 Flavi_E_C |
| ANB66182.1 Flavi_glycoprot | AYI50274.1 Flavi_E_C |
| ANC90428.1 Flavi_glycoprot | AYI50273.1 Flavi_E_C |
| ANF16414.1 Flavi_glycoprot | QCG74166.1 Flavi_E_C |
| ANK57897.1 Flavi_glycoprot | AMQ48982.1 Flavi_E_C |
| ANN44857.1 Flavi_glycoprot | QFZ95661.1 Flavi_E_C |
| AOG18296.1 Flavi_glycoprot | AZS35405.1 Flavi_E_C |
| AND01116.2 Flavi_glycoprot | AZS35403.1 Flavi_E_C |
| APH11492.1 Flavi_glycoprot | AXF50052.1 Flavi_E_C |
| AMM43326.1 Flavi_glycoprot | AXQ40002.1 Flavi_E_C |
| AMM43325.1 Flavi_glycoprot | QKX95861.1 Flavi_E_C |
| APO36913.1 Flavi_glycoprot | QKF93440.1 Flavi_E_C |
| AOO19564.1 Flavi_glycoprot | QLI34051.1 Flavi_E_C |
| AQV07165.1 Flavi_glycoprot | AMD61710.1 Flavi_E_C |
| AQW43708.1 Flavi_glycoprot | AVC63698.1 Flavi_E_C |
| AQW43707.1 Flavi_glycoprot | AVC63697.1 Flavi_E_C |
| AQZ41955.1 Flavi_glycoprot | APC60216.2 Flavi_glycoprot |
| AQZ41953.1 Flavi_glycoprot | AQS26698.1 Flavi_glycoprot |
| AQZ41952.1 Flavi_glycoprot | AOR51315.1 Flavi_glycoprot |
| AQZ41949.1 Flavi_glycoprot | ARM59240.1 Flavi_glycoprot |
| AMX81917.1 Flavi_glycoprot | UBI73854.1 envelope protein E |
| AMX81916.1 Flavi_glycoprot | ANC90427.1 envelope protein E |
| AMX81915.1 Flavi_glycoprot | ANW07474.2 envelope protein E |
| ARM59239.1 Flavi_glycoprot | AVZ47169.1 flavi_E_stem |
| AUI42329.1 Flavi_glycoprot | QKF93441.1 flavi_E_stem |
| AVG19275.1 Flavi_glycoprot | AMD61711.1 flavi_E_stem |
| BBC70847.1 Flavi_glycoprot | AQS26698.1 flavi_E_stem |
| AWF93619.1 Flavi_glycoprot | UBI73854.1 flavi_E_stem |
| AWF93618.1 Flavi_glycoprot | AMR39836.1 Flavi_E_C |
| AWH65849.1 Flavi_glycoprot | AMR39833.1 Flavi_E_C |
| ARU07075.1 Flavi_glycoprot | AMR39832.1 Flavi_E_C |
| ARU07074.1 Flavi_glycoprot | ANW07474.2 Flavi_E_C |
| AVV62004.1 Flavi_glycoprot | AUY62553.1 Flavi_E_C |
| AVG19203.1 Flavi_glycoprot | AUY62552.1 Flavi_E_C |
| AVG19202.1 Flavi_glycoprot | ASR91936.1 Flavi_E_C |
| AUF35022.1 Flavi_glycoprot | QDH45910.1 Flavi_E_C |
| AUF35021.1 Flavi_glycoprot | QPC45464.1 Flavi_E_C |
| AXF50052.1 Flavi_glycoprot | AUI42330.1 Flavi_glycoprot |
| AXF50016.1 Flavi_glycoprot | ARM59240.1 envelope protein E |
| AXF50014.1 Flavi_glycoprot | AVW85861.1 Flavi_glycoprot |
| AYI50275.1 Flavi_glycoprot | AVW85848.1 Flavi_glycoprot |
| AYI50274.1 Flavi_glycoprot | AVW85841.1 Flavi_glycoprot |
| AYI50273.1 Flavi_glycoprot | AVW85837.1 Flavi_glycoprot |
| AYV74931.1 Flavi_glycoprot | AVW85829.1 Flavi_glycoprot |
| AYV74930.1 Flavi_glycoprot | AVW85828.1 Flavi_glycoprot |
| AYV74929.1 Flavi_glycoprot | AVW85810.1 Flavi_glycoprot |
| AYV74928.1 Flavi_glycoprot | AVW85801.1 Flavi_glycoprot |
| AYV74927.1 Flavi_glycoprot | AWF93631.1 Flavi_glycoprot |
| AYV74925.1 Flavi_glycoprot | AWF93630.1 Flavi_glycoprot |
| AYV74924.1 Flavi_glycoprot | AWF93628.1 Flavi_glycoprot |
| AYV74923.1 Flavi_glycoprot | AWF93623.1 Flavi_glycoprot |
| AYV74922.1 Flavi_glycoprot | AYU74865.1 Flavi_glycoprot |
| AYV74921.1 Flavi_glycoprot | AVW85850.1 Flavi_glycoprot |
| AYV74920.1 Flavi_glycoprot | AVW85844.1 Flavi_glycoprot |
| AYV74919.1 Flavi_glycoprot | AVW85838.1 Flavi_glycoprot |
| AYV74918.1 Flavi_glycoprot | AVW85833.1 Flavi_glycoprot |
| AYV74917.1 Flavi_glycoprot | AVW85826.1 Flavi_glycoprot |
| AYV74916.1 Flavi_glycoprot | UBI73854.1 Flavi_E_C |
| YP_009428568.1 Flavi_glycoprot | AVW85862.1 Flavi_glycoprot |
| AHZ13508.1 Flavi_E_C | AVW85860.1 Flavi_glycoprot |
| ALU33341.1 Flavi_E_C | AVW85849.1 Flavi_glycoprot |
| AMC13911.1 Flavi_E_C | AVW85832.1 Flavi_glycoprot |
| AMB37295.1 Flavi_E_C | AVW85851.1 Flavi_glycoprot |
| AMD61711.1 Flavi_E_C | AVW85847.1 Flavi_glycoprot |
| AMB18850.1 Flavi_E_C | AVW85820.1 Flavi_glycoprot |
| AMH87239.1 Flavi_E_C | AVW85817.1 Flavi_glycoprot |
| AMD16557.1 Flavi_E_C | AVW85816.1 Flavi_glycoprot |
| AMM39806.1 Flavi_E_C | AWF93635.1 Flavi_glycoprot |
| AMN14620.1 Flavi_E_C | AWF93627.1 Flavi_glycoprot |
| AMN14619.1 Flavi_E_C | AWF93621.1 Flavi_glycoprot |
| AMQ34004.1 Flavi_E_C | AWF93620.1 Flavi_glycoprot |
| AMQ34003.1 Flavi_E_C | AVW85863.1 Flavi_glycoprot |
| AMQ48981.1 Flavi_E_C | AVW85852.1 Flavi_glycoprot |
| AMR39834.1 Flavi_E_C | AVW85845.1 Flavi_glycoprot |
| AMX81919.1 Flavi_E_C | AVW85843.1 Flavi_glycoprot |
| ANA12599.1 Flavi_E_C | AVW85835.1 Flavi_glycoprot |
| ANB66183.1 Flavi_E_C | AVW85831.1 Flavi_glycoprot |
| ANB66182.1 Flavi_E_C | AVW85824.1 Flavi_glycoprot |
| ANC90428.1 Flavi_E_C | AVW85821.1 Flavi_glycoprot |
| ANF16414.1 Flavi_E_C | AVW85812.1 Flavi_glycoprot |
| ANG09399.1 Flavi_E_C | AVW85811.1 Flavi_glycoprot |
| ANH10698.1 Flavi_E_C | AVW85804.1 Flavi_glycoprot |
| ANK57897.1 Flavi_E_C | AVW85803.1 Flavi_glycoprot |
| ANN44857.1 Flavi_E_C | AVW85836.1 Flavi_glycoprot |
| ANZ46736.1 Flavi_E_C | AVW85834.1 Flavi_glycoprot |
| AOG18296.1 Flavi_E_C | AVW85823.1 Flavi_glycoprot |
| AND01116.2 Flavi_E_C | AVW85805.1 Flavi_glycoprot |
| AOR82893.1 Flavi_E_C | AVW85800.1 Flavi_glycoprot |
| AOR82892.1 Flavi_E_C | AVW85858.1 Flavi_glycoprot |
| BAV82373.1 Flavi_E_C | AVW85856.1 Flavi_glycoprot |
| ANH22038.1 Flavi_E_C | AVW85839.1 Flavi_glycoprot |
| APC60216.2 Flavi_E_C | AVW85859.1 Flavi_glycoprot |
| APO08503.1 Flavi_E_C | AVW85830.1 Flavi_glycoprot |
| APH11492.1 Flavi_E_C | AVW85827.1 Flavi_glycoprot |
| AMM43326.1 Flavi_E_C | AVW85814.1 Flavi_glycoprot |
| AMM43325.1 Flavi_E_C | AWF93633.1 Flavi_glycoprot |
| APO36913.1 Flavi_E_C | AWF93629.1 Flavi_glycoprot |
| AOO19564.1 Flavi_E_C | AWF93624.1 Flavi_glycoprot |
| AQV07165.1 Flavi_E_C | AWF93622.1 Flavi_glycoprot |
| BAX00477.1 Flavi_E_C | ANW07477.1 envelope protein E |
| AQW43708.1 Flavi_E_C | AVW85841.1 envelope protein E |
| AQW43707.1 Flavi_E_C | AVW85829.1 envelope protein E |
| AQZ41956.1 Flavi_E_C | AVW85828.1 envelope protein E |
| AQZ41955.1 Flavi_E_C | AVW85810.1 envelope protein E |
| AQZ41954.1 Flavi_E_C | AVW85801.1 envelope protein E |
| AQZ41953.1 Flavi_E_C | AVW85850.1 envelope protein E |
| AQZ41952.1 Flavi_E_C | AVW85844.1 envelope protein E |
| AQZ41951.1 Flavi_E_C | AVW85838.1 envelope protein E |
| AQZ41950.1 Flavi_E_C | AVW85833.1 envelope protein E |
| AQZ41949.1 Flavi_E_C | AVW85826.1 envelope protein E |
| AQZ41948.1 Flavi_E_C | YP_002790881.1 envelope protein E |
| AQZ41947.1 Flavi_E_C | YP_009227198.1 envelope protein E |
| AMX81917.1 Flavi_E_C | AVW85857.1 Flavi_glycoprot |
| AMX81916.1 Flavi_E_C | AVW85854.1 Flavi_glycoprot |
| AMX81915.1 Flavi_E_C | AVW85842.1 Flavi_glycoprot |
| ARM59239.1 Flavi_E_C | AVW85825.1 Flavi_glycoprot |
| APW84877.1 Flavi_E_C | AVW85818.1 Flavi_glycoprot |
| APW84876.1 Flavi_E_C | AVW85808.1 Flavi_glycoprot |
| APW84875.1 Flavi_E_C | AVW85802.1 Flavi_glycoprot |
| APW84874.1 Flavi_E_C | AVW85807.1 Flavi_glycoprot |
| APW84873.1 Flavi_E_C | AVW85840.1 Flavi_glycoprot |
| APW84872.1 Flavi_E_C | AVW85819.1 Flavi_glycoprot |
| ASB32509.1 Flavi_E_C | AVW85806.1 Flavi_glycoprot |
| ASF57880.1 Flavi_E_C | AVW85855.1 Flavi_glycoprot |
| AQS26698.1 Flavi_E_C | AVW85815.1 Flavi_glycoprot |
| ATB53752.1 Flavi_E_C | AVW85809.1 Flavi_glycoprot |
| ARB08102.1 Flavi_E_C | AWF93634.1 Flavi_glycoprot |
| AUI42330.1 Flavi_E_C | AWF93632.1 Flavi_glycoprot |
| AUI42329.1 Flavi_E_C | AWF93625.1 Flavi_glycoprot |
| AVG19275.1 Flavi_E_C | AWF93626.1 Flavi_glycoprot |
| BBC70847.1 Flavi_E_C | AVW85862.1 envelope protein E |
| AVZ47169.1 Flavi_E_C | AVW85849.1 envelope protein E |
| AWF93619.1 Flavi_E_C | AVW85832.1 envelope protein E |
| AWF93618.1 Flavi_E_C | AVW85831.1 envelope protein E |
| AWF93617.1 Flavi_E_C | AVW85804.1 envelope protein E |
| AWH65849.1 Flavi_E_C | AVW85848.1 envelope protein E |
| AWH65848.1 Flavi_E_C | AVW85837.1 envelope protein E |
| ARU07076.1 Flavi_E_C | AWF93631.1 envelope protein E |
| ARU07075.1 Flavi_E_C | AWF93630.1 envelope protein E |
| ARU07074.1 Flavi_E_C | AWF93628.1 envelope protein E |
| AVV62004.1 Flavi_E_C | AWF93623.1 envelope protein E |
| AVG19203.1 Flavi_E_C | AVW85824.1 envelope protein E |
| AVG19202.1 Flavi_E_C | AVW85834.1 envelope protein E |
| AUF35022.1 Flavi_E_C | AVW85861.1 envelope protein E |
| AUF35021.1 Flavi_E_C | AVW85858.1 envelope protein E |
| ATL14618.1 Flavi_E_C | AVW85839.1 envelope protein E |
| AXF50016.1 Flavi_E_C | AVW85827.1 envelope protein E |
| AXF50015.1 Flavi_E_C | AWF93629.1 envelope protein E |
| AXF50014.1 Flavi_E_C | AWF93624.1 envelope protein E |
| ATB53754.1 Flavi_E_C | AWF93622.1 envelope protein E |
| ATB53753.1 Flavi_E_C | AVW85808.1 envelope protein E |
| AYV74937.1 Flavi_E_C | AVW85851.1 envelope protein E |
| AYV74936.1 Flavi_E_C | AVW85847.1 envelope protein E |
| AYV74935.1 Flavi_E_C | AVW85842.1 envelope protein E |
| AYV74934.1 Flavi_E_C | AVW85820.1 envelope protein E |
| AYV74933.1 Flavi_E_C | AVW85817.1 envelope protein E |
| AYV74932.1 Flavi_E_C | AVW85863.1 envelope protein E |
| AYV74931.1 Flavi_E_C | AVW85860.1 envelope protein E |
| AYV74930.1 Flavi_E_C | AVW85852.1 envelope protein E |
| AYV74929.1 Flavi_E_C | AVW85845.1 envelope protein E |
| AYV74928.1 Flavi_E_C | AVW85843.1 envelope protein E |
| AYV74927.1 Flavi_E_C | AVW85835.1 envelope protein E |
| AYV74925.1 Flavi_E_C | AVW85821.1 envelope protein E |
| AYV74924.1 Flavi_E_C | AVW85816.1 envelope protein E |
| AYV74923.1 Flavi_E_C | AVW85812.1 envelope protein E |
| AYV74922.1 Flavi_E_C | AVW85811.1 envelope protein E |
| AYV74921.1 Flavi_E_C | AVW85803.1 envelope protein E |
| AYV74920.1 Flavi_E_C | AVW85854.1 envelope protein E |
| AYV74919.1 Flavi_E_C | AVW85825.1 envelope protein E |
| AYV74918.1 Flavi_E_C | AVW85819.1 envelope protein E |
| AYV74917.1 Flavi_E_C | AVW85806.1 envelope protein E |
| AYV74916.1 Flavi_E_C | AVW85836.1 envelope protein E |
| AXQ40007.1 Flavi_E_C | AVW85823.1 envelope protein E |
| AXQ40006.1 Flavi_E_C | AVW85805.1 envelope protein E |
| AXQ40005.1 Flavi_E_C | AVW85800.1 envelope protein E |
| AXQ40004.1 Flavi_E_C | AWF93620.1 envelope protein E |
| AXQ40003.1 Flavi_E_C | AVW85830.1 envelope protein E |
| AXQ40001.1 Flavi_E_C | AVW85814.1 envelope protein E |
| AXQ40000.1 Flavi_E_C | AVW85807.1 envelope protein E |
| AXQ39999.1 Flavi_E_C | AVW85859.1 envelope protein E |
| AXQ39998.1 Flavi_E_C | AVW85856.1 envelope protein E |
| AXQ39997.1 Flavi_E_C | AVW85840.1 envelope protein E |
| AXQ39996.1 Flavi_E_C | AWF93632.1 envelope protein E |
| QDZ58856.1 Flavi_E_C | AVW85846.1 Flavi_glycoprot |
| QDZ58855.1 Flavi_E_C | AVW85853.1 Flavi_glycoprot |
| QDZ58854.1 Flavi_E_C | AVW85822.1 Flavi_glycoprot |
| QGA67056.1 Flavi_E_C | AVW85813.1 Flavi_glycoprot |
| QGA67055.1 Flavi_E_C | AVW85846.1 envelope protein E |
| QGA67054.1 Flavi_E_C | AWF93635.1 envelope protein E |
| QGA67053.1 Flavi_E_C | AWF93627.1 envelope protein E |
| QGA67052.1 Flavi_E_C | AWF93621.1 envelope protein E |
| QGA67051.1 Flavi_E_C | AVW85857.1 envelope protein E |
| QGA67050.1 Flavi_E_C | AVW85855.1 envelope protein E |
| QGA72951.1 Flavi_E_C | AVW85818.1 envelope protein E |
| AZS35404.1 Flavi_E_C | AVW85815.1 envelope protein E |
| QBM11757.1 Flavi_E_C | AVW85809.1 envelope protein E |
| QKM77301.1 Flavi_E_C | AWF93633.1 envelope protein E |
| QKM77300.1 Flavi_E_C | AWF93634.1 envelope protein E |
| QKM77299.1 Flavi_E_C | AWF93625.1 envelope protein E |
| QKM77298.1 Flavi_E_C | AWF93626.1 envelope protein E |
| QKU35968.1 Flavi_E_C | AAV34151.1 Flavi_E_C |
| QTA38994.1 Flavi_E_C | BAP47441.1 Flavi_E_C |
| QTA38993.1 Flavi_E_C | AMR39835.1 Flavi_E_C |
| QVL61761.1 Flavi_E_C | ANW07477.1 Flavi_E_C |
| QKF93438.1 Flavi_E_C | AOR51315.1 Flavi_E_C |
| QKF93437.1 Flavi_E_C | ARM59240.1 Flavi_E_C |
| QKF93436.1 Flavi_E_C | AVW85863.1 Flavi_E_C |
| QKF93434.1 Flavi_E_C | AVW85862.1 Flavi_E_C |
| QKF93433.1 Flavi_E_C | AVW85859.1 Flavi_E_C |
| QKF93432.1 Flavi_E_C | AVW85858.1 Flavi_E_C |
| QKF93431.1 Flavi_E_C | AVW85856.1 Flavi_E_C |
| QKF93430.1 Flavi_E_C | AVW85855.1 Flavi_E_C |
| QKF93429.1 Flavi_E_C | AVW85854.1 Flavi_E_C |
| QKF93428.1 Flavi_E_C | AVW85850.1 Flavi_E_C |
| UED15620.1 Flavi_E_C | AVW85849.1 Flavi_E_C |
| UHI99866.1 Flavi_E_C | AVW85846.1 Flavi_E_C |
| UHI99865.1 Flavi_E_C | AVW85844.1 Flavi_E_C |
| YP_009428568.1 Flavi_E_C | AVW85843.1 Flavi_E_C |
| YP_009430300.1 Flavi_E_C | AVW85842.1 Flavi_E_C |
| AHZ13508.1 flavi_E_stem | AVW85841.1 Flavi_E_C |
| ALU33341.1 flavi_E_stem | AVW85840.1 Flavi_E_C |
| AMC13911.1 flavi_E_stem | AVW85839.1 Flavi_E_C |
| AMB37295.1 flavi_E_stem | AVW85838.1 Flavi_E_C |
| AMB18850.1 flavi_E_stem | AVW85834.1 Flavi_E_C |
| AMH87239.1 flavi_E_stem | AVW85833.1 Flavi_E_C |
| AMD16557.1 flavi_E_stem | AVW85832.1 Flavi_E_C |
| AMN14620.1 flavi_E_stem | AVW85831.1 Flavi_E_C |
| AMN14619.1 flavi_E_stem | AVW85829.1 Flavi_E_C |
| AMQ34004.1 flavi_E_stem | AVW85828.1 Flavi_E_C |
| AMQ34003.1 flavi_E_stem | AVW85827.1 Flavi_E_C |
| AMQ48982.1 flavi_E_stem | AVW85826.1 Flavi_E_C |
| AMQ48981.1 flavi_E_stem | AVW85825.1 Flavi_E_C |
| AMQ48986.1 flavi_E_stem | AVW85824.1 Flavi_E_C |
| AMX81919.1 flavi_E_stem | AVW85819.1 Flavi_E_C |
| ANB66183.1 flavi_E_stem | AVW85816.1 Flavi_E_C |
| ANB66182.1 flavi_E_stem | AVW85815.1 Flavi_E_C |
| ANC90428.1 flavi_E_stem | AVW85810.1 Flavi_E_C |
| ANG09399.1 flavi_E_stem | AVW85809.1 Flavi_E_C |
| ANH10698.1 flavi_E_stem | AVW85808.1 Flavi_E_C |
| ANK57897.1 flavi_E_stem | AVW85807.1 Flavi_E_C |
| ANN44857.1 flavi_E_stem | AVW85806.1 Flavi_E_C |
| ANW07476.1 flavi_E_stem | AVW85804.1 Flavi_E_C |
| ANZ46736.1 flavi_E_stem | AVW85801.1 Flavi_E_C |
| AOG18296.1 flavi_E_stem | AWF93632.1 Flavi_E_C |
| AND01116.2 flavi_E_stem | AWF93629.1 Flavi_E_C |
| AOR82893.1 flavi_E_stem | AWF93624.1 Flavi_E_C |
| AOR82892.1 flavi_E_stem | AWF93622.1 Flavi_E_C |
| BAV82373.1 flavi_E_stem | AYU74865.1 Flavi_E_C |
| ANH22038.1 flavi_E_stem | YP_002790881.1 Flavi_E_C |
| APO08503.1 flavi_E_stem | YP_009227198.1 Flavi_E_C |
| APH11492.1 flavi_E_stem | AMK79469.1 Flavi_E_C |
| APO36913.1 flavi_E_stem | AVW85802.1 envelope protein E |
| AOO19564.1 flavi_E_stem | AVW85853.1 envelope protein E |
| AQV07165.1 flavi_E_stem | AVW85822.1 envelope protein E |
| BAX00477.1 flavi_E_stem | AVW85813.1 envelope protein E |
| AQW43708.1 flavi_E_stem | AAV34151.1 flavi_E_stem |
| AQW43707.1 flavi_E_stem | BAP47441.1 flavi_E_stem |
| AQZ41956.1 flavi_E_stem | AMR39836.1 flavi_E_stem |
| AQZ41955.1 flavi_E_stem | AMR39835.1 flavi_E_stem |
| AQZ41954.1 flavi_E_stem | AMR39833.1 flavi_E_stem |
| AQZ41953.1 flavi_E_stem | AMR39832.1 flavi_E_stem |
| AQZ41952.1 flavi_E_stem | ANW07477.1 flavi_E_stem |
| AQZ41951.1 flavi_E_stem | ANW07474.2 flavi_E_stem |
| AQZ41950.1 flavi_E_stem | AOR51315.1 flavi_E_stem |
| AQZ41949.1 flavi_E_stem | ARM59240.1 flavi_E_stem |
| AQZ41948.1 flavi_E_stem | AVW85862.1 flavi_E_stem |
| AQZ41947.1 flavi_E_stem | AVW85861.1 flavi_E_stem |
| AMX81917.1 flavi_E_stem | AVW85860.1 flavi_E_stem |
| AMX81916.1 flavi_E_stem | AVW85858.1 flavi_E_stem |
| AMX81915.1 flavi_E_stem | AVW85857.1 flavi_E_stem |
| ARM59239.1 flavi_E_stem | AVW85854.1 flavi_E_stem |
| APW84877.1 flavi_E_stem | AVW85853.1 flavi_E_stem |
| APW84876.1 flavi_E_stem | AVW85852.1 flavi_E_stem |
| APW84875.1 flavi_E_stem | AVW85851.1 flavi_E_stem |
| APW84874.1 flavi_E_stem | AVW85850.1 flavi_E_stem |
| APW84873.1 flavi_E_stem | AVW85849.1 flavi_E_stem |
| APW84872.1 flavi_E_stem | AVW85848.1 flavi_E_stem |
| ASB32509.1 flavi_E_stem | AVW85847.1 flavi_E_stem |
| ASF57880.1 flavi_E_stem | AVW85846.1 flavi_E_stem |
| ATB53752.1 flavi_E_stem | AVW85845.1 flavi_E_stem |
| ARB08102.1 flavi_E_stem | AVW85844.1 flavi_E_stem |
| AVG19275.1 flavi_E_stem | AVW85842.1 flavi_E_stem |
| BBC70847.1 flavi_E_stem | AVW85841.1 flavi_E_stem |
| AWF93619.1 flavi_E_stem | AVW85840.1 flavi_E_stem |
| AWF93618.1 flavi_E_stem | AVW85839.1 flavi_E_stem |
| AWF93617.1 flavi_E_stem | AVW85838.1 flavi_E_stem |
| AWH65849.1 flavi_E_stem | AVW85837.1 flavi_E_stem |
| ARU07076.1 flavi_E_stem | AVW85835.1 flavi_E_stem |
| ARU07075.1 flavi_E_stem | AVW85834.1 flavi_E_stem |
| ARU07074.1 flavi_E_stem | AVW85833.1 flavi_E_stem |
| AVV62004.1 flavi_E_stem | AVW85832.1 flavi_E_stem |
| AVG19203.1 flavi_E_stem | AVW85831.1 flavi_E_stem |
| AVG19202.1 flavi_E_stem | AVW85830.1 flavi_E_stem |
| AUF35022.1 flavi_E_stem | AVW85829.1 flavi_E_stem |
| AUF35021.1 flavi_E_stem | AVW85828.1 flavi_E_stem |
| ATL14618.1 flavi_E_stem | AVW85827.1 flavi_E_stem |
| AXF50052.1 flavi_E_stem | AVW85826.1 flavi_E_stem |
| AXF50016.1 flavi_E_stem | AVW85825.1 flavi_E_stem |
| AXF50015.1 flavi_E_stem | AVW85824.1 flavi_E_stem |
| AXF50014.1 flavi_E_stem | AVW85822.1 flavi_E_stem |
| AYI50275.1 flavi_E_stem | AVW85821.1 flavi_E_stem |
| AYI50274.1 flavi_E_stem | AVW85820.1 flavi_E_stem |
| AYI50273.1 flavi_E_stem | AVW85819.1 flavi_E_stem |
| ATB53754.1 flavi_E_stem | AVW85818.1 flavi_E_stem |
| ATB53753.1 flavi_E_stem | AVW85817.1 flavi_E_stem |
| AYV74937.1 flavi_E_stem | AVW85814.1 flavi_E_stem |
| AYV74936.1 flavi_E_stem | AVW85813.1 flavi_E_stem |
| AYV74935.1 flavi_E_stem | AVW85812.1 flavi_E_stem |
| AYV74934.1 flavi_E_stem | AVW85811.1 flavi_E_stem |
| AYV74933.1 flavi_E_stem | AVW85810.1 flavi_E_stem |
| AYV74932.1 flavi_E_stem | AVW85808.1 flavi_E_stem |
| AYV74931.1 flavi_E_stem | AVW85807.1 flavi_E_stem |
| AYV74930.1 flavi_E_stem | AVW85806.1 flavi_E_stem |
| AYV74929.1 flavi_E_stem | AVW85804.1 flavi_E_stem |
| AYV74928.1 flavi_E_stem | AVW85803.1 flavi_E_stem |
| AYV74927.1 flavi_E_stem | AVW85801.1 flavi_E_stem |
| AYV74925.1 flavi_E_stem | AVW85800.1 flavi_E_stem |
| AYV74924.1 flavi_E_stem | AWF93635.1 flavi_E_stem |
| AYV74923.1 flavi_E_stem | AWF93634.1 flavi_E_stem |
| AYV74922.1 flavi_E_stem | AWF93633.1 flavi_E_stem |
| AYV74921.1 flavi_E_stem | AWF93632.1 flavi_E_stem |
| AYV74920.1 flavi_E_stem | AWF93631.1 flavi_E_stem |
| AYV74919.1 flavi_E_stem | AWF93630.1 flavi_E_stem |
| AYV74918.1 flavi_E_stem | AWF93629.1 flavi_E_stem |
| AYV74917.1 flavi_E_stem | AWF93628.1 flavi_E_stem |
| AYV74916.1 flavi_E_stem | AWF93627.1 flavi_E_stem |
| QCG74166.1 flavi_E_stem | AWF93626.1 flavi_E_stem |
| AXQ40005.1 flavi_E_stem | AWF93625.1 flavi_E_stem |
| AXQ40004.1 flavi_E_stem | AWF93624.1 flavi_E_stem |
| AXQ40003.1 flavi_E_stem | AWF93623.1 flavi_E_stem |
| AXQ40002.1 flavi_E_stem | AWF93622.1 flavi_E_stem |
| AXQ40001.1 flavi_E_stem | AWF93621.1 flavi_E_stem |
| AXQ40000.1 flavi_E_stem | AWF93620.1 flavi_E_stem |
| AXQ39999.1 flavi_E_stem | AUY62553.1 flavi_E_stem |
| AXQ39998.1 flavi_E_stem | AUY62552.1 flavi_E_stem |
| AXQ39997.1 flavi_E_stem | AYU74865.1 flavi_E_stem |
| AXQ39996.1 flavi_E_stem | QDH45910.1 flavi_E_stem |
| QDZ58855.1 flavi_E_stem | QPC45464.1 flavi_E_stem |
| QDZ58854.1 flavi_E_stem | YP_002790881.1 flavi_E_stem |
| QFZ95661.1 flavi_E_stem | YP_009227198.1 flavi_E_stem |
| QGA67056.1 flavi_E_stem | AVW85836.1 flavi_E_stem |
| QGA67055.1 flavi_E_stem | AVW85823.1 flavi_E_stem |
| QGA67054.1 flavi_E_stem | AVW85805.1 flavi_E_stem |
| QGA67053.1 flavi_E_stem | AVW85860.1 Flavi_E_C |
| QGA67052.1 flavi_E_stem | AVW85857.1 Flavi_E_C |
| QGA67051.1 flavi_E_stem | AVW85853.1 Flavi_E_C |
| QGA67050.1 flavi_E_stem | AVW85852.1 Flavi_E_C |
| QGA72951.1 flavi_E_stem | AVW85851.1 Flavi_E_C |
| QBM11757.1 flavi_E_stem | AVW85848.1 Flavi_E_C |
| QKM77301.1 flavi_E_stem | AVW85847.1 Flavi_E_C |
| QKM77300.1 flavi_E_stem | AVW85845.1 Flavi_E_C |
| QKM77299.1 flavi_E_stem | AVW85837.1 Flavi_E_C |
| QKM77298.1 flavi_E_stem | AVW85836.1 Flavi_E_C |
| QKU35968.1 flavi_E_stem | AVW85835.1 Flavi_E_C |
| QKX95861.1 flavi_E_stem | AVW85830.1 Flavi_E_C |
| QLI34051.1 flavi_E_stem | AVW85823.1 Flavi_E_C |
| QTA38994.1 flavi_E_stem | AVW85822.1 Flavi_E_C |
| QTA38993.1 flavi_E_stem | AVW85821.1 Flavi_E_C |
| QVL61761.1 flavi_E_stem | AVW85820.1 Flavi_E_C |
| QKF93440.1 flavi_E_stem | AVW85818.1 Flavi_E_C |
| QKF93438.1 flavi_E_stem | AVW85817.1 Flavi_E_C |
| QKF93436.1 flavi_E_stem | AVW85814.1 Flavi_E_C |
| QKF93434.1 flavi_E_stem | AVW85813.1 Flavi_E_C |
| QKF93432.1 flavi_E_stem | AVW85812.1 Flavi_E_C |
| QKF93431.1 flavi_E_stem | AVW85811.1 Flavi_E_C |
| QKF93429.1 flavi_E_stem | AVW85805.1 Flavi_E_C |
| QKF93428.1 flavi_E_stem | AVW85803.1 Flavi_E_C |
| UED15620.1 flavi_E_stem | AVW85802.1 Flavi_E_C |
| UHI99866.1 flavi_E_stem | AVW85800.1 Flavi_E_C |
| UHI99865.1 flavi_E_stem | AWF93634.1 Flavi_E_C |
| YP_009428568.1 flavi_E_stem | AWF93626.1 Flavi_E_C |
| YP_009430300.1 flavi_E_stem | AWF93625.1 Flavi_E_C |
| ASB32509.1 envelope protein E | AWF93620.1 Flavi_E_C |
| QKF93431.1 envelope protein E | AWF93631.1 Flavi_E_C |
| QGA72951.1 envelope protein E | AWF93630.1 Flavi_E_C |
| AOC50654.1 envelope protein E | AWF93628.1 Flavi_E_C |
| AWF93617.1 envelope protein E | AWF93623.1 Flavi_E_C |
| AMQ48982.1 envelope protein E | AVW85861.1 Flavi_E_C |
| ANW07476.1 envelope protein E | ASR91936.1 flavi_E_stem |
| AYI50275.1 envelope protein E | AVW85863.1 flavi_E_stem |
| AYI50274.1 envelope protein E | AVW85843.1 flavi_E_stem |
| AYI50273.1 envelope protein E | AVW85802.1 flavi_E_stem |
| QKF93430.1 envelope protein E | AVW85859.1 flavi_E_stem |
| QKF93428.1 envelope protein E | AVW85856.1 flavi_E_stem |
| QKX95861.1 envelope protein E | AVW85855.1 flavi_E_stem |
| QGA67056.1 envelope protein E | AVW85816.1 flavi_E_stem |
| QKF93437.1 envelope protein E | AVW85815.1 flavi_E_stem |
| QTA38993.1 envelope protein E | AVW85809.1 flavi_E_stem |
| AXQ40002.1 envelope protein E | AWF93635.1 Flavi_E_C |
| AXQ40007.1 envelope protein E | AWF93633.1 Flavi_E_C |
| AXQ40000.1 envelope protein E | AWF93627.1 Flavi_E_C |
| ALU33341.1 Flavi_glycoprot | AWF93621.1 Flavi_E_C |
| ATB53754.1 Flavi_glycoprot | |

TABLE 1K
Non-limiting example Zika Protein M sequences by accession number

| | |
|---|---|
| AMQ48982.1 membrane glycoprotein precursor M | AMD61711.1 Flavivirus polyprotein propeptide |
| AMQ48981.1 membrane glycoprotein precursor M | QKF93432.1 membrane glycoprotein precursor M |
| APH11492.1 membrane glycoprotein precursor M | QKF93431.1 membrane glycoprotein precursor M |
| AXQ40007.1 membrane glycoprotein precursor M | QKM77301.1 membrane glycoprotein precursor M |
| AXQ40006.1 membrane glycoprotein precursor M | QKM77300.1 membrane glycoprotein precursor M |
| AXQ40004.1 membrane glycoprotein precursor M | QKM77298.1 membrane glycoprotein precursor M |
| AXQ40001.1 membrane glycoprotein precursor M | AXQ40002.1 Flavivirus polyprotein propeptide |
| AXQ40000.1 membrane glycoprotein precursor M | ATL14618.1 Flavivirus polyprotein propeptide |
| AXQ39998.1 membrane glycoprotein precursor M | QGA72951.1 Flavivirus polyprotein propeptide |
| AXQ39997.1 membrane glycoprotein precursor M | QOF88708.1 Flavivirus polyprotein propeptide |
| QFZ95661.1 membrane glycoprotein precursor M | QVL61761.1 Flavivirus polyprotein propeptide |
| QGA67056.1 membrane glycoprotein precursor M | QKF93441.1 Flavivirus polyprotein propeptide |
| QGA67055.1 membrane glycoprotein precursor M | QKF93440.1 Flavivirus polyprotein propeptide |
| QGA67054.1 membrane glycoprotein precursor M | QKF93439.1 Flavivirus polyprotein propeptide |
| QGA67053.1 membrane glycoprotein precursor M | QKF93438.1 Flavivirus polyprotein propeptide |
| QGA67052.1 membrane glycoprotein precursor M | QKF93437.1 Flavivirus polyprotein propeptide |
| QGA67051.1 membrane glycoprotein precursor M | QKF93436.1 Flavivirus polyprotein propeptide |
| QGA67050.1 membrane glycoprotein precursor M | QKF93435.1 Flavivirus polyprotein propeptide |
| QKU35968.1 membrane glycoprotein precursor M | QKF93434.1 Flavivirus polyprotein propeptide |
| UED15620.1 membrane glycoprotein precursor M | QKF93433.1 Flavivirus polyprotein propeptide |
| UHI99867.1 membrane glycoprotein precursor M | QKF93432.1 Flavivirus polyprotein propeptide |
| UHI99866.1 membrane glycoprotein precursor M | QKF93431.1 Flavivirus polyprotein propeptide |
| UHI99865.1 membrane glycoprotein precursor M | QKF93430.1 Flavivirus polyprotein propeptide |
| YP_009428568.1 membrane glycoprotein precursor M | QKF93429.1 Flavivirus polyprotein propeptide |
| YP_009430297.1 membrane glycoprotein precursor M | QKF93428.1 Flavivirus polyprotein propeptide |
| AMQ34004.1 protein pr | QKM77301.1 Flavivirus polyprotein propeptide |
| AMQ34003.1 protein pr | QKM77300.1 Flavivirus polyprotein propeptide |
| AMQ48982.1 protein pr | QKM77299.1 Flavivirus polyprotein propeptide |
| AMQ48981.1 protein pr | QKM77298.1 Flavivirus polyprotein propeptide |
| ANB66184.1 protein pr | QKF93432.1 membrane glycoprotein M |
| ANB66183.1 protein pr | QKF93431.1 membrane glycoprotein M |
| ANB66182.1 protein pr | QKM77301.1 membrane glycoprotein M |
| ANC90428.1 protein pr | QKM77300.1 membrane glycoprotein M |
| ANN83273.1 protein pr | QKM77298.1 membrane glycoprotein M |
| ANW07476.1 protein pr | AXQ40005.1 membrane glycoprotein M |
| AOC50654.1 protein pr | AMQ34003.1 membrane glycoprotein M |
| AOO19564.1 protein pr | AXQ39999.1 membrane glycoprotein M |
| AQV07165.1 protein pr | AYV74937.1 Flavivirus envelope glycoprotein M |
| AQZ41956.1 protein pr | AYV74936.1 Flavivirus envelope glycoprotein M |
| AQZ41955.1 protein pr | AYV74935.1 Flavivirus envelope glycoprotein M |
| AQZ41954.1 protein pr | AYV74934.1 Flavivirus envelope glycoprotein M |
| AQZ41953.1 protein pr | AYV74933.1 Flavivirus envelope glycoprotein M |
| AQZ41952.1 protein pr | AYV74932.1 Flavivirus envelope glycoprotein M |
| AQZ41951.1 protein pr | BAX00477.1 Flavivirus envelope glycoprotein M |
| AQZ41950.1 protein pr | AMD61711.1 Flavivirus envelope glycoprotein M |
| AQZ41949.1 protein pr | AMQ48986.1 Flavivirus envelope glycoprotein M |
| AQZ41948.1 protein pr | ANN44857.1 Flavivirus envelope glycoprotein M |
| AQZ41947.1 protein pr | AVG19275.1 Flavivirus envelope glycoprotein M |
| AWF93619.1 protein pr | AQW43708.1 Flavivirus envelope glycoprotein M |
| AWF93618.1 protein pr | AMQ34003.1 Flavivirus envelope glycoprotein M |
| AWF93617.1 protein pr | AXQ40005.1 membrane glycoprotein precursor M |
| AYI50275.1 protein pr | AXQ40005.1 protein pr |
| AYI50274.1 protein pr | ANW07475.1 protein pr |
| AYI50273.1 protein pr | AVZ47169.1 Flavivirus polyprotein propeptide |
| AXQ40007.1 protein pr | BAX00477.1 Flavivirus polyprotein propeptide |
| AXQ40006.1 protein pr | AMX81915.1 Flavivirus polyprotein propeptide |
| AXQ40004.1 protein pr | UBI73854.1 membrane glycoprotein precursor M |
| AXQ40001.1 protein pr | AXQ40005.1 Flavivirus polyprotein propeptide |
| AXQ40000.1 protein pr | AOC50653.1 protein pr |
| AXQ39999.1 protein pr | AMR39834.1 Flavivirus polyprotein propeptide |
| AXQ39998.1 protein pr | AWH65848.1 Flavivirus polyprotein propeptide |
| AXQ39997.1 protein pr | ANC90427.1 small envelope protein M |
| QFZ95661.1 protein pr | ANW07477.1 small envelope protein M |
| QGA67056.1 protein pr | ANW07474.2 small envelope protein M |
| QGA67055.1 protein pr | ARM59240.1 small envelope protein M |
| QGA67054.1 protein pr | AVW85863.1 small envelope protein M |
| QGA67053.1 protein pr | AVW85862.1 small envelope protein M |
| QGA67052.1 protein pr | AVW85861.1 small envelope protein M |
| QGA67051.1 protein pr | AVW85860.1 small envelope protein M |
| QGA67050.1 protein pr | AVW85859.1 small envelope protein M |
| QKU35968.1 protein pr | AVW85857.1 small envelope protein M |
| UED15620.1 protein pr | AVW85856.1 small envelope protein M |
| UHI99867.1 protein pr | AVW85855.1 small envelope protein M |
| UHI99866.1 protein pr | AVW85854.1 small envelope protein M |
| UHI99865.1 protein pr | AVW85853.1 small envelope protein M |
| YP_009428568.1 protein pr | AVW85852.1 small envelope protein M |
| ALU33341.1 Flavivirus polyprotein propeptide | AVW85851.1 small envelope protein M |
| AMB18850.1 Flavivirus polyprotein propeptide | AVW85850.1 small envelope protein M |
| AMM39806.1 Flavivirus polyprotein propeptide | AVW85849.1 small envelope protein M |
| ANA12599.1 Flavivirus polyprotein propeptide | AVW85848.1 small envelope protein M |
| ANB66182.1 Flavivirus polyprotein propeptide | AVW85847.1 small envelope protein M |
| ANC90428.1 Flavivirus polyprotein propeptide | AVW85846.1 small envelope protein M |
| ANF16414.1 Flavivirus polyprotein propeptide | AVW85845.1 small envelope protein M |
| ANK57897.1 Flavivirus polyprotein propeptide | AVW85844.1 small envelope protein M |
| ANN44857.1 Flavivirus polyprotein propeptide | AVW85843.1 small envelope protein M |
| ANW07476.1 Flavivirus polyprotein propeptide | AVW85842.1 small envelope protein M |
| AOG18296.1 Flavivirus polyprotein propeptide | AVW85841.1 small envelope protein M |
| AND01116.2 Flavivirus polyprotein propeptide | AVW85840.1 small envelope protein M |
| APC60216.2 Flavivirus polyprotein propeptide | AVW85839.1 small envelope protein M |
| APH11492.1 Flavivirus polyprotein propeptide | AVW85838.1 small envelope protein M |
| AMM43326.1 Flavivirus polyprotein propeptide | AVW85837.1 small envelope protein M |
| AMM43325.1 Flavivirus polyprotein propeptide | AVW85836.1 small envelope protein M |
| APO36913.1 Flavivirus polyprotein propeptide | AVW85835.1 small envelope protein M |
| AOO19564.1 Flavivirus polyprotein propeptide | AVW85834.1 small envelope protein M |
| AQV07165.1 Flavivirus polyprotein propeptide | AVW85833.1 small envelope protein M |
| AQW43708.1 Flavivirus polyprotein propeptide | AVW85832.1 small envelope protein M |
| AQW43707.1 Flavivirus polyprotein propeptide | AVW85831.1 small envelope protein M |
| AQZ41955.1 Flavivirus polyprotein propeptide | AVW85830.1 small envelope protein M |
| AQZ41953.1 Flavivirus polyprotein propeptide | AVW85829.1 small envelope protein M |
| AQZ41952.1 Flavivirus polyprotein propeptide | AVW85828.1 small envelope protein M |
| AQZ41949.1 Flavivirus polyprotein propeptide | AVW85827.1 small envelope protein M |
| ARM59239.1 Flavivirus polyprotein propeptide | AVW85826.1 small envelope protein M |
| AVG19275.1 Flavivirus polyprotein propeptide | AVW85825.1 small envelope protein M |
| AWF93619.1 Flavivirus polyprotein propeptide | AVW85824.1 small envelope protein M |
| AWF93618.1 Flavivirus polyprotein propeptide | AVW85823.1 small envelope protein M |
| AWF93617.1 Flavivirus polyprotein propeptide | AVW85822.1 small envelope protein M |
| AWH65849.1 Flavivirus polyprotein propeptide | AVW85820.1 small envelope protein M |
| ARU07076.1 Flavivirus polyprotein propeptide | AVW85819.1 small envelope protein M |
| ARU07075.1 Flavivirus polyprotein propeptide | AVW85818.1 small envelope protein M |
| ARU07074.1 Flavivirus polyprotein propeptide | AVW85816.1 small envelope protein M |
| AXF50052.1 Flavivirus polyprotein propeptide | AVW85815.1 small envelope protein M |
| AXF50016.1 Flavivirus polyprotein propeptide | AVW85814.1 small envelope protein M |
| AXF50014.1 Flavivirus polyprotein propeptide | AVW85813.1 small envelope protein M |
| AYI50275.1 Flavivirus polyprotein propeptide | AVW85810.1 small envelope protein M |
| AYI50274.1 Flavivirus polyprotein propeptide | AVW85809.1 small envelope protein M |
| AYI50273.1 Flavivirus polyprotein propeptide | AVW85808.1 small envelope protein M |
| ATB53754.1 Flavivirus polyprotein propeptide | AVW85806.1 small envelope protein M |
| AYV74937.1 Flavivirus polyprotein propeptide | AVW85805.1 small envelope protein M |
| AYV74936.1 Flavivirus polyprotein propeptide | AVW85804.1 small envelope protein M |
| AYV74935.1 Flavivirus polyprotein propeptide | AVW85802.1 small envelope protein M |
| AYV74934.1 Flavivirus polyprotein propeptide | AVW85801.1 small envelope protein M |
| AYV74933.1 Flavivirus polyprotein propeptide | AVW85800.1 small envelope protein M |
| AYV74932.1 Flavivirus polyprotein propeptide | AWF93635.1 small envelope protein M |
| AYV74931.1 Flavivirus polyprotein propeptide | AWF93634.1 small envelope protein M |
| AYV74930.1 Flavivirus polyprotein propeptide | AWF93633.1 small envelope protein M |
| AYV74929.1 Flavivirus polyprotein propeptide | AWF93632.1 small envelope protein M |
| AYV74928.1 Flavivirus polyprotein propeptide | AWF93631.1 small envelope protein M |
| AYV74927.1 Flavivirus polyprotein propeptide | AWF93630.1 small envelope protein M |
| AYV74925.1 Flavivirus polyprotein propeptide | AWF93629.1 small envelope protein M |
| AYV74924.1 Flavivirus polyprotein propeptide | AWF93628.1 small envelope protein M |
| AYV74923.1 Flavivirus polyprotein propeptide | AWF93627.1 small envelope protein M |
| AYV74922.1 Flavivirus polyprotein propeptide | AWF93626.1 small envelope protein M |
| AYV74921.1 Flavivirus polyprotein propeptide | AWF93625.1 small envelope protein M |
| AYV74920.1 Flavivirus polyprotein propeptide | AWF93624.1 small envelope protein M |
| AYV74919.1 Flavivirus polyprotein propeptide | AWF93623.1 small envelope protein M |
| AYV74918.1 Flavivirus polyprotein propeptide | AWF93622.1 small envelope protein M |
| AYV74917.1 Flavivirus polyprotein propeptide | AWF93621.1 small envelope protein M |
| AYV74916.1 Flavivirus polyprotein propeptide | AWF93620.1 small envelope protein M |
| QCG74166.1 Flavivirus polyprotein propeptide | YP_002790881.1 membrane glycoprotein M |
| YP_009428568.1 Flavivirus polyprotein propeptide | YP_009227208.1 membrane glycoprotein M |
| YP_009430297.1 Flavivirus polyprotein propeptide | BAP47441.1 Flavivirus envelope glycoprotein M |
| AMC13911.1 Flavivirus polyprotein propeptide | AMR39836.1 Flavivirus envelope glycoprotein M |
| AMB37295.1 Flavivirus polyprotein propeptide | AMR39835.1 Flavivirus envelope glycoprotein M |
| AMD16557.1 Flavivirus polyprotein propeptide | AMR39833.1 Flavivirus envelope glycoprotein M |
| AMK79469.1 Flavivirus polyprotein propeptide | ANW07477.1 Flavivirus envelope glycoprotein M |
| AMN14619.1 Flavivirus polyprotein propeptide | AOR51315.1 Flavivirus envelope glycoprotein M |
| AMQ34004.1 Flavivirus polyprotein propeptide | ARM59240.1 Flavivirus envelope glycoprotein M |
| AMQ34003.1 Flavivirus polyprotein propeptide | AVW85863.1 Flavivirus envelope glycoprotein M |
| AMQ48982.1 Flavivirus polyprotein propeptide | AVW85862.1 Flavivirus envelope glycoprotein M |
| AMQ48981.1 Flavivirus polyprotein propeptide | AVW85861.1 Flavivirus envelope glycoprotein M |
| AMQ48986.1 Flavivirus polyprotein propeptide | AVW85860.1 Flavivirus envelope glycoprotein M |
| AMX81919.1 Flavivirus polyprotein propeptide | AVW85859.1 Flavivirus envelope glycoprotein M |
| AXQ39996.1 Flavivirus polyprotein propeptide | AVW85857.1 Flavivirus envelope glycoprotein M |
| AHZ13508.1 Flavivirus polyprotein propeptide | AVW85856.1 Flavivirus envelope glycoprotein M |
| AMH87239.1 Flavivirus polyprotein propeptide | AVW85855.1 Flavivirus envelope glycoprotein M |
| AMN14620.1 Flavivirus polyprotein propeptide | AVW85854.1 Flavivirus envelope glycoprotein M |
| ANB66183.1 Flavivirus polyprotein propeptide | AVW85853.1 Flavivirus envelope glycoprotein M |
| ANG09399.1 Flavivirus polyprotein propeptide | AVW85852.1 Flavivirus envelope glycoprotein M |
| ANH10698.1 Flavivirus polyprotein propeptide | AVW85851.1 Flavivirus envelope glycoprotein M |
| ANZ46736.1 Flavivirus polyprotein propeptide | AVW85850.1 Flavivirus envelope glycoprotein M |
| AOR82893.1 Flavivirus polyprotein propeptide | AVW85849.1 Flavivirus envelope glycoprotein M |
| AOR82892.1 Flavivirus polyprotein propeptide | AVW85848.1 Flavivirus envelope glycoprotein M |
| BAV82373.1 Flavivirus polyprotein propeptide | AVW85847.1 Flavivirus envelope glycoprotein M |
| ANH22038.1 Flavivirus polyprotein propeptide | AVW85846.1 Flavivirus envelope glycoprotein M |
| APO08503.1 Flavivirus polyprotein propeptide | AVW85845.1 Flavivirus envelope glycoprotein M |
| AQZ41956.1 Flavivirus polyprotein propeptide | AVW85844.1 Flavivirus envelope glycoprotein M |
| AQZ41954.1 Flavivirus polyprotein propeptide | AVW85843.1 Flavivirus envelope glycoprotein M |
| AQZ41951.1 Flavivirus polyprotein propeptide | AVW85842.1 Flavivirus envelope glycoprotein M |
| AQZ41950.1 Flavivirus polyprotein propeptide | AVW85841.1 Flavivirus envelope glycoprotein M |
| AQZ41948.1 Flavivirus polyprotein propeptide | AVW85840.1 Flavivirus envelope glycoprotein M |
| AQZ41947.1 Flavivirus polyprotein propeptide | AVW85839.1 Flavivirus envelope glycoprotein M |
| APW84877.1 Flavivirus polyprotein propeptide | AVW85838.1 Flavivirus envelope glycoprotein M |
| APW84876.1 Flavivirus polyprotein propeptide | AVW85837.1 Flavivirus envelope glycoprotein M |
| APW84875.1 Flavivirus polyprotein propeptide | AVW85836.1 Flavivirus envelope glycoprotein M |
| APW84874.1 Flavivirus polyprotein propeptide | AVW85835.1 Flavivirus envelope glycoprotein M |
| APW84873.1 Flavivirus polyprotein propeptide | AVW85834.1 Flavivirus envelope glycoprotein M |
| APW84872.1 Flavivirus polyprotein propeptide | AVW85833.1 Flavivirus envelope glycoprotein M |
| ASB32509.1 Flavivirus polyprotein propeptide | AVW85832.1 Flavivirus envelope glycoprotein M |
| ASF57880.1 Flavivirus polyprotein propeptide | AVW85831.1 Flavivirus envelope glycoprotein M |
| ATB53752.1 Flavivirus polyprotein propeptide | AVW85830.1 Flavivirus envelope glycoprotein M |
| ARB08102.1 Flavivirus polyprotein propeptide | AVW85829.1 Flavivirus envelope glycoprotein M |
| AXF50015.1 Flavivirus polyprotein propeptide | AVW85828.1 Flavivirus envelope glycoprotein M |
| ATB53753.1 Flavivirus polyprotein propeptide | AVW85827.1 Flavivirus envelope glycoprotein M |
| AXQ40007.1 Flavivirus polyprotein propeptide | AVW85826.1 Flavivirus envelope glycoprotein M |
| AXQ40006.1 Flavivirus polyprotein propeptide | AVW85825.1 Flavivirus envelope glycoprotein M |
| AXQ40004.1 Flavivirus polyprotein propeptide | AVW85824.1 Flavivirus envelope glycoprotein M |
| AXQ40003.1 Flavivirus polyprotein propeptide | AVW85823.1 Flavivirus envelope glycoprotein M |
| AXQ40001.1 Flavivirus polyprotein propeptide | AVW85822.1 Flavivirus envelope glycoprotein M |
| AXQ40000.1 Flavivirus polyprotein propeptide | AVW85820.1 Flavivirus envelope glycoprotein M |
| AXQ39999.1 Flavivirus polyprotein propeptide | AVW85819.1 Flavivirus envelope glycoprotein M |
| AXQ39998.1 Flavivirus polyprotein propeptide | AVW85818.1 Flavivirus envelope glycoprotein M |
| AXQ39997.1 Flavivirus polyprotein propeptide | AVW85816.1 Flavivirus envelope glycoprotein M |
| QDZ58856.1 Flavivirus polyprotein propeptide | AVW85815.1 Flavivirus envelope glycoprotein M |
| QDZ58855.1 Flavivirus polyprotein propeptide | AVW85814.1 Flavivirus envelope glycoprotein M |
| QDZ58854.1 Flavivirus polyprotein propeptide | AVW85813.1 Flavivirus envelope glycoprotein M |
| QFZ95661.1 Flavivirus polyprotein propeptide | AVW85810.1 Flavivirus envelope glycoprotein M |
| QGA67056.1 Flavivirus polyprotein propeptide | AVW85809.1 Flavivirus envelope glycoprotein M |
| QGA67055.1 Flavivirus polyprotein propeptide | AVW85808.1 Flavivirus envelope glycoprotein M |
| QGA67054.1 Flavivirus polyprotein propeptide | AVW85806.1 Flavivirus envelope glycoprotein M |
| QGA67053.1 Flavivirus polyprotein propeptide | AVW85805.1 Flavivirus envelope glycoprotein M |
| QGA67052.1 Flavivirus polyprotein propeptide | AVW85804.1 Flavivirus envelope glycoprotein M |
| QGA67051.1 Flavivirus polyprotein propeptide | AVW85802.1 Flavivirus envelope glycoprotein M |
| QGA67050.1 Flavivirus polyprotein propeptide | AVW85801.1 Flavivirus envelope glycoprotein M |
| AZS35405.1 Flavivirus polyprotein propeptide | AVW85800.1 Flavivirus envelope glycoprotein M |
| AZS35404.1 Flavivirus polyprotein propeptide | AWF93635.1 Flavivirus envelope glycoprotein M |
| AZS35403.1 Flavivirus polyprotein propeptide | AWF93634.1 Flavivirus envelope glycoprotein M |
| QBM11757.1 Flavivirus polyprotein propeptide | AWF93633.1 Flavivirus envelope glycoprotein M |
| QKU35968.1 Flavivirus polyprotein propeptide | AWF93632.1 Flavivirus envelope glycoprotein M |
| QKX95861.1 Flavivirus polyprotein propeptide | AWF93631.1 Flavivirus envelope glycoprotein M |
| QLI34051.1 Flavivirus polyprotein propeptide | AWF93630.1 Flavivirus envelope glycoprotein M |
| QTA38994.1 Flavivirus polyprotein propeptide | AWF93629.1 Flavivirus envelope glycoprotein M |
| QTA38993.1 Flavivirus polyprotein propeptide | AWF93628.1 Flavivirus envelope glycoprotein M |
| UED15620.1 Flavivirus polyprotein propeptide | AWF93627.1 Flavivirus envelope glycoprotein M |
| UHI99866.1 Flavivirus polyprotein propeptide | AWF93626.1 Flavivirus envelope glycoprotein M |
| UHI99865.1 Flavivirus polyprotein propeptide | AWF93625.1 Flavivirus envelope glycoprotein M |
| AMQ34004.1 membrane glycoprotein M | AWF93624.1 Flavivirus envelope glycoprotein M |
| AMQ48982.1 membrane glycoprotein M | AWF93623.1 Flavivirus envelope glycoprotein M |
| AMQ48981.1 membrane glycoprotein M | AWF93622.1 Flavivirus envelope glycoprotein M |
| ANB66184.1 small envelope protein M | AWF93621.1 Flavivirus envelope glycoprotein M |
| ANB66183.1 small envelope protein M | AWF93620.1 Flavivirus envelope glycoprotein M |
| ANB66182.1 small envelope protein M | AUY62553.1 Flavivirus envelope glycoprotein M |
| ANC90428.1 small envelope protein M | ASR91936.1 Flavivirus envelope glycoprotein M |
| ANN83273.1 small envelope protein M | AYU74865.1 Flavivirus envelope glycoprotein M |
| ANN83272.1 small envelope protein M | YP_009227208.1 Flavivirus envelope glycoprotein M |
| ANW07476.1 small envelope protein M | YP_009227197.1 Flavivirus envelope glycoprotein M |
| ANW07475.1 small envelope protein M | UBI73854.1 Flavivirus polyprotein propeptide |
| AOC50654.1 small envelope protein M | AVW85817.1 small envelope protein M |
| AOC50653.1 small envelope protein M | AVW85821.1 small envelope protein M |
| AOC50652.1 small envelope protein M | AVW85812.1 small envelope protein M |
| AOO19564.1 membrane glycoprotein M | AVW85811.1 small envelope protein M |
| AQV07165.1 small envelope protein M | AVW85803.1 small envelope protein M |
| AQZ41956.1 small envelope protein M | AVW85858.1 small envelope protein M |
| AQZ41955.1 small envelope protein M | AVW85807.1 small envelope protein M |
| AQZ41954.1 small envelope protein M | UBI73854.1 protein pr |
| AQZ41953.1 small envelope protein M | AVW85817.1 Flavivirus envelope glycoprotein M |
| AQZ41952.1 small envelope protein M | AVW85821.1 Flavivirus envelope glycoprotein M |
| AQZ41951.1 small envelope protein M | AVW85812.1 Flavivirus envelope glycoprotein M |
| AQZ41950.1 small envelope protein M | AVW85811.1 Flavivirus envelope glycoprotein M |
| AQZ41949.1 small envelope protein M | AVW85803.1 Flavivirus envelope glycoprotein M |
| AQZ41948.1 small envelope protein M | AVW85858.1 Flavivirus envelope glycoprotein M |
| AQZ41947.1 small envelope protein M | AVW85807.1 Flavivirus envelope glycoprotein M |
| AWF93619.1 small envelope protein M | YP_009227197.1 membrane glycoprotein precursor M |
| AWF93618.1 small envelope protein M | AOR51315.1 Flavivirus polyprotein propeptide |
| AWF93617.1 small envelope protein M | ARM59240.1 Flavivirus polyprotein propeptide |
| AYI50275.1 small envelope protein M | AVW85863.1 Flavivirus polyprotein propeptide |
| AYI50274.1 small envelope protein M | AVW85861.1 Flavivirus polyprotein propeptide |
| AYI50273.1 small envelope protein M | AVW85859.1 Flavivirus polyprotein propeptide |
| AXQ40007.1 membrane glycoprotein M | AVW85857.1 Flavivirus polyprotein propeptide |
| AXQ40006.1 membrane glycoprotein M | AVW85856.1 Flavivirus polyprotein propeptide |
| AXQ40004.1 membrane glycoprotein M | AVW85855.1 Flavivirus polyprotein propeptide |
| AXQ40003.1 membrane glycoprotein M | AVW85853.1 Flavivirus polyprotein propeptide |
| AXQ40002.1 membrane glycoprotein M | AVW85852.1 Flavivirus polyprotein propeptide |
| AXQ40001.1 membrane glycoprotein M | AVW85851.1 Flavivirus polyprotein propeptide |
| AXQ40000.1 membrane glycoprotein M | AVW85848.1 Flavivirus polyprotein propeptide |
| AXQ39998.1 membrane glycoprotein M | AVW85847.1 Flavivirus polyprotein propeptide |
| AXQ39997.1 membrane glycoprotein M | AVW85845.1 Flavivirus polyprotein propeptide |
| AXQ39996.1 membrane glycoprotein M | AVW85843.1 Flavivirus polyprotein propeptide |
| QFZ95661.1 membrane glycoprotein M | AVW85837.1 Flavivirus polyprotein propeptide |
| QGA67056.1 membrane glycoprotein M | AVW85836.1 Flavivirus polyprotein propeptide |
| QGA67055.1 membrane glycoprotein M | AVW85835.1 Flavivirus polyprotein propeptide |
| QGA67054.1 membrane glycoprotein M | AVW85831.1 Flavivirus polyprotein propeptide |
| QGA67053.1 membrane glycoprotein M | AVW85830.1 Flavivirus polyprotein propeptide |
| QGA67052.1 membrane glycoprotein M | AVW85828.1 Flavivirus polyprotein propeptide |
| QGA67051.1 membrane glycoprotein M | AVW85827.1 Flavivirus polyprotein propeptide |
| QGA67050.1 membrane glycoprotein M | AVW85824.1 Flavivirus polyprotein propeptide |
| QGA72951.1 membrane glycoprotein M | AVW85823.1 Flavivirus polyprotein propeptide |
| QKM77299.1 membrane glycoprotein M | AVW85822.1 Flavivirus polyprotein propeptide |
| QKU35968.1 membrane glycoprotein M | AVW85821.1 Flavivirus polyprotein propeptide |
| QOF88708.1 membrane glycoprotein M | AVW85820.1 Flavivirus polyprotein propeptide |
| QKF93441.1 membrane glycoprotein M | AVW85819.1 Flavivirus polyprotein propeptide |
| QKF93440.1 membrane glycoprotein M | AVW85818.1 Flavivirus polyprotein propeptide |
| QKF93439.1 membrane glycoprotein M | AVW85817.1 Flavivirus polyprotein propeptide |
| QKF93438.1 membrane glycoprotein M | AVW85816.1 Flavivirus polyprotein propeptide |
| QKF93437.1 membrane glycoprotein M | AVW85815.1 Flavivirus polyprotein propeptide |
| QKF93436.1 membrane glycoprotein M | AVW85814.1 Flavivirus polyprotein propeptide |
| QKF93435.1 membrane glycoprotein M | AVW85813.1 Flavivirus polyprotein propeptide |
| QKF93434.1 membrane glycoprotein M | AVW85812.1 Flavivirus polyprotein propeptide |
| QKF93433.1 membrane glycoprotein M | AVW85811.1 Flavivirus polyprotein propeptide |
| QKF93430.1 membrane glycoprotein M | AVW85809.1 Flavivirus polyprotein propeptide |
| QKF93429.1 membrane glycoprotein M | AVW85808.1 Flavivirus polyprotein propeptide |
| QKF93428.1 membrane glycoprotein M | AVW85806.1 Flavivirus polyprotein propeptide |
| UBI73854.1 membrane glycoprotein M | AVW85805.1 Flavivirus polyprotein propeptide |
| UED15620.1 membrane glycoprotein M | AVW85804.1 Flavivirus polyprotein propeptide |
| UHI99867.1 membrane glycoprotein M | AVW85803.1 Flavivirus polyprotein propeptide |
| UHI99866.1 membrane glycoprotein M | AVW85802.1 Flavivirus polyprotein propeptide |
| UHI99865.1 membrane glycoprotein M | AVW85800.1 Flavivirus polyprotein propeptide |
| YP_009428568.1 membrane glycoprotein M | AWF93635.1 Flavivirus polyprotein propeptide |
| YP_009430299.1 membrane glycoprotein M | AWF93634.1 Flavivirus polyprotein propeptide |
| ALU33341.1 Flavivirus envelope glycoprotein M | AWF93633.1 Flavivirus polyprotein propeptide |
| AMC13911.1 Flavivirus envelope glycoprotein M | AWF93632.1 Flavivirus polyprotein propeptide |
| AMB37295.1 Flavivirus envelope glycoprotein M | AWF93629.1 Flavivirus polyprotein propeptide |
| AMD61710.1 Flavivirus envelope glycoprotein M | AWF93627.1 Flavivirus polyprotein propeptide |
| AMB18850.1 Flavivirus envelope glycoprotein M | AWF93626.1 Flavivirus polyprotein propeptide |
| AMD16557.1 Flavivirus envelope glycoprotein M | AWF93625.1 Flavivirus polyprotein propeptide |
| AMK79469.1 Flavivirus envelope glycoprotein M | AWF93624.1 Flavivirus polyprotein propeptide |
| AMM39806.1 Flavivirus envelope glycoprotein M | AWF93622.1 Flavivirus polyprotein propeptide |
| AMN14619.1 Flavivirus envelope glycoprotein M | AWF93621.1 Flavivirus polyprotein propeptide |
| AMQ34004.1 Flavivirus envelope glycoprotein M | AWF93620.1 Flavivirus polyprotein propeptide |
| AMQ48982.1 Flavivirus envelope glycoprotein M | AUY62553.1 Flavivirus polyprotein propeptide |
| AMQ48981.1 Flavivirus envelope glycoprotein M | ASR91936.1 Flavivirus polyprotein propeptide |
| AMX81919.1 Flavivirus envelope glycoprotein M | AYU74865.1 Flavivirus polyprotein propeptide |
| ANA12599.1 Flavivirus envelope glycoprotein M | BAP47441.1 Flavivirus polyprotein propeptide |
| ANB66182.1 Flavivirus envelope glycoprotein M | AMR39836.1 Flavivirus polyprotein propeptide |
| ANC90428.1 Flavivirus envelope glycoprotein M | AMR39835.1 Flavivirus polyprotein propeptide |
| ANF16414.1 Flavivirus envelope glycoprotein M | AMR39833.1 Flavivirus polyprotein propeptide |
| ANK57897.1 Flavivirus envelope glycoprotein M | ANW07477.1 Flavivirus polyprotein propeptide |
| AOG18296.1 Flavivirus envelope glycoprotein M | YP_009227197.1 Flavivirus polyprotein propeptide |
| AND01116.2 Flavivirus envelope glycoprotein M | ANC90427.1 protein pr |
| APC60216.2 Flavivirus envelope glycoprotein M | ANW07477.1 protein pr |
| APH11492.1 Flavivirus envelope glycoprotein M | ANW07474.2 protein pr |
| AMM43326.1 Flavivirus envelope glycoprotein M | ARM59240.1 protein pr |
| AMM43325.1 Flavivirus envelope glycoprotein M | AVW85863.1 protein pr |
| APO36913.1 Flavivirus envelope glycoprotein M | AVW85861.1 protein pr |
| AOO19564.1 Flavivirus envelope glycoprotein M | AVW85859.1 protein pr |
| AQV07165.1 Flavivirus envelope glycoprotein M | AVW85857.1 protein pr |
| AQW43707.1 Flavivirus envelope glycoprotein M | AVW85856.1 protein pr |
| AQZ41955.1 Flavivirus envelope glycoprotein M | AVW85855.1 protein pr |
| AQZ41953.1 Flavivirus envelope glycoprotein M | AVW85853.1 protein pr |
| AQZ41952.1 Flavivirus envelope glycoprotein M | AVW85852.1 protein pr |
| AQZ41949.1 Flavivirus envelope glycoprotein M | AVW85851.1 protein pr |
| AMX81917.1 Flavivirus envelope glycoprotein M | AVW85848.1 protein pr |
| AMX81916.1 Flavivirus envelope glycoprotein M | AVW85847.1 protein pr |
| AMX81915.1 Flavivirus envelope glycoprotein M | AVW85845.1 protein pr |
| ARM59239.1 Flavivirus envelope glycoprotein M | AVW85843.1 protein pr |
| AQS26698.1 Flavivirus envelope glycoprotein M | AVW85837.1 protein pr |
| AUI42330.1 Flavivirus envelope glycoprotein M | AVW85836.1 protein pr |
| AUI42329.1 Flavivirus envelope glycoprotein M | AVW85835.1 protein pr |
| BBC70847.1 Flavivirus envelope glycoprotein M | AVW85831.1 protein pr |
| AVZ47169.1 Flavivirus envelope glycoprotein M | AVW85830.1 protein pr |
| AWF93619.1 Flavivirus envelope glycoprotein M | AVW85828.1 protein pr |
| AWF93618.1 Flavivirus envelope glycoprotein M | AVW85827.1 protein pr |
| AWF93617.1 Flavivirus envelope glycoprotein M | AVW85824.1 protein pr |
| AWH65849.1 Flavivirus envelope glycoprotein M | AVW85823.1 protein pr |
| ARU07076.1 Flavivirus envelope glycoprotein M | AVW85822.1 protein pr |
| ARU07075.1 Flavivirus envelope glycoprotein M | AVW85821.1 protein pr |
| ARU07074.1 Flavivirus envelope glycoprotein M | AVW85820.1 protein pr |
| AVV62004.1 Flavivirus envelope glycoprotein M | AVW85819.1 protein pr |
| AVG19203.1 Flavivirus envelope glycoprotein M | AVW85818.1 protein pr |
| AVG19202.1 Flavivirus envelope glycoprotein M | AVW85817.1 protein pr |
| AUF35022.1 Flavivirus envelope glycoprotein M | AVW85816.1 protein pr |
| AUF35021.1 Flavivirus envelope glycoprotein M | AVW85815.1 protein pr |
| AXF50052.1 Flavivirus envelope glycoprotein M | AVW85814.1 protein pr |
| AXF50016.1 Flavivirus envelope glycoprotein M | AVW85813.1 protein pr |
| AXF50014.1 Flavivirus envelope glycoprotein M | AVW85812.1 protein pr |
| AYI50275.1 Flavivirus envelope glycoprotein M | AVW85811.1 protein pr |
| AYI50274.1 Flavivirus envelope glycoprotein M | AVW85809.1 protein pr |
| AYI50273.1 Flavivirus envelope glycoprotein M | AVW85808.1 protein pr |
| AVC63698.1 Flavivirus envelope glycoprotein M | AVW85806.1 protein pr |
| AVC63697.1 Flavivirus envelope glycoprotein M | AVW85805.1 protein pr |
| ATB53754.1 Flavivirus envelope glycoprotein M | AVW85804.1 protein pr |
| AYV74931.1 Flavivirus envelope glycoprotein M | AVW85803.1 protein pr |
| AYV74930.1 Flavivirus envelope glycoprotein M | AVW85802.1 protein pr |
| AYV74929.1 Flavivirus envelope glycoprotein M | AVW85800.1 protein pr |
| AYV74928.1 Flavivirus envelope glycoprotein M | AWF93635.1 protein pr |
| AYV74927.1 Flavivirus envelope glycoprotein M | AWF93634.1 protein pr |
| AYV74925.1 Flavivirus envelope glycoprotein M | AWF93633.1 protein pr |
| AYV74924.1 Flavivirus envelope glycoprotein M | AWF93632.1 protein pr |
| AYV74923.1 Flavivirus envelope glycoprotein M | AWF93629.1 protein pr |
| AYV74922.1 Flavivirus envelope glycoprotein M | AWF93627.1 protein pr |
| AYV74921.1 Flavivirus envelope glycoprotein M | AWF93626.1 protein pr |
| AYV74920.1 Flavivirus envelope glycoprotein M | AWF93625.1 protein pr |
| AYV74919.1 Flavivirus envelope glycoprotein M | AWF93624.1 protein pr |
| AYV74918.1 Flavivirus envelope glycoprotein M | AWF93622.1 protein pr |
| AYV74917.1 Flavivirus envelope glycoprotein M | AWF93621.1 protein pr |
| AYV74916.1 Flavivirus envelope glycoprotein M | AWF93620.1 protein pr |
| YP_009428568.1 Flavivirus envelope glycoprotein M | YP_002790881.1 protein pr |
| YP_009430297.1 Flavivirus envelope glycoprotein M | AAV34151.1 Flavivirus polyprotein propeptide |
| AXQ40002.1 membrane glycoprotein precursor M | AMR39832.1 Flavivirus polyprotein propeptide |
| AXQ40003.1 membrane glycoprotein precursor M | ANW07474.2 Flavivirus polyprotein propeptide |
| QGA72951.1 membrane glycoprotein precursor M | AUY62552.1 Flavivirus polyprotein propeptide |
| QKF93440.1 membrane glycoprotein precursor M | QDH45910.1 Flavivirus polyprotein propeptide |
| QKF93438.1 membrane glycoprotein precursor M | QPC45464.1 Flavivirus polyprotein propeptide |
| QKF93437.1 membrane glycoprotein precursor M | YP_002790881.1 Flavivirus polyprotein propeptide |
| QKF93436.1 membrane glycoprotein precursor M | AVW85862.1 Flavivirus polyprotein propeptide |
| QKF93435.1 membrane glycoprotein precursor M | AVW85860.1 Flavivirus polyprotein propeptide |
| QKF93434.1 membrane glycoprotein precursor M | AVW85858.1 Flavivirus polyprotein propeptide |
| QKF93433.1 membrane glycoprotein precursor M | AVW85854.1 Flavivirus polyprotein propeptide |
| QKF93430.1 membrane glycoprotein precursor M | AVW85850.1 Flavivirus polyprotein propeptide |
| QKF93429.1 membrane glycoprotein precursor M | AVW85849.1 Flavivirus polyprotein propeptide |
| QKF93428.1 membrane glycoprotein precursor M | AVW85846.1 Flavivirus polyprotein propeptide |
| QKM77299.1 membrane glycoprotein precursor M | AVW85844.1 Flavivirus polyprotein propeptide |
| AXQ39996.1 membrane glycoprotein precursor M | AVW85842.1 Flavivirus polyprotein propeptide |
| AXQ39999.1 membrane glycoprotein precursor M | AVW85841.1 Flavivirus polyprotein propeptide |
| QOF88708.1 membrane glycoprotein precursor M | AVW85840.1 Flavivirus polyprotein propeptide |
| QKF93441.1 membrane glycoprotein precursor M | AVW85839.1 Flavivirus polyprotein propeptide |
| QKF93439.1 membrane glycoprotein precursor M | AVW85838.1 Flavivirus polyprotein propeptide |
| AXQ40002.1 protein pr | AVW85834.1 Flavivirus polyprotein propeptide |
| AXQ40003.1 protein pr | AVW85833.1 Flavivirus polyprotein propeptide |
| AOC50652.1 protein pr | AVW85832.1 Flavivirus polyprotein propeptide |
| QGA72951.1 protein pr | AVW85829.1 Flavivirus polyprotein propeptide |
| QKF93440.1 protein pr | AVW85826.1 Flavivirus polyprotein propeptide |
| QKF93438.1 protein pr | AVW85825.1 Flavivirus polyprotein propeptide |
| QKF93437.1 protein pr | AVW85810.1 Flavivirus polyprotein propeptide |
| QKF93436.1 protein pr | AVW85807.1 Flavivirus polyprotein propeptide |
| QKF93435.1 protein pr | AVW85801.1 Flavivirus polyprotein propeptide |
| QKF93434.1 protein pr | AWF93631.1 Flavivirus polyprotein propeptide |
| QKF93433.1 protein pr | AWF93630.1 Flavivirus polyprotein propeptide |
| QKF93432.1 protein pr | AWF93628.1 Flavivirus polyprotein propeptide |
| QKF93431.1 protein pr | AWF93623.1 Flavivirus polyprotein propeptide |
| QKF93430.1 protein pr | AVW85862.1 protein pr |
| QKF93429.1 protein pr | AVW85860.1 protein pr |
| QKF93428.1 protein pr | AVW85858.1 protein pr |
| ANN83272.1 protein pr | AVW85854.1 protein pr |
| QKM77301.1 protein pr | AVW85850.1 protein pr |
| QKM77300.1 protein pr | AVW85849.1 protein pr |
| QKM77299.1 protein pr | AVW85846.1 protein pr |
| QKM77298.1 protein pr | AVW85844.1 protein pr |
| AXQ39996.1 protein pr | AVW85842.1 protein pr |
| QOF88708.1 protein pr | AVW85841.1 protein pr |
| QKF93441.1 protein pr | AVW85840.1 protein pr |
| QKF93439.1 protein pr | AVW85839.1 protein pr |
| AUI42330.1 Flavivirus polyprotein propeptide | AVW85838.1 protein pr |
| AUI42329.1 Flavivirus polyprotein propeptide | AVW85834.1 protein pr |
| AMD61710.1 Flavivirus polyprotein propeptide | AVW85833.1 protein pr |
| AMX81917.1 Flavivirus polyprotein propeptide | AVW85832.1 protein pr |
| AMX81916.1 Flavivirus polyprotein propeptide | AVW85829.1 protein pr |
| AQS26698.1 Flavivirus polyprotein propeptide | AVW85826.1 protein pr |
| BBC70847.1 Flavivirus polyprotein propeptide | AVW85825.1 protein pr |
| AVV62004.1 Flavivirus polyprotein propeptide | AVW85810.1 protein pr |
| AVG19203.1 Flavivirus polyprotein propeptide | AVW85807.1 protein pr |
| AVG19202.1 Flavivirus polyprotein propeptide | AVW85801.1 protein pr |
| AUF35022.1 Flavivirus polyprotein propeptide | AWF93631.1 protein pr |
| AUF35021.1 Flavivirus polyprotein propeptide | AWF93630.1 protein pr |
| AVC63698.1 Flavivirus polyprotein propeptide | AWF93628.1 protein pr |
| AVC63697.1 Flavivirus polyprotein propeptide | AWF93623.1 protein pr |

### EXAMPLES

### Example 1: In Vitro Transcription and RNA Transfection of a Construct Carrying Nluc and eGFP Construct in Vero Cell

A construct of a plasmid carrying sequences of RNA polymerase promoter, Hammer head ribozyme, 5′ UTRs, C-partial, 3′UTRs, HDV ribozyme and encoding GFP and Nluc luciferase was designed as shown generally in FIG. 5. See also Table 2.

RNA produced through in vitro transcription of linear plasmid was transfected in Vero cells. The transient transfection was carried out with Lipofectamine™ MessengerMAX™ (Thermo Scientific™ catalog #LMRNA001), after 24 hours, a fresh culture medium was added to the cells.

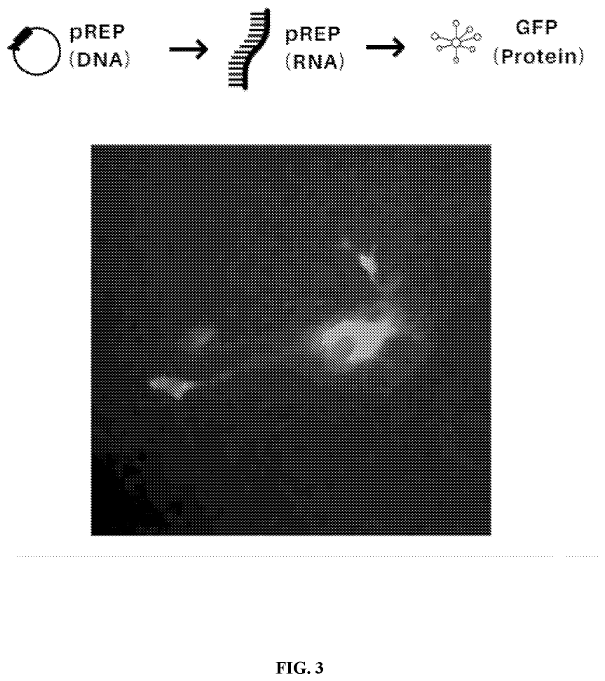

After 48 hours, the transfected Vero cells were imaged alive. GFP signal was detected with fluorescent microscopy (EVOS M5000 Imaging System, ThermoFisher™). As shown in FIG. 3, GFP signal was strong in a positively transfected Vero cell after GFP expression by a cytoplasmic RNA Replicon transfection.

In another experiment, Nluc luciferase activity was determined after capless RNA transfection.

In vitro transcription was carried out for the plasmid with Nluc activity. Using a kit MEGAscript (catalog #AM1330, Life Technologies), in vitro transcription was performed according to the user manuals. Briefly, 1 μg of linearized plasmid was used, and the reaction performed for 2 hours. After lithium chloride and isopropanol precipitation, RNA was resuspended in water and stored at −80° C.

The transcription products were pre-treated with Lipofectamine™ MessengerMAX™ (Thermo Scientific™ catalog #LMRNA001) under fabricant instruction conditions. Briefly, the complex of transcription products and Lipofectamine™ MessengerMAX™ were incubated with Vero cells (obtained from ATCC) and after 24 hours, a fresh culture medium was added to the cells.

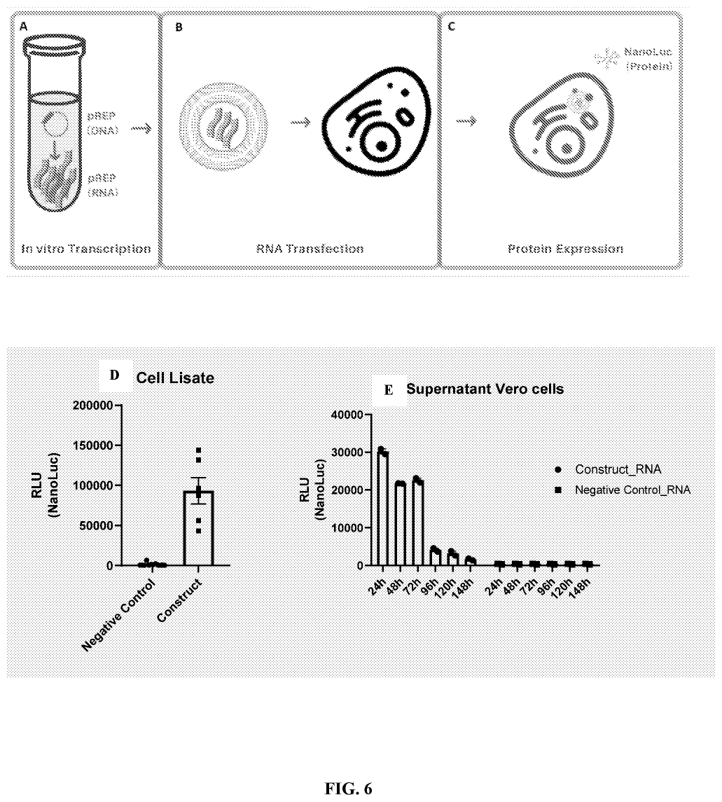

Supernatant and pellet of transfected cells were resuspended in 1 volume of NanoGlo detection reagent (Promega) previously diluted 50× in water. Luminescence was quantified for 0.5 seconds in a GloMax luminometer (Promega). As shown in FIG. 6D and FIG. 6E, high Nluc activities were observed in both the cell lysate and the supernatant of the Vero cells, demonstrating a capless RNA with ZIKV 5′UTR and 3′UTR is recognized by human translation machinery.

TABLE 2
FIG. 5 sequences.

| Component | |
|---|---|
| (SEQ ID NO) | Sequence |
| Eukaryotic | gacattgattattgactagttattaatagtaatcaattacggggtcattagttcatagcccatatatggagttcc |
| enhancer (2896) | gcgttacataacttacggtaaatggcccgcctggctgaccgcccaacgacccccgcccattgacgtcaat |
| | aatgacgtatgttcccatagtaacgccaatagggactttccattgacgtcaatgggtggactatttacggtaa |
| | actgcccacttggcagtacatcaagtgtatcatatgccaagtacgccccctattgacgtcaatgacggtaa |
| | atggcccgcctggcattatgcccagtacatgaccttatgggactttcctacttggcagtacatctacgtatta |
| | gtcatcgctattaccatg |
| Eukaryotic | gtgatgcggttttggcagtacatcaatgggcgtggatagcggtttgactcacggggatttccaagtctcca |
| promoter (2897) | ccccattgacgtcaatgggagtttgttttggcaccaaaatcaacgggactttccaaaatgtcgtaacaactc |
| | cgccccattgacgcaaatgggcggtaggcgtgtacggtgggaggtctatataagcagagct |
| RNA | taatacgactcactatagg |
| polymerase | |
| promoter (2898) | |
| Hammer head | gttaacacaactctgatgagtccgtgaggacgaaacgagtaagctcgtc |
| (2899) | |
| 5′ zika UTR | agttgttactgttgctgactcagactgcgacagttcgagtttgaagcgaaagctagcaacagtatcaacag |
| (2900) | gttttatttggatttggaaacgagagtttctggtc |
| C Zika (2901) | atgaaaaacccaaaaaagaaatccggaggattccggattgtcaatatgctaaaacgcggagtagcccgt |
| | gtgagcccctttgggggcttg |
| EGFP (2902) | atggtgagcaagggcgaggagctgttcaccggggtggtgcccatcctggtcgagctggacggcgacgt |
| | aaacggccacaagttcagcgtgtccggcgagggcgagggcgatgccacctacggcaagctgaccctg |
| | aagttcatctgcaccaccggcaagctgcccgtgccctggcccaccctcgtgaccaccctgacctacggc |
| | gtgcagtgcttcagccgctaccccgaccacatgaagcagcacgacttcttcaagtccgccatgcccgaa |
| | ggctacgtccaggagcgcaccatcttcttcaaggacgacggcaactacaagacccgcgccgaggtgaa |
| | gttcgagggcgacaccctggtgaaccgcatcgagctgaagggcatcgacttcaaggaggacggcaac |
| | atcctggggcacaagctggagtacaactacaacagccacaacgtctatatcatggccgacaagcagaag |
| | aacggcatcaaggtgaacttcaagatccgccacaacatcgaggacggcagcgtgcagctcgccgacca |
| | ctaccagcagaacacccccatcggcgacggccccgtgctgctgcccgacaaccactacctgagcaccc |
| | agtccgccctgagcaaagaccccaacgagaagcgcgatcacatggtcctgctggagttcgtgaccgcc |
| | gccgggatcactctcggcatggacgagctgtacaag |
| Nluc (2903) | gtcttcacactcgaagatttcgttggggactggcgacagacagccggctacaacctggaccaagtccttg |
| | aacagggaggtgtgtccagtttgtttcagaatctcggggtgtccgtaactccgatccaaaggattgtcctga |
| | gcggtgaaaatgggctgaagatcgacatccatgtcatcatcccgtatgaaggtctgagcggcgaccaaat |
| | gggccagatcgaaaaaatttttaaggtggtgtaccctgtggatgatcatcactttaaggtgatcctgcactat |
| | ggcacactggtaatcgacggggttacgccgaacatgatcgactatttcggacggccgtatgaaggcatc |
| | gccgtgttcgacggcaaaaagatcactgtaacagggaccctgtggaacggcaacaaaattatcgacgag |
| | cgcctgatcaaccccgacggctccctgctgttccgagtaaccatcaacggagtgaccggctggcggctg |
| | tgcgaacgcattctggcg |
| 6xHis (2904) | catcatcaccatcaccac |
| 3′ zika UTR | gcaccaatcttaatgttgtcaggcctgctagtcagccacagcttggggaaagctgtgcagcctgtgaccc |
| (2905) | ccccaggagaagctgggaaaccaagcctatagtcaggccgagaacgccatggcacggaagaagccat |
| | gctgcctgtgagcccctcagaggacactgagtcaaaaaaccccacgcgcttggaggcgcaggatggg |
| | aaaagaaggtggcgaccttccccacccttcaatctggggcctgaactggagatcagctgtggatctccag |
| | aagagggactagtggttagaggagaccccccggaaaacgcaaaacagcatattgacgctgggaaaga |
| | ccagagactccatgagtttccaccacgctggccgccaggcacaga |
| HDV ribozyme | tcgccgaatagcggcggccggtgtggggaaatccatgggtctggccggcatggtcccagcctcctcgct |
| (2906) | ggcgccggctgggcaacatgcttcggcatggcgaatgggac |
| bGH poly(A) | ctgtgccttctagttgccagccatctgttgtttgcccctcccccgtgccttccttgaccctggaaggtgccac |
| signal (2907) | tcccactgtcctttcctaataaaatgaggaaattgcatcgcattgtctgagtaggtgtcattctattctggggg |
| | gtggggggggcaggacagcaagggggaggattgggaagacaatagcaggcatgctggggatgcgg |
| | tgggctctatgg |

### Example 2: Recombinant Zika Virus Construction

A construct of a plasmid carrying sequences of Eukaryotic promoter, Hammer head ribozyme, 5′ UTRs, C-partial, 3′UTRs, HDV ribozyme and encoding GFP and Nluc luciferase was designed.

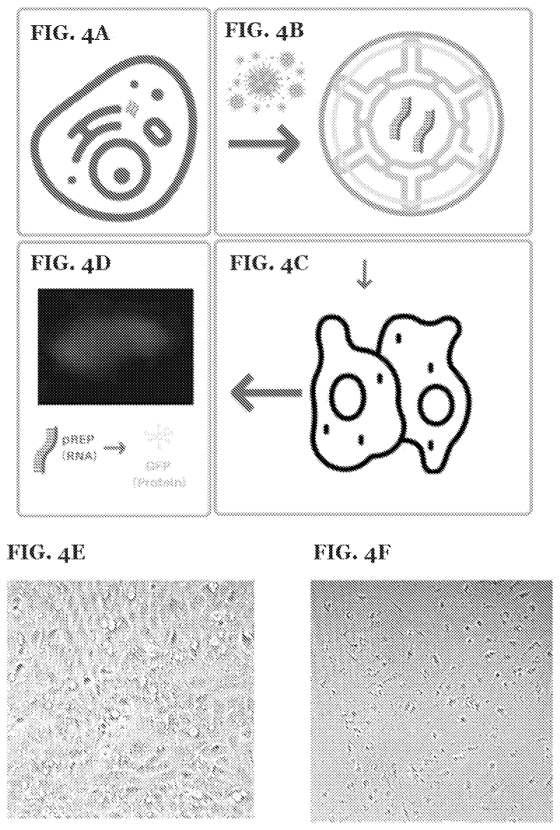

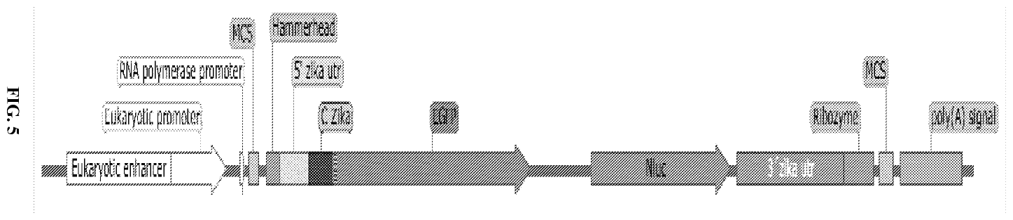

Vero cells were obtained from ATCC and were raised under DMEM high culture medium. After reaching 80% confluency, DNA linear plasmid was transfected. The transfection was carried out with lipofectamine P3000 (Thermo Scientific™ catalog #L3000015). After 24 hours, a fresh culture medium was added to the cells. To select stable expression of pREP, zeomicyn [1 mg/mL] was added to the medium. Therefore, Vero cells expressing the pREP construct (Vero_pREP) were obtained (see FIG. 4A).

As illustrated in FIG. 4B, Vero_pREP were infected with wild-type zika virus from the Brazilian strain isolated from a clinical case in Brazil, provided by the Evandro Chagas Institute in Belém, Pará, Brazil. The Vero_pREP cells were infected with a virus concentration of MOI 2. After 30 minutes of incubation period, the culture infection buffer was replaced with fresh media.

After 24, 48, and 72 hours, the recombinant Zika virus containing the viral vector (ZIKVV) was generated from the transfected Vero_pREP and collected from the media.

The culture supernatants containing ZIKVV were storage in −80° C.

Separately, medulloblastoma tumor cell line, Daoy, obtained from ATCC™ was cultured in a 6 well plate under ATCC culture conditions.

The medulloblastoma tumor cell line was infected with the viral vector generated from the transfected Vero_pREP (see FIG. 4C).

After 24 hours, the infected medulloblastoma tumor cell line (Daoy) was imaged alive. GFP signal was detected with fluorescent microscopy (EVOS M5000 Imaging System, ThermoFisher™). As shown in FIG. 4D, GFP fluorescence in the medulloblastoma cell line after the infection of the recombinant Zika Virus was observed.

FIG. 4E shows medulloblastoma cell line 48 hours pos-infection with wild-type Brazilian zika virus. FIG. 4F shows medulloblastoma cell line 48 hours pos-infection with recombinant zika virus with a low amount of adherent cell and cell viability, showing the enhanced oncolytic effect of recombinant zika virus when compared with the wild-type zika virus.

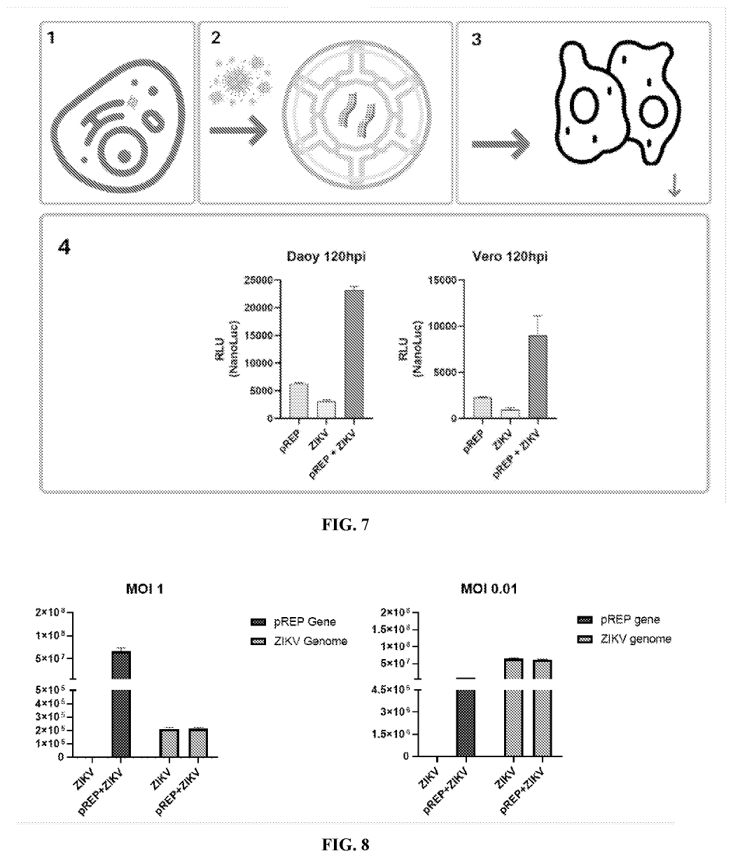

In another experiment, the vector of FIG. 5 was in vitro transcribed and transfected in Vero cells (step 1 of FIG. 7), followed by ZIKV infection (step 2 of FIG. 7). 120 hours after ZIKV infection, the culture supernatant, containing the viral vector (ZIKVV) produced by Vero cells, was collected, and concentrated with PEG centrifugation. To test the viral vector functionality, the concentrated supernatant was contacted with the target cells (Vero and Daoy cell lines) to confirm the Nanoluc delivery by ZIKVV (step 3 of FIG. 7). The graphs in step 4 of FIG. 7 show Nanoluc quantification in Daoy and Vero cells after contact with Viral vector (pREP+ZIKV), compared to pREP controls (submitted only to pREP transfection) and ZIKV (submitted only to ZIKV infection). A higher level of NanoLuc expression is shown with pREP+ZIKV when compared with the control groups. These results show that ZIKVV delivered NanoLuc to the target cells and confirmed the expression of the reporter gene in target cells.

### Example 3: Expression of Nluc or eGFP after Infection of a Recombinant Zika Virus

A medulloblastoma tumor cell line is infected with recombinant zika viruses, which are similarly prepared as Example 2. The viability rate and the in vitro transduction rate are measured. Briefly, the medulloblastoma cell lines are cultures in a 96 well plate and infected with the recombinant zika viruses in MOI 1. Cell viability and Nluc expression are measured using the CellTiter-Glo¬Æ 2.0 Cell Viability Assay (Promega—cat. G9242) and Nano-Glo¬Æ Luciferase Assay (Promega—cat. N1110), respectively, following fabricant instructions.

Separately, the full genome of the infected cell line is sequenced, and probably of insertional mutagenesis is evaluated.

Similarly, the recombinant zika viruses are injected into wild-type BalBc mice. Over several weeks following the injection, weight, activity, inflammation level near the injection site and systematic inflammation markers in the blood, possible morbidity rate are monitored. In vivo transduction rate, and the effectiveness of crossing blood brain barrier are also evaluated.

Primary T cells are collected from peripheral blood mononuclear cells (PBMCs) of healthy donor patients. After transducing the primary T cells with recombinant zika virus which are similarly prepared as Examples 1 or 2, the viability rate of the primary T cells after infection and the in-vitro transduction rate are measured.

### Example 4: Treatment of Alzheimer Disease with Recombinant Zika Viruses or Zika-Virus-Like Particles

An alzheimer mouse model is established.

Recombinant zika virus or zika-virus-like particles carrying a polynucleotide encoding a brain-derived neurotrophin factor (BDNF) are similarly prepared as Examples 1 or 2.

One month after the infection of the recombinant zika virus or the zika-virus-like particles with different titers, Morris water maze test and other behavior tests on memory formation and retention are tested on mice with BDNF treatment and mock treatment.

### Example 5: RNA Quantification from a Viral Vector Produced

RNA quantification was performed from different viral vector batches produced.

RT-PCT was performed with specific primers for ZIKV genome (gene ZIKV) and pREP (gene pREP), to compare the RNA amount of ZIKV genome and pREP after pREP transfection and ZIKV infection in Vero cells (FIG. 7, steps 1-3). When pREP RNA copies were compared to ZIKV genome RNA copies in one batch that was produced after ZIKV infection with MOI 1, the difference is ten times larger (FIG. 8). A second batch made following ZIKV infection with MOI 0.1 displays a pREP RNA copy number that is comparable to the ZIKV genome copy number (FIG. 8). These findings demonstrate that, at high MOIs, ZIKVV has 10 times as many copies of the pREP gene as the ZIKV genome, with the pREP gene being amplified by viral machinery.

### Example 5: Viral Vector In Vitro Tropism

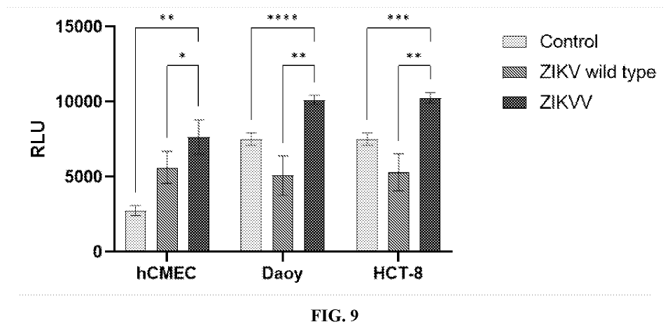

The in vitro tropism of ZIKVV (ZIKV genome and pREP RNA) was evaluated. Entry of ZIKVV, ZIKV wild type, or control (pREP mRNA) was tested in cells susceptible to wild-type ZIKV (hCMEC and Daoy) and not susceptible to wild-type ZIKV (HCT-8). Cell lines were combined with the wild-type virus (ZIKV wild type), the viral vector (ZIKVV), and pREP mRNA (control). If the viral vector retains the ability to enter cell lines as does wild-type ZIKV, the mRNA with reporter gene that codes the enzyme NanoLuc Luciferase will be delivered and translated, being detected through luminescence assay. ZIKVV could deliver the gene-of-interest to the cell lines, including the not susceptible HCT-8 cell lines, indicating a higher viral vector spectrum of action, not limited to neural and stem-like cells that are susceptible to wild-type ZIKV (FIG. 9).

### Example 6: Evaluation of ZIKV for Delivery of Various Genes of Interest

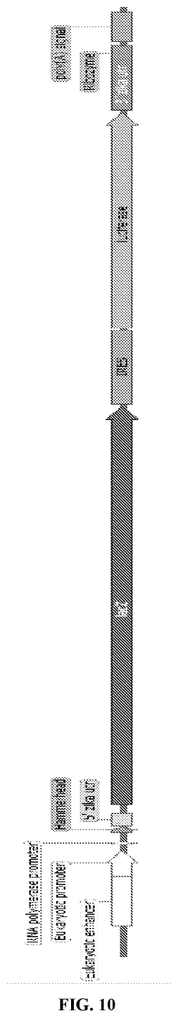

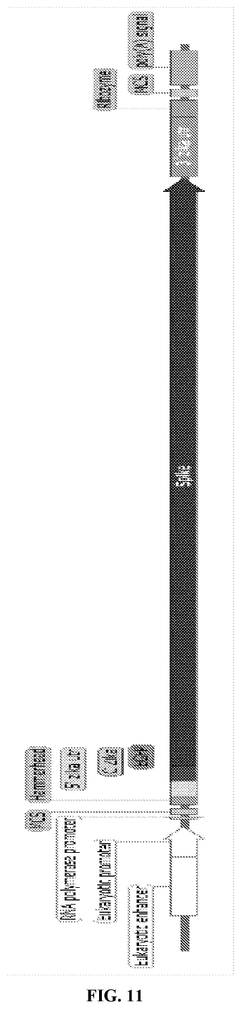

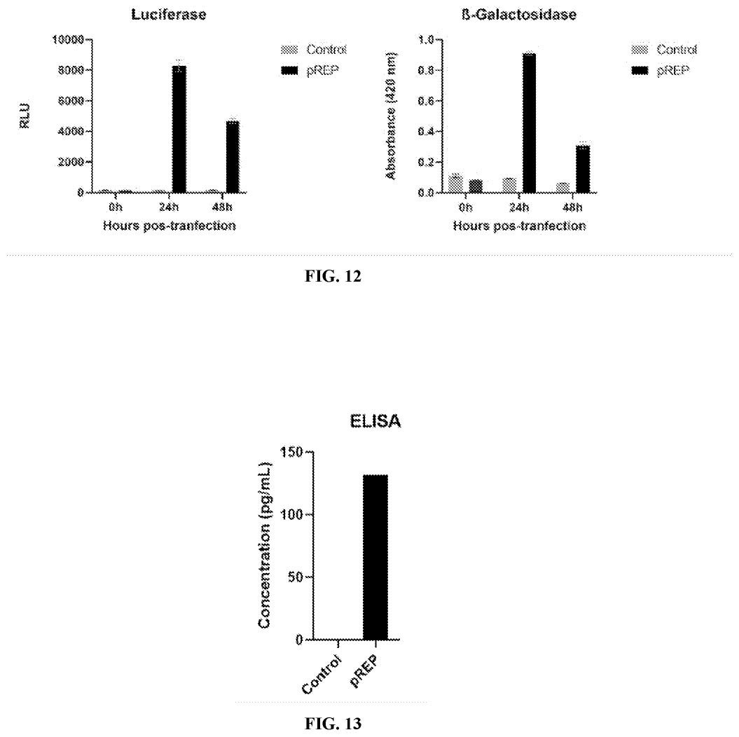

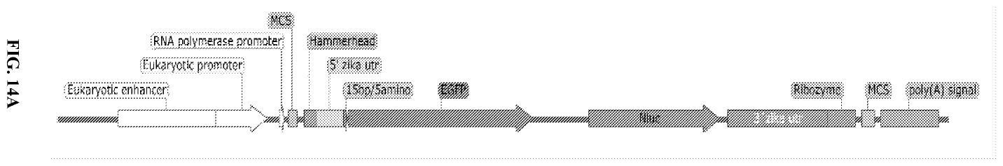

mRNA prepared from zika vectors having various genes of interest were evaluated. Example vectors are shown in FIGS. 10 (pREP-Luc/Gal) and 11 (pREP-Spike), where the genes in FIG. 10 are polycistronic luciferase and beta-galactosidase, and the gene in FIG. 11 encodes SARS-CoV-2 spike protein. Vero cells were transfected with the pREP-Luc/Gal, and expression of both reporter genes show the capability of the pREP construct to deliver and promote the expression of multiple genes with up to 6 Kb (FIG. 12). Furthermore, the SARS-CoV-2 Spike coding sequence was inserted into pREP plasmid to evaluate the strength of expression by ELISA (FIG. 13), showing that the pREP construct can deliver and promote the expression of other gene-of-interests and confirming use of pREP mRNA as vaccine product.

### Example 7: Evaluation of Zika Capsid Coding Sequence

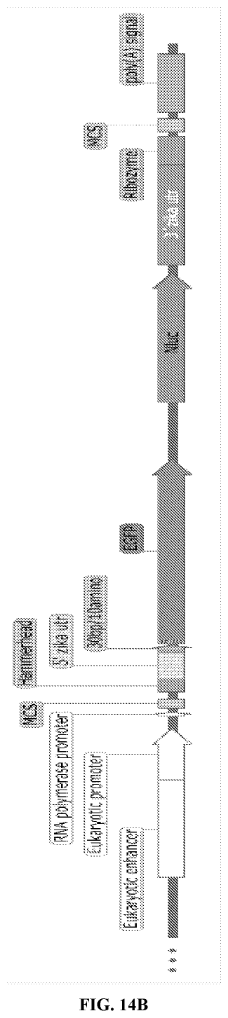

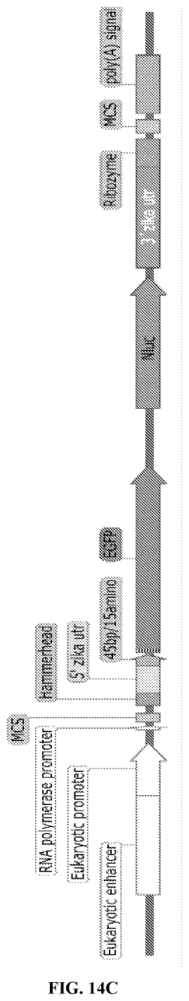

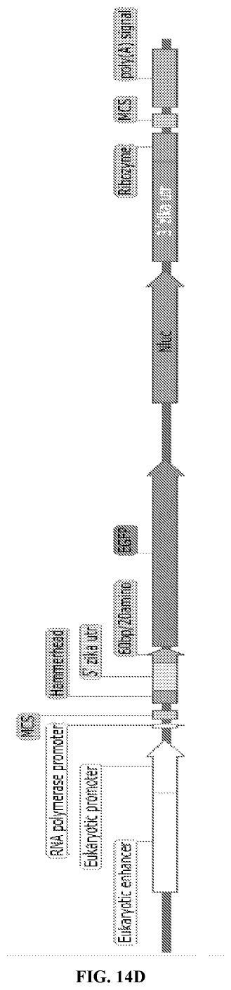

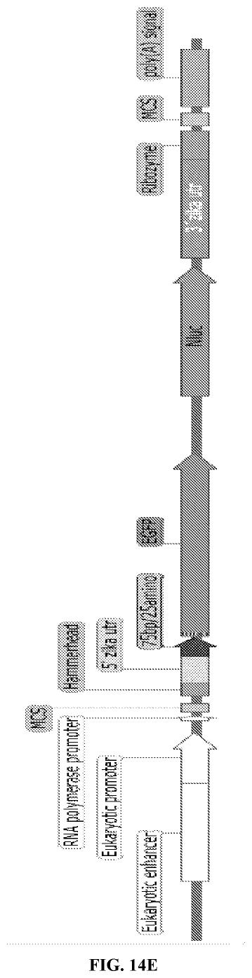

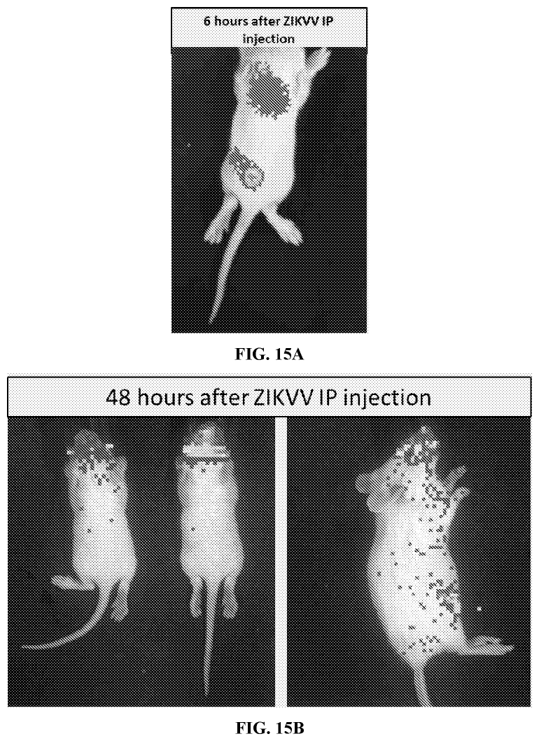

The evaluation of Zika capsid sequence necessary for pREP (FIG. 5) encapsulation was carried out with five constructs of a plasmid (FIGS. 14A-14E), carrying sequences of Eukaryotic promoter, RNA polymerase promoter, Hammerhead ribozyme, 5′ UTRs, C-partial (15, 30, 45, 60 or 75 bp), 3′UTRs, HDV ribozyme, and encoding GFP and Nluc luciferase was designed. See Table 2, and Table 3 below for the C-partial sequences.

Using a kit MEGAscript (catalog #AM1330, Life Technologies), in vitro transcription was performed for the vectors according to the user manuals.

Vero cells were obtained from ATCC and were raised under DMEM high culture medium. After reaching 80% confluency, RNA (FIGS. 14A-14E) and DNA (FIG. 14A and FIG. 14C) plasmid was transfected. The DNA transfection was carried out with lipofectamine P3000 (Thermo Scientific™ catalog #L3000015) and RNA transfection was carried out with Lipofectamine™ MessengerMAX™ (Thermo Scientific™ catalog #LMRNA001) under fabricant instruction conditions.

After 24 hours, Vero cells were infected with the wild-type zika virus. The Vero cells were infected with a virus concentration of MOI 0.01. After 30 minutes of the incubation period, the culture infection buffer was replaced with fresh media.

After 120 hours after ZIKV infection, the culture supernatant, containing the viral vectors (ZIKVV) produced by Vero cells, was collected and concentrated with PEG centrifugation.

RNA quantification was performed from different viral vectors generated (FIGS. 14A-14E).

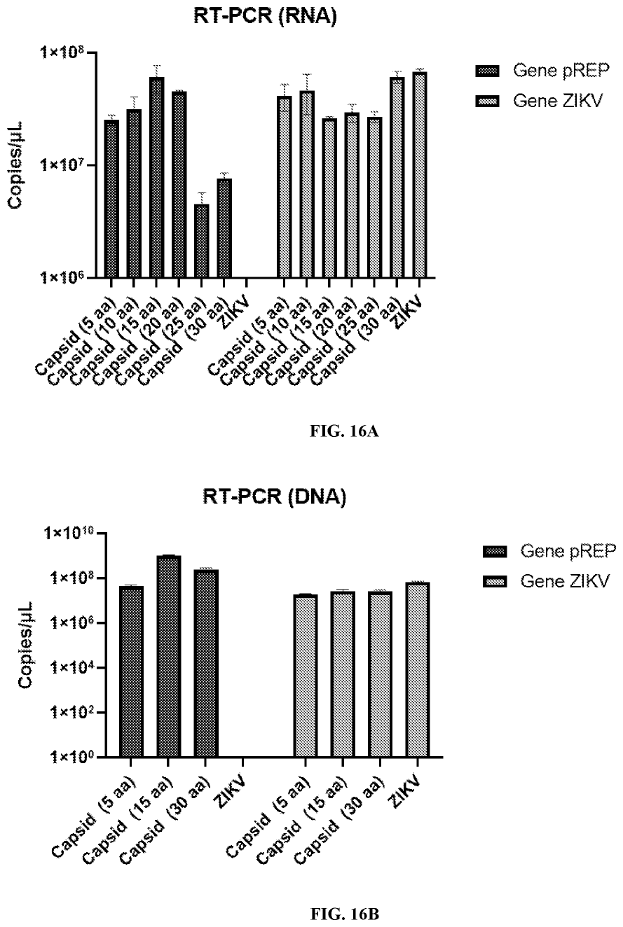

RT-PCT was performed with specific primers for the ZIKV genome (ZIKV gene) and pREP (pREP gene) to detect the RNA amount of the ZIKV genome and pREP after pREP transfection and ZIKV infection in Vero cells. pREP RNA copies were quantified from all pREP with different-sized capsid sequences (FIGS. 16A-16B). FIG. 16A shows pREP RNA encapsulation in all plasmids tested (C-partial with 15, 30, 45, 60 and 75 bp). The pREP RNA encapsulation was higher in plasmids with C-partial with 15, 30, 45, and 60 bp, indicating that ZIKVV carrying pREP RNA with Capsid with 20 or fewer amino acids is more efficiently encapsulated into the viral vector. FIG. 16B shows that the encapsulation derived from DNA transfection was efficient and the plasmid with C-partial 45 bp had a higher amount of pREP RNA.

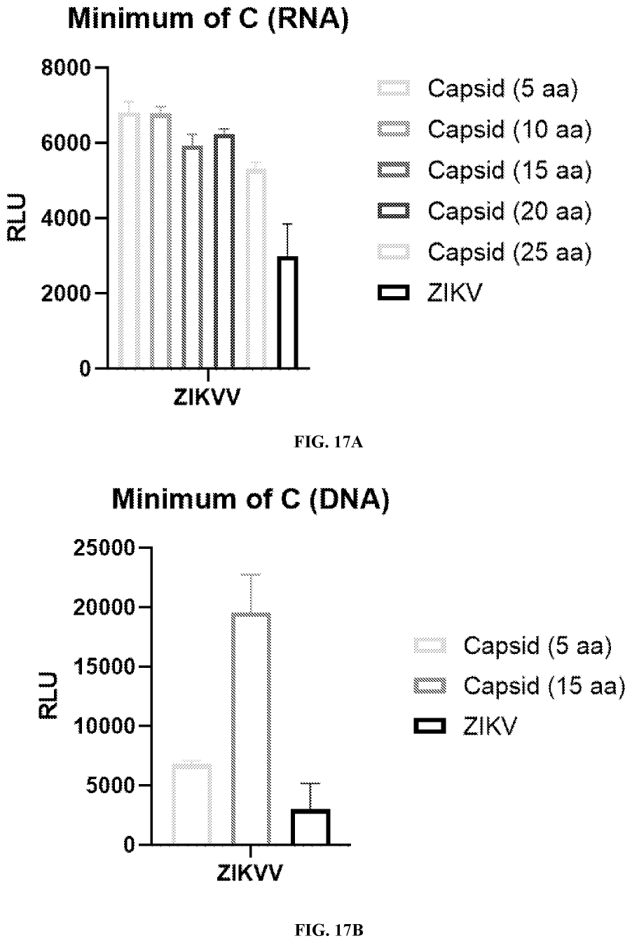

To test whether the viral vector encapsulates pREP with different size capsid sequences (FIGS. 14A-14E), the concentrated supernatant was contacted with Vero cell lines to confirm Nanoluc delivery by ZIKVV (step 3 of FIG. 7). The graphs in (FIG. 17A and FIG. 17B) shows Nanoluc quantification in Vero cells after contact with viral vector (pREP+ZIKV) carrying 5, 10, 15, 20 and 25 amino acids, derived from both RNA (FIG. 17A) and DNA (FIG. 17B) transfection, compared to ZIKV (only in ZIKV infection). A higher level of NanoLuc expression is demonstrated in viral vectors of all capsid sequence sizes compared to the control group. These results indicate that ZIKVV is able to encapsulate pREP RNA with C-partial (15, 30, 45, 60 and 75 bp) and delivery NanoLuc, confirmed by the expression of the reporter gene in the target cells.

TABLE 3
Zika capsid sequence for pREP (FIGS. 14A-14E) encapsulation.

| Component | Sequence (SEQ ID NO) |
|---|---|
| C Zika (15 bp/5amino) | atgaaaaacccaaaa (2908) |
| C Zika (30 bp/10amino) | atgaaaaacccaaaaaagaaatccggagga (2909) |
| C Zika (45 bp/15amino) | atgaaaaacccaaaaaagaaatccggaggattccggattgtcaat (2910) |
| C Zika (60 bp/20amino) | atgaaaaacccaaaaaagaaatccggaggattccggattgtcaatatgctaaaa |
| | cgcgga (2911) |
| C Zika (75 bp/25amino) | atgaaaaacccaaaaaagaaatccggaggattccggattgtcaatatgctaaaa |
| | cgcggagtagcccgtgtgagc (2912) |
| C Zika (15 bp/5amino) | MKNPK (2913) |
| C Zika (30 bp/10amino) | MKNPKKKSGG (2914) |
| C Zika (45 bp/15amino) | MKNPKKKSGGFRIVN (2915) |
| C Zika (60 bp/20amino) | MKNPKKKSGGFRIVNMLKRG (2916) |
| C Zika (75 bp/25amino) | MKNPKKKSGGFRIVNMLKRGVARVS (2917) |

### Example 8: In Vivo ZIKVV Biodistribution and Tropism

A feature of the present technology is the potential to generate a viral vector with CNS and ocular tropism. To track the in vivo biodistribution and tropism of ZIKVV, the bioluminescence imaging system was used based on the Nanoluc report gene inserted in the vector gene-of-interest. ZIKVV was systemically administered in Balb/C male mice, with age of 3 weeks, by intraperitoneal injections of ZIKVV produced as in Example 2 and carrying pREP RNA (FIG. 5). 50 ul of ZIKVV 10 times diluted was administered, with a total of 10\hat{ }4 particles. For bioluminescence imaging, the mice received the Nanoluc substrate (Promega—50× diluted) by intraperitoneal administration, in a volume of 50 ul, immediately before image acquisition at IVIS-Spectrum equipment (Perkin-Elmer). A strong signal of Nanoluc was observed in the brains and eye regions, 48 hours after ZIKVV injection in mice. This result confirms the ZIKVV in vivo tropism to eyes and brain, and vector selectively to deliver and transduce the gene-of-interest on these organs after systemic vector administration.

The preceding merely illustrates the principles of this disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of this disclosure and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

1. A method of delivering a polynucleotide to a target cell in a subject, the method comprising administering to the subject a zika virus or a zika-virus-like particle comprising the polynucleotide, wherein the polynucleotide is not a zika virus polynucleotide, and the target cell is a cell of the central nervous system.

2. A method of delivering a polynucleotide to a subject, the method comprising administering to the subject a zika virus or a zika-virus-like particle comprising the polynucleotide, wherein the polynucleotide is not a zika virus polynucleotide, and the zika virus or zika-virus-like particle does not comprise a replicating zika virus.

3. A method of delivering a polynucleotide to a subject, the method comprising administering to the subject a zika virus or a zika-virus-like particle comprising the polynucleotide, wherein the polynucleotide is not a zika virus polynucleotide, and the polynucleotide has a length up to about 10000 nucleotides.

4. The method of any one of claims 1-3, wherein the subject has a disease or disorder of the central nervous system.

5. The method of any one of claims 1-4, wherein the polynucleotide has a length greater than about 6000 nucleotides.

6. The method of any one of claims 1-5, wherein the subject is a human or non-human animal.

7. A zika virus or zika-virus-like particle comprising a polynucleotide exogenous to zika virus having a length of about 6,000 nucleotides to about 10,000 nucleotides, optionally wherein the polynucleotide does not encode a full-length viral structural protein or a full-length viral nonstructural protein.

8. The method of any one of claims 1-6 or the zika virus or zika-virus-like particle of claim 7, wherein the zika virus or zika-virus-like particle is produced from a nucleic acid comprising the polynucleotide.

9. The method or zika virus or zika-virus-like particle of claim 8, wherein the nucleic acid further comprises a zika virus 5′ UTR and a zika virus 3′ UTR.

10. The method or zika virus or zika-virus-like particle of claim 9, wherein the zika virus 5′ UTR is at least 90% homologous or identical to a zika virus 5′ UTR of Table 2, Table 1A, Table 1C, or Table 1H.

11. The method or zika virus or zika-virus-like particle of claim 9 or claim 10, wherein the zika virus 3′ UTR is at least 90% homologous or identical to a zika virus 3′ UTR of Table 2, Table 1A, Table 1B, or Table 1G.

12. The method or zika virus or zika-virus-like particle of any one of claims 7-11, wherein the nucleic acid further comprises a polynucleotide encoding a first ribozyme.

13. The method or zika virus or zika-virus-like particle of claim 12, wherein the nucleic acid further comprises a polynucleotide encoding a second ribozyme.

14. The method or zika virus or zika-virus-like particle of any one of claims 7-13, wherein the nucleic acid comprises a polynucleotide encoding a derivative of the zika virus C, wherein the derivative of the zika virus C is a truncation of about or less than about 5, 10, 15, 20, 25, or 30 amino acids of zika virus C.

15. The method or zika virus or zika-virus-like particle of any one of claims 7-13, wherein the nucleic acid does not comprise a polynucleotide encoding a zika virus capsid protein (C) or a derivative of the zika virus C.

16. The method or zika virus or zika-virus-like particle of any one of claims 7-15, wherein the nucleic acid does not comprise a polynucleotide encoding a zika virus viral membrane protein (prM/M) or a derivative of the zika virus prM/M.

17. The method or zika virus or zika-virus-like particle of any one of claims 7-16, wherein the nucleic acid does not comprise a polynucleotide encoding a zika virus viral envelope protein (E) or a derivative of the zika virus E.

18. The method or zika virus or zika-virus-like particle of any one of claims 7-17, wherein the nucleic acid does not comprise a polynucleotide encoding one or more non-structural (NS) proteins: (i) a NS1, (ii) a NS2A, (iii) a NS2B, (iv) a NS3, (v) a NS4A, (vi) a NS4B, (vii) a NS5, or (viii) two or more of (i)-(vii).

19. The method or zika virus or zika-virus-like particle of any one of claims 7-18, wherein the nucleic acid further comprises a promoter.

20. The method or zika virus or zika-virus-like particle of any one of claims 7-19, wherein the nucleic acid comprises a sequence of Tables 1A-1K, Table 2, or Table 3.

21. A nucleic acid composition comprising (i) a 5′ untranslated region (5′ UTR) of a first zika virus, (ii) a 3′ UTR of a second zika virus, wherein the first zika virus is optionally the same as the second zika virus, and (iii) a polynucleotide exogenous to the first zika virus and the second zika virus.

22. The nucleic acid composition of claim 21, wherein the polynucleotide is about 5000 bases to about 10000 bases in length.

23. The nucleic acid composition of claim 21 or claim 22, wherein the nucleic acid does not comprise a polynucleotide encoding a zika virus viral envelope protein (E) or a derivative of the zika virus E; and the nucleic acid does not comprise a polynucleotide encoding one or more non-structural (NS) proteins: (i) a NS1, (ii) a NS2A, (iii) a NS2B, (iv) a NS3, (v) a NS4A, (vi) a NS4B, (vii) a NS5, or (viii) two or more of (i)-(vii).

24. The nucleic acid composition of any one of claims 21-23, comprising a sequence of Tables 1A-1K, Table 2 or Table 3.
